(19) United States
(12) Patent Application Publication
(10) Pub. No.: US 2023/0165570 A1
(43) Pub. Date: Jun. 1, 2023

(54) NASAL-SPRAY SAMPLING

(71) Applicant: HERO SCIENTIFIC LTD., Jerusalem (IL)

(72) Inventors: Robert Eric LEVITZ, Beit Shemesh (IL); Lazar FRUCHTER, Efrat (IL); Avi FRUCHTER, Neve Daniel (IL); Arie Oscar HOLTZ, Jerusalem (IL); Jacob Isaac WOHLGELERNTER, Kamei Shomron (IL)

(73) Assignee: HERO SCIENTIFIC LTD., Jerusalem (IL)

(21) Appl. No.: 17/921,672

(22) PCT Filed: May 6, 2021
(86) PCT No.: PCT/IL2021/050519

§ 371 Date: Oct. 27, 2022

Related U.S. Application Data
(60) Provisional application No. 63/176,565, filed on Apr. 19, 2021.
(60) Provisional application No. 63/166,378, filed on Mar. 26, 2021.
(60) Provisional application No. 63/158,005, filed on Mar. 8, 2021.
(60) Provisional application No. 63/156,843, filed on Mar. 4, 2021.
(60) Provisional application No. 63/117,294, filed on Nov. 23, 2020.
(60) Provisional application No. 63/067,535, filed on Aug. 19, 2020.
(60) Provisional application No. 63/037,707, filed on Jun. 11, 2020.
(60) Provisional application No. 63/020,723, filed on May 6, 2020.

(51) Int. Cl. A61B 10/00; A61H 35/04; G01N 33/569

(52) U.S. Cl. CPC A61B 10/0051; A61H 35/04; G01N 33/56983; A61M 2202/206; A61M 2205/7509

(57) ABSTRACT

A method is provided that includes intranasally dispensing nasal wash fluid (26) into a nasal cavity (22) of a subject such that the nasal wash fluid (26) washes biological material into an oropharynx (30) of the subject from (a) the nasal cavity (22), (b) a nasopharynx (32) of the subject, or (c) the nasal cavity (22) and the nasopharynx (32). Thereafter, a specimen sample (24) is collected that passed out of an anterior opening (34) of an oral cavity (36) of the subject and contains at least a portion of the biological material washed into the oropharynx (30) by the nasal wash fluid (26). Other embodiments are also described.

### CROSS-REFERENCE TO RELATED APPLICATIONS

The present application claims priority from U.S. Provisional Application 63/020,723, filed May 6, 2020, U.S. Provisional Application 63/037,707, filed Jun. 11, 2020, U.S. Provisional Application 63/067,535, filed Aug. 19, 2020, U.S. Provisional Application 63/117,294, filed Nov. 23, 2020, U.S. Provisional Application 63/156,843, filed Mar. 4, 2021, U.S. Provisional Application 63/158,005, filed Mar. 8, 2021, U.S. Provisional Application 63/166,378, filed Mar. 26, 2021, and U.S. Provisional Application 63/176,565, filed Apr. 19, 2021, all of which are assigned to the assignee of the present application and incorporated herein by reference.

### FIELD OF THE APPLICATION

Applications of the present invention relate to collecting samples for testing for the presence of particulates, such as viruses.

### BACKGROUND OF THE APPLICATION

Known sampling methods for collecting a respiratory specimen sample include nasopharyngeal swabbing to sample the posterior nasal passages and the nasopharynx, nasal washes and aspirations to sample deeply into the nasal cavity, and throat swabbing to sample the pharynx. These procedures are often uncomfortable for the patient, require a healthcare professional to perform the sampling procedure, and expose the healthcare worker and others to contaminating particles.

Patients can self-collect alternative sample types such as gargled fluid, nasal washes, and saliva. However, these sample types tend to decrease test sensitivity compared to swab samples and are thus are not currently in widespread use.

### SUMMARY OF THE APPLICATION

In some applications of the present invention, a method is provided that comprises intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx. Thereafter, a specimen sample is collected that passed out of an anterior opening of an oral cavity of the subject and contains at least a portion of the biological material washed into the oropharynx by the nasal wash fluid. In some applications, collecting the specimen sample comprises expressing (e.g., spitting), by the subject, the specimen sample out of the anterior opening of the oral cavity. The specimen sample is typically collected into a collection receptacle.

In some applications of the present invention, a method is provided that comprises intranasally dispensing nasal wash fluid into a nasal cavity of a subject. The nasal wash fluid generally loosens biological material in the nasal cavity. Thereafter, a specimen sample is collected by inserting a specimen sampler into the nasal cavity, typically such that a farthest-inserted portion of the specimen sampler is positioned in an anterior naris of the nasal cavity, and collecting a specimen sample on the specimen sampler by contacting (typically rubbing) a wall of the anterior naris with the specimen sampler. Alternatively or additionally, the specimen sample is collected by performing one or more nasal swabs selected from the group consisting of: an anterior nares swab and a mid-turbinate swab, typically without performing a nasopharyngeal swab.

For some applications, before the specimen sample is collected by performing the nasal swab, before the nasal wash fluid is intranasally dispensed, the subject tilts his or her head back, typically until the neck is fully extended, and holds the head tilted back while the nasal wash fluid is intranasally dispensed. Alternatively, after intranasally dispensing nasal wash fluid, the subject tilts his or her head farther back than while intranasally dispensing the nasal wash fluid and farther back than a neutral position. In all of these cases, the tilting back may cause the nasal wash fluid to loosen biological material in a more posterior (deeper) portion of the nasal cavity, e.g., including in the nasopharynx. Optionally, after the subject tilts his or her head back and after the nasal wash fluid is intranasally dispensed, the subject tilts forward his or her head to a neutral or a tilted-forward position; this backward-followed-by-forward tilting may help move biological material anteriorly (i.e., toward the nostril) within the nasal cavity.

In some applications of the present invention, a method is provided that comprises collecting, from a subject, a liquid specimen sample that includes saliva and orally-dispensed oral wash fluid. The liquid specimen sample is passed through a filter to trap at least some of the saliva, the filter having a nominal pore size of between 30 microns and 1.5 mm. Testing for the presence of a virus in the saliva trapped by the filter is performed.

In experiments conducted by the inventors, the inventors found that filters having pore sizes substantially larger than the viral diameter were nevertheless able to trap virus in the saliva of gargled fluid in quantities sufficient to clinically identify the presence of the virus. The inventors hypothesize that the large-pore filters were able to trap the virus at least in part because of the viscosity of the saliva in typical gargled fluid samples, perhaps in particular because of the viscosity of the mucus and/or other large or aggregated salivary components of the saliva, which themselves contain viral particles, and/or because the viral particles adhered (e.g., directly adhered) to the material of the large-pore filters. Use of large-pore filters may enable a high flow rate and inhibit filter clogging despite high sample viscosity.

There is therefore provided, in accordance with an Inventive Concept 1 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and

thereafter, collecting a specimen sample that passed out of an anterior opening of an oral cavity of the subject and contains at least a portion of the biological material washed into the oropharynx by the nasal wash fluid.

Inventive Concept 2. The method according to Inventive Concept 1, wherein collecting the specimen sample includes collecting the specimen sample into a collection receptacle.
Inventive Concept 3. The method according to Inventive Concept 1, wherein the biological material includes one or more particulates selected from the group consisting of: a virus, a bacterium, a protozoan, and a fungus.
Inventive Concept 4. The method according to Inventive Concept 1, wherein intranasally dispensing includes intranasally dispensing, by the subject, the nasal wash fluid into the nasal cavity.
Inventive Concept 5. The method according to Inventive Concept 1, further including, before collecting the specimen sample, tilting back, by the subject, a head of the subject.
Inventive Concept 6. The method according to Inventive Concept 1, further including, while intranasally dispensing the nasal wash fluid, tilting back, by the subject, a head of the subject.
Inventive Concept 7. The method according to Inventive Concept 1, further including, before intranasally dispensing the nasal wash fluid, tilting back, by the subject, a head of the subject.
Inventive Concept 8. The method according to Inventive Concept 1, further including, after intranasally dispensing the nasal wash fluid, tilting a head of the subject farther back than while intranasally dispensing the nasal wash fluid and farther back than a neutral position.
Inventive Concept 9. The method according to Inventive Concept 1, wherein intranasally dispensing the nasal wash fluid includes intranasally dispensing the nasal wash fluid into the nasal cavity while a head of the subject is held in a neutral position or a tilted-forward position.
Inventive Concept 10. The method according to Inventive Concept 1, further including waiting for a period of time between (a) concluding intranasally dispensing the nasal wash fluid into the nasal cavity and (b) collecting the specimen sample, the period of time at least 5 minutes and less than 90 minutes.
Inventive Concept 11. The method according to Inventive Concept 1, wherein intranasally dispensing the nasal wash fluid includes intranasally dispensing the nasal wash fluid as one or more types of fluid discharge selected from the group consisting of: a fluid stream, a spray, a mist, a pressurized aerosol, and an atomized fluid.
Inventive Concept 12. The method according to Inventive Concept 1, wherein intranasally dispensing the nasal wash fluid includes intranasally dispensing the nasal wash fluid as steam.
Inventive Concept 13. The method according to Inventive Concept 1, wherein intranasally dispensing the nasal wash fluid includes intranasally dispensing the nasal wash fluid in a circular rotary motion to generate dispersion within the nasal cavity.
Inventive Concept 14. The method according to Inventive Concept 1, wherein intranasally dispensing the nasal wash fluid into the nasal cavity includes intranasally dispensing a total volume of 0.2-20 mL of the nasal wash fluid into the nasal cavity.
Inventive Concept 15. The method according to Inventive Concept 14, wherein intranasally dispensing the nasal wash fluid into the nasal cavity includes intranasally dispensing a total volume of 0.6-20 mL of the nasal wash fluid into the nasal cavity.
Inventive Concept 16. The method according to Inventive Concept 14, wherein intranasally dispensing the nasal wash fluid into the nasal cavity includes intranasally dispensing a total volume of 0.2-10 mL of the nasal wash fluid into the nasal cavity.
Inventive Concept 17. The method according to Inventive Concept 16, wherein intranasally dispensing the nasal wash fluid into the nasal cavity includes intranasally dispensing a total volume of 0.2-5 mL of the nasal wash fluid into the nasal cavity.
Inventive Concept 18. The method according to Inventive Concept 17, wherein intranasally dispensing the nasal wash fluid into the nasal cavity includes intranasally dispensing a total volume of 0.6-5 mL of the nasal wash fluid into the nasal cavity.
Inventive Concept 19. The method according to Inventive Concept 14, wherein intranasally dispensing the nasal wash fluid into the nasal cavity includes intranasally dispensing the total volume of the nasal wash fluid in a plurality of pulses.
Inventive Concept 20. The method according to Inventive Concept 19, wherein each of the pulses has a volume of 0.02-10 mL.
Inventive Concept 21. The method according to Inventive Concept 20, wherein each of the pulses has a volume of 0.02-1 mL.
Inventive Concept 22. The method according to Inventive Concept 1, wherein intranasally dispensing the nasal wash fluid into the nasal cavity includes intranasally dispensing the nasal wash fluid with a sprayed particle size of 5-50 microns.
Inventive Concept 23. The method according to Inventive Concept 22, wherein intranasally dispensing the nasal wash fluid includes intranasally dispensing the nasal wash fluid with a sprayed particle size of 5-15 microns.
Inventive Concept 24. The method according to Inventive Concept 22, wherein intranasally dispensing the nasal wash fluid includes intranasally dispensing the nasal wash fluid with a sprayed particle size of 15-20 microns.
Inventive Concept 25. The method according to Inventive Concept 1, wherein intranasally dispensing the nasal wash fluid into the nasal cavity includes intranasally dispensing the nasal wash fluid into the nasal cavity while the subject is lying supine.
Inventive Concept 26. The method according to any one of Inventive Concepts 1-25, wherein collecting the specimen sample includes expressing, by the subject, the specimen sample out of the oropharynx via the anterior opening of the oral cavity.
Inventive Concept 27. The method according to Inventive Concept 26, wherein expressing the specimen sample includes spitting, by the subject, the specimen sample out of the oropharynx via the anterior opening of the oral cavity.
Inventive Concept 28. The method according to Inventive Concept 26, wherein expressing the specimen sample includes expressing the specimen sample out of the oropharynx via the anterior opening of the oral cavity after sensing, by the subject, the nasal wash fluid in the oropharynx.
Inventive Concept 29. The method according to Inventive Concept 28, wherein expressing the specimen sample includes expressing the specimen sample out of the oropharynx via the anterior opening of the oral cavity after tasting, by the subject, the nasal wash fluid in the oropharynx.
Inventive Concept 30. The method according to Inventive Concept 29, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 31. The method according to Inventive Concept 26, wherein intranasally dispensing includes intranasally dispensing, by a healthcare worker, the nasal wash fluid into the nasal cavity.
Inventive Concept 32. The method according to any one of Inventive Concepts 1-25, wherein collecting the specimen sample includes drawing the specimen sample out of the oropharynx via the anterior opening of the oral cavity using an absorbent material, without swabbing the oral cavity or the oropharynx.
Inventive Concept 33. The method according to any one of Inventive Concepts 1-25, wherein intranasally dispensing includes intranasally dispensing, by a healthcare worker, the nasal wash fluid into the nasal cavity.
Inventive Concept 34. The method according to Inventive Concept 33, wherein collecting the specimen sample includes receiving the specimen sample by a healthcare worker from the subject.
Inventive Concept 35. The method according to Inventive Concept 34, wherein receiving the specimen sample includes receiving the specimen sample that was expressed out of the oropharynx via the anterior opening of the oral cavity.
Inventive Concept 36. The method according to Inventive Concept 35, wherein receiving the specimen sample includes receiving the specimen sample that was spit out of the oropharynx via the anterior opening of the oral cavity.
Inventive Concept 37. The method according to Inventive Concept 33, wherein collecting the specimen sample includes aspirating the specimen sample out of the oropharynx via the anterior opening of the oral cavity.
Inventive Concept 38. The method according to Inventive Concept 33, wherein collecting the specimen sample includes instructing the subject, by the healthcare worker, to express the specimen sample out of the oropharynx via the anterior opening of the oral cavity.
Inventive Concept 39. The method according to Inventive Concept 38, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 40. The method according to any one of Inventive Concepts 1-25, further including, after intranasally dispensing the nasal wash fluid and before collecting the specimen sample, sniffing, by the subject, the biological material with the nasal wash fluid from the nasal cavity posteriorly toward the nasopharynx.
Inventive Concept 41. The method according to any one of Inventive Concepts 1-25, further including, after intranasally dispensing the nasal wash fluid and before collecting the specimen sample, snorting, by the subject, the biological material with the nasal wash fluid from the nasal cavity toward the nasopharynx.
Inventive Concept 42. The method according to any one of Inventive Concepts 1-25, further including, after collecting the specimen sample, testing the specimen sample for the presence of a particulate present in at least the biological material washed into the oropharynx by the nasal wash fluid.
Inventive Concept 43. The method according to Inventive Concept 42, wherein testing the specimen sample for the presence of the particulate includes diagnosing a disease.
Inventive Concept 44. The method according to Inventive Concept 43, wherein diagnosing the disease includes diagnosing an infectious disease.
Inventive Concept 45. The method according to Inventive Concept 42, wherein testing the specimen sample for the presence of the particulate includes concentrating the specimen sample by filtration prior to testing for the presence of the particulate, by passing the specimen sample through one or more filters.
Inventive Concept 46. The method according to Inventive Concept 42, wherein testing the specimen sample for the presence of the particulate includes passing the specimen sample through one or more porous filters to produce a filtrate, and testing the filtrate for the presence of the particulate.
Inventive Concept 47. The method according to Inventive Concept 42, wherein testing the specimen sample for the presence of the particulate includes using spectroscopy to test the specimen sample for the presence of the particulate.
Inventive Concept 48. The method according to Inventive Concept 42, wherein testing the specimen sample for the presence of the particulate includes using microscopy to test the specimen sample for the presence of the particulate.
Inventive Concept 49. The method according to Inventive Concept 42, wherein testing the specimen sample for the presence of the particulate includes using image processing to test the specimen sample for the presence of the particulate.
Inventive Concept 50. The method according to Inventive Concept 42, wherein testing the specimen sample for the presence of the particulate includes using three-dimensional (3D) image sensing to test the specimen sample for the presence of the particulate.
Inventive Concept 51. The method according to Inventive Concept 42, wherein testing the specimen sample for the presence of the particulate includes using artificial intelligence to test the specimen sample for the presence of the particulate.
Inventive Concept 52. The method according to Inventive Concept 42, wherein testing the specimen sample for the presence of the particulate includes using a molecular-based assay to test the specimen sample for the presence of the particulate.
Inventive Concept 53. The method according to Inventive Concept 52, wherein testing the specimen sample for the presence of the particulate includes using nucleic acid amplification to test the specimen sample for the presence of the particulate.
Inventive Concept 54. The method according to Inventive Concept 53, wherein testing the specimen sample for the presence of the particulate includes using polymerase chain reaction (PCR) to test the specimen sample for the presence of the particulate.
Inventive Concept 55. The method according to Inventive Concept 53, wherein testing the specimen sample for the presence of the particulate includes using isothermal amplification (e.g., loop-mediated isothermal amplification (LAMP) or a nicking enzyme amplification (NEAR) reaction) to test the specimen sample for the presence of the particulate.
Inventive Concept 56. The method according to Inventive Concept 42, wherein testing the specimen sample for the presence of the particulate includes performing an immunoassay to test the specimen sample for the presence of the particulate.
Inventive Concept 57. The method according to Inventive Concept 42, wherein the particulate includes one or more particulates selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.
Inventive Concept 58. The method according to Inventive Concept 42, wherein the particulate includes one or more particulates selected from the group consisting of: a respiratory particulate and a particulate contained within droplets from lower airways of the subject.
Inventive Concept 59. The method according to Inventive Concept 42, wherein the particulate includes one or more particulates selected from the group consisting of: a virus, a bacterium, a protozoan, and a fungus.
Inventive Concept 60. The method according to Inventive Concept 42, wherein the particulate includes a virus.
Inventive Concept 61. The method according to Inventive Concept 60, wherein the virus is an Influenza virus.
Inventive Concept 62. The method according to Inventive Concept 60, wherein the virus is a coronavirus.
Inventive Concept 63. The method according to Inventive Concept 62, wherein the coronavirus is Severe acute respiratory syndrome coronavirus 2 (SARS-CoV-2).
Inventive Concept 64. The method according to Inventive Concept 42, wherein the particulate includes a bacterium.
Inventive Concept 65. The method according to Inventive Concept 64, wherein the bacterium is a Streptococcus bacterium.
Inventive Concept 66. The method according to Inventive Concept 42, wherein the particulate includes one or more antigens selected from the group consisting of: a viral antigen, a bacterial antigen, and a fungal antigen.
Inventive Concept 67. The method according to any one of Inventive Concepts 1-25, wherein intranasally dispensing the nasal wash fluid into the nasal cavity includes intranasally dispensing the nasal wash fluid unidirectionally within the nasal cavity.
Inventive Concept 68. The method according to Inventive Concept 67, wherein intranasally dispensing the nasal wash fluid unidirectionally within the nasal cavity includes intranasally dispensing the nasal wash fluid unidirectionally within the nasal cavity in a superior direction.
Inventive Concept 69. The method according to Inventive Concept 67, wherein intranasally dispensing the nasal wash fluid unidirectionally within the nasal cavity includes intranasally dispensing the nasal wash fluid unidirectionally within the nasal cavity in a superoposterior direction.
Inventive Concept 70. The method according to Inventive Concept 69, wherein intranasally dispensing the nasal wash fluid unidirectionally within the nasal cavity in a superoposterior direction includes intranasally dispensing the nasal wash fluid unidirectionally within the nasal cavity toward an olfactory area of the nasal cavity.
Inventive Concept 71. The method according to Inventive Concept 67, wherein intranasally dispensing the nasal wash fluid unidirectionally within the nasal cavity includes intranasally dispensing the nasal wash fluid unidirectionally within the nasal cavity in a posterior direction.
Inventive Concept 72. The method according to Inventive Concept 71, wherein intranasally dispensing the nasal wash fluid unidirectionally within the nasal cavity in a posterior direction includes intranasally dispensing the nasal wash fluid unidirectionally within the nasal cavity toward the nasopharynx.
Inventive Concept 73. The method according to any one of Inventive Concepts 1-25, wherein intranasally dispensing the nasal wash fluid into the nasal cavity includes intranasally dispensing the nasal wash fluid multidirectionally within the nasal cavity.
Inventive Concept 74. The method according to Inventive Concept 73, wherein intranasally dispensing the nasal wash fluid multidirectionally within the nasal cavity includes dispensing the nasal wash fluid into the nasal cavity:

in a posterior direction toward the nasopharynx, and

in a superoposterior direction toward an olfactory area of the nasal cavity.

Inventive Concept 75. The method according to Inventive Concept 74, wherein intranasally dispensing the nasal wash fluid multidirectionally within the nasal cavity includes dispensing the nasal wash fluid into the nasal cavity additionally in at least a superior direction toward an inferior border of upper lateral cartilage of the nasal cavity.
Inventive Concept 76. The method according to Inventive Concept 75, wherein intranasally dispensing the nasal wash fluid multidirectionally within the nasal cavity includes dispensing the nasal wash fluid into the nasal cavity additionally bilaterally toward a septum and a lateral wall of the nasal cavity.
Inventive Concept 77. The method according to Inventive Concept 73, wherein intranasally dispensing the nasal wash fluid multidirectionally within the nasal cavity includes intranasally dispensing the nasal wash fluid via a plurality of nozzles.
Inventive Concept 78. The method according to Inventive Concept 77, wherein intranasally dispensing the nasal wash fluid multidirectionally within the nasal cavity via the plurality of nozzles includes intranasally dispensing the nasal wash fluid into the nasal cavity:

in a posterior direction toward the nasopharynx via a first subset of one or more of the plurality of nozzles, and

in a superoposterior direction toward an olfactory area of the nasal cavity via a second subset of one or more of the plurality of nozzles, the first and the second subsets not including any nozzles common to both subsets.

Inventive Concept 79. The method according to Inventive Concept 78,

wherein the plurality of nozzles includes only first and second nozzles, and wherein the first and the second subsets consist of the first and the second nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle, and
in the superoposterior direction toward the olfactory area of the nasal cavity via the second nozzle.
   Inventive Concept 80. The method according to Inventive Concept 78, wherein intranasally dispensing the nasal wash fluid multidirectionally within the nasal cavity via the plurality of nozzles includes intranasally dispensing the nasal wash fluid into the nasal cavity:

in the posterior direction toward the nasopharynx via the first subset of one or more of the plurality of nozzles,

in the superoposterior direction toward the olfactory area of the nasal cavity via the second subset of one or more of the plurality of nozzles, and

at least in a superior direction toward an inferior border of upper lateral cartilage of the nasal cavity via a third subset of one or more of the plurality of nozzles, wherein none of the nozzles is included in more than one of the first, the second, and the third subsets.

Inventive Concept 81. The method according to Inventive Concept 80, wherein intranasally dispensing the nasal wash fluid in at least the superior direction toward the superior wall includes dispensing the nasal wash fluid into the nasal cavity additionally bilaterally toward a septum and a lateral wall of the nasal cavity via the third subset of one or more of the plurality of nozzles.
Inventive Concept 82. The method according to Inventive Concept 80,

wherein the plurality of nozzles includes only first, second, and third nozzles, and wherein the first, the second, and the third subsets consist of the first, the second, and the third nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle,
in the superoposterior direction toward the olfactory area of the nasal cavity via the second nozzle, and
at least in the superior direction toward an inferior border of upper lateral cartilage of the nasal cavity via the third nozzle.
   Inventive Concept 83. The method according to Inventive Concept 77, wherein intranasally dispensing the nasal wash fluid multidirectionally within the nasal cavity includes intranasally dispensing the nasal wash fluid within the nasal cavity via the plurality of nozzles with respective different fluid flow characteristics, the fluid flow characteristics including one or more characteristics selected from the group consisting of: volume, sprayed particle size, velocity, and flow rate.
   Inventive Concept 84. The method according to any one of Inventive Concepts 1-25, wherein intranasally dispensing the nasal wash fluid into the nasal cavity includes intranasally dispensing nasal wash fluid via both nostrils of the subject simultaneously.
   Inventive Concept 85. The method according to any one of Inventive Concepts 1-25, wherein the nasal wash fluid does not include a drug molecule.
   Inventive Concept 86. The method according to any one of Inventive Concepts 1-25, wherein the nasal wash fluid includes a non-irritant solution.
   Inventive Concept 87. The method according to Inventive Concept 86, wherein the non-irritant solution consists of water.
   Inventive Concept 88. The method according to Inventive Concept 86, wherein the non-irritant solution includes saline solution.
   Inventive Concept 89. The method according to Inventive Concept 88, wherein the non-irritant saline solution includes phosphate-buffered saline solution.
   Inventive Concept 90. The method according to any one of Inventive Concepts 1-25, wherein the nasal wash fluid includes a tastant.
   Inventive Concept 91. The method according to any one of Inventive Concepts 1-25, wherein the nasal wash fluid includes a color additive, and collecting the specimen sample includes confirming that the specimen sample contains at least a portion of the nasal wash fluid by visually ascertaining that the specimen sample includes the color additive.
   Inventive Concept 92. The method according to any one of Inventive Concepts 1-25, wherein the nasal wash fluid includes a chemical additive, and collecting the specimen sample includes confirming that the specimen sample contains at least a portion of the nasal wash fluid by ascertaining that the specimen sample includes the chemical additive.
   Inventive Concept 93. The method according to Inventive Concept 92,

further including, after collecting the specimen sample, testing the specimen sample for the presence of a particulate present in at least the biological material washed into the oropharynx by the nasal wash fluid, using a testing technique, and

wherein ascertaining that the specimen sample includes the chemical additive includes ascertaining that the specimen sample includes the chemical additive using the same testing technique used to test the specimen sample for the presence of the particulate.

Inventive Concept 94. The method according to any one of Inventive Concepts 1-25, further including, before collecting the specimen sample, intranasally dispensing a vapor into the nasal cavity to loosen the biological material in the nasal cavity.
Inventive Concept 95. The method according to Inventive Concept 94, wherein intranasally dispensing the vapor includes inhaling, by the subject, the vapor into the nasal cavity.
Inventive Concept 96. The method according to any one of Inventive Concepts 1-25, further including, after intranasally dispensing and before collecting the specimen sample, swishing in the oral cavity, by the subject, the nasal wash fluid washed into the oropharynx.
Inventive Concept 97. The method according to any one of Inventive Concepts 1-25, further including, after intranasally dispensing and before collecting the specimen sample, gargling, by the subject, the nasal wash fluid washed into the oropharynx.
Inventive Concept 98. The method according to Inventive Concept 97, wherein the method further includes, after gargling the nasal wash fluid and before collecting the specimen sample, while holding the nasal wash fluid in the oropharynx, sniffing an additional portion of the nasal wash fluid from the nasal cavity toward the oropharynx such that the additional portion of the nasal wash fluid is added to the nasal wash fluid already in the oropharynx.
Inventive Concept 99. The method according to Inventive Concept 98, wherein the method further includes, after sniffing the additional portion of the nasal wash fluid from the nasal cavity toward the oropharynx, again gargling the nasal wash fluid washed into the oropharynx.
Inventive Concept 100. The method according to any one of Inventive Concepts 1-25,

wherein the method further includes, before collecting the specimen sample:
orally dispensing oral wash fluid into the oral cavity via the anterior opening of the oral cavity; and
swishing, by the subject, the oral wash fluid in the oral cavity, and

wherein collecting includes collecting the specimen sample that passed out of the anterior opening of the oral cavity and contains at least a portion of the oral wash fluid and the at least a portion of the biological material washed into the oropharynx with the nasal wash fluid.

Inventive Concept 101. The method according to any one of Inventive Concepts 1-25,

wherein the method further includes, before collecting the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein collecting includes collecting the specimen sample that passed out of the anterior opening of the oral cavity and contains at least a portion of the mixture and the at least a portion of the biological material washed into the oropharynx with the nasal wash fluid.

Inventive Concept 102. The method according to any one of Inventive Concepts 1-25,

wherein the method further includes, before collecting the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein collecting includes collecting the specimen sample that passed out of the anterior opening of the oral cavity and contains at least a portion of the mixture and the at least a portion of the biological material washed into the oropharynx with the nasal wash fluid.

Inventive Concept 103. The method according to any one of Inventive Concepts 1-25,

wherein the method further includes, before collecting the specimen sample:
orally dispensing oral wash fluid into the oral cavity via the anterior opening of the oral cavity; and
gargling, by the subject, the oral wash fluid, and

wherein collecting includes collecting the specimen sample that passed out of the anterior opening of the oral cavity and contains at least a portion of the oral wash fluid and the at least a portion of the biological material washed into the oropharynx with the nasal wash fluid.

Inventive Concept 104. The method according to any one of Inventive Concepts 101, 102, and 103, wherein orally dispensing the oral wash fluid into the oral cavity includes orally dispensing the oral wash fluid into the oral cavity prior to intranasally dispensing the nasal wash fluid into the nasal cavity.
Inventive Concept 105. The method according to any one of Inventive Concepts 101, 102, and 103, wherein orally dispensing the oral wash fluid into the oral cavity includes orally dispensing the oral wash fluid into the oral cavity after intranasally dispensing the nasal wash fluid into the nasal cavity.
Inventive Concept 106. The method according to Inventive Concept 105, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 107. The method according to any one of Inventive Concepts 101, 102, and 103,

wherein the specimen sample is a first specimen sample, and the mixture is a first mixture,

wherein the method further includes, after collecting the first specimen sample:
waiting for a period of time, the period of time at least 5 minutes and less than 90 minutes;
thereafter, orally dispensing additional oral wash fluid into the oral cavity via the anterior opening of the oral cavity;
thereafter, gargling, by the subject; and
thereafter, collecting a second specimen sample that passed out of the anterior opening of the oral cavity and contains at least a portion of the additional oral wash fluid and the at least a portion of the biological material washed into the oropharynx with the nasal wash fluid, and

wherein the method does not include intranasally dispensing nasal wash fluid into the nasal cavity after collecting the first specimen sample.

Inventive Concept 108. The method according to any one of Inventive Concepts 101, 102, and 103,

wherein the nasal wash fluid includes a first color additive,

wherein the oral wash fluid includes a second colored additive, the first and the second color additives having different colors, and

wherein collecting the specimen sample includes confirming that the specimen sample contains at least a portion of the nasal wash fluid and at least a portion of the oral wash fluid by visually ascertaining that the specimen sample has a color produced by a combination of the first and the second color additives.

Inventive Concept 109. The method according to any one of Inventive Concepts 101, 102, and 103, wherein the oral wash fluid includes a non-irritant solution.
Inventive Concept 110. The method according to Inventive Concept 109, wherein the non-irritant solution consists of water.
Inventive Concept 111. The method according to Inventive Concept 109, wherein the non-irritant solution includes a tastant.
Inventive Concept 112. The method according to Inventive Concept 109, wherein the non-irritant solution includes saline solution.
Inventive Concept 113. The method according to Inventive Concept 112, wherein the non-irritant saline solution includes phosphate-buffered saline solution.
Inventive Concept 114. The method according to any one of Inventive Concepts 101 and 102, wherein the method further includes, after gargling the mixture and before collecting the specimen sample, while holding the mixture in the oropharynx, sniffing an additional portion of the nasal wash fluid from the nasal cavity toward the oropharynx such that the additional portion of the nasal wash fluid is added to the mixture in the oropharynx.
Inventive Concept 115. The method according to Inventive Concept 114, wherein the method further includes, after sniffing the additional portion of the nasal wash fluid from the nasal cavity toward the oropharynx, again gargling the mixture.
Inventive Concept 116. The method according to Inventive Concept 114, wherein gargling the mixture and thereafter sniffing the additional portion of the nasal wash fluid includes repeating the steps of gargling and sniffing at least two times while at least a portion of the nasal wash fluid remains in the oropharynx.
Inventive Concept 117. The method according to any one of Inventive Concepts 101 and 102, wherein the method further includes, after gargling the mixture and before collecting the specimen sample, while holding the mixture in the oropharynx, snorting an additional portion of the nasal wash fluid from the nasal cavity toward the oropharynx such that the additional portion of the nasal wash fluid is added to the mixture in the oropharynx.
Inventive Concept 118. The method according to Inventive Concept 117, wherein the method further includes, after snorting the additional portion of the nasal wash fluid from the nasal cavity toward the oropharynx, again gargling the mixture.
Inventive Concept 119. The method according to Inventive Concept 117, wherein gargling the mixture and thereafter snorting the additional portion of the nasal wash fluid includes repeating the steps of gargling and snorting at least two times while at least a portion of the nasal wash fluid remains in the oropharynx.
Inventive Concept 120. The method according to any one of Inventive Concepts 101, 102, and 103, further including, before collecting the specimen sample, spraying, via the anterior opening of the oral cavity, the oropharynx with oropharyngeal fluid wash.
Inventive Concept 121. The method according to any one of Inventive Concepts 101 and 102, further including swishing, by the subject, the mixture in the oral cavity.
Inventive Concept 122. The method according to any one of Inventive Concepts 101, 102, and 103, further including, after collecting the specimen sample, testing the specimen sample for the presence of a particulate.
Inventive Concept 123. The method according to Inventive Concept 122, wherein testing the specimen sample for the presence of the particulate includes passing the specimen sample through one or more porous filters to produce a filtrate, and testing the filtrate for the presence of the particulate.
Inventive Concept 124. The method according to Inventive Concept 122, wherein testing the specimen sample for the presence of the particulate includes using spectroscopy to test the specimen sample for the presence of the particulate.
Inventive Concept 125. The method according to Inventive Concept 122, wherein testing the specimen sample for the presence of the particulate includes using microscopy to test the specimen sample for the presence of the particulate.
Inventive Concept 126. The method according to Inventive Concept 122, wherein testing the specimen sample for the presence of the particulate includes using image processing to test the specimen sample for the presence of the particulate.
Inventive Concept 127. The method according to Inventive Concept 122, wherein testing the specimen sample for the presence of the particulate includes using three-dimensional (3D) image sensing to test the specimen sample for the presence of the particulate.
Inventive Concept 128. The method according to Inventive Concept 122, wherein testing the specimen sample for the presence of the particulate includes using artificial intelligence to test the specimen sample for the presence of the particulate.
Inventive Concept 129. The method according to Inventive Concept 122, wherein testing the specimen sample for the presence of the particulate includes using a molecular-based assay to test the specimen sample for the presence of the particulate.
Inventive Concept 130. The method according to Inventive Concept 129, wherein testing the specimen sample for the presence of the particulate includes using nucleic acid amplification to test the specimen sample for the presence of the particulate.
Inventive Concept 131. The method according to Inventive Concept 130, wherein testing the specimen sample for the presence of the particulate includes using polymerase chain reaction (PCR) to test the specimen sample for the presence of the particulate.
Inventive Concept 132. The method according to Inventive Concept 130, wherein testing the specimen sample for the presence of the particulate includes using isothermal amplification (e.g., loop-mediated isothermal amplification (LAMP) or a nicking enzyme amplification (NEAR) reaction) to test the specimen sample for the presence of the particulate.
Inventive Concept 133. The method according to Inventive Concept 122, wherein testing the specimen sample for the presence of the particulate includes performing an immunoassay to test the specimen sample for the presence of the particulate.
Inventive Concept 134. The method according to any one of Inventive Concepts 1-25, further including, before collecting the specimen sample, spraying, via the anterior opening of the oral cavity, the oropharynx with oropharyngeal fluid wash.
Inventive Concept 135. The method according to any one of Inventive Concepts 1-25,

wherein the specimen sample is a first specimen sample that contains at least a first portion of the nasal wash fluid and at least a first portion of the biological material washed into the oropharynx, and

wherein the method further includes:
collecting from the nasal cavity, via one or two nostrils of the subject, a second specimen sample that contains at least a second portion of the nasal wash fluid and at least a second portion of the biological material in the nasal cavity; and
combining the first and the second specimen samples to produce a combined specimen sample.
   Inventive Concept 136. The method according to Inventive Concept 135, further including testing the combined specimen sample for the presence of a particulate.
   Inventive Concept 137. The method according to Inventive Concept 135, wherein collecting the second specimen sample includes aspirating the second specimen sample via the one or two nostrils.
   Inventive Concept 138. The method according to Inventive Concept 135, wherein collecting the second specimen sample includes blowing, by the subject, the second specimen sample out of the nasal cavity via the one or two nostrils.
   Inventive Concept 139. The method according to Inventive Concept 135, wherein collecting the second specimen sample includes collecting the second specimen sample that was blown, by the subject, out of the nasal cavity via the one or two nostrils.
   Inventive Concept 140. The method according to Inventive Concept 135, wherein collecting the second specimen sample includes swabbing the nasal cavity.
   Inventive Concept 141. The method according to any one of Inventive Concepts 1-25,

wherein intranasally dispensing the nasal wash fluid into the nasal cavity includes inserting a tubular applicator of a nasal wash fluid dispenser into a nostril of the subject and intranasally dispensing nasal wash fluid from the tubular applicator, and

wherein the nasal wash fluid dispenser is shaped so as to guide introduction of the tubular applicator into the nostril at a specific orientation with respect to a maxilla of the subject.

Inventive Concept 142. The method according to Inventive Concept 141, wherein the dispensing container is shaped so as to guide the introduction of the tubular applicator into the nostril at the specific orientation with respect to the maxilla.
Inventive Concept 143. The method according to Inventive Concept 141, wherein the nasal wash fluid dispenser includes a housing that is shaped so as to guide the introduction of the tubular applicator into the nostril at the specific orientation with respect to the maxilla.
Inventive Concept 144. The method according to Inventive Concept 141,

wherein the nasal wash fluid dispenser is shaped so as to define a concave surface that faces in a posterior direction and generally conforms to an outer surface of an upper lip of the subject, and

wherein inserting the tubular applicator into the nostril includes pressing the concave surface against the upper lip so as to stabilize the nasal wash fluid dispenser with respect to the maxilla.

Inventive Concept 145. The method according to Inventive Concept 144, wherein the concave surface has a lateral width of between 2.5 and 7 cm.
Inventive Concept 146. The method according to Inventive Concept 144, wherein the concave surface is curved.
Inventive Concept 147. The method according to Inventive Concept 144, wherein the concave surface is concave about an axis and a flat in a direction along the axis.
Inventive Concept 148. The method according to Inventive Concept 144, wherein the dispensing container is shaped so as to define the concave surface.
Inventive Concept 149. The method according to Inventive Concept 144, wherein the nasal wash fluid dispenser includes a housing that is shaped so as to define the concave surface.
Inventive Concept 150. The method according to any one of Inventive Concepts 1-25, wherein the method does not include swabbing the nasal cavity.
Inventive Concept 151. The method according to any one of Inventive Concepts 1-25,

wherein the method does not include swabbing the nasopharynx, and

wherein collecting the specimen sample includes swabbing the oropharynx.

Inventive Concept 152. The method according to Inventive Concept 151, wherein the method does not include swabbing any portion of the nasal cavity.
Inventive Concept 153. The method according to Inventive Concept 151, wherein collecting the specimen sample includes swabbing the oropharynx and swabbing one or both anterior nares of the nasal cavity.
Inventive Concept 154. The method according to Inventive Concept 151, wherein collecting the specimen sample includes swabbing the oropharynx and swabbing one or more turbinates of the nasal cavity selected from the group consisting of: a middle turbinate and an inferior turbinate.
Inventive Concept 155. The method according to any one of Inventive Concepts 1-25, wherein collecting the specimen sample that passed out of the anterior opening of the oral cavity includes collecting a specimen sample that passed out of the anterior opening of the oral cavity and was not swabbed from oropharynx.
Inventive Concept 156. The method according to any one of Inventive Concepts 1-25, wherein the method does not include swabbing the oropharynx and does not include swabbing the nasopharynx.
Inventive Concept 157. The method according to any one of Inventive Concepts 1-25, wherein collecting the specimen sample includes drawing the specimen sample out of the oral cavity via the anterior opening of the oral cavity using an absorbent material.
Inventive Concept 158. The method according to Inventive Concept 157, wherein the absorbent material is located on a tip of a collector shaft, and wherein drawing the specimen sample out of the oral cavity via the anterior opening of the oral cavity using the absorbent material includes inserting the tip of the collector shaft into the oral cavity.
Inventive Concept 159. The method according to Inventive Concept 158, wherein drawing the specimen sample out of the oral cavity via the anterior opening of the oral cavity using the absorbent material includes sucking, by the subject, on the absorbent material.
Inventive Concept 160. The method according to Inventive Concept 157, wherein drawing the specimen sample out of the oral cavity includes contacting one or more portions of the oral cavity with the absorbent material, without swabbing the oropharynx.
Inventive Concept 161. The method according to Inventive Concept 160, wherein the one or more portions of the oral cavity include one or more portions selected from the group consisting of: buccal mucosa, a tongue, gums, and palatal mucosa.
Inventive Concept 162. The method according to any one of Inventive Concepts 1-25, wherein the method does not include swabbing any portion of the subject.

There is further provided, in accordance with an Inventive Concept 163 of the present invention, a nasal wash fluid dispenser for intranasally dispensing nasal wash fluid into a nasal cavity of a subject, the nasal wash fluid dispenser including:

a dispensing container, which contains the nasal wash fluid; and

a tubular applicator, which (a) extends distally from and is in fluid communication with the dispensing container, (b) which is sized and shaped to be insertable into a nostril of the subject, and (c) which includes first and second nozzles, which are configured to dispense the nasal wash fluid in first and second fluid discharges, respectively, having first and second centerlines, respectively, the first and the second centerlines defining an offset angle therebetween of between 10 and 90 degrees, such that, when the tubular applicator is inserted into the nostril with the first nozzle oriented in a posterior direction and the second nozzle oriented in a superoposterior direction, the first nozzle directs the first fluid discharge toward a nasopharynx of the subject and the second nozzle directs the second fluid discharge toward an olfactory area of the nasal cavity, such that the nasal wash fluid washes biological material into an oropharynx of the subject from (i) the nasal cavity, (ii) the nasopharynx, or (iii) the nasal cavity and the nasopharynx.

Inventive Concept 164. The nasal wash fluid dispenser according to Inventive Concept 163, wherein the offset angle is between 20 and 80 degrees.
Inventive Concept 165. The nasal wash fluid dispenser according to Inventive Concept 164, wherein the offset angle is between 60 and 70 degrees.
Inventive Concept 166. The nasal wash fluid dispenser according to Inventive Concept 163, wherein the offset angle is between 30 and 60 degrees
167. The nasal wash fluid dispenser according to Inventive Concept 166, wherein the offset angle is between 40 and 50 degrees.
Inventive Concept 168. The nasal wash fluid dispenser according to Inventive Concept 163, wherein the first and the second nozzles are configured to dispense the first and the second fluid discharges of the nasal wash fluid with the same volume, sprayed particle size, velocity, and flow rate.
Inventive Concept 169. The nasal wash fluid dispenser according to Inventive Concept 163, wherein the first and the second nozzles are configured to dispense the first and the second fluid discharges of the nasal wash fluid with respective different fluid flow characteristics, the fluid flow characteristics including one or more characteristics selected from the group consisting of: volume, sprayed particle size, velocity, and flow rate.
Inventive Concept 170. The nasal wash fluid dispenser according to Inventive Concept 163, wherein one of the first and the second nozzles is located at a distal end of the tubular applicator, and the other of the first and the second nozzles is located is located away from the distal end of the tubular applicator.
Inventive Concept 171. The nasal wash fluid dispenser according to Inventive Concept 170, wherein the tubular applicator is shaped so as to define a distal-most central longitudinal axis through a distal end of the tubular applicator, and wherein the one of the first and the second nozzles located at the distal end of the tubular applicator is oriented such that the centerline thereof is coaxial with the distal-most central longitudinal axis.
Inventive Concept 172. The nasal wash fluid dispenser according to Inventive Concept 163, wherein the tubular applicator does not include any nozzles other than the first and the second nozzles.
Inventive Concept 173. The nasal wash fluid dispenser according to Inventive Concept 163, wherein the nasal wash fluid dispenser is configured to dispense the nasal wash fluid by manual compression of the fluid dispenser.
Inventive Concept 174. The nasal wash fluid dispenser according to Inventive Concept 163, wherein the nasal wash fluid dispenser includes an atomizer that is configured to dispense the nasal wash fluid.
Inventive Concept 175. The nasal wash fluid dispenser according to Inventive Concept 163, wherein the nasal wash fluid dispenser includes a pump that is configured to dispense the nasal wash fluid.
Inventive Concept 176. The nasal wash fluid dispenser according to Inventive Concept 163, wherein the nasal wash fluid dispenser includes a pressurized aerosol generator that is configured to dispense the nasal wash fluid.
Inventive Concept 177. The nasal wash fluid dispenser according to Inventive Concept 163, wherein the tubular applicator is malleable and compressible to fit into and occlude the nostril.
Inventive Concept 178. The nasal wash fluid dispenser according to Inventive Concept 163, wherein the tubular applicator includes a curved portion.
Inventive Concept 179. The nasal wash fluid dispenser according to Inventive Concept 163,

wherein the tubular applicator is shaped so as to define an applicator centerline that defines a plane, and

wherein the first and the second nozzles are configured to dispense the nasal wash fluid in the first and the second fluid discharges, respectively, such that the first and the second centerlines, respectively, fall in the plane.

Inventive Concept 180. The nasal wash fluid dispenser according to Inventive Concept 163, wherein the tubular applicator is shaped so as to define distal-most and proximal-most central longitudinal axes through distal and proximal ends of the tubular applicator, respectively, and wherein the distal-most and the proximal-most central longitudinal axes form a bend angle of between 100 and 175 degrees therebetween.
Inventive Concept 181. The nasal wash fluid dispenser according to Inventive Concept 163, wherein at least one of the first and the second nozzles includes a fan mechanism configured to dispense the nasal wash fluid in a circular rotary motion to generate dispersion within the nasal cavity.
Inventive Concept 182. The nasal wash fluid dispenser according to Inventive Concept 163, wherein the first and the second nozzles are configured to dispense a total volume of 0.2-20 mL of the nasal wash fluid into the nostril.
Inventive Concept 183. The nasal wash fluid dispenser according to Inventive Concept 182, wherein the first and the second nozzles are configured to dispense a total volume of 0.6-10 mL of the nasal wash fluid into the nostril.
Inventive Concept 184. The nasal wash fluid dispenser according to Inventive Concept 183, wherein the first and the second nozzles are configured to dispense a total volume of 0.2-10 mL of the nasal wash fluid into the nostril.
Inventive Concept 185. The nasal wash fluid dispenser according to Inventive Concept 184, wherein the first and the second nozzles are configured to dispense a total volume of 0.2-5 mL of the nasal wash fluid into the nostril.
Inventive Concept 186. The nasal wash fluid dispenser according to Inventive Concept 185, wherein the first and the second nozzles are configured to dispense a total volume of 0.6-5 mL of the nasal wash fluid into the nostril.
Inventive Concept 187. The nasal wash fluid dispenser according to Inventive Concept 163, wherein at least one of the first and the second nozzles is configured to dispense the nasal wash fluid with a sprayed particle size of 5-50 microns.
Inventive Concept 188. The nasal wash fluid dispenser according to Inventive Concept 187, wherein the at least one of the first and the second nozzles is configured to dispense the nasal wash fluid a sprayed particle size of 5-15 microns.
Inventive Concept 189. The nasal wash fluid dispenser according to Inventive Concept 187, wherein the at least one of the first and the second nozzles is configured to dispense the nasal wash fluid with a sprayed particle size of 15-20 microns.
Inventive Concept 190. The nasal wash fluid dispenser according to any one of Inventive Concepts 163-189,

wherein the nostril is a first nostril of the subject, and wherein the tubular applicator is a first tubular applicator that is sized and shaped to be insertable into the first nostril, and

wherein the nasal wash fluid dispenser further includes a second tubular applicator, which extends distally from and is in fluid communication with the dispensing container, which is sized and shaped to be insertable into a second nostril of the subject, and which includes third and fourth nozzles, which are configured to dispense the nasal wash fluid in third and fourth fluid discharges, respectively, having third and fourth centerlines, respectively, the third and the fourth centerlines defining the offset angle therebetween of between 10 and 90 degrees.

Inventive Concept 191. The nasal wash fluid dispenser according to Inventive Concept 190,

wherein the first tubular applicator does not include any nozzles other than the first and the second nozzles,

wherein the second tubular applicator does not include any nozzles other than the third and the fourth nozzles, and

wherein the nasal wash fluid dispenser does not include any nozzles other than the first, the second, the third, and the fourth nozzles.

Inventive Concept 192. The nasal wash fluid dispenser according to any one of Inventive Concepts 163-189, wherein the first nozzle is configured to dispense the first discharge with a first spray angle of between 5 and 30 degrees, measured at 3 mm from the first nozzle.
Inventive Concept 193. The nasal wash fluid dispenser according to Inventive Concept 192, wherein the first spray angle is between 5 and 15 degrees.
Inventive Concept 194. The nasal wash fluid dispenser according to any one of Inventive Concepts 163-189,

wherein the tubular applicator, including the first and the second nozzles, is configured to dispense, via the first and the second nozzles, respectively, first and second fractions of a total volume of the nasal wash fluid dispensed via the tubular applicator, and

wherein the second fraction equals at least two times the first fraction.

Inventive Concept 195. The nasal wash fluid dispenser according to Inventive Concept 194, wherein the second fraction equals at least five times the first fraction.
Inventive Concept 196. The nasal wash fluid dispenser according to Inventive Concept 195, wherein the second fraction equals at least eight times the first fraction.
Inventive Concept 197. A kit including the nasal wash fluid dispenser according to any one of Inventive Concepts 163-189, the kit further including:

the nasal wash fluid, contained in the dispensing container; and

a test for testing for the presence of a particulate in a specimen sample collected using the nasal wash fluid dispenser.

Inventive Concept 198. The kit according to Inventive Concept 197, wherein the test includes an immunoassay, which is configured to detect the presence of the particulate.
Inventive Concept 199. The kit according to Inventive Concept 198, wherein the immunoassay includes a lateral flow immunoassay test strip, which is configured to detect the presence of the particulate.
Inventive Concept 200. The kit according to Inventive Concept 199, wherein the immunoassay includes a chromatographic digital immunoassay, which is configured to detect the presence of the particulate.
Inventive Concept 201. The kit according to Inventive Concept 197, wherein the test is configured to test for the presence of the particulate to aid in diagnosis of a disease.
Inventive Concept 202. A kit including the nasal wash fluid dispenser according to any one of Inventive Concepts 163-189, the kit further including:

the nasal wash fluid, contained in the dispensing container;

a vial; and

a liquid for bathing at least a portion of a specimen sample collected using the nasal wash fluid dispenser in the vial, the liquid selected from the group consisting of: a lysis buffer, saline solution, and transport medium.

Inventive Concept 203. The nasal wash fluid dispenser according to any one of Inventive Concepts 163-189, wherein the nasal wash fluid dispenser is shaped so as to guide introduction of the tubular applicator into the nostril at a specific orientation with respect to a maxilla of the subject.
Inventive Concept 204. The nasal wash fluid dispenser according to Inventive Concept 203, wherein the dispensing container is shaped so as to guide the introduction of the tubular applicator into the nostril at the specific orientation with respect to the maxilla.
Inventive Concept 205. The nasal wash fluid dispenser according to Inventive Concept 203, wherein the nasal wash fluid dispenser includes a housing that is shaped so as to guide the introduction of the tubular applicator into the nostril at the specific orientation with respect to the maxilla.
Inventive Concept 206. The nasal wash fluid dispenser according to Inventive Concept 203, wherein the nasal wash fluid dispenser is shaped so as to define a concave surface that faces in the posterior direction and generally conforms to an outer surface of an upper lip of the subject, and is configured to stabilize the nasal wash fluid dispenser with respect to the maxilla when the concave surface is pressed against the upper lip.
Inventive Concept 207. The nasal wash fluid dispenser according to Inventive Concept 206, wherein the concave surface has a lateral width of between 2.5 and 7 cm.
Inventive Concept 208. The nasal wash fluid dispenser according to Inventive Concept 206, wherein the concave surface is curved.
Inventive Concept 209. The nasal wash fluid dispenser according to Inventive Concept 206, wherein the concave surface is concave about an axis and flat in a direction along the axis.
Inventive Concept 210. The nasal wash fluid dispenser according to Inventive Concept 209, wherein the axis forms an angle of between 75 and 90 degrees with the first centerline of the first fluid discharge.
Inventive Concept 211. The nasal wash fluid dispenser according to Inventive Concept 210, wherein the angle is between 85 and 90 degrees.
Inventive Concept 212. The nasal wash fluid dispenser according to Inventive Concept 206, wherein the dispensing container is shaped so as to define the concave surface.
Inventive Concept 213. The nasal wash fluid dispenser according to Inventive Concept 206, wherein the nasal wash fluid dispenser includes a housing that is shaped so as to define the concave surface.
Inventive Concept 214. The nasal wash fluid dispenser according to any one of Inventive Concepts 163-189, wherein the first and the second nozzles are configured to dispense nasal wash fluid such that the first and the second fluid discharges, respectively, are respective first and second types of fluid discharges selected from the group consisting of: a fluid stream, a spray, a mist, a pressurized aerosol, and an atomized fluid.
Inventive Concept 215. The nasal wash fluid dispenser according to Inventive Concept 214, wherein the first and the second types of fluid discharges are the same type.
Inventive Concept 216. The nasal wash fluid dispenser according to Inventive Concept 214, wherein the first and the second types of fluid discharges are different types.
Inventive Concept 217. The nasal wash fluid dispenser according to any one of Inventive Concepts 163-189, wherein at least one of the first and the second nozzles includes a flat nozzle.
Inventive Concept 218. The nasal wash fluid dispenser according to Inventive Concept 217, wherein the first nozzle includes a first flat nozzle, and the second nozzle includes a second flat nozzle.
Inventive Concept 219. The nasal wash fluid dispenser according to Inventive Concept 217, wherein the flat nozzle includes a flat tapered nozzle.
Inventive Concept 220. The nasal wash fluid dispenser according to any one of Inventive Concepts 163-189, wherein the nasal wash fluid dispenser is configured to dispense the nasal wash fluid in a plurality of pulses.
Inventive Concept 221. The nasal wash fluid dispenser according to Inventive Concept 220, wherein the nasal wash fluid dispenser is configured to dispense the nasal wash fluid in a plurality of pulses having a volume of 0.02-10 mL per pulse.
Inventive Concept 222. The nasal wash fluid dispenser according to Inventive Concept 221, wherein the nasal wash fluid dispenser is configured to dispense the nasal wash fluid in a plurality of pulses having a volume of 0.02-1 mL per pulse.
Inventive Concept 223. The nasal wash fluid dispenser according to any one of Inventive Concepts 163-171 and 173-189, wherein the tubular applicator further includes a third nozzle, which is configured to dispense the nasal wash fluid in a third fluid discharge, having a third centerline, the first and the third centerlines defining an offset angle therebetween of between 75 and 105 degrees, such that, when the tubular applicator is inserted into the nostril with the first nozzle oriented in the posterior direction and the second nozzle oriented in the superoposterior direction, the third nozzle directs the third fluid discharge at least in a superior direction toward an inferior border of upper lateral cartilage of the nasal cavity.
Inventive Concept 224. The nasal wash fluid dispenser according to Inventive Concept 223, wherein the third nozzle is configured to dispense the nasal wash fluid in the third fluid discharge, such that, when the tubular applicator is inserted into the nostril with the first nozzle oriented in the posterior direction and the second nozzle oriented in the superoposterior direction, the third nozzle directs the third fluid discharge additionally bilaterally toward a septum and a lateral wall of the nasal cavity.
Inventive Concept 225. The nasal wash fluid dispenser according to Inventive Concept 223, wherein the third nozzle is configured to dispense the third discharge with a third spray angle of between 45 and 135 degrees, measured at 3 mm from the third nozzle.
Inventive Concept 226. The nasal wash fluid dispenser according to Inventive Concept 223, wherein the tubular applicator does not include any nozzles other than the first, the second, and the third nozzles.
Inventive Concept 227. A nasal wash fluid system including the nasal wash fluid dispenser according to any one of Inventive Concepts 163-189, wherein the nasal wash fluid system further includes the nasal wash fluid, contained in the dispensing container.
Inventive Concept 228. The nasal wash fluid system according to Inventive Concept 227, wherein the nasal wash fluid does not include a drug molecule.
Inventive Concept 229. The nasal wash fluid system according to Inventive Concept 227, wherein the nasal wash fluid includes a non-irritant solution.
Inventive Concept 230. The nasal wash fluid system according to Inventive Concept 229, wherein the non-irritant solution consists of water.
Inventive Concept 231. The nasal wash fluid system according to Inventive Concept 229, wherein the non-irritant solution includes saline solution.
Inventive Concept 232. The nasal wash fluid system according to Inventive Concept 231, wherein the non-irritant saline solution includes a phosphate-buffered saline solution.
Inventive Concept 233. The nasal wash fluid system according to Inventive Concept 227, wherein the nasal wash fluid includes a tastant.
Inventive Concept 234. The nasal wash fluid system according to Inventive Concept 227, wherein the nasal wash fluid includes a color additive.
Inventive Concept 235. The nasal wash fluid system according to Inventive Concept 227,

wherein the nasal wash fluid includes a first color additive, and

wherein the nasal wash fluid system further includes an oral wash fluid including a second colored additive, the first and the second color additives having different colors.

Inventive Concept 236. A testing system including the nasal wash fluid dispenser according to any one of Inventive Concepts 163-189, the testing system further including a diagnostic test kit, which is configured to test, for the presence of a particulate, a specimen sample that passed out of an oral cavity of the subject and contains at least a portion of the biological material washed into the oropharynx with the nasal wash fluid.
Inventive Concept 237. The testing system according to Inventive Concept 236, wherein the diagnostic test kit further includes one or more filters configured to concentrate the specimen sample by filtration prior to testing for the presence of the particulate, by passing the specimen sample through one or more filters.
Inventive Concept 238. The testing system according to Inventive Concept 236, wherein the particulate includes one or more particulates selected from the group consisting of: a pathogenic microorganism, a non-pathogenic microorganism, an antigen, a human cell, a cellular biomarker, a hormone, a chemical mediator from a cell, a pollen, a respiratory particle, a particle contained within droplets from lower airways of the subject, a nucleic acid, and a chemical originating from an external vapor.
Inventive Concept 239. The testing system according to Inventive Concept 236, wherein the particulate includes one or more particulates selected from the group consisting of: a virus, a bacterium, a protozoan, and a fungus.
Inventive Concept 240. The testing system according to Inventive Concept 236, wherein the particulate includes a virus.
Inventive Concept 241. The testing system according to Inventive Concept 240, wherein the virus is an Influenza virus.
Inventive Concept 242. The testing system according to Inventive Concept 240, wherein the virus is a coronavirus.
Inventive Concept 243. The testing system according to Inventive Concept 242, wherein the coronavirus is Severe acute respiratory syndrome coronavirus 2 (SARS-CoV-2).
Inventive Concept 244. The testing system according to Inventive Concept 236, wherein the particulate includes a bacterium.
Inventive Concept 245. The testing system according to Inventive Concept 244, wherein the bacterium is a Streptococcus bacterium.
Inventive Concept 246. The testing system according to Inventive Concept 236, wherein the particulate includes one or more antigens selected from the group consisting of: a viral antigen, a bacterial antigen, and a fungal antigen.

There is still further provided, in accordance with an Inventive Concept 247 of the present invention, a nasal wash fluid dispenser for intranasally dispensing nasal wash fluid into a nasal cavity of a subject, the nasal wash fluid dispenser including:

a dispensing container, which contains the nasal wash fluid; and

a tubular applicator, which extends distally from and is in fluid communication with the dispensing container, which is sized and shaped to be insertable into a nostril of the subject, and which includes a nozzle, which is configured to dispense the nasal wash fluid in a fluid discharge having a centerline, such that, when the tubular applicator is inserted into the nostril with the nozzle oriented in a posterior direction, the nozzle directs the fluid discharge toward a nasopharynx of the subject, so as to wash biological material, with some of the nasal wash fluid, from the nasal cavity into an oropharynx of the subject via the nasopharynx,

wherein the nasal wash fluid dispenser is shaped so as to guide introduction of the tubular applicator into the nostril at a specific orientation with respect to a maxilla of the subject.

Inventive Concept 248. The nasal wash fluid dispenser according to Inventive Concept 247, wherein the dispensing container is shaped so as to guide the introduction of the tubular applicator into the nostril at the specific orientation with respect to the maxilla.
Inventive Concept 249. The nasal wash fluid dispenser according to Inventive Concept 247, wherein the nasal wash fluid dispenser includes a housing that is shaped so as to guide the introduction of the tubular applicator into the nostril at the specific orientation with respect to the maxilla.
Inventive Concept 250. The nasal wash fluid dispenser according to Inventive Concept 247, wherein the nasal wash fluid dispenser is shaped so as to define a concave surface that faces in the posterior direction and generally conforms to an outer surface of an upper lip of the subject, and is configured to stabilize the nasal wash fluid dispenser with respect to the maxilla when the concave surface is pressed against the upper lip.
Inventive Concept 251. The nasal wash fluid dispenser according to Inventive Concept 250, wherein the concave surface has a lateral width of between 2.5 and 7 cm.
Inventive Concept 252. The nasal wash fluid dispenser according to Inventive Concept 250, wherein the concave surface is curved.
Inventive Concept 253. The nasal wash fluid dispenser according to Inventive Concept 250, wherein the concave surface is concave about an axis and flat in a direction along the axis.
Inventive Concept 254. The nasal wash fluid dispenser according to Inventive Concept 253, wherein the axis forms an angle of between 75 and 90 degrees with the centerline of the fluid discharge.
Inventive Concept 255. The nasal wash fluid dispenser according to Inventive Concept 254, wherein the angle is between 85 and 90 degrees.
Inventive Concept 256. The nasal wash fluid dispenser according to Inventive Concept 250, wherein the dispensing container is shaped so as to define the concave surface.
Inventive Concept 257. The nasal wash fluid dispenser according to Inventive Concept 250, wherein the nasal wash fluid dispenser includes a housing that is shaped so as to define the concave surface.
Inventive Concept 258. The nasal wash fluid dispenser according to any one of Inventive Concepts 247-257,

wherein the nostril is a first nostril of the subject, and wherein the tubular applicator is a first tubular applicator that is sized and shaped to be insertable into the first nostril, and

wherein the nasal wash fluid dispenser further includes a second tubular applicator, which extends distally from and is in fluid communication with the dispensing container, which is sized and shaped to be insertable into a second nostril of the subject, and which include a second nozzle, which is configured to dispense the nasal wash fluid in a second fluid discharge, having a second centerline.

Inventive Concept 259. The nasal wash fluid dispenser according to any one of Inventive Concepts 247-257, wherein the tubular applicator is shaped so as to define distal-most and proximal-most central longitudinal axes through distal and proximal ends of the tubular applicator, respectively, and wherein the distal-most and the proximal-most central longitudinal axes form a bend angle of between 100 and 175 degrees therebetween.

There is additionally provided, in accordance with an Inventive Concept 260 of the present invention, a nasal wash fluid system, including:

a nasal wash fluid, which includes a color additive; and

a nasal wash fluid dispenser, which includes:
a dispensing container, which contains the nasal wash fluid; and
a tubular applicator, which extends distally from and is in fluid communication with the dispensing container, and which is sized and shaped to be insertable into a nostril of a subject.
   Inventive Concept 261. The nasal wash fluid system according to Inventive Concept 260, wherein the nasal wash fluid does not include a drug molecule.
   Inventive Concept 262. The nasal wash fluid system according to Inventive Concept 260, wherein the color additive has a visually-perceptible color that is dominated by a non-white color other than yellow.
   Inventive Concept 263. The nasal wash fluid system according to Inventive Concept 260,

wherein the color additive is a first color additive, and

wherein the nasal wash fluid system further includes:
an oral wash fluid including a second colored additive, the first and the second color additives having different colors; and
an oral-wash-fluid container, which contains the oral wash fluid.

There is also provided, in accordance with an Inventive Concept 264 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and

thereafter, collecting a specimen sample by drawing the specimen sample out of an anterior opening of an oral cavity of the subject using an absorbent material, the specimen sample containing at least a portion of the biological material washed into the oropharynx by the nasal wash fluid.

There is further provided, in accordance with an Inventive Concept 265 of the present invention, a method including:

intranasally dispensing nasal wash fluid into a nasal cavity of a subject such that the nasal wash fluid passes from the nasal cavity into an oropharynx of the subject via a nasopharynx of the subject; and

thereafter, collecting a specimen sample by drawing the specimen sample out of an anterior opening of an oral cavity of the subject using an absorbent material, the specimen sample containing at least a portion of the nasal wash fluid.

Inventive Concept 266. The method according to any one of Inventive Concepts 264 and 265, wherein the absorbent material is located on a tip of a collector shaft, and wherein drawing the specimen sample out of the anterior opening of the oral cavity using the absorbent material includes inserting the tip of the collector shaft into the oral cavity.
Inventive Concept 267. The method according to Inventive Concept 266, wherein drawing the specimen sample out of the anterior opening of the oral cavity using the absorbent material includes sucking, by the subject, on the absorbent material.
Inventive Concept 268. The method according to any one of Inventive Concepts 264 and 265, wherein drawing the specimen sample out of the anterior opening of the oral cavity includes contacting one or more portions of the oral cavity with the absorbent material, without swabbing the oropharynx.
Inventive Concept 269. The method according to Inventive Concept 268, wherein the one or more portions of the oral cavity include one or more portions selected from the group consisting of: buccal mucosa, a tongue, gums, and palatal mucosa.
Inventive Concept 270. The method according to any one of Inventive Concepts 264 and 265, wherein collecting the specimen sample includes collecting the specimen sample after sensing, by the subject, the nasal wash fluid in the oropharynx.
Inventive Concept 271. The method according to Inventive Concept 270, wherein collecting the specimen sample includes collecting the specimen sample after tasting, by the subject, the nasal wash fluid in the oropharynx.
Inventive Concept 272. The method according to Inventive Concept 271, wherein the nasal wash fluid includes a tastant, and wherein collecting the specimen sample includes collecting the specimen sample after tasting, by the subject, the tastant in the oropharynx.
Any of Inventive Concepts 264-272 may be performed in combination with any of Inventive Concepts 1-25, 40-99, 122-150, and 156.

There is still further provided, in accordance with an Inventive Concept 273 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and

thereafter, collecting a specimen sample by swabbing the oropharynx via an anterior opening of an oral cavity of the subject, the specimen sample containing at least a portion of the biological material washed into the oropharynx by the nasal wash fluid,

wherein the method does not include swabbing the nasopharynx.

There is additionally provided, in accordance with an Inventive Concept 274 of the present invention, a method including:

intranasally dispensing nasal wash fluid into a nasal cavity of a subject such that the nasal wash fluid passes from the nasal cavity into an oropharynx of the subject via a nasopharynx of the subject; and

thereafter, collecting a specimen sample by swabbing the oropharynx via an anterior opening of an oral cavity of the subject, the specimen sample containing at least a portion of the nasal wash fluid.

Inventive Concept 275. The method according to any one of Inventive Concepts 273 and 274, wherein the method does not include swabbing the nasal cavity.
Inventive Concept 276. The method according to any one of Inventive Concepts 273 and 274, wherein collecting the specimen sample includes swabbing the oropharynx and swabbing one or both anterior nares of the nasal cavity.
Inventive Concept 277. The method according to any one of Inventive Concepts 273 and 274, wherein collecting the specimen sample includes swabbing the oropharynx and swabbing one or more turbinates of the nasal cavity selected from the group consisting of: a middle turbinate and an inferior turbinate.
Inventive Concept 278. The method according to any one of Inventive Concepts 273 and 274, wherein collecting the specimen sample includes collecting the specimen sample after sensing, by the subject, the nasal wash fluid in the oropharynx.
Inventive Concept 279. The method according to Inventive Concept 278, wherein collecting the specimen sample includes collecting the specimen sample after tasting, by the subject, the nasal wash fluid in the oropharynx.
Inventive Concept 280. The method according to Inventive Concept 279, wherein the nasal wash fluid includes a tastant, and wherein collecting the specimen sample includes collecting the specimen sample after tasting, by the subject, the tastant in the oropharynx.
Any of Inventive Concepts 273-280 may be performed in combination with any of Inventive Concepts 1-25, 40-99, and 122-150.

There is further provided, in accordance with an Inventive Concept 281 of the present invention, a method including:

intranasally dispensing nasal wash fluid into a nasal cavity of a subject such that the nasal wash fluid passes from the nasal cavity into an oropharynx of the subject via a nasopharynx of the subject; and

thereafter, collecting a specimen sample that passed out of an anterior opening of an oral cavity of the subject and contains at least a portion of the nasal wash fluid.

Inventive Concept 282. The method according to Inventive Concept 281, wherein collecting the specimen sample includes collecting the specimen sample into a collection receptacle.
Inventive Concept 283. The method according to Inventive Concept 281, wherein intranasally dispensing includes intranasally dispensing, by the subject, the nasal wash fluid into the nasal cavity.
Inventive Concept 284. The method according to Inventive Concept 281, wherein collecting the specimen sample includes expressing, by the subject, the specimen sample out of the oropharynx via the anterior opening of the oral cavity.
Inventive Concept 285. The method according to Inventive Concept 284, wherein expressing the specimen sample includes spitting, by the subject, the specimen sample out of the oropharynx via the anterior opening of the oral cavity.
Inventive Concept 286. The method according to Inventive Concept 284, wherein expressing the specimen sample includes expressing the specimen sample out of the oropharynx via the anterior opening of the oral cavity after sensing, by the subject, the nasal wash fluid in the oropharynx.
Inventive Concept 287. The method according to Inventive Concept 286, wherein expressing the specimen sample includes expressing the specimen sample out of the oropharynx via the anterior opening of the oral cavity after tasting, by the subject, the nasal wash fluid in the oropharynx.
Inventive Concept 288. The method according to Inventive Concept 287, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 289. The method according to Inventive Concept 284, wherein intranasally dispensing includes intranasally dispensing, by a healthcare worker, the nasal wash fluid into the nasal cavity.
Inventive Concept 290. The method according to Inventive Concept 281, further including, before collecting the specimen sample, tilting back, by the subject, a head of the subject.
Inventive Concept 291. The method according to Inventive Concept 281, further including, while intranasally dispensing the nasal wash fluid, tilting back, by the subject, a head of the subject.
Inventive Concept 292. The method according to Inventive Concept 281, further including, before intranasally dispensing the nasal wash fluid, tilting back, by the subject, a head of the subject.
Inventive Concept 293. The method according to Inventive Concept 281, further including, after intranasally dispensing the nasal wash fluid, tilting a head of the subject farther back than while intranasally dispensing the nasal wash fluid and farther back than a neutral position.
Inventive Concept 294. The method according to Inventive Concept 281, wherein intranasally dispensing the nasal wash fluid includes intranasally dispensing the nasal wash fluid into the nasal cavity while a head of the subject is held in a neutral position or a tilted-forward position.
Inventive Concept 295. The method according to Inventive Concept 281, further including waiting for a period of time between (a) concluding intranasally dispensing the nasal wash fluid into the nasal cavity and (b) collecting the specimen sample, the period of time at least 5 minutes and less than 90 minutes.
Inventive Concept 296. The method according to Inventive Concept 281, wherein intranasally dispensing the nasal wash fluid includes intranasally dispensing the nasal wash fluid as one or more types of fluid discharge selected from the group consisting of: a fluid stream, a spray, a mist, a pressurized aerosol, and an atomized fluid.
Inventive Concept 297. The method according to Inventive Concept 281, wherein intranasally dispensing the nasal wash fluid includes intranasally dispensing the nasal wash fluid as steam.
Inventive Concept 298. The method according to Inventive Concept 281, wherein intranasally dispensing the nasal wash fluid includes intranasally dispensing the nasal wash fluid in a circular rotary motion to generate dispersion within the nasal cavity.
Inventive Concept 299. The method according to Inventive Concept 281, wherein intranasally dispensing the nasal wash fluid into the nasal cavity includes intranasally dispensing a total volume of 0.2-20 mL of the nasal wash fluid into the nasal cavity.
Inventive Concept 300. The method according to Inventive Concept 299, wherein intranasally dispensing the nasal wash fluid into the nasal cavity includes intranasally dispensing a total volume of 0.6-20 mL of the nasal wash fluid into the nasal cavity.
Inventive Concept 301. The method according to Inventive Concept 299, wherein intranasally dispensing the nasal wash fluid into the nasal cavity includes intranasally dispensing a total volume of 0.2-40 mL of the nasal wash fluid into the nasal cavity.
Inventive Concept 302. The method according to Inventive Concept 301, wherein intranasally dispensing the nasal wash fluid into the nasal cavity includes intranasally dispensing a total volume of 0.2-5 mL of the nasal wash fluid into the nasal cavity.
Inventive Concept 303. The method according to Inventive Concept 302, wherein intranasally dispensing the nasal wash fluid into the nasal cavity includes intranasally dispensing a total volume of 0.6-5 mL of the nasal wash fluid into the nasal cavity.
Inventive Concept 304. The method according to Inventive Concept 299, wherein intranasally dispensing the nasal wash fluid into the nasal cavity includes intranasally dispensing the total volume of the nasal wash fluid in a plurality of pulses.
Inventive Concept 305. The method according to Inventive Concept 304, wherein each of the pulses has a volume of 0.02-10 mL.
Inventive Concept 306. The method according to Inventive Concept 305, wherein each of the pulses has a volume of 0.02-1 mL.
Inventive Concept 307. The method according to Inventive Concept 281, wherein intranasally dispensing the nasal wash fluid into the nasal cavity includes intranasally dispensing the nasal wash fluid with a sprayed particle size of 5-50 microns.
Inventive Concept 308. The method according to Inventive Concept 307, wherein intranasally dispensing the nasal wash fluid includes intranasally dispensing the nasal wash fluid with a sprayed particle size of 5-15 microns.
Inventive Concept 309. The method according to Inventive Concept 307, wherein intranasally dispensing the nasal wash fluid includes intranasally dispensing the nasal wash fluid with a sprayed particle size of 15-20 microns.
Inventive Concept 310. The method according to Inventive Concept 281, wherein intranasally dispensing the nasal wash fluid into the nasal cavity includes intranasally dispensing the nasal wash fluid into the nasal cavity while the subject is lying supine.
Inventive Concept 311. The method according to Inventive Concept 281, wherein collecting the specimen sample includes drawing the specimen sample out of the oropharynx via the anterior opening of the oral cavity using an absorbent material, without swabbing the oral cavity or the oropharynx.
Inventive Concept 312. The method according to any one of Inventive Concepts 281-311, wherein intranasally dispensing includes intranasally dispensing, by a healthcare worker, the nasal wash fluid into the nasal cavity.
Inventive Concept 313. The method according to Inventive Concept 312, wherein collecting the specimen sample includes receiving the specimen sample by a healthcare worker from the subject.
Inventive Concept 314. The method according to Inventive Concept 313, wherein receiving the specimen sample includes receiving the specimen sample that was expressed out of the oropharynx via the anterior opening of the oral cavity.
Inventive Concept 315. The method according to Inventive Concept 314, wherein receiving the specimen sample includes receiving the specimen sample that was spit out of the oropharynx via the anterior opening of the oral cavity.
Inventive Concept 316. The method according to Inventive Concept 312, wherein collecting the specimen sample includes aspirating the specimen sample out of the oropharynx via the anterior opening of the oral cavity.
Inventive Concept 317. The method according to Inventive Concept 312, wherein collecting the specimen sample includes instructing the subject, by the healthcare worker, to express the specimen sample out of the oropharynx via the anterior opening of the oral cavity.
Inventive Concept 318. The method according to Inventive Concept 317, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 319. The method according to any one of Inventive Concepts 281-311, further including, after intranasally dispensing the nasal wash fluid and before collecting the specimen sample, sniffing, by the subject, the nasal wash fluid from the nasal cavity posteriorly toward the nasopharynx.
Inventive Concept 320. The method according to any one of Inventive Concepts 281-311, further including, after intranasally dispensing the nasal wash fluid and before collecting the specimen sample, snorting, by the subject, the nasal wash fluid from the nasal cavity toward the nasopharynx.
Inventive Concept 321. The method according to any one of Inventive Concepts 281-311, further including, after collecting the specimen sample, testing the specimen sample for the presence of a particulate.
Inventive Concept 322. The method according to Inventive Concept 321, wherein testing the specimen sample for the presence of the particulate includes diagnosing a disease.
Inventive Concept 323. The method according to Inventive Concept 322, wherein diagnosing the disease includes diagnosing an infectious disease.
Inventive Concept 324. The method according to Inventive Concept 321, wherein testing the specimen sample for the presence of the particulate includes concentrating the specimen sample by filtration prior to testing for the presence of the particulate, by passing the specimen sample through one or more filters.
Inventive Concept 325. The method according to Inventive Concept 321, wherein the particulate includes one or more particulates selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.
Inventive Concept 326. The method according to Inventive Concept 321, wherein the particulate includes one or more particulates selected from the group consisting of: a respiratory particulate and a particulate contained within droplets from lower airways of the subject.
Inventive Concept 327. The method according to Inventive Concept 321, wherein the particulate includes one or more particulates selected from the group consisting of: a virus, a bacterium, a protozoan, and a fungus.
Inventive Concept 328. The method according to Inventive Concept 321, wherein the particulate includes a virus.
Inventive Concept 329. The method according to Inventive Concept 328, wherein the virus is an Influenza virus.
Inventive Concept 330. The method according to Inventive Concept 328, wherein the virus is a coronavirus.
Inventive Concept 331. The method according to Inventive Concept 330, wherein the coronavirus is Severe acute respiratory syndrome coronavirus 2 (SARS-CoV-2).
Inventive Concept 332. The method according to Inventive Concept 321, wherein the particulate includes a bacterium.
Inventive Concept 333. The method according to Inventive Concept 332, wherein the bacterium is a Streptococcus bacterium.
Inventive Concept 334. The method according to Inventive Concept 321, wherein the particulate includes one or more antigens selected from the group consisting of: a viral antigen, a bacterial antigen, and a fungal antigen.
Inventive Concept 335. The method according to any one of Inventive Concepts 281-311, wherein intranasally dispensing the nasal wash fluid into the nasal cavity includes intranasally dispensing the nasal wash fluid unidirectionally within the nasal cavity.
Inventive Concept 336. The method according to Inventive Concept 335, wherein intranasally dispensing the nasal wash fluid unidirectionally within the nasal cavity includes intranasally dispensing the nasal wash fluid unidirectionally within the nasal cavity in a superior direction.
Inventive Concept 337. The method according to Inventive Concept 335, wherein intranasally dispensing the nasal wash fluid unidirectionally within the nasal cavity includes intranasally dispensing the nasal wash fluid unidirectionally within the nasal cavity in a superoposterior direction.
Inventive Concept 338. The method according to Inventive Concept 337, wherein intranasally dispensing the nasal wash fluid unidirectionally within the nasal cavity in a superoposterior direction includes intranasally dispensing the nasal wash fluid unidirectionally within the nasal cavity toward an olfactory area of the nasal cavity.
Inventive Concept 339. The method according to Inventive Concept 335, wherein intranasally dispensing the nasal wash fluid unidirectionally within the nasal cavity includes intranasally dispensing the nasal wash fluid unidirectionally within the nasal cavity in a posterior direction.
Inventive Concept 340. The method according to Inventive Concept 339, wherein intranasally dispensing the nasal wash fluid unidirectionally within the nasal cavity in a posterior direction includes intranasally dispensing the nasal wash fluid unidirectionally within the nasal cavity toward the nasopharynx.
Inventive Concept 341. The method according to any one of Inventive Concepts 281-311, wherein intranasally dispensing the nasal wash fluid into the nasal cavity includes intranasally dispensing the nasal wash fluid multidirectionally within the nasal cavity.
Inventive Concept 342. The method according to Inventive Concept 341, wherein intranasally dispensing the nasal wash fluid multidirectionally within the nasal cavity includes dispensing the nasal wash fluid into the nasal cavity:

in a posterior direction toward the nasopharynx, and

in a superoposterior direction toward an olfactory area of the nasal cavity.

Inventive Concept 343. The method according to Inventive Concept 342, wherein intranasally dispensing the nasal wash fluid multidirectionally within the nasal cavity includes dispensing the nasal wash fluid into the nasal cavity additionally in at least a superior direction toward an inferior border of upper lateral cartilage of the nasal cavity.
Inventive Concept 344. The method according to Inventive Concept 343, wherein intranasally dispensing the nasal wash fluid multidirectionally within the nasal cavity includes dispensing the nasal wash fluid into the nasal cavity additionally bilaterally toward a septum and a lateral wall of the nasal cavity.
Inventive Concept 345. The method according to Inventive Concept 341, wherein intranasally dispensing the nasal wash fluid multidirectionally within the nasal cavity includes intranasally dispensing the nasal wash fluid via a plurality of nozzles.
Inventive Concept 346. The method according to Inventive Concept 345, wherein intranasally dispensing the nasal wash fluid multidirectionally within the nasal cavity via the plurality of nozzles includes intranasally dispensing the nasal wash fluid into the nasal cavity:

in a posterior direction toward the nasopharynx via a first subset of one or more of the plurality of nozzles, and

in a superoposterior direction toward an olfactory area of the nasal cavity via a second subset of one or more of the plurality of nozzles, the first and the second subsets not including any nozzles common to both subsets.

Inventive Concept 347. The method according to Inventive Concept 346,

wherein the plurality of nozzles includes only first and second nozzles, and wherein the first and the second subsets consist of the first and the second nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle, and
in the superoposterior direction toward the olfactory area of the nasal cavity via the second nozzle.
   Inventive Concept 348. The method according to Inventive Concept 346, wherein intranasally dispensing the nasal wash fluid multidirectionally within the nasal cavity via the plurality of nozzles includes intranasally dispensing the nasal wash fluid into the nasal cavity:

in the posterior direction toward the nasopharynx via the first subset of one or more of the plurality of nozzles,

in the superoposterior direction toward the olfactory area of the nasal cavity via the second subset of one or more of the plurality of nozzles, and

at least in a superior direction toward an inferior border of upper lateral cartilage of the nasal cavity via a third subset of one or more of the plurality of nozzles, wherein none of the nozzles is included in more than one of the first, the second, and the third subsets.

Inventive Concept 349. The method according to Inventive Concept 348, wherein intranasally dispensing the nasal wash fluid in at least the superior direction toward the inferior border of upper lateral cartilage includes dispensing the nasal wash fluid into the nasal cavity additionally bilaterally toward a septum and a lateral wall of the nasal cavity via the third subset of one or more of the plurality of nozzles.
Inventive Concept 350. The method according to Inventive Concept 348,

wherein the plurality of nozzles includes only first, second, and third nozzles, and wherein the first, the second, and the third subsets consist of the first, the second, and the third nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle,
in the superoposterior direction toward the olfactory area of the nasal cavity via the second nozzle, and
at least in the superior direction toward the inferior border of upper lateral cartilage of the nasal cavity via the third nozzle.
   Inventive Concept 351. The method according to Inventive Concept 345, wherein intranasally dispensing the nasal wash fluid multidirectionally within the nasal cavity includes intranasally dispensing the nasal wash fluid within the nasal cavity via the plurality of nozzles with respective different fluid flow characteristics, the fluid flow characteristics including one or more characteristics selected from the group consisting of: volume, sprayed particle size, velocity, and flow rate.
   Inventive Concept 352. The method according to any one of Inventive Concepts 281-311, wherein intranasally dispensing the nasal wash fluid into the nasal cavity includes intranasally dispensing the nasal wash fluid via both nostrils of the subject simultaneously.
   Inventive Concept 353. The method according to any one of Inventive Concepts 281-311, wherein the nasal wash fluid does not include a drug molecule.
   Inventive Concept 354. The method according to any one of Inventive Concepts 281-311, wherein the nasal wash fluid includes a non-irritant solution.
   Inventive Concept 355. The method according to Inventive Concept 354, wherein the non-irritant solution consists of water.
   Inventive Concept 356. The method according to Inventive Concept 354, wherein the non-irritant solution includes saline solution.
   Inventive Concept 357. The method according to Inventive Concept 356, wherein the non-irritant saline solution includes phosphate-buffered saline solution.
   Inventive Concept 358. The method according to any one of Inventive Concepts 281-311, wherein the nasal wash fluid includes a tastant.
   Inventive Concept 359. The method according to any one of Inventive Concepts 281-311, wherein the nasal wash fluid includes a color additive, and collecting the specimen sample includes confirming that the specimen sample contains at least a portion of the nasal wash fluid by visually ascertaining that the specimen sample includes the color additive.
   Inventive Concept 360. The method according to any one of Inventive Concepts 281-311, wherein the nasal wash fluid includes a chemical additive, and collecting the specimen sample includes confirming that the specimen sample contains at least a portion of the nasal wash fluid by ascertaining that the specimen sample includes the chemical additive.
   Inventive Concept 361. The method according to Inventive Concept 360,

further including, after collecting the specimen sample, testing the specimen sample for the presence of a particulate present, using a testing technique, and

wherein ascertaining that the specimen sample includes the chemical additive includes ascertaining that the specimen sample includes the chemical additive using the same testing technique used to test the specimen sample for the presence of the particulate.

Inventive Concept 362. The method according to any one of Inventive Concepts 281-311, further including, after intranasally dispensing and before collecting the specimen sample, swishing in the oral cavity, by the subject, the nasal wash fluid washed into the oropharynx.
Inventive Concept 363. The method according to any one of Inventive Concepts 281-311, further including, after intranasally dispensing and before collecting the specimen sample, gargling, by the subject, the nasal wash fluid washed into the oropharynx.
Inventive Concept 364. The method according to Inventive Concept 363, wherein the method further includes, after gargling the nasal wash fluid and before collecting the specimen sample, while holding the nasal wash fluid in the oropharynx, sniffing an additional portion of the nasal wash fluid from the nasal cavity toward the oropharynx such that the additional portion of the nasal wash fluid is added to the nasal wash fluid already in the oropharynx.
Inventive Concept 365. The method according to Inventive Concept 364, wherein the method further includes, after sniffing the additional portion of the nasal wash fluid from the nasal cavity toward the oropharynx, again gargling the nasal wash fluid washed into the oropharynx.
Inventive Concept 366. The method according to any one of Inventive Concepts 281-311,

wherein the method further includes, before collecting the specimen sample:
orally dispensing oral wash fluid into the oral cavity via the anterior opening of the oral cavity; and
swishing, by the subject, the oral wash fluid in the oral cavity, and

wherein collecting includes collecting the specimen sample that passed out of the anterior opening of the oral cavity and contains at least a portion of the oral wash fluid and at least a portion of the nasal wash fluid.

Inventive Concept 367. The method according to any one of Inventive Concepts 281-311,

wherein the method further includes, before collecting the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein collecting includes collecting the specimen sample that passed out of the anterior opening of the oral cavity and contains at least a portion of the mixture.

Inventive Concept 368. The method according to any one of Inventive Concepts 281-311,

wherein the method further includes, before collecting the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein collecting includes collecting the specimen sample that passed out of the anterior opening of the oral cavity and contains at least a portion of the mixture.

Inventive Concept 369. The method according to any one of Inventive Concepts 281-311,

wherein the method further includes, before collecting the specimen sample:
orally dispensing oral wash fluid into the oral cavity via the anterior opening of the oral cavity; and
gargling, by the subject, the oral wash fluid, and

wherein collecting includes collecting the specimen sample that passed out of the anterior opening of the oral cavity and contains at least a portion of the oral wash fluid and at least a portion of the nasal wash fluid.

Inventive Concept 370. The method according to any one of Inventive Concepts 367, 368, and 369, wherein orally dispensing the oral wash fluid into the oral cavity includes orally dispensing the oral wash fluid into the oral cavity prior to intranasally dispensing the nasal wash fluid into the nasal cavity.
Inventive Concept 371. The method according to any one of Inventive Concepts 367, 368, and 369, wherein orally dispensing the oral wash fluid into the oral cavity includes orally dispensing the oral wash fluid into the oral cavity after intranasally dispensing the nasal wash fluid into the nasal cavity.
Inventive Concept 372. The method according to Inventive Concept 371, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 373. The method according to any one of Inventive Concepts 367, 368, and 369,

wherein the specimen sample is a first specimen sample, and the mixture is a first mixture,

wherein the method further includes, after collecting the first specimen sample:
waiting for a period of time, the period of time at least 5 minutes and less than 90 minutes;
thereafter, orally dispensing additional oral wash fluid into the oral cavity via the anterior opening of the oral cavity; and
thereafter, gargling, by the subject,

wherein collecting includes collecting a second specimen sample that passed out of the anterior opening of the oral cavity and contains at least a portion of the additional oral wash fluid, and

wherein the method does not include intranasally dispensing nasal wash fluid into the nasal cavity after collecting the first specimen sample.

Inventive Concept 374. The method according to any one of Inventive Concepts 367, 368, and 369,

wherein the nasal wash fluid includes a first color additive,

wherein the oral wash fluid includes a second colored additive, the first and the second color additives having different colors, and

wherein collecting the specimen sample includes confirming that the specimen sample contains at least a portion of the nasal wash fluid and at least a portion of the oral wash fluid by visually ascertaining that the specimen sample has a color produced by a combination of the first and the second color additives.

Inventive Concept 375. The method according to any one of Inventive Concepts 367, 368, and 369, wherein the oral wash fluid includes a non-irritant solution.
Inventive Concept 376. The method according to Inventive Concept 375, wherein the non-irritant solution consists of water.
Inventive Concept 377. The method according to Inventive Concept 375, wherein the non-irritant solution includes a tastant.
Inventive Concept 378. The method according to Inventive Concept 375, wherein the non-irritant solution includes saline solution.
Inventive Concept 379. The method according to Inventive Concept 378, wherein the non-irritant saline solution includes phosphate-buffered saline solution.
Inventive Concept 380. The method according to any one of Inventive Concepts 367 and 368, wherein the method further includes, after gargling the mixture and before collecting the specimen sample, while holding the mixture in the oropharynx, sniffing an additional portion of the nasal wash fluid from the nasal cavity toward the oropharynx such that the additional portion of the nasal wash fluid is added to the mixture in the oropharynx.
Inventive Concept 381. The method according to Inventive Concept 380, wherein the method further includes, after sniffing the additional portion of the nasal wash fluid from the nasal cavity toward the oropharynx, again gargling the mixture.
Inventive Concept 382. The method according to Inventive Concept 380, wherein gargling the mixture and thereafter sniffing the additional portion of the nasal wash fluid includes repeating the steps of gargling and sniffing at least two times while at least a portion of the nasal wash fluid remains in the oropharynx.
Inventive Concept 383. The method according to any one of Inventive Concepts 367 and 368, wherein the method further includes, after gargling the mixture and before collecting the specimen sample, while holding the mixture in the oropharynx, snorting an additional portion of the nasal wash fluid from the nasal cavity toward the oropharynx such that the additional portion of the nasal wash fluid is added to the mixture in the oropharynx.
Inventive Concept 384. The method according to Inventive Concept 383, wherein the method further includes, after snorting the additional portion of the nasal wash fluid from the nasal cavity toward the oropharynx, again gargling the mixture.
Inventive Concept 385. The method according to Inventive Concept 383, wherein gargling the mixture and thereafter snorting the additional portion of the nasal wash fluid includes repeating the steps of gargling and snorting at least two times while at least a portion of the nasal wash fluid remains in the oropharynx.
Inventive Concept 386. The method according to any one of Inventive Concepts 367, 368, and 369, further including, before collecting the specimen sample, spraying, via the anterior opening of the oral cavity, the oropharynx with oropharyngeal fluid wash.
Inventive Concept 387. The method according to any one of Inventive Concepts 367, 368, and 369, further including swishing, by the subject, the mixture in the oral cavity.
Inventive Concept 388. The method according to any one of Inventive Concepts 367, 368, and 369, further including, after collecting the specimen sample, testing the specimen sample for the presence of a particulate.
Inventive Concept 389. The method according to Inventive Concept 388, wherein testing the specimen sample for the presence of the particulate includes passing the specimen sample through one or more porous filters to produce a filtrate, and testing the filtrate for the presence of the particulate.
Inventive Concept 390. The method according to Inventive Concept 388, wherein testing the specimen sample for the presence of the particulate includes using spectroscopy to test the specimen sample for the presence of the particulate.
Inventive Concept 391. The method according to Inventive Concept 388, wherein testing the specimen sample for the presence of the particulate includes using microscopy to test the specimen sample for the presence of the particulate.
Inventive Concept 392. The method according to Inventive Concept 388, wherein testing the specimen sample for the presence of the particulate includes using image processing to test the specimen sample for the presence of the particulate.
Inventive Concept 393. The method according to Inventive Concept 388, wherein testing the specimen sample for the presence of the particulate includes using three-dimensional (3D) image sensing to test the specimen sample for the presence of the particulate.
Inventive Concept 394. The method according to Inventive Concept 388, wherein testing the specimen sample for the presence of the particulate includes using artificial intelligence to test the specimen sample for the presence of the particulate.
Inventive Concept 395. The method according to Inventive Concept 388, wherein testing the specimen sample for the presence of the particulate includes using a molecular-based assay to test the specimen sample for the presence of the particulate.
Inventive Concept 396. The method according to Inventive Concept 395, wherein testing the specimen sample for the presence of the particulate includes using nucleic acid amplification to test the specimen sample for the presence of the particulate.
Inventive Concept 397. The method according to Inventive Concept 396, wherein testing the specimen sample for the presence of the particulate includes using polymerase chain reaction (PCR) to test the specimen sample for the presence of the particulate.
Inventive Concept 398. The method according to Inventive Concept 396, wherein testing the specimen sample for the presence of the particulate includes using isothermal amplification (e.g., loop-mediated isothermal amplification (LAMP) or a nicking enzyme amplification (NEAR) reaction) to test the specimen sample for the presence of the particulate.
Inventive Concept 399. The method according to Inventive Concept 388, wherein testing the specimen sample for the presence of the particulate includes performing an immunoassay to test the specimen sample for the presence of the particulate.
Inventive Concept 400. The method according to any one of Inventive Concepts 281-311, further including, before collecting the specimen sample, spraying, via the anterior opening of the oral cavity, the oropharynx with oropharyngeal fluid wash.
Inventive Concept 401. The method according to any one of Inventive Concepts 281-311,

wherein intranasally dispensing the nasal wash fluid into the nasal cavity includes inserting a tubular applicator of a nasal wash fluid dispenser into a nostril of the subject and intranasally dispensing the nasal wash fluid from the tubular applicator, and

wherein the nasal wash fluid dispenser is shaped so as to guide introduction of the tubular applicator into the nostril at a specific orientation with respect to a maxilla of the subject.

Inventive Concept 402. The method according to Inventive Concept 401, wherein the dispensing container is shaped so as to guide the introduction of the tubular applicator into the nostril at the specific orientation with respect to the maxilla.
Inventive Concept 403. The method according to Inventive Concept 401, wherein the nasal wash fluid dispenser includes a housing that is shaped so as to guide the introduction of the tubular applicator into the nostril at the specific orientation with respect to the maxilla.
Inventive Concept 404. The method according to Inventive Concept 401,

wherein the nasal wash fluid dispenser is shaped so as to define a concave surface that faces in a posterior direction and generally conforms to an outer surface of an upper lip of the subject, and

wherein inserting the tubular applicator into the nostril includes pressing the concave surface against the upper lip so as to stabilize the nasal wash fluid dispenser with respect to the maxilla.

Inventive Concept 405. The method according to Inventive Concept 404, wherein the concave surface has a lateral width of between 2.5 and 7 cm.
Inventive Concept 406. The method according to Inventive Concept 404, wherein the concave surface is curved.
Inventive Concept 407. The method according to Inventive Concept 404, wherein the concave surface is concave about an axis and flat in a direction along the axis.
Inventive Concept 408. The method according to Inventive Concept 404, wherein the dispensing container is shaped so as to define the concave surface.
Inventive Concept 409. The method according to Inventive Concept 404, wherein the nasal wash fluid dispenser includes a housing that is shaped so as to define the concave surface.
Inventive Concept 410. The method according to any one of Inventive Concepts 281-311, wherein the method does not include swabbing the nasal cavity.
Inventive Concept 411. The method according to any one of Inventive Concepts 281-311,

wherein the method does not include swabbing the nasopharynx, and

wherein collecting the specimen sample includes swabbing the oropharynx.

Inventive Concept 412. The method according to Inventive Concept 411, wherein the method does not include swabbing any portion of the nasal cavity.
Inventive Concept 413. The method according to Inventive Concept 411, wherein collecting the specimen sample includes swabbing the oropharynx and swabbing one or both anterior nares of the nasal cavity.
Inventive Concept 414. The method according to Inventive Concept 411, wherein collecting the specimen sample includes swabbing the oropharynx and swabbing one or more turbinates of the nasal cavity selected from the group consisting of: a middle turbinate and an inferior turbinate.
Inventive Concept 415. The method according to any one of Inventive Concepts 281-311, wherein the method does not include swabbing the oropharynx and does not include swabbing the nasopharynx.
Inventive Concept 416. The method according to any one of Inventive Concepts 281-311, wherein collecting the specimen sample includes drawing the specimen sample out of the oral cavity via the anterior opening of the oral cavity using an absorbent material.
Inventive Concept 417. The method according to Inventive Concept 416, wherein the absorbent material is located on a tip of a collector shaft, and wherein drawing the specimen sample out of the oral cavity via the anterior opening of the oral cavity using the absorbent material includes inserting the tip of the collector shaft into the oral cavity.
Inventive Concept 418. The method according to Inventive Concept 417, wherein drawing the specimen sample out of the oral cavity via the anterior opening of the oral cavity using the absorbent material includes sucking, by the subject, on the absorbent material.
Inventive Concept 419. The method according to Inventive Concept 416, wherein drawing the specimen sample out of the oral cavity includes contacting one or more portions of the oral cavity with the absorbent material, without swabbing the oropharynx.
Inventive Concept 420. The method according to Inventive Concept 419, wherein the one or more portions of the oral cavity include one or more portions selected from the group consisting of: buccal mucosa, a tongue, gums, and palatal mucosa.
Inventive Concept 421. The method according to any one of Inventive Concepts 281-311, wherein the method does not include swabbing any portion of the subject.

There is yet additionally provided, in accordance with an Inventive Concept 422 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and

thereafter, expressing, by the subject, out of an anterior opening of an oral cavity of the subject, a specimen sample that contains at least a portion of the biological material washed into the oropharynx by the nasal wash fluid.

Inventive Concept 423. The method according to Inventive Concept 422, wherein expressing the specimen sample includes expressing the specimen sample, by the subject, into a collection receptacle.
Inventive Concept 424. The method according to Inventive Concept 422, wherein the biological material includes one or more particulates selected from the group consisting of: a virus, a bacterium, a protozoan, and a fungus.
Inventive Concept 425. The method according to Inventive Concept 422, wherein expressing the specimen sample includes spitting, by the subject, the specimen sample out of the oropharynx via the anterior opening of the oral cavity.
Inventive Concept 426. The method according to Inventive Concept 422, wherein expressing the specimen sample includes expressing the specimen sample out of the oropharynx via the anterior opening of the oral cavity after sensing, by the subject, the nasal wash fluid in the oropharynx.
Inventive Concept 427. The method according to Inventive Concept 426, wherein expressing the specimen sample includes expressing the specimen sample out of the oropharynx via the anterior opening of the oral cavity after tasting, by the subject, the nasal wash fluid in the oropharynx.
Inventive Concept 428. The method according to Inventive Concept 427, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 429. The method according to Inventive Concept 422, wherein intranasally dispensing includes intranasally dispensing, by the subject, the nasal wash fluid into the nasal cavity.
Inventive Concept 430. The method according to Inventive Concept 429, further including, before expressing the specimen sample, tilting back, by the subject, a head of the subject.
Inventive Concept 431. The method according to Inventive Concept 422, further including, while intranasally dispensing the nasal wash fluid, tilting back, by the subject, a head of the subject.
Inventive Concept 432. The method according to Inventive Concept 422, further including, before intranasally dispensing the nasal wash fluid, tilting back, by the subject, a head of the subject.
Inventive Concept 433. The method according to Inventive Concept 422, further including, after intranasally dispensing the nasal wash fluid, tilting a head of the subject farther back than while intranasally dispensing the nasal wash fluid and farther back than a neutral position.
Inventive Concept 434. The method according to Inventive Concept 422, wherein intranasally dispensing the nasal wash fluid includes intranasally dispensing the nasal wash fluid into the nasal cavity while a head of the subject is held in a neutral position or a tilted-forward position.
Inventive Concept 435. The method according to Inventive Concept 422, wherein intranasally dispensing includes intranasally dispensing, by a healthcare worker, the nasal wash fluid into the nasal cavity.
Inventive Concept 436. The method according to Inventive Concept 422, further including waiting for a period of time between (a) concluding intranasally dispensing the nasal wash fluid into the nasal cavity and (b) expressing the specimen sample, the period of time at least 10 minutes.
Inventive Concept 437. The method according to Inventive Concept 422, wherein intranasally dispensing the nasal wash fluid into the nasal cavity includes intranasally dispensing the nasal wash fluid into the nasal cavity while the subject is lying supine.
Inventive Concept 438. The method according to any one of Inventive Concepts 422-437, further including, after intranasally dispensing the nasal wash fluid and before expressing the specimen sample, sniffing, by the subject, the biological material with the nasal wash fluid from the nasal cavity posteriorly toward the nasopharynx.
Inventive Concept 439. The method according to any one of Inventive Concepts 422-437, further including, after intranasally dispensing the nasal wash fluid and before expressing the specimen sample, snorting, by the subject, the biological material with the nasal wash fluid from the nasal cavity toward the nasopharynx.
Inventive Concept 440. The method according to any one of Inventive Concepts 422-437,

wherein the method further includes, before expressing the specimen sample:
orally dispensing oral wash fluid into the oral cavity via the anterior opening of the oral cavity; and
swishing, by the subject, the oral wash fluid in the oral cavity, and

wherein expressing includes expressing, by the subject, out of the anterior opening of the oral cavity, the specimen sample that contains at least a portion of the oral wash fluid and the at least a portion of the biological material washed into the oropharynx with the nasal wash fluid.

Inventive Concept 441. The method according to any one of Inventive Concepts 422-437,

wherein the method further includes, before expressing the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein expressing includes expressing, by the subject, out of the anterior opening of the oral cavity, the specimen sample that contains at least a portion of the mixture and the at least a portion of the biological material washed into the oropharynx with the nasal wash fluid.

Inventive Concept 442. The method according to any one of Inventive Concepts 422-437,

wherein the method further includes, before expressing the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein expressing includes expressing, by the subject, out of the anterior opening of the oral cavity, the specimen sample that contains at least a portion of the mixture and the at least a portion of the biological material washed into the oropharynx with the nasal wash fluid.

Inventive Concept 443. The method according to any one of Inventive Concepts 422-437,

wherein the method further includes, before expressing the specimen sample:
orally dispensing oral wash fluid into the oral cavity via the anterior opening of the oral cavity; and
gargling, by the subject, the oral wash fluid, and

wherein expressing includes expressing, by the subject, out of the anterior opening of the oral cavity, the specimen sample that contains at least a portion of the oral wash fluid and the at least a portion of the biological material washed into the oropharynx with the nasal wash fluid.

Inventive Concept 444. The method according to any one of Inventive Concepts 441, 442, and 443, wherein orally dispensing the oral wash fluid into the oral cavity includes orally dispensing the oral wash fluid into the oral cavity after intranasally dispensing the nasal wash fluid into the nasal cavity.
Inventive Concept 445. The method according to Inventive Concept 444, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 446. The method according to any one of Inventive Concepts 441, 442, and 443, further including swishing, by the subject, the mixture in the oral cavity.
Inventive Concept 447. The method according to any one of Inventive Concepts 422-437, further including, after expressing the specimen sample, testing the specimen sample for the presence of a particulate present in at least the biological material washed into the oropharynx by the nasal wash fluid.
Inventive Concept 448. The method according to any one of Inventive Concepts 422-437, wherein the method does not include swabbing the oropharynx and does not include swabbing the nasopharynx.
Inventive Concept 449. The method according to any one of Inventive Concepts 422-437, wherein the method does not include swabbing any portion of the subject.
Any of Inventive Concepts 422-449 may be performed in combination with any of Inventive Concepts 2-162.

There is also provided, in accordance with an Inventive Concept 450 of the present invention, a method including:

intranasally dispensing a vapor into a nasal cavity of a subject to loosen biological material in the nasal cavity; and

thereafter, collecting a specimen sample that passed out of an anterior opening of an oral cavity of the subject and contains at least a portion of the biological material.

Inventive Concept 451. The method according to Inventive Concept 450, wherein intranasally dispensing the vapor includes inhaling, by the subject, the vapor into the nasal cavity.
Inventive Concept 452. The method according to Inventive Concept 451,

wherein inhaling the vapor into the nasal cavity includes inhaling the vapor into the nasal cavity and into an oropharynx of the subject via a nasopharynx of the subject, such that the vapor washes the biological material into the oropharynx from (a) the nasal cavity, (b) the nasopharynx, or (c) the nasal cavity and the nasopharynx, and

wherein collecting the specimen sample includes collecting the specimen sample that contains at least a portion of the biological material washed into the oropharynx by the vapor.

There is also provided, in accordance with an Inventive Concept 453 of the present invention, a method including:

spraying, via an anterior opening of an oral cavity of a subject, an oropharynx of a subject with oropharyngeal fluid wash; and

thereafter, collecting a specimen sample that passed out of the anterior opening of the oral cavity and contains at least a portion of biological material washed off a pharyngeal wall of the subject into an oropharynx of the subject by the oropharyngeal fluid wash.

Inventive Concept 454. The method according to Inventive Concept 453, wherein the method further includes, before collecting the specimen sample, gargling the oropharyngeal fluid wash by the subject.
Inventive Concept 455. The method according to Inventive Concept 453, wherein the method further includes, before collecting the specimen sample, swishing, by the subject, the oropharyngeal fluid in the oral cavity.
Inventive Concept 456. The method according to Inventive Concept 453, wherein the method does not include gargling the oropharyngeal fluid wash by the subject.
Inventive Concept 457. The method according to Inventive Concept 453,

wherein the method further includes, before collecting the specimen sample:
orally dispensing oral wash fluid into the oral cavity via the anterior opening of the oral cavity such that the oral wash fluid forms a mixture with the oropharyngeal fluid wash in the oropharynx; and
gargling, by the subject, the mixture, and

wherein collecting includes collecting the specimen sample that passed out of the anterior opening of the oral cavity and contains at least a portion of the mixture and the at least a portion of the biological material washed off the pharyngeal wall into the oropharynx by the mixture.

Inventive Concept 458. The method according to Inventive Concept 453, wherein spraying includes spraying while the subject says “ah” or “eh.”
459. The method according to Inventive Concept 453, wherein the biological material includes one or more particulates selected from the group consisting of: a virus, a bacterium, a protozoan, and a fungus.
Inventive Concept 460. The method according to Inventive Concept 453, wherein spraying includes spraying, by the subject, the oropharynx with the oropharyngeal fluid wash.
Inventive Concept 461. The method according to Inventive Concept 453, wherein spraying includes spraying a total volume of 2-15 mL of the oropharyngeal fluid wash.
Inventive Concept 462. The method according to Inventive Concept 453, wherein spraying includes spraying the oropharynx with the oropharyngeal fluid wash in a plurality of pulses.
Inventive Concept 463. The method according to Inventive Concept 462, wherein each of the pulses has a volume of 0.05-5 mL.
Inventive Concept 464. The method according to any one of Inventive Concepts 453-463, wherein collecting the specimen sample includes expressing, by the subject, the specimen sample out of the oropharynx via the anterior opening of the oral cavity.
Inventive Concept 465. The method according to Inventive Concept 464, wherein expressing the specimen sample includes spitting, by the subject, the specimen sample out of the oropharynx via the anterior opening of the oral cavity.
Inventive Concept 466. The method according to Inventive Concept 464, wherein spraying includes spraying, by a healthcare worker, the oropharynx with the oropharyngeal fluid wash.
Inventive Concept 467. The method according to any one of Inventive Concepts 453-463, wherein collecting the specimen sample includes drawing the specimen sample out of the oropharynx via the anterior opening of the oral cavity using an absorbent material, without swabbing the oral cavity or the oropharynx.
Inventive Concept 468. The method according to any one of Inventive Concepts 453-463, wherein spraying includes spraying, by a healthcare worker, the oropharynx with the oropharyngeal fluid wash.
Inventive Concept 469. The method according to Inventive Concept 468, wherein collecting the specimen sample includes receiving the specimen sample by a healthcare worker from the subject.
Inventive Concept 470. The method according to Inventive Concept 469, wherein receiving the specimen sample includes receiving the specimen sample that was expressed out of the oropharynx via the anterior opening of the oral cavity.
Inventive Concept 471. The method according to Inventive Concept 470, wherein receiving the specimen sample includes receiving the specimen sample that was spit out of the oropharynx via the anterior opening of the oral cavity.
Inventive Concept 472. The method according to Inventive Concept 468, wherein collecting the specimen sample includes aspirating the specimen sample out of the oropharynx via the anterior opening of the oral cavity.
Inventive Concept 473. The method according to Inventive Concept 468, wherein collecting the specimen sample includes instructing the subject, by the healthcare worker, to express the specimen sample out of the oropharynx via the anterior opening of the oral cavity.
Inventive Concept 474. The method according to Inventive Concept 473, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 475. The method according to any one of Inventive Concepts 453-463, further including, after collecting the specimen sample, testing the specimen sample for the presence of a particulate present in at least the biological material washed off the pharyngeal wall into the oropharynx by the oropharyngeal fluid wash.
Inventive Concept 476. The method according to Inventive Concept 475, wherein testing the specimen sample for the presence of the particulate includes diagnosing a disease.
Inventive Concept 477. The method according to Inventive Concept 476, wherein diagnosing the disease includes diagnosing an infectious disease.
Inventive Concept 478. The method according to Inventive Concept 475, wherein testing the specimen sample for the presence of the particulate includes concentrating the specimen sample by filtration prior to testing for the presence of the particulate, by passing the specimen sample through one or more filters.
Inventive Concept 479. The method according to Inventive Concept 475, wherein testing the specimen sample for the presence of the particulate includes passing the specimen sample through one or more porous filters to produce a filtrate, and testing the filtrate for the presence of the particulate.
Inventive Concept 480. The method according to Inventive Concept 475, wherein testing the specimen sample for the presence of the particulate includes using spectroscopy to test the specimen sample for the presence of the particulate.
Inventive Concept 481. The method according to Inventive Concept 475, wherein testing the specimen sample for the presence of the particulate includes using microscopy to test the specimen sample for the presence of the particulate.
Inventive Concept 482. The method according to Inventive Concept 475, wherein testing the specimen sample for the presence of the particulate includes using image processing to test the specimen sample for the presence of the particulate.
Inventive Concept 483. The method according to Inventive Concept 475, wherein testing the specimen sample for the presence of the particulate includes using three-dimensional (3D) image sensing to test the specimen sample for the presence of the particulate.
Inventive Concept 484. The method according to Inventive Concept 475, wherein testing the specimen sample for the presence of the particulate includes using artificial intelligence to test the specimen sample for the presence of the particulate.
Inventive Concept 485. The method according to Inventive Concept 475, wherein testing the specimen sample for the presence of the particulate includes using a molecular-based assay to test the specimen sample for the presence of the particulate.
Inventive Concept 486. The method according to Inventive Concept 485, wherein testing the specimen sample for the presence of the particulate includes using nucleic acid amplification to test the specimen sample for the presence of the particulate.
Inventive Concept 487. The method according to Inventive Concept 486, wherein testing the specimen sample for the presence of the particulate includes using polymerase chain reaction (PCR) to test the specimen sample for the presence of the particulate.
Inventive Concept 488. The method according to Inventive Concept 486, wherein testing the specimen sample for the presence of the particulate includes using isothermal amplification (e.g., loop-mediated isothermal amplification (LAMP) or a nicking enzyme amplification (NEAR) reaction) to test the specimen sample for the presence of the particulate.
Inventive Concept 489. The method according to Inventive Concept 475, wherein testing the specimen sample for the presence of the particulate includes performing an immunoassay to test the specimen sample for the presence of the particulate.
Inventive Concept 490. The method according to Inventive Concept 475, wherein the particulate includes one or more particulates selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.
Inventive Concept 491. The method according to Inventive Concept 475, wherein the particulate includes one or more particulates selected from the group consisting of: a respiratory particulate and a particulate contained within droplets from lower airways of the subject.
Inventive Concept 492. The method according to Inventive Concept 475, wherein the particulate includes one or more particulates selected from the group consisting of: a virus, a bacterium, a protozoan, and a fungus.
Inventive Concept 493. The method according to Inventive Concept 475, wherein the particulate includes a virus.
Inventive Concept 494. The method according to Inventive Concept 493, wherein the virus is an Influenza virus.
Inventive Concept 495. The method according to Inventive Concept 493, wherein the virus is a coronavirus.
Inventive Concept 496. The method according to Inventive Concept 495, wherein the coronavirus is Severe acute respiratory syndrome coronavirus 2 (SARS-CoV-2).
Inventive Concept 497. The method according to Inventive Concept 475, wherein the particulate includes a bacterium.
Inventive Concept 498. The method according to Inventive Concept 497, wherein the bacterium is a Streptococcus bacterium.
Inventive Concept 499. The method according to Inventive Concept 475, wherein the particulate includes one or more antigens selected from the group consisting of: a viral antigen, a bacterial antigen, and a fungal antigen.
Inventive Concept 500. The method according to any one of Inventive Concepts 453-463, wherein the oropharyngeal fluid wash includes a non-irritant solution.
Inventive Concept 501. The method according to Inventive Concept 500, wherein the non-irritant solution consists of water.
Inventive Concept 502. The method according to Inventive Concept 500, wherein the non-irritant solution includes saline solution.
Inventive Concept 503. The method according to Inventive Concept 502, wherein the non-irritant saline solution includes phosphate-buffered saline solution.
Inventive Concept 504. The method according to any one of Inventive Concepts 453-463, wherein the method does not include swabbing the oropharynx and does not include swabbing a nasopharynx of the subject.
Inventive Concept 505. The method according to any one of Inventive Concepts 453-463, wherein the method does not include swabbing any portion of the subject.

There is further provided, in accordance with an Inventive Concept 506 of the present invention, a method including:

spraying, via an anterior opening of an oral cavity of a subject, an oropharynx of a subject with oropharyngeal fluid wash; and

thereafter, expressing, by the subject, out of the anterior opening of the oral cavity, a specimen sample that contains at least a portion of biological material washed off a pharyngeal wall of the subject into an oropharynx of the subject by the oropharyngeal fluid wash.

Inventive Concept 507. The method according to Inventive Concept 506, wherein the method further includes, before expressing the specimen sample, gargling the oropharyngeal fluid wash by the subject.
Inventive Concept 508. The method according to Inventive Concept 506, wherein the method further includes, before expressing the specimen sample, swishing, by the subject, the oropharyngeal fluid wash in the oral cavity.
Inventive Concept 509. The method according to Inventive Concept 453, wherein the method does not include gargling the oropharyngeal fluid wash by the subject.
Inventive Concept 510. The method according to Inventive Concept 453,

wherein the method further includes, before expressing the specimen sample:
orally dispensing oral wash fluid into the oral cavity via the anterior opening of the oral cavity such that the oral wash fluid forms a mixture with the oropharyngeal fluid wash in the oropharynx; and
gargling, by the subject, the mixture, and

wherein expressing includes expressing, through the anterior opening the oral cavity, the specimen sample that contains at least a portion of the mixture and the at least a portion of the biological material washed off the pharyngeal wall into the oropharynx by the mixture.

Inventive Concept 511. The method according to Inventive Concept 453, wherein spraying includes spraying while the subject says “ah” or “eh.”
Any of Inventive Concepts 506-511 may be performed in combination with any of Inventive Concepts 453-505.

There is still further provided, in accordance with an Inventive Concept 512 of the present invention, a method including:

collecting, from a subject, a liquid specimen sample that includes an orally-dispensed oral wash fluid and potentially contains a particulate selected from the group consisting of: a virus, a bacterium, a protozoan, a fungus, an antibody, an antigen, and a leukocyte;

passing the liquid specimen sample through one or more porous filters to produce a filtrate; and

testing the filtrate for the presence of the particulate.

Inventive Concept 513. The method according to Inventive Concept 512, wherein collecting the liquid specimen sample includes:

gargling, by the subject, the oral wash fluid to produce the liquid specimen sample that includes the oral wash fluid and potentially contains the particulate; and

thereafter, collecting the liquid specimen sample from the subject.

Inventive Concept 514. The method according to Inventive Concept 512, wherein collecting the liquid specimen sample includes:

swishing, by the subject, the oral wash fluid in an oral cavity of the subject to produce the liquid specimen sample that includes the oral wash fluid and potentially contains the particulate; and

thereafter, collecting the liquid specimen sample from the subject.

Inventive Concept 515. The method according to Inventive Concept 512, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 516. The method according to Inventive Concept 512, wherein testing for the presence of the particulate includes using spectroscopy to test for the presence of the particulate.
Inventive Concept 517. The method according to Inventive Concept 512, wherein testing for the presence of the particulate includes using microscopy to test for the presence of the particulate.
Inventive Concept 518. The method according to Inventive Concept 512, wherein testing for the presence of the particulate includes using image processing to test for the presence of the particulate.
Inventive Concept 519. The method according to Inventive Concept 512, wherein testing for the presence of the particulate includes using three-dimensional (3D) image sensing to test for the presence of the particulate.
Inventive Concept 520. The method according to any one of Inventive Concepts 512-519, wherein testing for the presence of the particulate includes using artificial intelligence to test for the presence of the particulate.
Inventive Concept 521. The method according to any one of Inventive Concepts 512-519, wherein testing for the presence of the particulate includes using a molecular-based assay to test for the presence of the particulate.
Inventive Concept 522. The method according to Inventive Concept 521, wherein testing for the presence of the particulate includes using nucleic acid amplification to test for the presence of the particulate.
Inventive Concept 523. The method according to Inventive Concept 522, wherein testing for the presence of the particulate includes using polymerase chain reaction (PCR) to test for the presence of the particulate.
Inventive Concept 524. The method according to Inventive Concept 522, wherein testing for the presence of the particulate includes using isothermal amplification (e.g., loop-mediated isothermal amplification (LAMP) or a nicking enzyme amplification (NEAR) reaction) to test for the presence of the particulate.
Inventive Concept 525. The method according to any one of Inventive Concepts 512-519, wherein testing for the presence of the particulate includes performing an immunoassay to test for the presence of the particulate.
Inventive Concept 526. The method according to any one of Inventive Concepts 512-519, wherein testing the filtrate for the presence of the particulate includes diagnosing a disease.
Inventive Concept 527. The method according to any one of Inventive Concepts 512-519, wherein the particulate includes a virus.
Inventive Concept 528. The method according to Inventive Concept 527, wherein the virus is an Influenza virus.
Inventive Concept 529. The method according to Inventive Concept 527, wherein the virus is a coronavirus.
Inventive Concept 530. The method according to Inventive Concept 529, wherein the coronavirus is Severe acute respiratory syndrome coronavirus 2 (SARS-CoV-2).
Inventive Concept 531. The method according to any one of Inventive Concepts 512-519, wherein the one or more porous filters include at least one filter having an average absolute pore size of between 0.2 and 3 microns.
Inventive Concept 532. The method according to Inventive Concept 531, wherein the average absolute pore size is between 0.2 and 2 microns.
Inventive Concept 533. The method according to Inventive Concept 532, wherein the average absolute pore size is between 1 and 2 microns.
Inventive Concept 534. The method according to any one of Inventive Concepts 512-519, wherein the one or more porous filters include at least one filter having an average nominal pore size of between 0.2 and 3 microns.
Inventive Concept 535. The method according to Inventive Concept 534, wherein the average nominal pore size is between 0.2 and 2 microns.
Inventive Concept 536. The method according to Inventive Concept 535, wherein the average nominal pore size is between 1 and 2 microns.
Inventive Concept 537. The method according to any one of Inventive Concepts 512-519, wherein the method does not include centrifuging the liquid specimen sample before passing the liquid specimen sample through the one or more porous filters.
Inventive Concept 538. The method according to any one of Inventive Concepts 512-519, wherein the method does not include centrifuging the filtrate before testing the filtrate for the presence of the particulate.
Inventive Concept 539. The method according to any one of Inventive Concepts 512-519, wherein the method does not include culturing the liquid specimen sample before passing the liquid specimen sample through the one or more porous filters.
Inventive Concept 540. The method according to any one of Inventive Concepts 512-519, wherein the method does not include culturing the filtrate before testing the filtrate for the presence of the particulate.

There is additionally provided, in accordance with an Inventive Concept 541 of the present invention, a method including:

collecting, from a subject, a liquid specimen sample that includes an orally-dispensed oral wash fluid and potentially contains a particulate selected from the group consisting of: a virus, a bacterium, a protozoan, a fungus, an antibody, an antigen, and a leukocyte; and

testing the liquid specimen sample for the presence of the particulate using spectroscopy.

There is yet additionally provided, in accordance with an Inventive Concept 542 of the present invention, a method including:

collecting, from a subject, a liquid specimen sample that includes an orally-dispensed oral wash fluid and potentially contains a particulate selected from the group consisting of: a virus, a bacterium, a protozoan, and fungus, an antibody, an antigen, and a leukocyte; and

testing the liquid specimen sample for the presence of the particulate using microscopy.

Inventive Concept 543. The method according to Inventive Concept 542, wherein test testing the liquid specimen sample for the presence of the particulate using microscopy includes testing the liquid specimen sample for the presence of the particulate using automated digital microscopy.

There is also provided, in accordance with an Inventive Concept 544 of the present invention, a method including:

collecting, from a subject, a liquid specimen sample that includes an orally-dispensed oral wash fluid and potentially contains a particulate selected from the group consisting of: a virus, a bacterium, a protozoan, and fungus, an antibody, an antigen, and a leukocyte; and

testing the liquid specimen sample for the presence of the particulate using image processing.

There is further provided, in accordance with an Inventive Concept 545 of the present invention, a method including:

collecting, from a subject, a liquid specimen sample that includes an orally-dispensed oral wash fluid and potentially contains a particulate selected from the group consisting of: a virus, a bacterium, a protozoan, and fungus, an antibody, an antigen, and a leukocyte; and

testing the liquid specimen sample for the presence of the particulate using three-dimensional (3D) image sensing.

There is still further provided, in accordance with an Inventive Concept 546 of the present invention, a method including:

collecting, from a subject, a liquid specimen sample that includes an orally-dispensed oral wash fluid and potentially contains a particulate selected from the group consisting of: a virus, a bacterium, a protozoan, and fungus, an antibody, an antigen, and a leukocyte; and

testing the liquid specimen sample for the presence of the particulate using artificial intelligence.

Inventive Concept 547. The method according to any one of Inventive Concepts 541, 542, 544, and 545, wherein collecting the liquid specimen sample includes:

gargling, by the subject, the oral wash fluid to produce the liquid specimen sample that includes the oral wash fluid and potentially contains the particulate; and

thereafter, collecting the liquid specimen sample from the subject.

Inventive Concept 548. The method according to any one of Inventive Concepts 541, 542, 544, and 545, wherein collecting the liquid specimen sample includes:

swishing, by the subject, the oral wash fluid in an oral cavity of the subject to produce the liquid specimen sample that includes the oral wash fluid and potentially contains the particulate; and

thereafter, collecting the liquid specimen sample from the subject.

Inventive Concept 549. The method according to any one of Inventive Concepts 541, 542, 544, and 545, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 550. The method according to any one of Inventive Concepts 541, 542, 544, and 545, wherein testing for the presence of the particulate includes using artificial intelligence to test for the presence of the particulate.
Inventive Concept 551. The method according to any one of Inventive Concepts 541, 542, 544, 545 and 546, wherein testing for the presence of the particulate includes diagnosing a disease.
Inventive Concept 552. The method according to any one of Inventive Concepts 541, 542, 544, 545 and 546, wherein the particulate includes a virus.
Inventive Concept 553. The method according to Inventive Concept 552, wherein the virus is an Influenza virus.
Inventive Concept 554. The method according to Inventive Concept 552, wherein the virus is a coronavirus.
Inventive Concept 555. The method according to Inventive Concept 554, wherein the coronavirus is Severe acute respiratory syndrome coronavirus 2 (SARS-CoV-2).
Inventive Concept 556. The method according to any one of Inventive Concepts 541, 542, 544, 545 and 546, wherein testing the liquid specimen sample for the presence of the particulate includes passing the liquid specimen sample through one or more porous filters to produce a filtrate, and testing the filtrate for the presence of the particulate.
Inventive Concept 557. The method according to any one of Inventive Concepts 541, 542, 544, 545 and 546, wherein testing the liquid specimen sample for the presence of the particulate includes concentrating the liquid specimen sample by filtration prior to testing for the presence of the particulate, by passing the liquid specimen sample through one or more filters.
Inventive Concept 558. The method according to any one of Inventive Concepts 541, 542, 544, 545 and 546, wherein the method does not include centrifuging the liquid specimen sample before testing for the presence of the particulate.
Inventive Concept 559. The method according to any one of Inventive Concepts 541, 542, 544, 545 and 546, wherein the method does not include culturing the liquid specimen sample before testing for the presence of the particulate.

There is additionally provided, in accordance with an Inventive Concept 560 of the present invention, a method including:

collecting, from a subject, a liquid specimen sample that includes saliva and an orally-dispensed oral wash fluid;

passing the liquid specimen sample through a filter to trap at least some of the saliva, the filter having a nominal pore size of between 30 microns and 1.5 mm, the nominal pore size representative of a minimum size of spherical particles necessary for the filter to retain 85% of the spherical particles when H2O containing the spherical particles is passed through the filter at 20 degrees C. under pressure supplied by a 10 cm water column; and

testing for the presence of a virus in the saliva trapped by the filter.

There is yet additionally provided, in accordance with an Inventive Concept 561 of the present invention, a method including:

collecting, from a subject, a liquid specimen sample;

passing the liquid specimen sample through a filter having a nominal pore size of between 30 microns and 1.5 mm, the nominal pore size representative of a minimum size of spherical particles necessary for the filter to retain 85% of the spherical particles when H2O containing the spherical particles is passed through the filter at 20 degrees C. under pressure supplied by a 10 cm water column; and

testing for the presence of a virus that adhered to the filter during the passing of the liquid specimen sample through the filter.

Inventive Concept 562. The method according to any one of Inventive Concepts 560 and 561, wherein the nominal pore size is at least 40 microns.
Inventive Concept 563. The method according to Inventive Concept 562, wherein the nominal pore size is at least 50 microns.
Inventive Concept 564. The method according to Inventive Concept 563, wherein the nominal pore size is at least 60 microns.
Inventive Concept 565. The method according to Inventive Concept 564, wherein the nominal pore size is at least 100 microns.
Inventive Concept 566. The method according to Inventive Concept 565, wherein the nominal pore size is at least 120 microns.
Inventive Concept 567. The method according to Inventive Concept 566, wherein the nominal pore size is at least 150 microns.
Inventive Concept 568. The method according to Inventive Concept 567, wherein the nominal pore size is at least 200 microns.
Inventive Concept 569. The method according to Inventive Concept 568, wherein the nominal pore size is at least 500 microns.
Inventive Concept 570. The method according to any one of Inventive Concepts 560 and 561, wherein the nominal pore size is less than 1 mm.
Inventive Concept 571. The method according to Inventive Concept 570, wherein the nominal pore size is less than 750 microns.
Inventive Concept 572. The method according to Inventive Concept 571, wherein the nominal pore size less than 500 microns.
Inventive Concept 573. The method according to Inventive Concept 572, wherein the nominal pore size less than 250 microns.
Inventive Concept 574. The method according to Inventive Concept 573, wherein the nominal pore size less than 200 microns.
Inventive Concept 575. The method according to any one of Inventive Concepts 560 and 561, wherein the nominal pore size is between 50 and 200 microns.
Inventive Concept 576. The method according to Inventive Concept 575, wherein the nominal pore size is between 50 and 150 microns.
Inventive Concept 577. The method according to Inventive Concept 576, wherein the nominal pore size is between 100 and 150 microns.
Inventive Concept 578. The method according to any one of Inventive Concepts 560 and 561, wherein the method does not include centrifuging the liquid specimen sample.
Inventive Concept 579. The method according to any one of Inventive Concepts 560 and 561, wherein the method does not include adding to the liquid specimen sample a concentration agent that binds to the virus.
Inventive Concept 580. The method according to any one of Inventive Concepts 560 and 561, wherein the method does not include centrifuging the liquid specimen sample and the method does not include adding to the liquid specimen sample a concentration agent that binds to the virus.
Inventive Concept 581. The method according to Inventive Concept 560,

wherein the filter is one of a plurality of filters arranged in a series of filters, the filters having respective nominal pore sizes, all of which are between 30 microns and 1.5 mm,

wherein passing the liquid specimen sample through the filter includes passing the liquid specimen sample through the series of filters, without passing the liquid specimen sample through any other filters, and

wherein testing for the presence of the virus in the saliva includes testing for the presence of the virus in the saliva trapped by at least one of the plurality of filters.

Inventive Concept 582. The method according to Inventive Concept 581, wherein testing for the presence of the virus in the saliva includes testing for the presence of the virus in the saliva trapped by at least two of the plurality of filters.
Inventive Concept 583. The method according to Inventive Concept 561,

wherein the filter is one of a plurality of filters arranged in a series of filters, the filters having respective nominal pore sizes, all of which are between 30 microns and 1.5 mm,

wherein passing the liquid specimen sample through the filter includes passing the liquid specimen sample through the series of filters, without passing the liquid specimen sample through any other filters, and

wherein testing for the presence of the virus that adhered to the filter includes testing for the presence of the virus that adhered to at least one of the plurality of filters.

Inventive Concept 584. The method according to Inventive Concept 583, wherein testing for the presence of the virus that adhered to the at least one of the filters includes testing for the presence of the virus that adhered to at least two of the plurality of filters.
Inventive Concept 585. The method according to Inventive Concept 560,

wherein the filter is a first filter, wherein passing the liquid specimen sample through the filter includes passing the liquid specimen sample through the first filter to produce a filtrate, and wherein testing for the presence of the virus in the saliva trapped by the filter includes testing for the presence of the virus in the saliva trapped by the first filter, and

wherein the method further includes:
passing the filtrate through a second filter to trap at least some of the saliva, and
testing for the presence of the virus in the saliva trapped by the second filter.
   Inventive Concept 586. The method according to Inventive Concept 585, wherein a nominal pore size of the second filter equals the nominal pore size of the first filter.
   Inventive Concept 587. The method according to Inventive Concept 585, wherein a nominal pore size of the second filter is between 0.03 and 21 microns.
   Inventive Concept 588. The method according to Inventive Concept 587, wherein the nominal pore size of the second filter is between 0.8 and 1.5 microns.
   Inventive Concept 589. The method according to Inventive Concept 560, wherein passing the liquid specimen sample through the filter includes passing the liquid specimen sample through the filter to trap at least some of the saliva by size-based filtration.
   Inventive Concept 590. The method according to Inventive Concept 560, wherein passing the liquid specimen sample through the filter includes passing the liquid specimen sample through the filter to trap at least some of the saliva by adhesion of the saliva to the filter.
   Inventive Concept 591. The method according to Inventive Concept 590, wherein passing the liquid specimen sample through the filter to trap at least some of the saliva by adhesion of the saliva to the filter by interactions that are not specific to the virus.
   Inventive Concept 592. The method according to any one of Inventive Concepts 560 and 561, wherein the filter is a depth filter.
   Inventive Concept 593. The method according to any one of Inventive Concepts 560 and 561, wherein the filter includes fibers.
   Inventive Concept 594. The method according to Inventive Concept 593, wherein the filter includes crisscrossing fibers.
   Inventive Concept 595. The method according to any one of Inventive Concepts 560 and 561, wherein the filter includes a mesh filter.
   Inventive Concept 596. The method according to Inventive Concept 595, wherein the mesh filter includes woven mesh filter.
   Inventive Concept 597. The method according to Inventive Concept 595, wherein the mesh filter includes a non-woven mesh filter.
   Inventive Concept 598. The method according to any one of Inventive Concepts 560 and 561, wherein the filter includes a screen.
   Inventive Concept 599. The method according to Inventive Concept 598, wherein the screen includes a woven screen.
   Inventive Concept 600. The method according to Inventive Concept 598, wherein the screen includes a non-woven screen.
   Inventive Concept 601. The method according to any one of Inventive Concepts 560 and 561, wherein the filter includes a polymer.
   Inventive Concept 602. The method according to Inventive Concept 601, wherein the polymer includes a polyester.
   Inventive Concept 603. The method according to any one of Inventive Concepts 560 and 561, wherein passing the liquid specimen sample through the filter includes passing the liquid specimen sample through exactly one filter.
   Inventive Concept 604. The method according to Inventive Concept 560, wherein testing for the presence of the virus in the saliva trapped by the filter includes testing the filter for the presence of the virus in the saliva.
   Inventive Concept 605. The method according to Inventive Concept 561, wherein testing for the presence of the virus that adhered to the filter includes testing the filter for the presence of the virus that adhered to the filter.
   Inventive Concept 606. The method according to Inventive Concept 560, wherein testing for the presence of the virus in the saliva trapped by the filter includes inserting the filter into a testing machine.
   Inventive Concept 607. The method according to Inventive Concept 561, wherein testing for the presence of the virus that adhered to the filter includes inserting the filter into a testing machine.
   Inventive Concept 608. The method according to any one of Inventive Concepts 606 and 607, wherein the testing machine is selected from the group consisting of: a thermal cycler and an isothermal amplification instrument (e.g., loop-mediated isothermal amplification (LAMP) or a nicking enzyme amplification (NEAR) reaction).
   Inventive Concept 609. The method according to any one of Inventive Concepts 560 and 561, wherein testing for the presence of the virus includes performing an immunoassay.
   Inventive Concept 610. The method according to Inventive Concept 609, wherein performing the immunoassay includes performing a lateral flow immunoassay.
   Inventive Concept 611. The method according to Inventive Concept 610, wherein performing the immunoassay includes performing a chromatographic digital immunoassay.
   Inventive Concept 612. The method according to Inventive Concept 560, wherein testing for the presence of the virus in the saliva trapped by the filter includes, after finishing passing the liquid specimen sample through the filter, bringing the filter, the saliva, and a liquid medium into contact, and testing the liquid medium for the presence of the virus.
   Inventive Concept 613. The method according to Inventive Concept 561, wherein testing for the presence of the virus that adhered to the filter includes, after finishing passing the liquid specimen sample through the filter, bringing the filter and a liquid medium into contact, and testing the liquid medium for the presence of the virus.
   Inventive Concept 614. The method according to any one of Inventive Concepts 612 and 613, wherein testing the liquid medium for the presence of the virus includes separating the liquid medium from the filter before testing the liquid medium for the presence of the virus.
   Inventive Concept 615. The method according to any one of Inventive Concepts 612 and 613, wherein testing for the presence of the virus trapped by the filter includes agitating the filter in the liquid medium.
   Inventive Concept 616. The method according to Inventive Concept 615, wherein agitating the filter in the liquid medium includes mixing the filter in the liquid medium.
   Inventive Concept 617. The method according to any one of Inventive Concepts 612 and 613, wherein testing for the presence of the virus trapped by the filter includes transferring at least a portion of the liquid medium into a testing machine.
   Inventive Concept 618. The method according to Inventive Concept 617, wherein the testing machine is selected from the group consisting of: a thermal cycler and an isothermal amplification instrument (e.g., loop-mediated isothermal amplification (LAMP) or a nicking enzyme amplification (NEAR) reaction).
   Inventive Concept 619. The method according to any one of Inventive Concepts 612 and 613, wherein the liquid medium is selected from the group consisting of: a transport medium, a culture medium, a purification agent, a stabilizing agent, a lysing buffer, and an extraction agent.
   Inventive Concept 620. The method according to any one of Inventive Concepts 560 and 561, wherein testing for the presence of the virus includes using spectroscopy.
   Inventive Concept 621. The method according to any one of Inventive Concepts 560 and 561, wherein testing for the presence of the virus includes using microscopy.
   Inventive Concept 622. The method according to any one of Inventive Concepts 560 and 561, wherein testing for the presence of the virus includes using image processing.
   Inventive Concept 623. The method according to any one of Inventive Concepts 560 and 561, wherein testing for the presence of the virus includes using three-dimensional (3D) image sensing.
   Inventive Concept 624. The method according to any one of Inventive Concepts 560 and 561, wherein testing for the presence of the virus includes using artificial intelligence.
   Inventive Concept 625. The method according to any one of Inventive Concepts 560 and 561, wherein testing for the presence of the virus includes using a molecular-based assay.
   Inventive Concept 626. The method according to Inventive Concept 625, wherein testing for the presence of the virus includes using nucleic acid amplification.
   Inventive Concept 627. The method according to Inventive Concept 626, wherein testing for the presence of the virus includes using polymerase chain reaction (PCR).
   Inventive Concept 628. The method according to Inventive Concept 626, wherein testing for the presence of the virus includes using isothermal amplification (e.g., loop-mediated isothermal amplification (LAMP) or a nicking enzyme amplification (NEAR) reaction).
   Inventive Concept 629. The method according to any one of Inventive Concepts 560 and 561, wherein testing for the presence of the virus includes performing an immunoassay.
   Inventive Concept 630. The method according to Inventive Concept 560, further including, before testing for the presence of the virus in the saliva trapped by the filter, transporting the filter while the filter is at least partially immersed in a liquid medium.
   Inventive Concept 631. The method according to Inventive Concept 630, wherein testing for the presence of the virus in the saliva trapped by the filter includes inserting at least a portion of the liquid medium into a thermal cycler.
   Inventive Concept 632. The method according to Inventive Concept 561, further including, before testing for the presence of the virus that adhered to the filter, transporting the filter while the filter is at least partially immersed in a liquid medium.
   Inventive Concept 633. The method according to Inventive Concept 632, wherein testing for the presence of the virus that adhered to the filter includes inserting at least a portion of the liquid medium into a thermal cycler.
   Inventive Concept 634. The method according to any one of Inventive Concepts 560 and 561, wherein collecting the liquid specimen sample includes receiving the liquid specimen sample by a healthcare worker from the subject.
   Inventive Concept 635. The method according to any one of Inventive Concepts 560 and 561, wherein the method does not include swabbing a nasal cavity of the subject.
   Inventive Concept 636. The method according to any one of Inventive Concepts 560 and 561, wherein the method does not include swabbing a pharynx of the subject.
   Inventive Concept 637. The method according to any one of Inventive Concepts 560 and 561, wherein the method does not include swabbing a nasal cavity of the subject and does not include swabbing a pharynx of the subject.
   Inventive Concept 638. The method according to any one of Inventive Concepts 560 and 561, wherein the method does not include swabbing any portion of the subject.
   Inventive Concept 639. The method according to any one of Inventive Concepts 560 and 561, wherein collecting the liquid specimen sample includes collecting the liquid specimen sample that was expressed via an anterior opening of an oral cavity of the subject.
   Inventive Concept 640. The method according to any one of Inventive Concepts 560 and 561, wherein collecting the liquid specimen sample includes aspirating the liquid specimen sample via an anterior opening of an oral cavity of the subject.
   Inventive Concept 641. The method according to Inventive Concept 560, wherein collecting the liquid specimen sample includes:

gargling, by the subject, the oral wash fluid to produce the liquid specimen sample that includes the oral wash fluid and the saliva; and

thereafter, collecting the liquid specimen sample from the subject.

Inventive Concept 642. The method according to Inventive Concept 560, wherein collecting the liquid specimen sample includes:

gargling, by the subject, the oral wash fluid to produce the liquid specimen sample that includes the oral wash fluid and the saliva; and

thereafter, expressing, by the subject, out of an anterior opening of an oral cavity of the subject, the liquid specimen sample.

Inventive Concept 643. The method according to Inventive Concept 560, wherein collecting the liquid specimen sample includes:

swishing, by the subject, the oral wash fluid in an oral cavity of the subject to produce the liquid specimen sample that includes the oral wash fluid and the saliva; and

thereafter, collecting the liquid specimen sample from the subject.

Inventive Concept 644. The method according to Inventive Concept 560, wherein collecting the liquid specimen sample includes:

swishing, by the subject, the oral wash fluid in an oral cavity of the subject to produce the liquid specimen sample that includes the oral wash fluid and the saliva; and

thereafter, expressing, by the subject, out of an anterior opening of the oral cavity, the liquid specimen sample.

Inventive Concept 645. The method according to any one of Inventive Concepts 560 and 561, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 646. The method according to Inventive Concept 560, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, collecting the liquid specimen sample that passed out of an anterior opening of an oral cavity of the subject and includes the oral wash fluid, the nasal wash fluid, and at least a portion of the biological material washed into the oropharynx by the nasal wash fluid.

Inventive Concept 647. The method according to Inventive Concept 646, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

collecting the liquid specimen sample that passed out of the anterior opening of the oral cavity.

Inventive Concept 648. The method according to Inventive Concept 646, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

collecting the liquid specimen sample that passed out of the anterior opening of the oral cavity.

Inventive Concept 649. The method according to Inventive Concept 560, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, expressing, by the subject, out of an anterior opening of an oral cavity of the subject, the liquid specimen sample that includes the oral wash fluid, the nasal wash fluid, and at least a portion of the biological material washed into the oropharynx by the nasal wash fluid.

Inventive Concept 650. The method according to Inventive Concept 649, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

expressing, by the subject, the liquid specimen sample out of the anterior opening of the oral cavity.

Inventive Concept 651. The method according to Inventive Concept 649, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

expressing, by the subject, the liquid specimen sample out of the anterior opening of the oral cavity.

Inventive Concept 652. The method according to any one of Inventive Concepts 646 and 649, wherein intranasally dispensing includes intranasally dispensing, by the subject, the nasal wash fluid into the nasal cavity.
Inventive Concept 653. The method according to any one of Inventive Concepts 646 and 649, wherein intranasally dispensing includes intranasally dispensing, by a healthcare worker, the nasal wash fluid into the nasal cavity.
Inventive Concept 653b. The method according to any one of Inventive Concepts 560 and 561, wherein the virus is an Influenza virus, and wherein testing for the presence of the virus includes testing for the presence of the Influenza virus.
Inventive Concept 654. The method according to any one of Inventive Concepts 560 and 561, wherein the virus is a coronavirus, and wherein testing for the presence of the virus includes testing for the presence of the coronavirus.
Inventive Concept 655. The method according to Inventive Concept 654, wherein the coronavirus is Severe acute respiratory syndrome coronavirus 2 (SARS-CoV-2).
Inventive Concept 656. The method according to any one of Inventive Concepts 560 and 561, wherein the virus is an Influenza virus, and wherein testing for the presence of the virus includes testing for the presence of the Influenza virus.
Inventive Concept 657. The method according to Inventive Concept 560,

wherein the subject is one of a plurality of subjects, and wherein the liquid specimen sample is one of a plurality of liquid specimen samples,

wherein collecting the liquid specimen sample from the subject includes collecting, from the plurality of subjects, the plurality of liquid specimen samples, respectively, which include respective oral wash fluids and saliva, and

wherein passing the liquid specimen sample through the filter includes:
combining the plurality of liquid specimen samples; and
passing the combined plurality of liquid specimen samples through the filter.
   Inventive Concept 658. The method according to Inventive Concept 657, wherein the plurality of liquid specimen samples includes at least five liquid specimen samples from at least five subjects, respectively.
   Inventive Concept 659. The method according to Inventive Concept 658, wherein the plurality of liquid specimen samples includes at least ten liquid specimen samples from at least ten subjects, respectively.
   Inventive Concept 660. The method according to Inventive Concept 657, wherein the plurality of liquid specimen samples have a combined volume of at least 25 cc, and wherein collecting, from the plurality of subjects, the plurality of liquid specimen samples, respectively, includes collecting, from the plurality of subjects, the plurality of liquid specimen samples, respectively, having the combined volume.
   Inventive Concept 661. The method according to Inventive Concept 660, wherein the combined volume is at least 50 cc.
   Inventive Concept 662. The method according to Inventive Concept 661, wherein the combined volume is at least 100 cc.
   Inventive Concept 663. The method according to Inventive Concept 657, wherein combining the plurality of liquid specimen samples includes mixing together the plurality of liquid specimen samples.

There is also provided, in accordance with an Inventive Concept 664 of the present invention, a method including:

collecting, from a subject, a liquid specimen sample that includes saliva and an orally-dispensed oral wash fluid;

passing the liquid specimen sample through a filter to trap at least some of the saliva, the filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and

testing for the presence of a virus in the saliva trapped by the filter.

There is further provided, in accordance with an Inventive Concept 665 of the present invention, a method including:

collecting, from a subject, a liquid specimen sample;

passing the liquid specimen sample through a filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and

testing for the presence of a virus that adhered to the filter during the passing of the liquid specimen sample through the filter.

Inventive Concept 666. The method according to any one of Inventive Concepts 664 and 653 wherein the bubble-point nominal pore size is at least 40 microns.
Inventive Concept 667. The method according to Inventive Concept 666, wherein the bubble-point nominal pore size is at least 50 microns.
Inventive Concept 668. The method according to Inventive Concept 667, wherein the bubble-point nominal pore size is at least 60 microns.
Inventive Concept 669. The method according to Inventive Concept 668, wherein the bubble-point nominal pore size is at least 100 microns.
Inventive Concept 670. The method according to Inventive Concept 669, wherein the bubble-point nominal pore size is at least 120 microns.
Inventive Concept 671. The method according to any one of Inventive Concepts 664 and 665, wherein the bubble-point nominal pore size less than 300 microns.
Inventive Concept 672. The method according to Inventive Concept 671, wherein the bubble-point nominal pore size less than 200 microns.
Inventive Concept 673. The method according to any one of Inventive Concepts 664 and 665, wherein the bubble-point nominal pore size is between 50 and 200 microns.
Inventive Concept 674. The method according to Inventive Concept 673, wherein the bubble-point nominal pore size is between 50 and 150 microns.
Inventive Concept 675. The method according to Inventive Concept 674, wherein the bubble-point nominal pore size is between 100 and 150 microns.
Inventive Concept 676. The method according to any one of Inventive Concepts 664 and 665, wherein the method does not include centrifuging the liquid specimen sample.
Inventive Concept 677. The method according to any one of Inventive Concepts 664 and 665, wherein the method does not include adding to the liquid specimen sample a concentration agent that binds to the virus.
Inventive Concept 678. The method according to any one of Inventive Concepts 664 and 665, wherein the method does not include centrifuging the liquid specimen sample and the method does not include adding to the liquid specimen sample a concentration agent that binds to the virus.
Inventive Concept 679. The method according to Inventive Concept 664,

wherein the filter is one of a plurality of filters arranged in a series of filters, the filters having respective bubble-point nominal pore sizes, all of which are between 30 microns and 1.5 mm,

wherein passing the liquid specimen sample through the filter includes passing the liquid specimen sample through the series of filters, without passing the liquid specimen sample through any other filters, and

wherein testing for the presence of the virus in the saliva includes testing for the presence of the virus in the saliva trapped by at least one of the plurality of filters.

Inventive Concept 680. The method according to Inventive Concept 679, wherein testing for the presence of the virus in the saliva includes testing for the presence of the virus in the saliva trapped by at least two of the plurality of filters.
Inventive Concept 681. The method according to Inventive Concept 665,

wherein the filter is one of a plurality of filters arranged in a series of filters, the filters having respective bubble-point nominal pore sizes, all of which are between 30 microns and 1.5 mm, wherein passing the liquid specimen sample through the filter includes passing the liquid specimen sample through the series of filters, without passing the liquid specimen sample through any other filters, and wherein testing for the presence of the virus that adhered to the filter includes testing for the presence of the virus that adhered to at least one of the plurality of filters.

Inventive Concept 682. The method according to Inventive Concept 681, wherein testing for the presence of the virus that adhered to the at least one of the filters includes testing for the presence of the virus that adhered to at least two of the plurality of filters.
Inventive Concept 683. The method according to Inventive Concept 664,

wherein the filter is a first filter, wherein passing the liquid specimen sample through the filter includes passing the liquid specimen sample through the first filter to produce a filtrate, and wherein testing for the presence of the virus in the saliva trapped by the filter includes testing for the presence of the virus in the saliva trapped by the first filter, and

wherein the method further includes:
passing the filtrate through a second filter to trap at least some of the saliva, and
testing for the presence of the virus in the saliva trapped by the second filter.
   Inventive Concept 684. The method according to Inventive Concept 683, wherein a bubble-point nominal pore size of the second filter equals the bubble-point nominal pore size of the first filter.
   Inventive Concept 685. The method according to Inventive Concept 683, wherein a bubble-point nominal pore size of the second filter is between 0.03 and 21 microns.
   Inventive Concept 686. The method according to Inventive Concept 685, wherein the bubble-point nominal pore size of the second filter is between 0.8 and 1.5 microns.
   Inventive Concept 687. The method according to Inventive Concept 664, wherein passing the liquid specimen sample through the filter includes passing the liquid specimen sample through the filter to trap at least some of the saliva by size-based filtration.
   Inventive Concept 688. The method according to Inventive Concept 664, wherein passing the liquid specimen sample through the filter includes passing the liquid specimen sample through the filter to trap at least some of the saliva by adhesion of the saliva to the filter.
   Inventive Concept 689. The method according to Inventive Concept 688, wherein passing the liquid specimen sample through the filter to trap at least some of the saliva by adhesion of the saliva to the filter by interactions that are not specific to the virus.
   Inventive Concept 690. The method according to any one of Inventive Concepts 664 and 665, wherein the filter is a depth filter.
   Inventive Concept 691. The method according to any one of Inventive Concepts 664 and 665, wherein the filter includes fibers.
   Inventive Concept 692. The method according to Inventive Concept 691, wherein the filter includes crisscrossing fibers.
   Inventive Concept 693. The method according to any one of Inventive Concepts 664 and 665, wherein the filter includes a mesh filter.
   Inventive Concept 694. The method according to Inventive Concept 693, wherein the mesh filter includes a woven mesh filter.
   Inventive Concept 695. The method according to Inventive Concept 693, wherein the mesh filter includes a non-woven mesh filter.
   Inventive Concept 696. The method according to any one of Inventive Concepts 664 and 665, wherein the filter includes a screen.
   Inventive Concept 697. The method according to Inventive Concept 696, wherein the screen includes a woven screen.
   Inventive Concept 698. The method according to Inventive Concept 696, wherein the screen includes a non-woven screen.
   Inventive Concept 699. The method according to any one of Inventive Concepts 664 and 665, wherein the filter includes a polymer.
   Inventive Concept 700. The method according to Inventive Concept 699, wherein the polymer includes a polyester.
   Inventive Concept 701. The method according to any one of Inventive Concepts 664 and 665, wherein passing the liquid specimen sample through the filter includes passing the liquid specimen sample through exactly one filter.
   Inventive Concept 702. The method according to Inventive Concept 664, wherein testing for the presence of the virus in the saliva trapped by the filter includes testing the filter for the presence of the virus in the saliva.
   Inventive Concept 703. The method according to Inventive Concept 665, wherein testing for the presence of the virus that adhered to the filter includes testing the filter for the presence of the virus that adhered to the filter.
   Inventive Concept 704. The method according to Inventive Concept 664, wherein testing for the presence of the virus in the saliva trapped by the filter includes inserting the filter into a testing machine.
   Inventive Concept 705. The method according to Inventive Concept 665, wherein testing for the presence of the virus that adhered to the filter includes inserting the filter into a testing machine.
   Inventive Concept 706. The method according to any one of Inventive Concepts 704 and 705, wherein the testing machine is selected from the group consisting of: a thermal cycler and an isothermal amplification instrument (e.g., loop-mediated isothermal amplification (LAMP) or a nicking enzyme amplification (NEAR) reaction).
   Inventive Concept 707. The method according to any one of Inventive Concepts 664 and 665, wherein testing for the presence of the virus includes performing an immunoassay.
   Inventive Concept 708. The method according to Inventive Concept 707, wherein performing the immunoassay includes performing a lateral flow immunoassay.
   Inventive Concept 709. The method according to Inventive Concept 708, wherein performing the immunoassay includes performing a chromatographic digital immunoassay.
   Inventive Concept 710. The method according to Inventive Concept 664, wherein testing for the presence of the virus in the saliva trapped by the filter includes, after finishing passing the liquid specimen sample through the filter, bringing the filter, the saliva, and a liquid medium into contact, and testing the liquid medium for the presence of the virus.
   Inventive Concept 711. The method according to Inventive Concept 665, wherein testing for the presence of the virus that adhered to the filter includes, after finishing passing the liquid specimen sample through the filter, bringing the filter and a liquid medium into contact, and testing the liquid medium for the presence of the virus.
   Inventive Concept 712. The method according to any one of Inventive Concepts 664 and 665, wherein the testing machine is selected from the group consisting of: a thermal cycler and an isothermal amplification instrument (e.g., loop-mediated isothermal amplification (LAMP) or a nicking enzyme amplification (NEAR) reaction).
   Inventive Concept 713. The method according to any one of Inventive Concepts 664 and 665, wherein testing the liquid medium for the presence of the virus includes separating the liquid medium from the filter before testing the liquid medium for the presence of the virus.
   Inventive Concept 714. The method according to any one of Inventive Concepts 664 and 665, wherein testing for the presence of the virus trapped by the filter includes agitating the filter in the liquid medium.
   Inventive Concept 715. The method according to Inventive Concept 714, wherein agitating the filter in the liquid medium includes mixing the filter in the liquid medium.
   Inventive Concept 716. The method according to any one of Inventive Concepts 710 and 711, wherein testing for the presence of the virus trapped by the filter includes transferring at least a portion of the liquid medium into a testing machine.
   Inventive Concept 717. The method according to any one of Inventive Concepts 710 and 711, wherein the liquid medium is selected from the group consisting of: a transport medium, a culture medium, a purification agent, a stabilizing agent, a lysing buffer, and an extraction agent.
   Inventive Concept 718. The method according to any one of Inventive Concepts 664 and 665, wherein testing for the presence of the virus includes using spectroscopy.
   Inventive Concept 719. The method according to any one of Inventive Concepts 664 and 665, wherein testing for the presence of the virus includes using microscopy.
   Inventive Concept 720. The method according to any one of Inventive Concepts 664 and 665, wherein testing for the presence of the virus includes using image processing.
   Inventive Concept 721. The method according to any one of Inventive Concepts 664 and 665, wherein testing for the presence of the virus includes using three-dimensional (3D) image sensing.
   Inventive Concept 722. The method according to any one of Inventive Concepts 664 and 665, wherein testing for the presence of the virus includes using artificial intelligence.
   Inventive Concept 723. The method according to any one of Inventive Concepts 664 and 665, wherein testing for the presence of the virus includes using a molecular-based assay.
   Inventive Concept 724. The method according to Inventive Concept 723, wherein testing for the presence of the virus includes using nucleic acid amplification.
   Inventive Concept 725. The method according to Inventive Concept 724, wherein testing for the presence of the virus includes using polymerase chain reaction (PCR).
   Inventive Concept 726. The method according to Inventive Concept 724, wherein testing for the presence of the virus includes using isothermal amplification.
   Inventive Concept 726b. The method according to Inventive Concept 726, wherein testing for the presence of the virus includes using loop-mediated isothermal amplification (LAMP).
   Inventive Concept 726c. The method according to Inventive Concept 726, wherein testing for the presence of the virus includes using a nicking enzyme amplification (NEAR) reaction.
   Inventive Concept 727. The method according to Inventive Concept 664, further including, before testing for the presence of the virus in the saliva trapped by the filter, transporting the filter while the filter is at least partially immersed in a liquid medium.
   Inventive Concept 728. The method according to Inventive Concept 727, wherein testing for the presence of the virus in the saliva trapped by the filter includes inserting at least a portion of the liquid medium into a thermal cycler.
   Inventive Concept 729. The method according to Inventive Concept 665, further including, before testing for the presence of the virus that adhered to the filter, transporting the filter while the filter is at least partially immersed in a liquid medium.
   Inventive Concept 730. The method according to Inventive Concept 729, wherein testing for the presence of the virus that adhered to the filter includes inserting at least a portion of the liquid medium into a thermal cycler.
   Inventive Concept 731. The method according to any one of Inventive Concepts 664 and 665, wherein collecting the liquid specimen sample includes receiving the liquid specimen sample by a healthcare worker from the subject.
   Inventive Concept 732. The method according to any one of Inventive Concepts 664 and 665, wherein the method does not include swabbing a nasal cavity of the subject.
   Inventive Concept 733. The method according to any one of Inventive Concepts 664 and 665, wherein the method does not include swabbing a pharynx of the subject.
   Inventive Concept 734. The method according to any one of Inventive Concepts 664 and 665, wherein the method does not include swabbing a nasal cavity of the subject and does not include swabbing a pharynx of the subject.
   Inventive Concept 735. The method according to any one of Inventive Concepts 664 and 665, wherein the method does not include swabbing any portion of the subject.
   Inventive Concept 736. The method according to any one of Inventive Concepts 664 and 665, wherein collecting the liquid specimen sample includes collecting the liquid specimen sample that was expressed via an anterior opening of an oral cavity of the subject.
   Inventive Concept 737. The method according to any one of Inventive Concepts 664 and 665, wherein collecting the liquid specimen sample includes aspirating the liquid specimen sample via an anterior opening of an oral cavity of the subject.
   Inventive Concept 738. The method according to Inventive Concept 664, wherein collecting the liquid specimen sample includes:

gargling, by the subject, the oral wash fluid to produce the liquid specimen sample that includes the oral wash fluid and the saliva; and

thereafter, collecting the liquid specimen sample from the subject.

Inventive Concept 739. The method according to Inventive Concept 664, wherein collecting the liquid specimen sample includes:

gargling, by the subject, the oral wash fluid to produce the liquid specimen sample that includes the oral wash fluid and the saliva; and

thereafter, expressing, by the subject, out of an anterior opening of an oral cavity of the subject, the liquid specimen sample.

Inventive Concept 740. The method according to Inventive Concept 664, wherein collecting the liquid specimen sample includes:

swishing, by the subject, the oral wash fluid in an oral cavity of the subject to produce the liquid specimen sample that includes the oral wash fluid and the saliva; and

thereafter, collecting the liquid specimen sample from the subject.

Inventive Concept 741. The method according to Inventive Concept 664, wherein collecting the liquid specimen sample includes:

swishing, by the subject, the oral wash fluid in an oral cavity of the subject to produce the liquid specimen sample that includes the oral wash fluid and the saliva; and

thereafter, expressing, by the subject, out of an anterior opening of the oral cavity, the liquid specimen sample.

Inventive Concept 742. The method according to any one of Inventive Concepts 664 and 665, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 743. The method according to Inventive Concept 664, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, collecting the liquid specimen sample that passed out of an anterior opening of an oral cavity of the subject and includes the oral wash fluid, the nasal wash fluid, and at least a portion of the biological material washed into the oropharynx by the nasal wash fluid.

Inventive Concept 744. The method according to Inventive Concept 743, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

collecting the liquid specimen sample that passed out of the anterior opening of the oral cavity.

Inventive Concept 745. The method according to Inventive Concept 743, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

collecting the liquid specimen sample that passed out of the anterior opening of the oral cavity.

Inventive Concept 746. The method according to Inventive Concept 664, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, expressing, by the subject, out of an anterior opening of an oral cavity of the subject, the liquid specimen sample that includes the oral wash fluid, the nasal wash fluid, and at least a portion of the biological material washed into the oropharynx by the nasal wash fluid.

Inventive Concept 747. The method according to Inventive Concept 746, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

expressing, by the subject, the liquid specimen sample out of the anterior opening of the oral cavity.

Inventive Concept 748. The method according to Inventive Concept 746, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

expressing, by the subject, the liquid specimen sample out of the anterior opening of the oral cavity.

Inventive Concept 749. The method according to any one of Inventive Concepts 743 and 746, wherein intranasally dispensing includes intranasally dispensing, by the subject, the nasal wash fluid into the nasal cavity.
Inventive Concept 750. The method according to any one of Inventive Concepts 743 and 746, wherein intranasally dispensing includes intranasally dispensing, by a healthcare worker, the nasal wash fluid into the nasal cavity.
Inventive Concept 750b. The method according to any one of Inventive Concepts 664 and 665, wherein the virus is an Influenza virus, and wherein testing for the presence of the virus includes testing for the presence of the Influenza virus.
751. The method according to any one of Inventive Concepts 664 and 665, wherein the virus is a coronavirus, and wherein testing for the presence of the virus includes testing for the presence of the coronavirus.
Inventive Concept 752. The method according to Inventive Concept 751, wherein the coronavirus is Severe acute respiratory syndrome coronavirus 2 (SARS-CoV-2).
Inventive Concept 753. The method according to any one of Inventive Concepts 664 and 665, wherein the virus is an Influenza virus, and wherein testing for the presence of the virus includes testing for the presence of the Influenza virus.
Inventive Concept 754. The method according to Inventive Concept 664,

wherein the subject is one of a plurality of subjects, and wherein the liquid specimen sample is one of a plurality of liquid specimen samples,

wherein collecting the liquid specimen sample from the subject includes collecting, from the plurality of subjects, the plurality of liquid specimen samples, respectively, which include respective oral wash fluids and saliva, and

wherein passing the liquid specimen sample through the filter includes:
combining the plurality of liquid specimen samples; and
passing the combined plurality of liquid specimen samples through the filter.
   Inventive Concept 755. The method according to Inventive Concept 754, wherein the plurality of liquid specimen samples includes at least five liquid specimen samples from at least five subjects, respectively.
   Inventive Concept 756. The method according to Inventive Concept 755, wherein the plurality of liquid specimen samples includes at least ten liquid specimen samples from at least ten subjects, respectively.
   Inventive Concept 757. The method according to Inventive Concept 754, wherein the plurality of liquid specimen samples have a combined volume of at least 25 cc, and wherein collecting, from the plurality of subjects, the plurality of liquid specimen samples, respectively, includes collecting, from the plurality of subjects, the plurality of liquid specimen samples, respectively, having the combined volume.
   Inventive Concept 758. The method according to Inventive Concept 757, wherein the combined volume is at least 50 cc.
   Inventive Concept 759. The method according to Inventive Concept 758, wherein the combined volume is at least 100 cc.
   Inventive Concept 760. The method according to Inventive Concept 754, wherein combining the plurality of liquid specimen samples includes mixing together the plurality of liquid specimen samples.

There is still further provided, in accordance with an Inventive Concept 761 of the present invention, a method including:

intranasally dispensing nasal wash fluid into a nasal cavity of a subject;

thereafter, collecting a specimen sample by performing one or more nasal swabs selected from the group consisting of: an anterior nares swab and a mid-turbinate swab; and

testing the specimen sample for the presence of a virus using a lateral flow immunoassay test strip.

Inventive Concept 762. The method according to Inventive Concept 761, wherein the virus is Severe acute respiratory syndrome coronavirus 2 (SARS-CoV-2).
Inventive Concept 762b. The method according to Inventive Concept 761, wherein the virus is an Influenza virus.

There is additionally provided, in accordance with an Inventive Concept 763 of the present invention, a method including:

intranasally dispensing nasal wash fluid into a nasal cavity of a subject;

thereafter, tilting a head of the subject farther back than while intranasally dispensing the nasal wash fluid and farther back than a neutral position; and

thereafter, collecting a specimen sample by performing one or more nasal swabs selected from the group consisting of: an anterior nares swab and a mid-turbinate swab.

There is yet additionally provided, in accordance with an Inventive Concept 764 of the present invention, a method including:

intranasally dispensing nasal wash fluid into a nasal cavity of a subject;

tilting a head of the subject back until a neck of the subject is fully extended; and

thereafter, collecting a specimen sample by performing one or more nasal swabs selected from the group consisting of: an anterior nares swab and a mid-turbinate swab.

Inventive Concept 765. The method according to any one of Inventive Concepts 761, 763, and 764, wherein collecting the specimen sample includes performing the one or more nasal swabs selected from the group consisting of: the anterior nares swab and the mid-turbinate swab, without performing a nasopharyngeal swab.
Inventive Concept 766. The method according to any one of Inventive Concepts 761, 763, and 764, wherein performing the one or more nasal swabs includes performing the mid-turbinate swab.
Inventive Concept 767. The method according to Inventive Concept 766, wherein collecting the specimen sample includes performing the mid-turbinate swab, without performing a nasopharyngeal swab.
Inventive Concept 768. The method according to any one of Inventive Concepts 761, 763, and 764, wherein performing the one or more nasal swabs includes performing the anterior nares swab.
Inventive Concept 769. The method according to Inventive Concept 768,

wherein intranasally dispensing the nasal wash fluid into the nasal cavity includes:
introducing, into a nostril of the subject, a swab tip of a nasal sampling device, the swab tip shaped so as to define (i) one or more nozzles, (ii) a proximal opening, and (iii) one or more channels within the swab tip that connect the one or more nozzles in fluid communication with the proximal opening; and
thereafter, intranasally dispensing the nasal wash fluid into the nasal cavity from the one or more nozzles, by introducing the nasal wash fluid into the proximal opening, and

wherein performing the anterior nares swab includes performing the anterior nares swab by contacting a wall of an anterior naris of the nasal cavity with an absorbent material of the swab tip.

Inventive Concept 770. The method according to Inventive Concept 768, wherein collecting the specimen sample includes performing the anterior nares swab, without performing the mid-turbinate swab.
Inventive Concept 771. The method according to Inventive Concept 768, wherein collecting the specimen sample includes performing the anterior nares swab, without performing a nasopharyngeal swab.
Inventive Concept 772. The method according to Inventive Concept 768, wherein collecting the specimen sample includes performing the anterior nares swab, without performing the mid-turbinate swab and without performing a nasopharyngeal swab.
Inventive Concept 773. The method according to any one of Inventive Concepts 761, 763, and 764, wherein collecting the specimen sample includes collecting between 25 and 150 microliters of material on a single swab.
Inventive Concept 774. The method according to Inventive Concept 773, wherein collecting the specimen sample includes collecting between 25 and 125 microliters of material on the single swab.
Inventive Concept 775. The method according to Inventive Concept 773, wherein the single swab includes a flocked swab tip, and wherein collecting the specimen sample includes collecting between 25 and 150 microliters of the material on the flocked swab tip.

There is also provided, in accordance with an Inventive Concept 776 of the present invention, a method including:

intranasally dispensing nasal wash fluid into a nasal cavity of a subject;

thereafter, inserting a specimen sampler into the nasal cavity such that a farthest-inserted portion of the specimen sampler is positioned in an anterior naris of the nasal cavity; and

collecting a specimen sample on the specimen sampler by rubbing a wall of the anterior naris with the specimen sampler.

Inventive Concept 777. The method according to Inventive Concept 776, wherein rubbing the wall of the anterior naris includes rotating the specimen sampler while rubbing the wall of the anterior naris with the specimen sampler.
Inventive Concept 778. The method according to Inventive Concept 776, wherein the specimen sampler includes a head including an absorbent material, and wherein rubbing the wall of the anterior naris with the specimen sampler includes rubbing the wall of the anterior naris with the absorbent material of the head of the specimen sampler.

There is further provided, in accordance with an Inventive Concept 779 of the present invention, a method including:

intranasally dispensing nasal wash fluid into a nasal cavity of a subject;

inserting a specimen sampler distally into the nasal cavity such that a farthest-inserted portion of the specimen sampler is positioned in an anterior naris of the nasal cavity;

collecting a specimen sample on the specimen sampler by contacting a wall of the anterior naris with the specimen sampler; and

testing the specimen sample for the presence of a virus using a lateral flow immunoassay test strip.

Inventive Concept 780. The method according to Inventive Concept 779, wherein the virus is Severe acute respiratory syndrome coronavirus 2 (SARS-CoV-2).
Inventive Concept 781. The method according to Inventive Concept 779, wherein the virus is an Influenza virus.
Inventive Concept 782. The method according to any one of Inventive Concepts 761-779, further including, after intranasally dispensing the nasal wash fluid and before collecting the specimen sample, tilting a head of the subject farther back than while intranasally dispensing the nasal wash fluid and farther back than a neutral position.
Inventive Concept 783. The method according to Inventive Concept 782, further including, after tilting the head farther back and before collecting the specimen sample, tilting the head forward to the neutral position or a tilted-forward position.
Inventive Concept 8784. The method according to Inventive Concept 783, further including, while the subject holds the head tilted back, waiting for a period of time between (a) tilting the head farther back and (b) tilting the head forward to the neutral or the tilted-forward position, the period of time at least 2 seconds.
Inventive Concept 785. The method according to Inventive Concept 784, wherein the period of time is at least 5 seconds.
Inventive Concept 786. The method according to Inventive Concept 785, wherein the period of time is at least 10 seconds.
Inventive Concept 787. The method according to Inventive Concept 784, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 788. The method according to Inventive Concept 783, further including, after tilting the head forward to the neutral or the tilted-forward position, waiting for a period of time before collecting the specimen sample, the period of time at least 5 seconds.
Inventive Concept 789. The method according to Inventive Concept 788, wherein the period of time is at least 10 seconds.
Inventive Concept 790. The method according to Inventive Concept 783, wherein tilting the head forward includes tilting the head forward to the neutral or the tilted-forward position after sensing, by the subject, the nasal wash fluid in an oropharynx of the subject.
Inventive Concept 791. The method according to any one of Inventive Concepts 761-779, wherein intranasally dispensing the nasal wash fluid includes:

tilting a head of the subject back until a neck of the subject is fully extended; and

while the subject holds the head tilted back with the neck fully extended, intranasally dispensing nasal wash fluid into a nasal cavity of the subject.

Inventive Concept 792. The method according to Inventive Concept 791, further including, after intranasally dispensing the nasal wash, tilting the head forward to a neutral position or a tilted-forward position.
Inventive Concept 793. The method according to Inventive Concept 792, further including, while the subject holds the head tilted back with the neck fully extended, waiting for a period of time between (a) concluding intranasally dispensing the nasal wash fluid into the nasal cavity and (b) tilting the head forward to the neutral or the tilted-forward position, the period of time at least 2 seconds.
Inventive Concept 794. The method according to Inventive Concept 793, wherein the period of time is at least 5 seconds.
Inventive Concept 795. The method according to Inventive Concept 794, wherein the period of time is at least 10 seconds.
Inventive Concept 796. The method according to Inventive Concept 793, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 797. The method according to Inventive Concept 792, further including, after tilting the head forward to the neutral or the tilted-forward position, waiting for a period of time before collecting the specimen sample, the period of time at least 5 seconds.
Inventive Concept 798. The method according to Inventive Concept 797, wherein the period of time is at least 10 seconds.
Inventive Concept 799. The method according to any one of Inventive Concepts 761-779, wherein intranasally dispensing the nasal wash fluid includes intranasally dispensing the nasal wash fluid into the nasal cavity while a head of the subject is held in a neutral position or a tilted-forward position.
Inventive Concept 800. The method according to any one of Inventive Concepts 761-779, further including, after intranasally dispensing the nasal wash fluid and before collecting the specimen sample, sniffing, by the subject, the nasal wash fluid.

There is still further provided, in accordance with an Inventive Concept 801 of the present invention, a method including:

intranasally dispensing nasal wash fluid into a nasal cavity of a subject;

thereafter, tilting a head of the subject farther back than while intranasally dispensing the nasal wash fluid and farther back than a neutral position;

inserting a specimen sampler into the nasal cavity such that a farthest-inserted portion of the specimen sampler is positioned in an anterior naris of the nasal cavity; and

collecting a specimen sample on the specimen sampler by contacting a wall of the anterior naris with the specimen sampler.

Inventive Concept 802. The method according to Inventive Concept 801, wherein tilting the head farther back includes tilting the head back until a neck of the subject is fully extended.
Inventive Concept 803. The method according to Inventive Concept 801, further including, after tilting the head farther back and before collecting the specimen sample, tilting the head forward to the neutral position or a tilted-forward position.
Inventive Concept 804. The method according to Inventive Concept 803, further including, while the subject holds the head tilted back, waiting for a period of time between (a) tilting the head farther back and (b) tilting the head forward to the neutral or the tilted-forward position, the period of time at least 2 seconds.
Inventive Concept 805. The method according to Inventive Concept 804, wherein the period of time is at least 5 seconds.
Inventive Concept 806. The method according to Inventive Concept 805, wherein the period of time is at least 10 seconds.
Inventive Concept 807. The method according to Inventive Concept 804, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 808. The method according to Inventive Concept 803, further including, after tilting the head forward to the neutral or the tilted-forward position, waiting for a period of time before collecting the specimen sample, the period of time at least 5 seconds.
Inventive Concept 809. The method according to Inventive Concept 808, wherein the period of time is at least 10 seconds.
Inventive Concept 810. The method according to Inventive Concept 803, wherein tilting the head forward includes tilting the head forward to the neutral or the tilted-forward position after sensing, by the subject, the nasal wash fluid in an oropharynx of the subject.
Inventive Concept 811. The method according to Inventive Concept 801, wherein intranasally dispensing the nasal wash fluid includes intranasally dispensing the nasal wash fluid into the nasal cavity while the head is held in the neutral position or a tilted-forward position.
Inventive Concept 812. The method according to Inventive Concept 801, further including, after intranasally dispensing the nasal wash fluid and before collecting the specimen sample, sniffing, by the subject, the nasal wash fluid.

There is additionally provided, in accordance with an Inventive Concept 813 of the present invention, a method including:

tilting a head of a subject back until a neck of the subject is fully extended;

while the subject holds the head tilted back with the neck fully extended, intranasally dispensing nasal wash fluid into a nasal cavity of the subject;

inserting a specimen sampler into the nasal cavity such that a farthest-inserted portion of the specimen sampler is positioned in an anterior naris of the nasal cavity; and

collecting a specimen sample on the specimen sampler by contacting a wall of the anterior naris with the specimen sampler.

Inventive Concept 814. The method according to Inventive Concept 813, further including, after intranasally dispensing the nasal wash, tilting the head forward to a neutral position or a tilted-forward position.
Inventive Concept 815. The method according to Inventive Concept 814, further including, while the subject holds the head tilted back with the neck fully extended, waiting for a period of time between (a) concluding intranasally dispensing the nasal wash fluid into the nasal cavity and (b) tilting the head forward to the neutral or the tilted-forward position, the period of time at least 2 seconds.
Inventive Concept 816. The method according to Inventive Concept 815, wherein the period of time is at least 5 seconds.
Inventive Concept 817. The method according to Inventive Concept 816, wherein the period of time is at least 10 seconds.
Inventive Concept 818. The method according to Inventive Concept 815, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 819. The method according to Inventive Concept 814, further including, after tilting the head forward to the neutral or the tilted-forward position, waiting for a period of time before collecting the specimen sample, the period of time at least 5 seconds.
Inventive Concept 820. The method according to Inventive Concept 819, wherein the period of time is at least 10 seconds.
Inventive Concept 821. The method according to Inventive Concept 813, further including, after intranasally dispensing the nasal wash fluid and before collecting the specimen sample, sniffing, by the subject, the nasal wash fluid.
Inventive Concept 822. The method according to any one of Inventive Concepts 779-821, wherein the specimen sampler includes a head including an absorbent material, and wherein contacting the wall of the anterior naris with the specimen sampler includes contacting the wall of the anterior naris with the absorbent material of the head of the specimen sampler.
Inventive Concept 823. The method according to any one of Inventive Concepts 776-821, wherein the specimen sampler includes a swab.
Inventive Concept 824. The method according to Inventive Concept 823, wherein the swab includes a flocked swab tip.
Inventive Concept 825. The method according to Inventive Concept 823, wherein the swab includes a foam swab tip.
Inventive Concept 826. The method according to Inventive Concept 823, wherein the swab includes a tip having a greatest diameter of between 2 and 5 mm.
Inventive Concept 827. The method according to Inventive Concept 823, wherein the swab includes a tip having a collection surface area of between 200 and 300 mm2.
Inventive Concept 828. The method according to Inventive Concept 823, wherein the swab includes a tip having a volume of between 200 and 300 mm3.
Inventive Concept 829. The method according to Inventive Concept 823, wherein the swab includes a tip having a length of between 12 and 20 mm.
Inventive Concept 830. The method according to any one of Inventive Concepts 776-821, wherein the specimen sampler includes a brush.
Inventive Concept 831. The method according to any one of Inventive Concepts 776-821, wherein the specimen sampler includes a sponge.
Inventive Concept 832. The method according to any one of Inventive Concepts 776-821, wherein the specimen sampler includes a nasal plug.
Inventive Concept 833. The method according to any one of Inventive Concepts 776-821, wherein the specimen sampler is shaped so as not to define any nozzles.
Inventive Concept 834. The method according to any one of Inventive Concepts 776-821, wherein collecting the specimen sample includes collecting the specimen sample on the specimen sampler without using suction.
Inventive Concept 835. The method according to any one of Inventive Concepts 761-821, wherein intranasally dispensing includes intranasally dispensing, by the subject, the nasal wash fluid into the nasal cavity.
Inventive Concept 836. The method according to any one of Inventive Concepts 761-821, wherein collecting the specimen sample includes collecting the specimen sample by the subject.
Inventive Concept 837. The method according to any one of Inventive Concepts 761-821, further including waiting for a period of time between (a) concluding intranasally dispensing the nasal wash fluid into the nasal cavity and (b) collecting the specimen sample, the period of time at least 2 seconds and no more than 30 minutes.
Inventive Concept 838. The method according to any one of Inventive Concepts 761-821, wherein intranasally dispensing the nasal wash fluid includes intranasally dispensing the nasal wash fluid without blowing, by the subject, a nose of the subject immediately prior to intranasally dispensing the nasal wash fluid.
Inventive Concept 839. The method according to any one of Inventive Concepts 761-821, wherein intranasally dispensing the nasal wash fluid includes intranasally dispensing the nasal wash fluid into each of one or both nostrils of the subject in a total volume of between 0.15 and 0.5 mL per nostril.
Inventive Concept 840. The method according to any one of Inventive Concepts 776-821, wherein collecting the specimen sample on the specimen sampler includes collecting between 25 and 150 microliters of material on the specimen sampler.
Inventive Concept 841. The method according to Inventive Concept 840, wherein collecting the specimen sample on the specimen sampler includes collecting between 25 and 125 microliters of material on the specimen sampler.
Inventive Concept 842. The method according to Inventive Concept 840, wherein the specimen sampler includes a swab including a flocked swab tip, and wherein collecting the specimen sample on the specimen sampler includes collecting between 25 and 150 microliters of the material on the flocked swab tip.
Inventive Concept 843. The method according to any one of Inventive Concepts 761-821, further including, after intranasally dispensing the nasal wash fluid into the nasal cavity:

orally dispensing oral wash fluid into an oral cavity of the subject via an anterior opening of the oral cavity; and

gargling, by the subject, the oral wash fluid.

Inventive Concept 844. The method according to any one of Inventive Concepts 761-821, wherein intranasally dispensing the nasal wash fluid includes intranasally dispensing the nasal wash fluid as one or more types of fluid discharge selected from the group consisting of: a spray, a mist, a pressurized aerosol, steam, and an atomized fluid.
Inventive Concept 845. The method according to any one of Inventive Concepts 761-821, wherein intranasally dispensing the nasal wash fluid includes intranasally dispensing the nasal wash fluid as a fluid stream.
Inventive Concept 846. The method according to any one of Inventive Concepts 761-821, wherein collecting the specimen sample includes collecting the specimen sample after sensing, by the subject, the nasal wash fluid in an oropharynx of the subject.
Inventive Concept 847. The method according to Inventive Concept 846, wherein collecting the specimen sample includes collecting the specimen sample after tasting, by the subject, the nasal wash fluid in the oropharynx.
Inventive Concept 848. The method according to Inventive Concept 847, wherein the nasal wash fluid includes a tastant, and wherein collecting the specimen sample includes collecting the specimen sample after tasting, by the subject, the tastant in the oropharynx.
Inventive Concept 849. The method according to any one of Inventive Concepts 763-778 and 801-821, further including testing the specimen sample for the presence of a particulate.
Inventive Concept 850. The method according to Inventive Concept 849, wherein the particulate is selected from the group consisting of: a virus, a bacterium, a protozoan, and a fungus.
Inventive Concept 851. The method according to Inventive Concept 850, wherein the particulate is the virus.
Inventive Concept 852. The method according to Inventive Concept 851, wherein the virus is Severe acute respiratory syndrome coronavirus 2 (SARS-CoV-2).
Inventive Concept 853. The method according to Inventive Concept 851, wherein the virus is an Influenza virus.
Inventive Concept 854. The method according to Inventive Concept 851, wherein testing for the presence of the virus includes using nucleic acid amplification.
Inventive Concept 855. The method according to Inventive Concept 854, wherein testing for the presence of the virus includes using polymerase chain reaction (PCR).
Inventive Concept 856. The method according to Inventive Concept 855, wherein testing for the presence of the virus includes using quantitative PCR (qPCR).
Inventive Concept 857. The method according to Inventive Concept 851, wherein testing for the presence of the virus includes performing an immunoassay.
Inventive Concept 858. The method according to Inventive Concept 857, wherein performing the immunoassay includes using a lateral flow immunoassay test strip.
Inventive Concept 859. The method according to Inventive Concept 858, wherein performing the immunoassay includes performing a chromatographic digital immunoassay.

There is also provided, in accordance with an Inventive Concept 860 of the present invention, a method including:

intranasally dispensing nasal wash fluid into a nasal cavity of a subject; and

collecting a specimen sample by performing one or more nasal swabs selected from the group consisting of: an anterior nares swab, a mid-turbinate swab, and a nasopharyngeal swab.

There is further provided, in accordance with an Inventive Concept 861 of the present invention, a nasal sampling device for collecting a specimen sample, the nasal sampling device including:

a dispensing container, which contains nasal wash fluid; and

a nasal applicator, which is shaped so as to define (i) one or more nozzles, (ii) a proximal opening, and (iii) one or more channels within the nasal applicator that connect the one or more nozzles in fluid communication with the proximal opening, wherein at least a portion of an external surface of nasal applicator includes an absorbent material for collecting the specimen sample,

wherein the nasal applicator is disposed distal to the dispensing container, with the proximal opening of the nasal applicator in fluid communication with the dispensing container, and

wherein the nasal sampling device is configured to intranasally dispense the nasal wash fluid from the dispensing container into a nasal cavity of a subject via the one or more nozzles of the nasal applicator.

Inventive Concept 862. The nasal sampling device according to Inventive Concept 861, wherein the nasal sampling device includes a pump that is configured to dispense the nasal wash fluid.
Inventive Concept 863. The nasal sampling device according to Inventive Concept 861, wherein the nasal sampling device is configured to dispense the nasal wash fluid by manual compression of the dispensing container.
Inventive Concept 864. The nasal sampling device according to Inventive Concept 861, wherein the nasal sampling device includes an atomizer that is configured to dispense the nasal wash fluid.
Inventive Concept 865. The nasal sampling device according to Inventive Concept 861, wherein the nasal sampling device includes a pressurized aerosol generator that is configured to dispense the nasal wash fluid.
Inventive Concept 866. The nasal sampling device according to Inventive Concept 861, wherein the nasal sampling device is configured to intranasally dispenses the nasal wash fluid from the one or more nozzles as one or more types of fluid discharge selected from the group consisting of: a spray, a mist, a pressurized aerosol, steam, and an atomized fluid.
Inventive Concept 867. The nasal sampling device according to Inventive Concept 861, wherein the absorbent material is selected from the group consisting of: flocking, rayon, cotton, polyester, polyurethane foam, polyethylene terephthalate (PET), foam, sponge, a brush, and nanofibers.
Inventive Concept 868. The nasal sampling device according to any one of Inventive Concepts 861-867, wherein a proximal end of the nasal applicator is in direct physical contact with the dispensing container.
Inventive Concept 869. The nasal sampling device according to Inventive Concept 868, wherein the proximal end of the nasal applicator is fixed in the direct physical contact with the dispensing container.
Inventive Concept 870. The nasal sampling device according to any one of Inventive Concepts 861-867, further including a tube, which physically connects a proximal end of the nasal applicator to the dispensing container, wherein the proximal opening of the nasal applicator is in fluid communication with the dispensing container via the tube.
Inventive Concept 871. The nasal sampling device according to Inventive Concept 870, wherein the tube extends distally through at least a portion of the nasal applicator, so as to define at least a portion of the one or more channels within the nasal applicator.
Inventive Concept 872. The nasal sampling device according to Inventive Concept 870, wherein the tube physically connects the proximal end of the nasal applicator to the dispensing container such that a distance between the proximal end of the nasal applicator and a distal-most point of the dispensing container is between 1 and 13 cm.
Inventive Concept 873. The nasal sampling device according to any one of Inventive Concepts 861-867, wherein the nasal applicator is removable from the dispensing container.
Inventive Concept 874. The nasal sampling device according to any one of Inventive Concepts 861-867,

wherein the nasal sampling device includes a user control that is coupled to the dispensing container and configured to cause the dispensing container to dispense the nasal wash fluid, and

wherein a proximal end of the nasal applicator is fixed to a distal portion of the user control, and wherein at least the distal portion of the user control is removable from the dispensing container, so as to remove the nasal applicator from the dispensing container.

Inventive Concept 875. A kit including the nasal sampling device according to Inventive Concept 874,

wherein the kit further includes a vial, and a liquid for bathing at least a portion of the nasal applicator in the vial, and

wherein the distal portion of the user control is configured, after removal from the dispensing container, to be coupled to an opening of the vial, such that the nasal applicator is disposed within the vial and the distal portion serves as a cap for the vial.

Inventive Concept 876. A kit including the nasal sampling device according to any one of Inventive Concepts 861-867, the kit further including a test for testing for the presence of a particulate in a specimen sample collected using the nasal sampling device.
Inventive Concept 877. The kit according to Inventive Concept 876, wherein the test includes an immunoassay, which is configured to detect the presence of the particulate.
Inventive Concept 878. The kit according to Inventive Concept 877, wherein the immunoassay includes a lateral flow immunoassay test strip, which is configured to detect the presence of the particulate.
Inventive Concept 879. The kit according to Inventive Concept 878, wherein the immunoassay includes a chromatographic digital immunoassay, which is configured to detect the presence of the particulate.
Inventive Concept 880. The kit according to Inventive Concept 876, wherein the test is configured to test for the presence of the particulate to aid in diagnosis of a disease.
Inventive Concept 881. A kit including the nasal sampling device according to any one of Inventive Concepts 861-867, the kit further including:

a vial; and

a liquid for bathing at least a portion of a specimen sample collected using the nasal sampling device in the vial, the liquid selected from the group consisting of: a lysis buffer, saline solution, and transport medium.

There is still further provided, in accordance with an Inventive Concept 882 of the present invention, a method for collecting a specimen sample from a nasal cavity of a subject using a nasal sampling device, the method including:

introducing, into a nostril of the subject, a nasal applicator of the nasal sampling device, the nasal applicator shaped so as to define (i) one or more nozzles, (ii) a proximal opening, and (iii) one or more channels within the nasal applicator that connect the one or more nozzles in fluid communication with the proximal opening;

thereafter, intranasally dispensing nasal wash fluid into the nasal cavity from the one or more nozzles, by introducing the nasal wash fluid into the proximal opening; and

thereafter, collecting the specimen sample from the nasal cavity by rubbing a wall of an anterior naris of the nasal cavity with an absorbent material of at least a portion of an external surface of the nasal applicator.

Inventive Concept 883. The method according to Inventive Concept 882, wherein introducing the nasal applicator includes introducing the nasal applicator into the nostril no more deeply than the anterior naris.
Inventive Concept 884. The method according to Inventive Concept 882, wherein collecting the specimen sample includes collecting the specimen sample from the nasal cavity by rubbing the wall of the anterior naris with the absorbent material of the nasal applicator, without rubbing any portion of the nostril deeper than the anterior naris with the absorbent material of the nasal applicator.
Inventive Concept 885. The method according to Inventive Concept 882, wherein collecting the specimen sample from the nasal cavity includes collecting the specimen sample without using suction.
Inventive Concept 886. The method according to any one of Inventive Concepts 882-885, wherein intranasally dispensing the nasal wash fluid includes:

tilting a head of the subject back until a neck of the subject is fully extended; and

while the subject holds the head tilted back with the neck fully extended, intranasally dispensing nasal wash fluid into a nasal cavity of the subject.

Inventive Concept 887. The method according to Inventive Concept 886, further including, after intranasally dispensing the nasal wash, tilting the head forward to a neutral or a tilted-forward position.
Inventive Concept 888. The method according to Inventive Concept 887, further including, while the subject holds the head tilted back with the neck fully extended, waiting for a period of time between (a) concluding intranasally dispensing the nasal wash fluid into the nasal cavity and (b) tilting the head forward to the neutral or the tilted-forward position, the period of time at least 2 seconds.
Inventive Concept 889. The method according to Inventive Concept 888, wherein the period of time is at least 5 seconds.
Inventive Concept 890. The method according to Inventive Concept 889, wherein the period of time is at least 10 seconds.
Inventive Concept 891. The method according to Inventive Concept 888, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 892. The method according to Inventive Concept 887, wherein tilting the head forward includes tilting the head forward to the neutral or the tilted-forward position after sensing, by the subject, the nasal wash fluid in an oropharynx of the subject.
Inventive Concept 893. The method according to any one of Inventive Concepts 882-885, further including, after intranasally dispensing the nasal wash fluid and before collecting the specimen sample, tilting a head of the subject farther back than while intranasally dispensing the nasal wash fluid and farther back than a neutral position.
Inventive Concept 894. The method according to Inventive Concept 893, further including, after tilting the head farther back and before collecting the specimen sample, tilting the head forward to the neutral position or a tilted-forward position.
Inventive Concept 895. The method according to Inventive Concept 894, further including, while the subject holds the head tilted back, waiting for a period of time between (a) tilting the head farther back and (b) tilting the head forward to the neutral or the tilted-forward position, the period of time at least 2 seconds.
Inventive Concept 896. The method according to Inventive Concept 895, wherein the period of time is at least 5 seconds.
Inventive Concept 897. The method according to Inventive Concept 896, wherein the period of time is at least 10 seconds.
Inventive Concept 898. The method according to Inventive Concept 895, wherein the period of time is a first period of time, and wherein the method further includes, after tilting the head forward to the neutral or the tilted-forward position, waiting for a second period of time before collecting the specimen sample, the period of time at least 5 seconds.
Inventive Concept 899. The method according to Inventive Concept 894, further including, after tilting the head forward to the neutral or the tilted-forward position, waiting for a period of time before collecting the specimen sample, the period of time at least 5 seconds.
Inventive Concept 900. The method according to Inventive Concept 899, wherein the period of time is at least 10 seconds.
Inventive Concept 901. The method according to Inventive Concept 894, wherein tilting the head forward includes tilting the head forward to the neutral or the tilted-forward position after sensing, by the subject, the nasal wash fluid in an oropharynx of the subject.
Inventive Concept 902. The method according to any one of Inventive Concepts 882-885, wherein intranasally dispensing the nasal wash fluid includes intranasally dispensing the nasal wash fluid into the nasal cavity while a head of the subject is held in a neutral position or a tilted-forward position.
Inventive Concept 903. The method according to any one of Inventive Concepts 882-885, wherein intranasally dispensing includes intranasally dispensing, by the subject, the nasal wash fluid into the nasal cavity.
Inventive Concept 904. The method according to any one of Inventive Concepts 882-885, wherein collecting the specimen sample includes collecting the specimen sample by the subject.
Inventive Concept 905. The method according to any one of Inventive Concepts 882-885, further including waiting for a period of time between (a) concluding intranasally dispensing the nasal wash fluid into the nasal cavity and (b) collecting the specimen sample, the period of time at least 2 seconds and no more than 30 minutes.
Inventive Concept 906. The method according to any one of Inventive Concepts 882-885, wherein intranasally dispensing the nasal wash fluid includes intranasally dispensing the nasal wash fluid from the one or more nozzles as one or more types of fluid discharge selected from the group consisting of: a spray, a mist, a pressurized aerosol, steam, and an atomized fluid.
Inventive Concept 907. The method according to any one of Inventive Concepts 882-885, wherein intranasally dispensing the nasal wash fluid includes intranasally dispensing the nasal wash fluid from the one or more nozzles as a fluid stream.
Inventive Concept 908. The method according to any one of Inventive Concepts 882-885, wherein collecting the specimen sample includes collecting the specimen sample after sensing, by the subject, the nasal wash fluid in an oropharynx of the subject.
Inventive Concept 909. The method according to Inventive Concept 908, wherein collecting the specimen sample includes collecting the specimen sample after tasting, by the subject, the nasal wash fluid in the oropharynx.
Inventive Concept 910. The method according to Inventive Concept 909, wherein the nasal wash fluid includes a tastant, and wherein collecting the specimen sample includes collecting the specimen sample after tasting, by the subject, the tastant in the oropharynx.
Inventive Concept 911. The method according to any one of Inventive Concepts 882-885, further including testing the specimen sample for the presence of a particulate.
Inventive Concept 912. The method according to Inventive Concept 911, wherein the particulate is selected from the group consisting of: a virus, a bacterium, a protozoan, and a fungus.
Inventive Concept 913. The method according to Inventive Concept 912, wherein the particulate is the virus.
Inventive Concept 914. The method according to Inventive Concept 913, wherein the virus is Severe acute respiratory syndrome coronavirus 2 (SARS-CoV-2).
Inventive Concept 915. The method according to Inventive Concept 913, wherein the virus is an Influenza virus.
Inventive Concept 916. The method according to Inventive Concept 913, wherein testing for the presence of the virus includes using nucleic acid amplification.
Inventive Concept 917. The method according to Inventive Concept 916, wherein testing for the presence of the virus includes using polymerase chain reaction (PCR).
Inventive Concept 918. The method according to Inventive Concept 917, wherein testing for the presence of the virus includes using quantitative PCR (qPCR).
Inventive Concept 919. The method according to Inventive Concept 913, wherein testing for the presence of the virus includes performing an immunoassay.
Inventive Concept 920. The method according to Inventive Concept 919, wherein performing the immunoassay includes performing a lateral flow immunoassay.
Inventive Concept 921. The method according to Inventive Concept 920, wherein performing the immunoassay includes performing a chromatographic digital immunoassay.
Inventive Concept 922. The method according to any one of Inventive Concepts 882-885,

wherein the nasal applicator is disposed distal to a dispensing container of the nasal sampling device, with the proximal opening of the nasal applicator in fluid communication with the dispensing container, and

wherein intranasally dispensing the nasal wash fluid includes intranasally dispensing the nasal wash fluid from the dispensing container into the nasal cavity via the one or more nozzles of the nasal applicator.

Inventive Concept 923. The method according to Inventive Concept 922, wherein a proximal end of the nasal applicator is in direct physical contact with the dispensing container.
Inventive Concept 924. The method according to Inventive Concept 923, wherein the proximal end of the nasal applicator is fixed in the direct physical contact with the dispensing container.
Inventive Concept 925. The method according to Inventive Concept 922, wherein the nasal sampling device further includes a tube, which physically connects a proximal end of the nasal applicator to the dispensing container, and wherein the proximal opening of the nasal applicator is in fluid communication with the dispensing container via the tube.
Inventive Concept 926. The method according to Inventive Concept 925, wherein the tube extends distally through at least a portion of the nasal applicator, so as to define at least a portion of the one or more channels within the nasal applicator.
Inventive Concept 927. The method according to Inventive Concept 925, wherein the tube physically connects the proximal end of the nasal applicator to the dispensing container such that a distance between the proximal end of the nasal applicator and a distal-most point of the dispensing container is between 1 and 13 cm.
Inventive Concept 928. The method according to Inventive Concept 922, wherein the nasal applicator is removable from the dispensing container, and wherein the method further includes, after collecting the specimen sample, removing the nasal applicator from the dispensing container.
Inventive Concept 929. The method according to Inventive Concept 922,

wherein the nasal sampling device includes a user control that is coupled to the dispensing container and configured to cause the dispensing container to dispense the nasal wash fluid, and wherein intranasally dispensing nasal wash fluid into the nasal cavity includes activating the user control, and

wherein the nasal applicator is fixed to a distal portion of the user control, wherein at least the distal portion of the user control is removable from the dispensing container, so as to remove the nasal applicator from the dispensing container, and wherein the method further includes, after collecting the specimen sample, removing the nasal applicator from the dispensing container by removing at least the distal portion of the user control from the dispensing container.

Inventive Concept 930. The method according to Inventive Concept 929, further including, after removing at least the distal portion of the user control from the dispensing container, coupling the distal portion of the user control to an opening of a vial such that the nasal applicator is disposed within a liquid in the vial and the distal portion serves as a cap for the vial.
Inventive Concept 931. The method according to Inventive Concept 922, wherein the nasal sampling device includes a pump that is configured to dispense the nasal wash fluid, and wherein intranasally dispensing nasal wash fluid into the nasal cavity includes activating the pump to dispense the nasal wash fluid.
Inventive Concept 932. The method according to Inventive Concept 922, wherein the nasal sampling device is configured to dispense the nasal wash fluid by manual compression of the dispensing container, and wherein intranasally dispensing nasal wash fluid into the nasal cavity includes manually compressing the dispensing container.
Inventive Concept 933. The method according to Inventive Concept 922, wherein the nasal sampling device includes an atomizer that is configured to dispense the nasal wash fluid, and wherein intranasally dispensing nasal wash fluid into the nasal cavity includes activating the atomizer to dispense the nasal wash fluid.
Inventive Concept 934. The method according to Inventive Concept 922, wherein the nasal sampling device includes a pressurized aerosol generator that is configured to dispense the nasal wash fluid, and wherein intranasally dispensing nasal wash fluid into the nasal cavity includes activating the pressurized aerosol generator to dispense the nasal wash fluid.
Inventive Concept 935. The method according to Inventive Concept 922, wherein the absorbent material is selected from the group consisting of: flocking, rayon, cotton, polyester, polyurethane foam, polyethylene terephthalate (PET), foam, sponge, a brush, and nanofibers.

There is additionally provided, in accordance with an Inventive Concept 936 of the present invention, a testing kit for testing a specimen sample, the testing kit including:

a vial shaped so as to define a proximal opening;

a liquid reagent;

a cap, which is configured to be sealingly coupled, at a distal side thereof, to the proximal opening of the vial;

an elongate test-strip holder, which is coupled to a proximal side of the cap; and

a lateral flow test strip, which is at least partially disposed within the elongate test-strip holder, at least partially visible from outside the test-strip holder,

wherein the cap is shaped so as to define an aperture that is in fluid communication with (a) the distal side of cap and (b) the lateral flow test strip within the elongate test-strip holder, and

wherein the cap includes an absorbent material that is disposed on the distal side of the cap and is in fluid communication (a) with the lateral flow test strip via the aperture, and (b) when the cap is sealing coupled to the proximal opening of the vial, with an interior of the vial.

Inventive Concept 937. The kit according to Inventive Concept 936, wherein the lateral flow test strip includes a lateral flow immunoassay test strip.
Inventive Concept 938. The kit according to Inventive Concept 936, wherein the liquid reagent includes a lysing buffer.

The present invention will be understood from the following detailed description of embodiments thereof, taken together with the drawings, in which:

### BRIEF DESCRIPTION OF THE DRAWINGS

FIG. 1 is a schematic illustration of a nasal wash fluid dispenser, in accordance with an application of the present invention;

FIGS. 2A-D are schematic cross-sectional illustrations of a method of collecting a specimen sample from a subject, in accordance with an application of the present invention;

FIGS. 3A-B are schematic cross-sectional illustrations of the nasal wash fluid dispenser of FIG. 1 inserted into a nasal cavity, in accordance with an application of the present invention;

FIG. 4 is a schematic cross-sectional illustration of a portion of a method for collecting a specimen sample from the subject, in accordance with an application of the present invention;

FIGS. 5A-B are schematic illustrations of a portion of a method for collecting a specimen sample from a subject, in accordance with an application of the present invention;

FIG. 6 is a schematic illustration of the nasal wash fluid dispenser of FIG. 1 with a tubular applicator thereof inserted into a nostril, in accordance with an application of the present invention;

FIGS. 7A-K are several schematic illustrations of the nasal wash fluid dispenser of FIG. 1 viewed from several different directions, in accordance with an application of the present invention;

FIGS. 8A-C are schematic illustrations of another nasal wash fluid dispenser, in accordance with an application of the present invention;

FIGS. 9A-F are schematic illustrations of yet another nasal wash fluid dispenser, in accordance with an application of the present invention;

FIG. 10 is a schematic cross-sectional illustration of the nasal wash fluid dispenser of FIGS. 9A-F inserted into the nasal cavity, in accordance with an application of the present invention;

FIG. 11 is a schematic cross-sectional illustration of a tubular applicator of the nasal wash fluid dispenser of FIGS. 9A-F, in accordance with an application of the present invention;

FIG. 12 is a schematic illustration of a testing kit, in accordance with an application of the present invention;

FIG. 13 is a schematic illustration of another testing kit, in accordance with an application of the present invention;

FIGS. 14A-B are schematic illustrations of filter units for testing a liquid specimen sample that includes saliva for the presence of a particulate, in accordance with respective applications of the present invention;

FIG. 15 is a schematic illustration of a testing kit, in accordance with an application of the present invention;

FIG. 16 is a schematic illustration of another testing kit, in accordance with an application of the present invention;

FIGS. 17A-B are schematic illustrations of a method for collecting a specimen sample from a subject, in accordance with an application of the present invention;

FIG. 18 is a schematic illustration of a testing kit, in accordance with an application of the present invention;

FIG. 19 is a schematic illustration of another testing kit, in accordance with an application of the present invention;

FIGS. 20A-B and 21A-D are schematic illustrations of respective configurations of a nasal sampling device for collecting a specimen sample, in accordance with respective applications of the present invention;

FIG. 22 is a schematic illustration of the use of nasal sampling device of FIGS. 20A-B and 21A-B for collecting a specimen sample from a nasal cavity, in accordance with an application of the present invention;

FIGS. 23A and 23B are schematic illustrations of kits that comprise portions of the nasal sampling devices of FIGS. 20A-B and 21A-B, respectively, and a vial, in accordance with respective applications of the present invention;

FIGS. 24A and 24B are schematic illustrations of another testing kit, in accordance with an application of the present invention;

FIGS. 25A-B are charts and FIG. 25C is a table that present results of an experiment conducted on behalf of the inventors, described hereinbelow as Experiment 8, in accordance with an application of the present invention;

FIG. 26A is a chart and FIG. 26B is a table that present results of the experiment conducted on behalf of the inventors, described hereinbelow as Experiment 8, in accordance with an application of the present invention; and

FIGS. 27A-B are charts that present results of the experiment conducted on behalf of the inventors, described hereinbelow as Experiment 8, in accordance with an application of the present invention.

### DETAILED DESCRIPTION OF APPLICATIONS

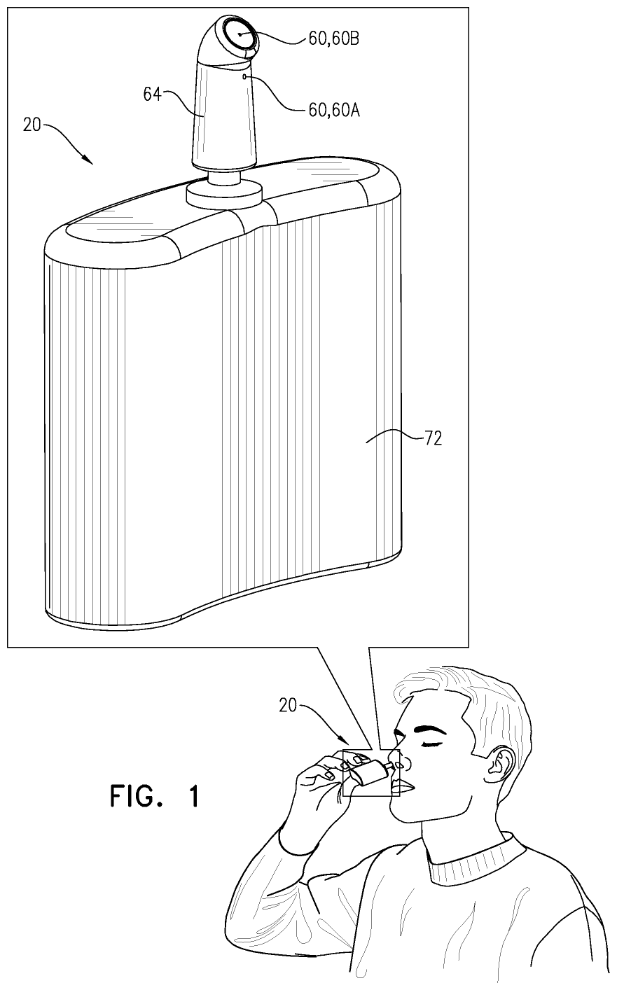

Reference is made to FIG. 1, which is a schematic illustration of a nasal wash fluid dispenser 20, in accordance with an application of the present invention.

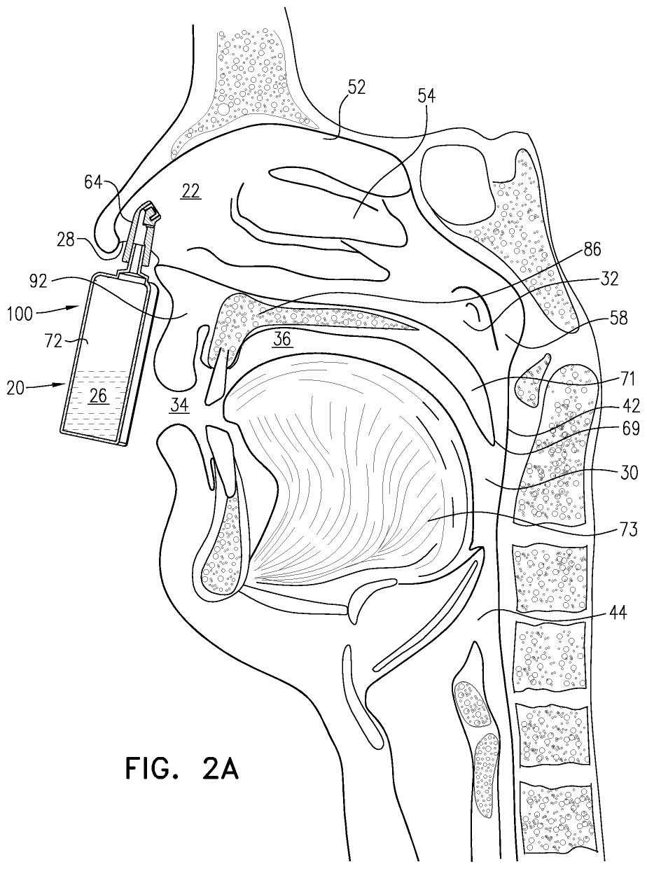

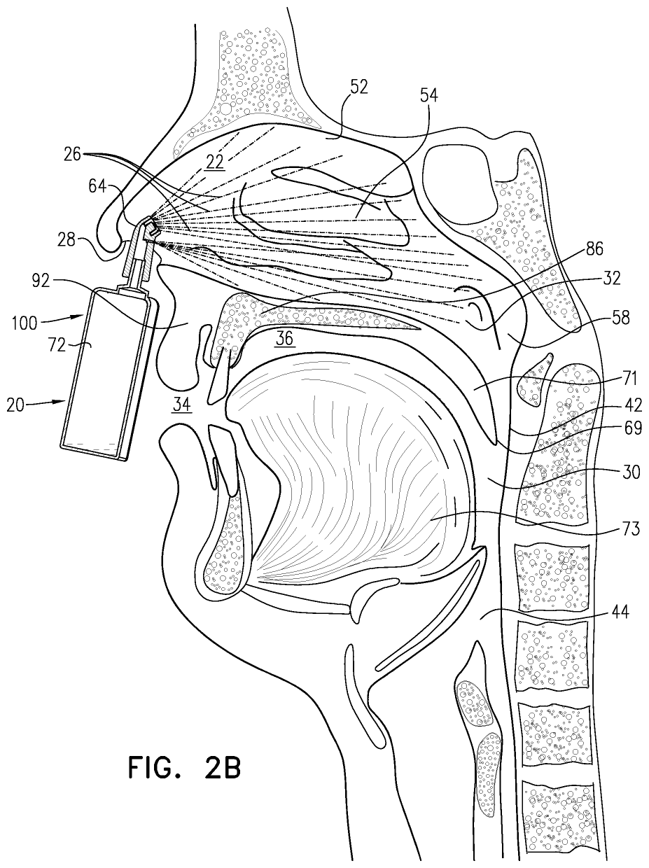

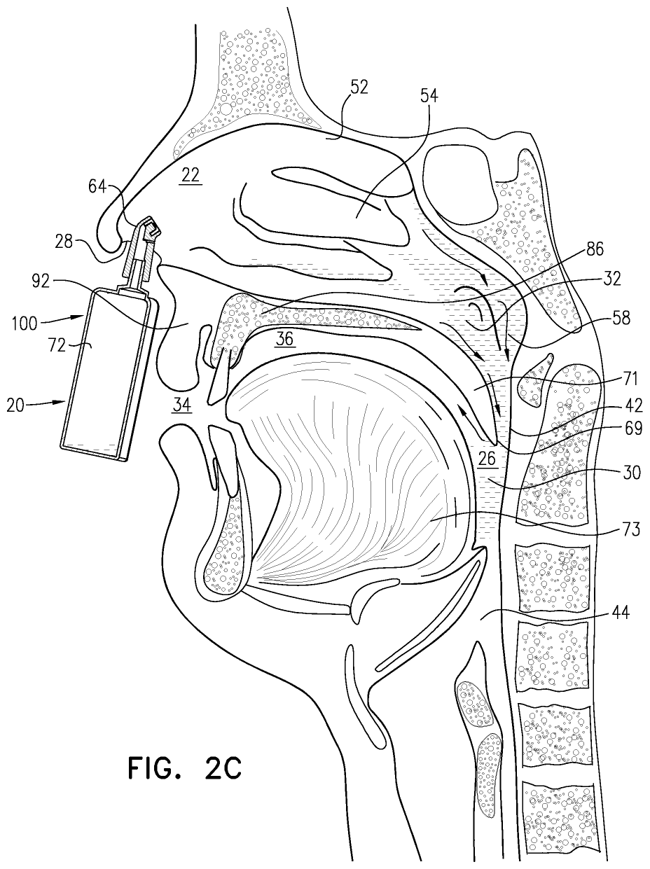

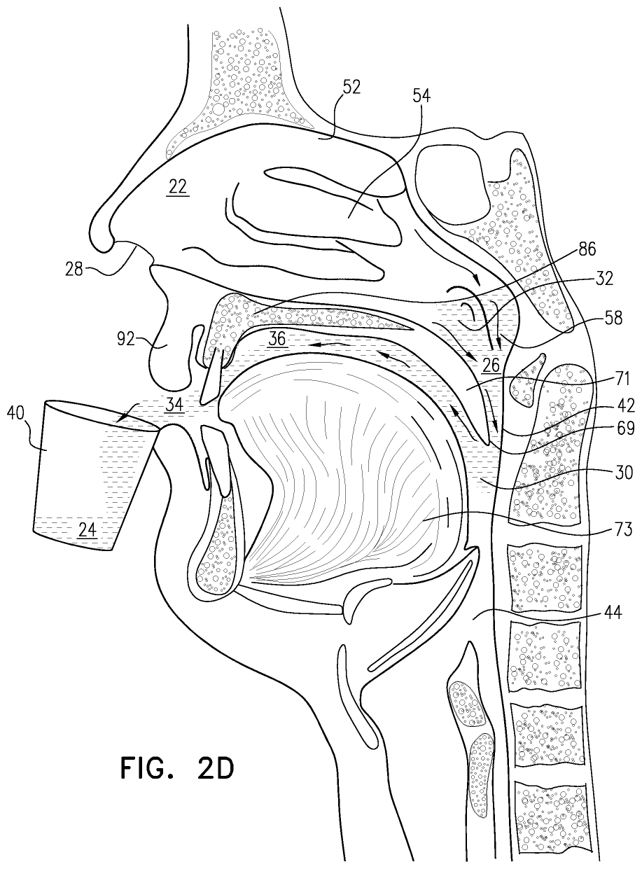

Reference is also made to FIGS. 2A-D, which are schematic cross-sectional illustrations of a method of collecting a specimen sample 24 from a subject, for example one or more specimen samples 24, in accordance with an application of the present invention.

As shown in FIGS. 2A-B, nasal wash fluid 26 is intranasally dispensed into a nasal cavity 22 of the subject, via one or both nostrils 28. In order to intranasally dispense nasal wash fluid 26, a nasal dispenser is inserted into one or both nostrils 28. For example, the nasal dispenser may comprise a conventional nasal dispenser known in the art (also known as a nasal applicator), a conventional nasal dropper known in the art, or a conventional ampoule known in the art, all of which are inserted into one nostril at a time. Alternatively, the nasal dispenser may comprise one of the nasal dispensers described herein, such as nasal wash fluid dispenser 20 or the other nasal dispensers described hereinbelow, which may be inserted into one or both nostrils 28. FIG. 2A shows nasal wash fluid dispenser 20 inserted into nasal cavity 22 via one of nostrils 28.

As shown in FIG. 2C, nasal wash fluid 26 is intranasally dispensed such that nasal wash fluid 26 washes biological material (typically, but necessarily, including saliva, which contains inter alia mucus) into an oropharynx 30 from (a) nasal cavity 22, (b) a nasopharynx 32, or (c) nasal cavity 22 and nasopharynx 32. Typically, nasal wash fluid 26 loosens the biological material in nasal cavity 22, nasopharynx 32, and/or oropharynx 30.

As shown in FIG. 2D, the method further comprises, thereafter, collecting, via an anterior opening 34 of an oral cavity 36 of the subject, specimen sample 24 that contains at least a portion of the biological material washed into oropharynx 30 by nasal wash fluid 26. (Anterior opening 34 is the opening of the mouth between the lips, between outside oral cavity 36 and inside oral cavity 36.)

Specimen sample 24 is typically collected into a collection receptacle 40, as shown in FIG. 2D.

For some applications, collection receptacle 40 may have a surface that is treated so as not to allow sticking of a particulate.

In some applications, collection receptacle 40 used to collect specimen sample 24 comprises a stabilizer (e.g., to help maintain enzyme activity), an inhibitor (e.g., to help prevent RNA degradation), and/or a purifier. Alternatively or additionally, in some applications, after collection, specimen sample 24 is transferred to a liquid container of a testing device, such as any of the testing devices described in the patent applications incorporated herein by reference below; the liquid container of the testing device comprises a stabilizer, an inhibitor, and/or a purifier.

This method comprehensively samples multiple respiratory sites of the upper respiratory tract to collect biological material in a specimen sample 24 and optionally can be performed by the subject (patient) alone without exposing others to contagion. Alternatively, the method can be performed entirely or partially by somebody other than the subject, for example a healthcare provider or a relative of the subject. The method is simpler to perform than nasopharyngeal and pharyngeal swabbing conventionally performed for sample specimen collection, which are heavily dependent on the skill of the healthcare provider and are believed to result in many false negatives because of poor techniques. In addition, conventional swabbing cannot be performed by most subjects on themselves.

In some applications, the method comprises collection of one or more specimen samples 24 containing biological material from one or more of the following locations: nasal cavity 22, nasopharynx 32, and oropharynx 30, and, in some applications (such as when the method further comprises gargling and/or swishing, as described hereinbelow), a posterior pharynx 42, a hypopharynx 44 and lower airways, and in some applications, oral cavity 36, thereby sampling the entire area where pathogens are found in the upper aerodigestive tract.

In applications in which the particulate includes a virus, the virus may include free virions (i.e., extracellular virions) and/or intracellular virions, which, optionally are released from cells (e.g., epithelial cells) of the biological material after collection of specimen sample 24 and before testing the presence of the virus. For example, testing techniques may be used that are described in U.S. Provisional Application 62/988,259, filed Mar. 11, 2020, and/or International Application PCT/IB2021/052055, filed Mar. 11, 2021, which are assigned to the assignee of the present application and incorporated herein by reference.

Reference is still made to FIGS. 2A-D. As used in the present application, including in the claims and Inventive Concepts, “biological material” includes, but is not limited to, microorganisms, antigens, human cells, extracellular soluble and non-soluble particles, cellular components and products, blood products, cellular biomarkers, hormones, vitamins, electrolytes, chemical mediators from cells such as mediators of inflammation, pollens, mucus, saliva, sputum, respiratory particles, droplets derived from the upper and lower airways, nucleic acids including DNA and RNA, and chemicals originating from external vapors. As used in the present application, including in the claims and Inventive Concepts, microorganisms may include either pathogenic microorganisms or non-pathogenic microorganisms or both, for example, viruses, bacteria, protozoa, and fungi.

As used in the present application, including in the claims and Inventive Concepts, human cells may include epithelial cells, for example, goblet cells and/or columnar epithelial cells primarily derived from nasal cavity 22 and/or squamous epithelial cells primarily derived from oral cavity 36. As used in the present application, including in the claims and Inventive Concepts, human cells may also include cellular responders of the immune system, for example, leukocytes (e.g., neutrophils, eosinophils, lymphocytes, or monocytes), macrophages, mast cells, and histocytes. As used in the present application, including in the claims and Inventive Concepts, cellular components and products include lipids, proteins, glycoproteins, proteoglycans, mucins, and nucleic acids such as DNA and RNA.

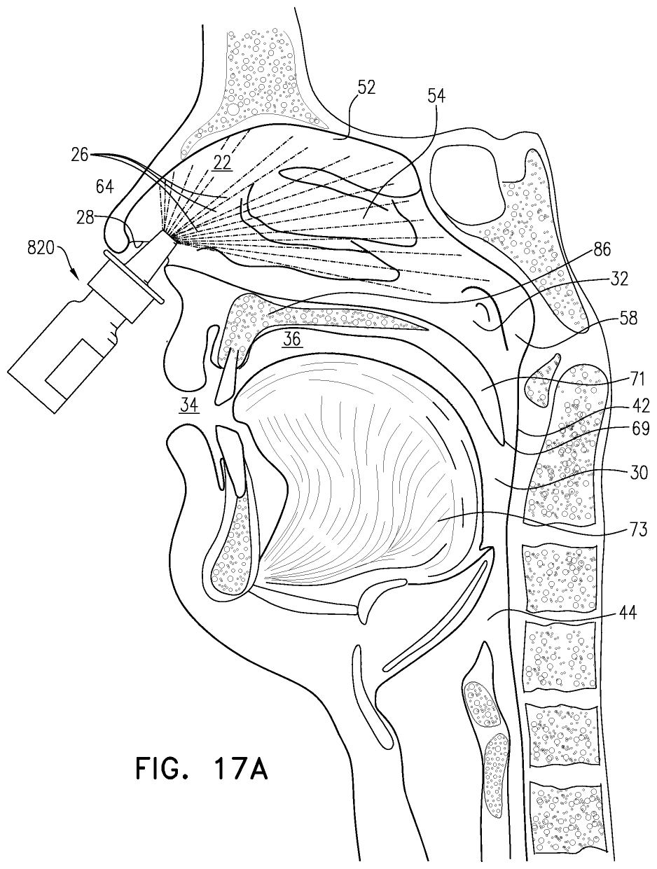

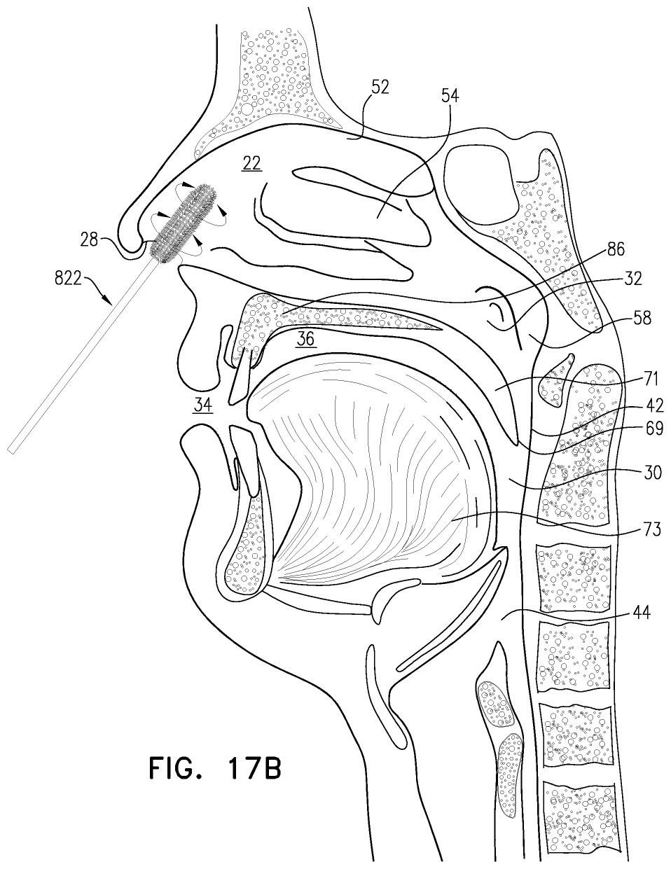

Reference is again made to FIG. 2C. For some applications, the subject tilts his or her head back before, while, and/or after nasal wash fluid 26 is intranasally dispensed into nasal cavity 22, e.g., such as described in more detail hereinbelow with reference to FIGS. 17A-B. Alternatively, the subject holds his or her head in a neutral position or tilted forward while nasal wash fluid 26 is intranasally dispensed into nasal cavity 22, and optionally tilts his or her head back after nasal wash fluid 26 is intranasally dispensed into nasal cavity 22. Although tilting of the subject's head back and forward is generally described herein as being performed by the subject himself or herself, the subject's head may alternatively be tilted by somebody else, such as if the subject is too young or otherwise unable to tilt his or her own head.

For any of the applications described herein, intranasally dispensing nasal wash fluid 26 into nasal cavity 22 may comprise intranasally dispensing nasal wash fluid 26 into nasal cavity 22 while the subject is lying supine. For example, this position may be particularly appropriate when a conventional nasal dropper or a conventional ampoule is used for dispending nasal wash fluid 26.

In some applications, the method further comprises waiting for a period of time between (a) concluding intranasally dispensing nasal wash fluid 26 into nasal cavity 22 and (b) collecting specimen sample 24, the period of time at least 1 minute, e.g., at least 2 minutes, at least 3 minutes, or at least 5 minutes, such as at least 10 minutes. Optionally, the period of time is at least 15 minutes, or at least 30 minutes. The inventors found that waiting for a period of time less than 90 minutes between intranasally dispensing nasal wash fluid 26 into nasal cavity 22 and collecting specimen sample 24 increased the quantity of virus in the collected specimen sample. The inventors hypothesize that waiting for the period of time provides enough time for the subject's natural nasal mucociliary clearance (NMC) system to transport nasal wash fluid 26 containing biological material (typically including mucus) from nasal cavity 22 into oropharynx 30.

Reference is again made to FIG. 2B. In some applications, the method comprises dispensing intranasally a total volume of 0.2-20 mL of a nasal wash fluid 26, for example, 0.2-10 mL, for example, 0.2-5 mL, for example, 0.6-5 mL, into nasal cavity 22. The total nasal wash fluid 26 volume of 0.2-20 mL describes the total volume introduced into the entire nasal cavity 22 through either one or both nostrils 28. For example, 0.1-10 mL may be dispensed into each nostril 28, for a total volume of 0.2-20 mL, or for example, 0.2-20 mL may be dispensed into one nostril 28. Experiments performed by the inventors showed that fluid drained into oropharynx 30 when 1 mL of fluid was dispensed into nasal cavity 22, and did not drain into oropharynx 30 enough to be expressed out of the mouth when only 0.5 mL of fluid was dispensed into nasal cavity 22. However, the inventors hypothesize that dispensing a smaller volume of nasal wash fluid 26, such as as little as 0.2 mL, into nasal cavity 22 may be adequate for draining into oropharynx 30 and expressing out of the mouth if nasal wash fluid 26 is dispensed at a higher pressure and/or is better aimed toward nasopharynx 32 than in the experiments performed.

In some applications, nasal wash fluid 26 is intranasally dispensed as one or more types of fluid discharge selected from the group consisting of: a fluid stream, a spray, a mist, a pressurized aerosol, steam, and an atomized fluid.

In some applications, nasal wash fluid 26 is dispensed intranasally using a circular rotary motion that generates dispersion within nasal cavity 22.

In some applications, nasal wash fluid 26 is intranasally dispensed in a plurality of pulses so as to increase volume without sacrificing comfort; for example, each of the pulses may have a volume of 0.02-10 mL, for example, 0.02-1 mL. Lowering the volume or the pressure of the fluid pulses reduces discomfort and unintentional swallowing, and a plurality of pulses increases volume and surface area sampled, thereby increasing the amount of total particulate collected in specimen sample 24, for improved sampling.

In some applications, nasal wash fluid 26 comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution.

Typically, nasal wash fluid 26 does not comprise a drug molecule. Alternatively, nasal wash fluid 26 does comprise a drug molecule.

For some applications, nasal wash fluid 26 comprises an odorant.

Reference is made to FIGS. 2B and 2C. In some applications, nasal wash fluid 26 is intranasally dispensed into nasal cavity 22 (either continuously or in a plurality of pulses) until the subject senses (e.g., by feeling and/or tasting) nasal wash fluid 26 in oropharynx 30. For applications which comprise gargling and/or swishing, as described herein, the subject typically gargles and/or swishes after sensing nasal wash fluid 26 in oropharynx 30.

For some of these applications, nasal wash fluid 26 comprises a tastant, for example, menthol flavoring, to monitor fluid passage and the movement of the biological material and nasal wash fluid 26 from nasal cavity 22 and nasopharynx 32 into oropharynx 30. Optionally, as mentioned above, the subject tilts his or her head back before, while, and/or after nasal wash fluid 26 is intranasally dispensed into nasal cavity 22, e.g., such as described in more detail hereinbelow with reference to FIGS. 17A-B. Further optionally, for applications in which nasal wash fluid 26 is separately intranasally dispensed into both nostrils, after the subject senses, in oropharynx 30, nasal wash fluid 26 administered to one of the nostrils, nasal wash fluid is intranasally dispensed into the other nostril, until the subject senses the additional nasal wash fluid in oropharynx 30.

As used in the present application, including in the claims and Inventive Concepts, a “tastant” is a taste-provoking chemical molecule that is dissolved in a fluid, and stimulates the sense of taste. For example, the tastant may comprise a non-salty flavor, such as menthol flavoring, a sweet flavor (e.g., comprising a sugar), a bitter flavor; a salty flavor, e.g., saline solution; and/or a non-bitter flavor.

Alternatively or additionally, in some applications, nasal wash fluid 26 comprises a color additive to monitor fluid passage and the movement of the biological material and nasal wash fluid 26 from nasal cavity 22 and nasopharynx 32 into oropharynx 30, and collecting specimen sample 24 comprises confirming that specimen sample 24 contains at least a portion of nasal wash fluid 26 by visually ascertaining that specimen sample 24 comprises the color additive. For some applications, visually ascertaining is performed by the human eye. Alternatively, for some applications, visually ascertaining is performed using spectroscopy, image processing, and/or three-dimensional (3D) image sensing; any of these techniques may optionally utilize artificial intelligence. Such confirmation may identify an inadvertent and/or a deliberate lack of administration of the nasal wash and/or passage of the nasal wash into oropharynx 30.

Alternatively or additionally, in some applications, nasal wash fluid 26 comprises a chemical additive (which may or may not have a color) to monitor fluid passage and the movement of the biological material and nasal wash fluid 26 from nasal cavity 22 and nasopharynx 32 into oropharynx 30, and collecting specimen sample 24 comprises confirming that specimen sample 24 contains at least a portion of nasal wash fluid 26 by ascertaining that specimen sample 24 includes the chemical additive. For some applications, ascertaining is performed using spectroscopy, image processing, and/or three-dimensional (3D) image sensing; any of these techniques may optionally utilize artificial intelligence. For some applications, ascertaining is performed using the same technique used to test for the presence of the particulate, and the chemical additive is configured to be detectable during the test (for example, the chemical additive may be a marker, optionally including DNA and/or RNA). For example, ascertaining may be performed using an immunoassay (for example, a lateral-flow immunochromatographic assay, a chromatographic digital immunoassay, or an enzyme-linked immunosorbent assay (ELISA)); or a molecular-based assay (for example, nucleic acid hybridization, or for example, nucleic acid amplification, including polymerase chain reaction (PCR) amplification, real-time quantitative PCR (qPCR) amplification, reverse transcriptase PCR (RT-PCR) amplification, and isothermal amplification, such as loop-mediated isothermal amplification (LAMP)). Such confirmation may identify an inadvertent and/or a deliberate lack of administration of the nasal wash and/or passage of the nasal wash into oropharynx 30.

Further alternatively or additionally, in some applications, nasal wash fluid 26 comprises a chemical that increases the amount of biological material in specimen sample 24 by enhancing the clearance of the mucus as well as dislodging the biological material lining the mucosa, for example, a mucolytic agent such as acetylcysteine, or for example, a detergent such as polysorbate, or for example, a gas such as pressurized carbon dioxide to generate bubbles or nitrous oxide to increases the speed of mucociliary clearance.

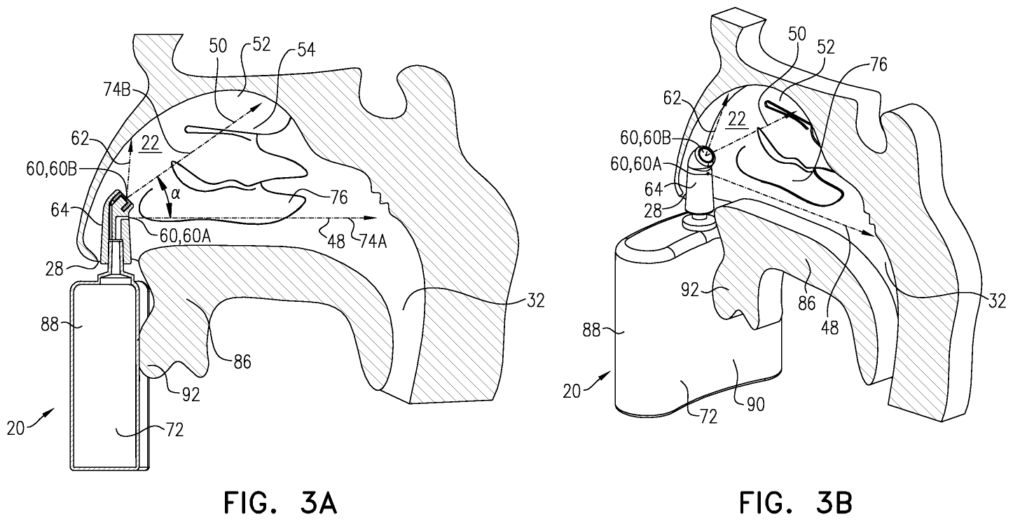

Reference is made to FIGS. 3A-B, which are schematic cross-sectional illustrations of nasal wash fluid dispenser 20 inserted into nasal cavity 22, in accordance with an application of the present invention. FIG. 3B shows nasal wash fluid dispenser 20 rotated slightly along a lateral axis compared to FIG. 3A, so as to provide an additional perspective.

In some applications, the method described hereinabove with reference to FIGS. 2A-D comprises dispensing nasal wash fluid 26 multidirectionally into each of the one or two fossae of nasal cavity 22. In some applications, intranasally dispensing nasal wash fluid 26 multidirectionally within nasal cavity 22 comprises dispensing nasal wash fluid 26 into nasal cavity 22 in a posterior direction 48 toward nasopharynx 32 and in a superoposterior direction 50 toward an olfactory area 52 of nasal cavity 22 above a superior nasal concha 54, for example, through a plurality of nozzles 60, for example, through exactly two nozzles 60.

As used in the present application, including in the claims and Inventive Concepts, a “nozzle” is a device designed to control the direction or characteristics of a fluid flow as it exits an enclosed chamber or tube.

In some applications, intranasally dispensing nasal wash fluid 26 multidirectionally within nasal cavity 22 comprises intranasally dispensing nasal wash fluid 26 within nasal cavity 22 via the plurality of nozzles 60 with respective different fluid flow characteristics, the fluid flow characteristics including one or more characteristics selected from the group consisting of: volume, sprayed particle size, velocity, and flow rate. For example, in some applications, the volume of nasal wash fluid 26 dispensed in posterior direction 48 may be greater than the volume of nasal wash fluid 26 dispensed in superoposterior direction 50 so as to increase the amount of biological material collected in specimen sample 24, since the majority of the biological material contained within nasal cavity 22 drains towards nasopharynx 32 and therefore is believed to be a prime site for sampling. In other applications, for example, the volume of nasal wash fluid 26 dispensed in superoposterior direction 50 may be greater than the volume of nasal wash fluid 26 dispensed in posterior direction 48. These applications may be beneficial since the inventors hypothesize that some viruses may be more concentrated in olfactory area 52, and that reflexive swallowing or premature spitting of nasal wash fluid 26 by the subject may sometimes be induced if a large volume of fluid is dispensed posteriorly.

In some applications, intranasally dispensing nasal wash fluid 26 multidirectionally within nasal cavity 22 comprises intranasally dispensing nasal wash fluid 26 within nasal cavity 22 via the plurality of nozzles 60 with equal fluid flow characteristics, the fluid flow characteristics including one or more characteristics selected from the group consisting of: volume, sprayed particle size, velocity, and flow rate.

In some applications, intranasally dispensing nasal wash fluid 26 multidirectionally within nasal cavity 22 comprises intranasally dispensing nasal wash fluid 26 within nasal cavity 22 via the plurality of nozzles 60 either simultaneously or sequentially.

In some applications, the method described hereinabove with reference to FIGS. 2A-D comprises dispensing nasal wash fluid 26 unidirectionally into both nostrils 28 (and thus into both fossae of the nasal cavity), for example, through a single nozzle 60. In some applications, intranasally dispensing nasal wash fluid 26 unidirectionally within nasal cavity 22 comprises dispensing nasal wash fluid 26 into nasal cavity 22 in posterior direction 48 toward nasopharynx 32 near a pharyngeal recess 58, or in superoposterior direction 50 toward olfactory area 52, or in a superior direction 62. For these applications, nasal wash fluid dispenser 20 may comprise only a single nozzle 60 (configuration not shown), or a conventional nasal dispenser known in the art may be used.

Dispensing nasal wash fluid 26 to target the posterior aspect of nasal cavity 22 toward nasopharynx 32 may increase the quality of the collected specimen sample 24 because a majority of the biological material contained within nasal cavity 22 drains toward nasopharynx 32, which is therefore a prime site for sampling. Alternatively or additionally, dispensing nasal wash fluid 26 to target the superoposterior aspect of nasal cavity 22 toward olfactory area 52 may increase the quality of the collected specimen sample 24 because olfactory area 52 contains high levels of infectious particles in infected individuals, such as with viruses, for example, SARS-CoV-2, in infected individuals with or without symptoms of olfactory dysfunction. A case study analyzed MRIs of a patient with confirmed COVID-19 who had experienced symptomatic olfactory dysfunction and determined that there were no anomalies present in the olfactory bulb and track, but that there was significant bilateral inflammation of the olfactory cleft in the nasal cavity. (Eliezer M, Hautefort C, Hamel A, et al. (2020). “Sudden and Complete Olfactory Loss Function as a Possible Symptom of COVID-19.” JAMA Otolaryngol Head Neck Surg. Published online Apr. 8, 2020. doi:10.1001/jamaoto.2020.0832). This case study supports the hypothesis that olfactory area 52 around the olfactory cleft above superior nasal concha 54 in nasal cavity 22 may contain high levels of infectious particles in individuals infected with SARS-CoV-2.

In some applications, intranasally dispensing nasal wash fluid into nasal cavity 22 comprises inserting a tubular applicator 64 of nasal wash fluid dispenser 20 into a nostril 28 of the subject, for example, inserting two tubular applicators 64 of nasal wash fluid dispenser 20 into both nostrils 28 of the subject.

Reference is again made to FIGS. 2B-C. In some applications, after nasal wash fluid 26 is dispensed into nasal cavity 22, the subject then sniffs or snorts, one or more times, the intranasally-introduced nasal wash fluid 26 posteriorly further into nasal cavity 22, allowing nasal wash fluid 26 to reach nasopharynx 32, from where nasal wash fluid 26 then drains into oropharynx 30. In other words, the sniffing or snorting moves nasal wash fluid 26 within nasal cavity 22 posteriorly toward nasopharynx 32 from where it subsequently drains to oropharynx 30. The sniffing or snorting comprises swiftly drawing air through the nose in short, usually audible, inhalations, that are more sudden and stronger than ordinary breathing. The sniffing or snorting does not necessarily comprise smelling. Alternatively, the intranasally-introduced nasal wash fluid 26 moves posteriorly further into nasal cavity 22 to reach nasopharynx 32 through normal mucociliary movement, without any special action by the subject, such as by sniffing or snorting. Nasal wash fluid 26 then drains from nasopharynx 32 into oropharynx 30, such as shown in FIG. 2C. In other words, the normal mucociliary movement moves nasal wash fluid 26 within nasal cavity 22 posteriorly toward nasopharynx 32, from where nasal wash fluid 26 subsequently drains to oropharynx 30.

In some applications, collecting specimen sample 24 from oral cavity 36 of the subject is performed by the subject alone, while in other applications, collecting specimen sample 24 from oral cavity 36 of the subject is performed by someone other than the subject, for example, by a healthcare provider or relative of the subject.

Reference is again made to FIG. 2D. In some applications, collecting specimen sample 24 comprises collecting specimen sample 24 from oral cavity 36 of the subject by the subject expressing, e.g., spitting, out the contents of oral cavity 36 from oral cavity 36 via anterior opening 34. Typically, the contents contain at least a portion of the intranasally-introduced nasal wash fluid 26 and at least a portion of the biological material. For example, the subject may spit out at least a portion of the intranasally-introduced nasal wash fluid 26 and at least a portion of an orally-introduced oral wash fluid 68 from oral cavity 36. In some applications, collecting the one or more specimen samples 24 comprises collecting a specimen sample 24 that was spit, by the subject, out of oral cavity 36 via anterior opening 34, which typically contains at least a portion of the intranasally-introduced nasal wash fluid 26 and at least a portion of the biological material.

Typically, in applications in which specimen sample 24 is collected by the subject expressing, e.g., spitting, out the contents of oral cavity 36, specimen sample 24 is collected into collection receptacle 40, such as by the subject directly expressing, e.g., spitting, specimen sample 24 into collection receptacle 40. Collection receptacle 40 may, for example, comprise a cup, as shown, or may alternatively comprise a collection receptacle of a sample processing device or sampling testing device, such as described, for example, in one or more of the patent applications incorporated hereinbelow by reference, or otherwise known in the art.

For any of the applications described herein that comprise spitting by the subject, the spitting optionally comprises hawking, i.e., forcefully spitting after forcing material up from the throat, e.g., by making a harsh guttural sound.

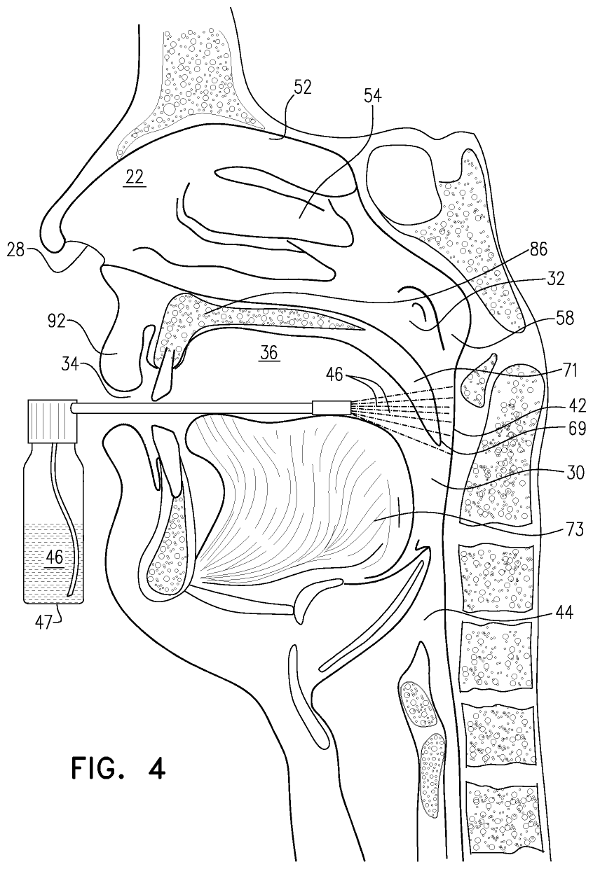

Reference is now made to FIG. 4, which is a schematic cross-sectional illustration of a portion of a method for collecting specimen sample 24 from the subject, in accordance with an application of the present invention. In some applications, the method of FIGS. 2A-D further comprises, before collecting specimen sample 24, spraying, via anterior opening 34 of oral cavity 36, oropharynx 30 with oropharyngeal fluid wash 46. This spraying may help loosen and increase the collection of biological material from the walls of oropharynx 30 (e.g., the walls of posterior pharynx 42), and/or may provide better coverage of posterior pharynx 42 than gargling and/or swishing alone, and thus result in better sampling of posterior pharynx 42 than gargling and/or swishing alone. This spraying is typically performed after intranasally dispensing nasal wash fluid 26 into nasal cavity 22, but may alternatively be performed prior to intranasally dispensing nasal wash fluid 26 into nasal cavity 22.

Reference is again made to FIGS. 2A-D. In some applications, the nasally-introduced nasal wash fluid 26, after it has been washed into oropharynx 30, such as shown in FIG. 2C, is gargled by the subject, swished by the subject in oral cavity 36, or both gargled and swished by the subject in oral cavity 36, prior to collecting specimen sample 24 from oral cavity 36, such as shown in FIG. 2D. In some of these applications, the subject alternates multiple times between (a) gargling and/or swishing and (b) sniffing or snorting, before collecting specimen sample 24 from oral cavity 36. Typically, the alternation between (a) gargling and/or swishing and (b) sniffing or snorting occurs while nasal wash fluid 26 remains in oropharynx 30 of the subject. In general, the subject tilts his or her head back during all of the gargling and/or swishing steps of the methods described and claimed herein. As used in the present application, including in the claims and Inventive Concepts, “gargling” means holding a liquid in the throat (typically in the oropharynx) and agitating with air expelled from the lungs. In general, during gargling, the subject avoids swallowing to the extent reasonably possible.

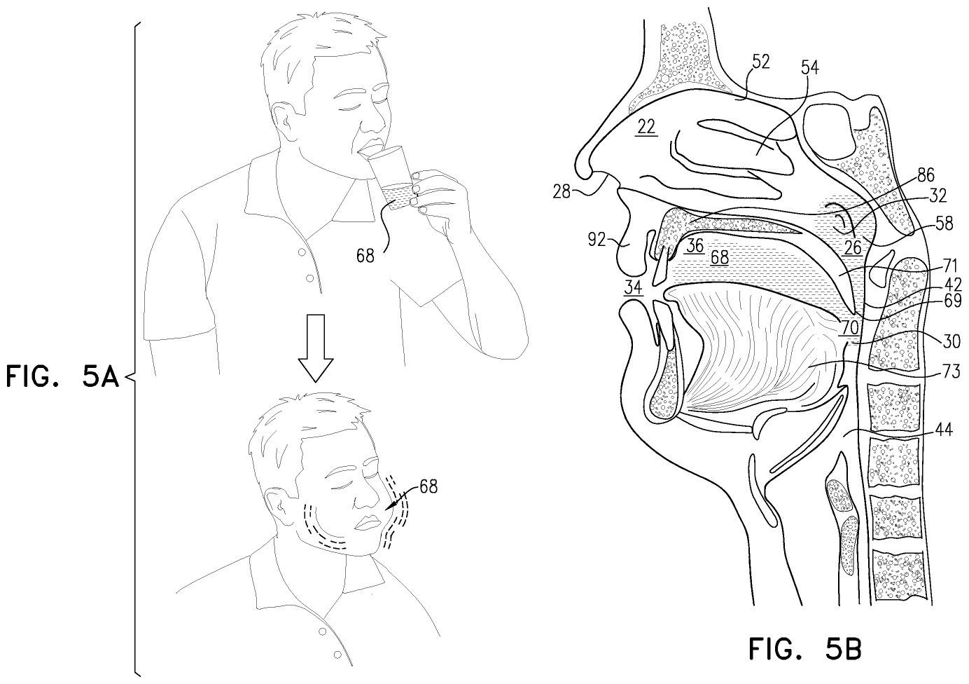

Reference is still made to FIGS. 2A-D and is additionally made to FIGS. 5A-B, which are schematic illustrations of a portion of a method for collecting specimen sample 24 from the subject, in accordance with an application of the present invention. In some applications, the method of FIGS. 2A-D further comprises, before collecting specimen sample 24, orally dispensing oral wash fluid 68 into oral cavity 36 via anterior opening 34 (typically by the subject), such as shown in FIG. 5A, before or after intranasally introducing nasal wash fluid 26, to combine with the intranasally-introduced nasal wash fluid 26 to form a mixture 70 in oropharynx 30, such as shown in FIG. 5B. Thereafter, the subject gargles mixture 70, and specimen sample 24 is collected from oral cavity 36. Specimen sample 24 contains at least a portion of mixture 70 and the at least a portion of the biological material washed into oropharynx 30 with nasal wash fluid 26. Optionally, the subject additionally swishes mixture 70 in oral cavity 36.

In some applications, the method of FIGS. 2A-D further comprises, before collecting specimen sample 24, orally dispensing oral wash fluid 68 into oral cavity 36 via anterior opening 34 (typically by the subject), such as shown in FIG. 5A, before or after intranasally introducing nasal wash fluid 26. The subject gargles oral wash fluid 68 such that oral wash fluid 68 forms mixture 70 with the intranasally-introduced nasal wash fluid 26 in oropharynx 30 and/or oral cavity 36, such as shown in FIG. 5B. Specimen sample 24 contains at least a portion of mixture 70 and the at least a portion of the biological material washed into oropharynx 30 with nasal wash fluid 26. Optionally, the subject additionally swishes mixture 70 in oral cavity 36.

In some applications, the method of FIGS. 2A-D further comprises, before collecting specimen sample 24, orally dispensing oral wash fluid 68 into oral cavity 36 via anterior opening 34 (typically by the subject), such as shown in FIG. 5A, before or after intranasally introducing nasal wash fluid 26. The subject gargles oral wash fluid 68. Specimen sample 24 contains at least a portion of oral wash fluid 68, the at least a portion of the biological material washed into the oropharynx with the nasal wash fluid, and typically, but not necessarily, a portion of nasal wash fluid 26. Optionally, the subject additionally swishes oral wash fluid 68 in oral cavity 36.

In some applications, the method of FIGS. 2A-D further comprises, before collecting specimen sample 24, orally dispensing oral wash fluid 68 into oral cavity 36 via anterior opening 34 (typically by the subject), such as shown in FIG. 5A, before or after intranasally introducing nasal wash fluid 26. The subject swishes oral wash fluid 68 in oral cavity 36. Specimen sample 24 contains at least a portion of oral wash fluid 68, the at least a portion of the biological material washed into the oropharynx with the nasal wash fluid, and typically, but not necessarily, a portion of nasal wash fluid 26.

In some applications, collecting specimen sample 24 comprises collecting specimen sample 24 from oral cavity 36 of the subject by the subject expressing, e.g., spitting, out the contents of oral cavity 36 from oral cavity 36 via anterior opening 34. For example, the subject may spit out at least a portion of the intranasally-introduced nasal wash fluid 26 and at least a portion of orally-introduced oral wash fluid 68 from oral cavity 36.

Experiment 8, described hereinbelow, provides evidence of the efficacy of these techniques for collecting specimen samples for detection of SARS-CoV-2 using quantitative PCR (qPCR).

For example, 5-10 mL of oral wash fluid 68 may be orally dispensed into oral cavity 36 via anterior opening 34.

In some applications, the subject alternates multiple times between gargling and sniffing or snorting, before collecting specimen sample 24 from oral cavity 36. Typically, the alternation between gargling and sniffing or snorting occurs while nasal wash fluid 26 and/or oral wash fluid 68 remains in oropharynx 30 of the subject.

For any of the applications described herein, orally dispensing oral wash fluid 68 into oral cavity 36 via anterior opening 34 of oral cavity 36 may comprise orally dispensing oral wash fluid 68 into oral cavity 36 via anterior opening 34 of oral cavity 36 while the subject is sitting or standing in a vertical position.

In some applications, oral wash fluid 68 comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution.

Typically, oral wash fluid 68 does not comprise a drug molecule. Alternatively, oral wash fluid 68 comprises a drug molecule.

In some applications, oral wash fluid 68 comprises a tastant, for example, menthol flavoring. In some applications, nasal wash fluid 26 and oral wash fluid 68 contain different chemical components that, when combined, generate an indicator. For example, the indicator may be bubbling that is produced by a chemical reaction generated by combining the different chemical components of nasal wash fluid 26 and oral wash fluid 68.

In some applications, nasal wash fluid 26 comprises a first color additive, and oral wash fluid 68 comprises a second colored additive, and when combined, a third color is generated, for example, by mixing of the two color additives, or for example, by a chemical reaction. In some applications, collecting specimen sample 24 comprises confirming that specimen sample 24 contains at least a portion of nasal wash fluid 26 and at least a portion of oral wash fluid 68 by visually ascertaining that specimen sample 24 has an indicator produced by a combination of nasal wash fluid 26 and oral wash fluid 68, for example, a third color produced by a combination of the first and the second color additives. For some applications, visually ascertaining is performed by the human eye. Alternatively, for some applications, visually ascertaining is performed using spectroscopy, image processing, and/or three-dimensional (3D) image sensing; any of these techniques may optionally utilize artificial intelligence.

Alternatively or additionally, in some applications, oral wash fluid 68 comprises a chemical that will increase the amount of biological material in specimen sample 24 by enhancing the clearance of the mucus as well as dislodging the biological material lining the mucosa, for example, a mucolytic agent such as acetylcysteine, or for example, a detergent such as polysorbate, or for example, a gas such as pressurized carbon dioxide to generate bubbles.

Alternatively, in some applications, collecting specimen sample 24 comprises collecting a specimen sample from oral cavity 36 of the subject by aspirating out (using suction) at least a portion of nasal wash fluid 26 and at least a portion of the biological material from oral cavity 36 via anterior opening 34, for example, using a catheter. For example, the at least a portion of the intranasally-introduced nasal wash fluid and orally introduced oral wash fluid 68 may be aspirated out from oral cavity 36 after gargling by the subject. Alternatively or additionally, in some applications, collecting a specimen sample comprises collecting a specimen sample from oral cavity 36 of the subject by drawing out at least a portion of nasal wash fluid 26 and at least a portion of the biological material from oral cavity 36, using absorbent material. For example, the at least a portion of the intranasally-introduced nasal wash fluid and orally introduced oral wash fluid 68 may be drawn from oral cavity 36 using absorbent material after gargling by the subject.

Gargling nasal wash fluid 26 or mixture 70 of nasal wash fluid 26 and oral wash fluid 68 predominately bathes the anterior oropharynx, including the tonsils, tonsillar pillars, uvula 69, soft palate 71, and posterior tongue 73. In this application, the method thus allows at least a portion of the intranasally-introduced nasal wash fluid that sampled nasal cavity 22 and nasopharynx 32 to also sample oropharynx 30 via gargling and to then be collected from oral cavity 36. Additionally, gargling may also allow for collection of respiratory particles and droplets expressed from or found on posterior pharynx 42, hypopharynx 44, oral cavity 36, and lower airways since the act of gargling is a process of exhaling. Therefore, specimen sample 24 collected by this method may contain droplets and particles from the entire respiratory tract including both upper and lower airways. Typically, the collected specimen sample contains at least portion of the nasally-introduced nasal wash fluid solution and at least a portion of the orally introduced oral wash fluid 68 solution and at least a portion of the biological material that originated from the upper aerodigestive tract. Alternatively, the collected specimen sample contains at least portion of the nasally-introduced nasal wash fluid solution and at least a portion of the biological material that originated from the upper aerodigestive tract.

In some applications, the biological material that originated from the upper aerodigestive tract includes any of the biological material listed hereinabove with reference to FIGS. 2A-D. multiple biological materials or particulates that may include: microorganisms, antigens, human cells, extracellular soluble and non-soluble particles, cellular components and products, blood products cellular biomarkers, hormones, vitamins, electrolytes, chemical mediators from cells such as mediators of inflammation, pollens, mucus, saliva, sputum, respiratory particles, droplets derived from the upper and lower airways, nucleic acids including DNA and RNA, and chemicals originating from external vapors. Typically, microorganisms collected in specimen sample 24 may include either pathogenic microorganisms or non-pathogenic microorganisms or both, for example, viruses, bacteria, protozoa, and fungi. Typically, human cells collected in specimen sample 24 may include epithelial cells, for example, columnar epithelial cells primarily derived from the nasal cavity and squamous epithelial cells primarily derived from the oral cavity. Typically, human cells collected in specimen sample 24 may also include cellular responders of the immune system, for example, neutrophils, eosinophils, lymphocytes, monocytes, macrophages, mast cells, and histocytes.

For some applications, the method further comprises waiting for a period of time between (a) concluding intranasally dispensing nasal wash fluid 26 into nasal cavity 22 and (b) orally dispensing oral wash fluid 68 into oral cavity 36, the period of time at least 1 minute, e.g., at least 2 minutes, at least 3 minutes, or at least 5 minutes, such as at least 10 minutes. Optionally, the period of time is at least 15 minutes, or at least 30 minutes. The inventors found that waiting for a period of time less than 90 minutes between intranasally dispensing nasal wash fluid 26 into nasal cavity 22 and orally dispensing oral wash fluid 68 into oral cavity 36 increased the quantity of virus in the collected sample.

For some applications, specimen sample 24 is a first specimen sample, and mixture 70 is a first mixture, and the method further comprises, after collecting the first specimen sample, waiting for a period of time, the period of time at least 1 minute, e.g., at least 2 minutes, at least 3 minutes, or at least 5 minutes, such as at least 10 minutes. Optionally, the period of time is at least 15 minutes, or at least 30 minutes. After waiting, additional oral wash fluid 68 is orally dispensed into oral cavity 36 via anterior opening 34 of oral cavity 36 such that the additional oral wash fluid 68 forms a second mixture with nasal wash fluid 26 in oropharynx 30. Thereafter, the subject gargles the second mixture. Thereafter, a second specimen sample is collected that passed out of anterior opening 34 of oral cavity 36 and contains at least a portion of the second mixture and the at least a portion of the biological material washed into oropharynx 30 with nasal wash fluid 26. The method described in this paragraph typically does not comprise intranasally dispensing nasal wash fluid into nasal cavity 22 after collecting the first specimen sample. Optionally, the period of time is at least 15 minutes, or at least 30 minutes. The inventors found that virus was detected in both the first and second specimen samples, even though additional nasal wash was not intranasally dispensed before the additional oral wash fluid 68 was dispensed and gargled.

Reference is again made to FIG. 2D. In some applications, collecting the one or more specimen samples 24 comprises collecting one or more fluid specimen samples 24. In some applications, collecting one or more specimen samples 24 consists collecting at least two specimen samples, for example, collecting exactly two specimen samples. In some applications, collecting the at least two specimen samples comprises collecting further specimen samples in addition to collecting specimen sample 24 from oral cavity 36, for example, collecting an additional specimen sample by swabbing nasal cavity 22 or by collecting an additional specimen sample by inserting absorbent material into nasal cavity 22, typically after collecting fluid specimen sample 24 from oral cavity 36. In some applications, first specimen sample 24 contains at least a first portion of nasal wash fluid 26 and at least a first portion of the biological material, for example collected from oral cavity 36, and a second specimen sample contains at least a second portion of the portion of nasal wash fluid 26 and at least a second portion of the biological material, for example collected from nasal cavity 22.

In some applications, the two or more specimen samples are combined to produce a combined specimen sample.

In some applications, collecting the second specimen sample comprises collecting from nasal cavity 22 by the subject blowing specimen sample 24 out of nasal cavity 22 via the one or two nostrils 28. In some applications, collecting the second specimen sample comprises collecting the second specimen sample that was blown, by the subject, out of nasal cavity 22 via the one or two nostrils 28.

In other applications, collecting the second specimen sample comprises collecting from nasal cavity 22 by aspirating specimen sample 24 out of nasal cavity 22 via the one or two nostrils 28, for example, using a catheter.

In some applications, collecting the second specimen sample by aspirating specimen sample 24 out from nasal cavity 22 via the one or two nostrils 28 comprises using a negative pressure vacuum generated by nasal wash fluid dispenser 20 after releasing nasal wash fluid 26, for example, by using nasal wash fluid dispenser 20 in accordance with an application of the present invention.

In some applications, collecting a second specimen sample from nasal cavity 22 comprises, after collecting a specimen sample from oral cavity 36 in collection receptacle 40, collecting residual biological material and nasal wash fluid 26 from nasal cavity 22 by blowing out of the nose of the subject into a plastic bag that is attached to collection receptacle 40. For example, the plastic bag may have a distal opening that is hermetically sealed to the proximal opening of collection receptacle 40 and may have a proximal opening that has a sealable closure mechanism, to allow for specimen sample 24 blown out of the nose of the subject into the plastic bag to join with specimen sample 24 collected from oral cavity 36 in collection receptacle 40. There may be a sealable opening or spout in collection receptacle 40 or in the plastic bag that is openable to allow exit of the combined specimen sample for testing.

Reference is again made to FIG. 2B. In some applications, instead of or in addition to intranasally dispensing nasal wash fluid 26 into nasal cavity 22, a vapor (e.g., a warm vapor) is intranasally dispensed into nasal cavity 22 to loosen the biological material in nasal cavity 22. Typically, intranasally dispensing the vapor comprises inhaling, by the subject, the vapor into nasal cavity 22, and, typically, into oropharynx 30 via nasopharynx 32, such that the vapor washes the biological material into oropharynx 30 from (a) nasal cavity 22, (b) nasopharynx 32, or (c) nasal cavity 22 and nasopharynx 32. Typically, specimen sample 24 contains at least a portion of the biological material washed into oropharynx 30 by the vapor.

Reference is made to FIGS. 2A-5B. The sampling methods described herein with reference to FIGS. 2A-5B typically do not comprise swabbing any portion of the subject, unless the methods are specifically described as comprising swabbing a portion of the subject. Alternatively, the sampling methods described herein may optionally comprise swabbing a portion of the subject.

The sampling methods described herein with reference to FIGS. 2A-5B typically do not comprise swabbing nasal cavity 22, unless the methods are specifically described as comprising swabbing nasal cavity 22. Alternatively, the sampling methods described herein may optionally comprise swabbing nasal cavity 22.

The sampling methods described herein with reference to FIGS. 2A-5B typically do not comprise swabbing nasopharynx 32, unless the methods are specifically described as comprising swabbing nasopharynx 32. Alternatively, the sampling methods described herein may optionally further comprise swabbing nasopharynx 32.

In some applications of the present invention, the methods with reference to FIGS. 2A-5B do not comprise swabbing nasopharynx 32, and specimen sample 24 is collected by swabbing oropharynx 30. For some of these applications, the methods do not comprise swabbing any portion of nasal cavity 22. Alternatively, for some of these applications, collecting specimen sample 24 comprises swabbing oropharynx 30 and swabbing one or both anterior nares of nasal cavity 22. Alternatively or additionally, for some of these applications, collecting specimen sample 24 comprises swabbing oropharynx 30 and swabbing one or more turbinates of nasal cavity 22 selected from the group consisting of: a middle turbinate and an inferior turbinate. As is known in the art, the anterior naris is the portion of the nasal cavity anterior to the internal nasal valve.

The sampling methods described herein with reference to FIGS. 2A-5B typically do not comprise swabbing oropharynx 30 and do not comprise swabbing nasopharynx 32, unless the methods are specifically described as comprising swabbing oropharynx 30 and/or nasopharynx 32. Alternatively, the sampling methods described herein may optionally further comprise swabbing oropharynx 30 and/or nasopharynx 32.

For some applications, collecting specimen sample 24 comprises drawing specimen sample 24 out of oral cavity 36 via anterior opening 34 of oral cavity 36 using an absorbent material (e.g., a flocked or cotton swab, or a sponge). (For example, an ORAcollect®⋅RNA Saliva Collection Device (DNA Genotek Inc., a subsidiary of OraSure Technologies, Inc. (Bethlehem, Pa., USA)) may be used.) Optionally, the absorbent material is located on a tip of a collector shaft, and specimen sample 24 is drawn out of oral cavity 36 via anterior opening 34 of oral cavity 36 using the absorbent material by inserting the tip of the collector shaft into oral cavity 36. For some of these applications, specimen sample 24 is drawn out of oral cavity 36 via anterior opening 34 of oral cavity 36 using the absorbent material by the subject sucking on the absorbent material.

For some applications in which the method does not comprise swabbing oropharynx 30 and does not comprise swabbing nasopharynx 32, specimen sample 24 is collected by drawing specimen sample 24 out of oral cavity 36 via anterior opening 34 of oral cavity 36 by contacting one or more portions of oral cavity 36 with an absorbent material (e.g., at a tip of a collector shaft), without swabbing oropharynx 30. (For example, the above-mentioned ORAcollect®⋅RNA Saliva Collection Device may be used.) For example, the one or more portions of oral cavity 36 may include one or more of buccal mucosa, the tongue (e.g., under the tongue), the gums (e.g., the lower gums), and/or the palatal mucosa. For example, for swabbing the lower gums, absorbent material (e.g., at a tip of a collector shaft) may be rubbed back and forth along the lower gums several times.

In applications of the present invention that comprise swabbing the subject, specimen sample 24 may be collected into collection receptacle 40 by placing the swab (or swabs) in collection receptacle 40.

In some applications, the methods described comprise testing the one or more specimen samples for the presence of a particulate, for example, testing the combined specimen sample for the presence of a particulate. Typically, but not necessarily, the particulate is present in at least the biological material washed from nasal cavity 22 and/or nasopharynx 32 to oropharynx 30, and, optionally, also in other biological material from elsewhere in the respiratory tract.

In some applications, testing the one or more specimen samples for the presence of a particulate comprises first concentrating specimen sample 24 by filtration prior to testing for the presence of a particulate (by passing specimen sample 24 through one or more filters).

In some applications, testing the one or more specimen samples for the presence of a particulate comprises testing to aid in the diagnosis of disease, for example, aiding in the diagnosis of infectious diseases or allergies. In some applications, testing the one or more specimen samples for the presence of a particulate comprises testing to aid in the diagnosis of a biological status or inflammatory status. In some applications, testing for the presence of a particulate comprises testing for the presence of one or more of the following soluble or insoluble particulates: a pathogenic microorganism, a non-pathogenic microorganism, an antigen, a human cell, a cellular biomarker, a hormone, a chemical mediator from cells such as a mediator of inflammation, a pollen, a respiratory particle, a particle contained within droplets from the lower airways of the subject, a nucleic acid including DNA and RNA, and a chemical originating from an external vapor. In some applications, the testing for the presence of a particulate comprises testing for the presence of one or more of the following microorganisms: viruses, bacteria, and fungi, e.g., testing for the presence of bacteria, for example, Streptococcus bacteria, such as Streptococcus pyogenes (Strep A), or, for example, testing for the presence of viruses such as Influenza viruses, or for example, coronaviruses, such as SARS-CoV-2. In some applications, specimen sample 24 is tested for the presence of cerebrospinal fluid (CSF).

In some applications, the testing for the presence of a particulate comprises testing for the presence of one or more of the following antigens: viral antigens, bacterial antigens, and fungal antigens.

In some applications, the testing for the presence of a particulate comprises microscopic imaging, fluorescence activated cell sorting (FACS), spectroscopy, microscopy (e.g., automated digital microscopy), image processing, three-dimensional (3D) image sensing, and/or CRISPR diagnostic testing.

In some applications, the testing for the presence of a particulate comprises artificial intelligence. Any of the above-mentioned testing techniques may optionally utilize artificial intelligence.

In some applications, the testing for the presence of a particulate comprises culturing at least a portion of specimen sample 24.

In some applications, the testing for the presence of a particulate comprises performing a particulate detection test. In some applications, the particulate detection test is an immunoassay, for example, a lateral-flow immunochromatographic assay, a chromatographic digital immunoassay, or an enzyme-linked immunosorbent assay (ELISA). In some applications, the particulate detection test is a molecular-based assay, for example, nucleic acid hybridization, or for example, nucleic acid amplification, including polymerase chain reaction (PCR) amplification, real-time quantitative PCR (qPCR) amplification, reverse transcriptase PCR (RT-PCR) amplification, and isothermal amplification, such as loop-mediated isothermal amplification (LAMP). For some of these applications, specimen sample 24 is transported from the site of collection to a remote site, such as a remote laboratory, for testing. For these applications, specimen sample 24, or a portion thereof, may be stored in a liquid, such as a lysis buffer, saline solution (e.g., phosphate buffered saline (PBS)), transport medium (e.g., universal transport medium or a viral transport medium), or a composition comprising a detergent and a buffering agent for stabilizing RNA (e.g., distributed by DNA Genotek Inc., a subsidiary of OraSure Technologies, Inc. (Bethlehem, Pa., USA), for example, ORAcollect®⋅RNA Saliva Collection Device for SARS CoV-2). The liquid is optionally contained in collection receptacle 40, described hereinabove with reference to FIG. 2D.

In some applications, the testing for the presence of the particulate comprises performing a pretesting step prior to testing for the presence of the particulate, comprising one or more of the following pretesting steps: chemical treatment, purification, filtration, enrichment, and/or centrifugation. In other applications, specimen sample 24 is tested directly for the presence of a particulate without any pretesting steps.

Reference is again made to FIGS. 2A-D. In some applications of the present invention, a method is provided that comprises intranasally dispensing nasal wash fluid into nasal cavity 22 such that nasal wash fluid 26 passes from nasal cavity 22 into oropharynx 30 via nasopharynx 32. The method further comprises, thereafter, collecting a specimen sample that passed out of anterior opening 34 of oral cavity 36 and contains at least a portion of nasal wash fluid 26. In this method, biological material of interest for subsequent testing is not necessarily washed from nasal cavity 22 or nasopharynx 32 into oropharynx 30, although, of course, some biological material, such as mucus, will be thus washed. Instead, the biological material of interest for subsequent testing comes from elsewhere in the respiratory tract, typically at least oropharynx 30, and optionally lower in the respiratory tract. In this method, nasal wash fluid 26, upon arriving in oropharynx 30, may help loosen and increase the collection of biological material from the walls (e.g., of posterior pharynx 42) of oropharynx 30, and/or may provide better coverage of posterior pharynx 42 than gargling alone, and thus result in better sampling of posterior pharynx 42 than gargling alone. Thus, this method may be useful for detecting particulates present in the respiratory tract only at the level of oropharynx 30 or lower, but not in nasal cavity 22 or nasopharynx 32.

This method may be practiced in combination with the other methods described herein, including, but not limited to gargling and/or spraying, via anterior opening 34 of oral cavity 36, oropharynx 30 with oropharyngeal fluid wash.

In some applications, specimen samples 24 collected using the collection techniques described herein are tested for a particulate using one or more of the techniques described in the patent applications incorporated hereinbelow by reference, or a technique known in the art. The particulate may be a virus, a bacterium, any of the other particulates described hereinabove, or any of the other biological materials described hereinabove.

Reference is again made to FIG. 4. In some applications, a method is provided that comprises spraying (either by a healthcare worker or the subject), via anterior opening 34 of oral cavity 36, oropharynx 30 with oropharyngeal fluid wash 46. Thereafter, a specimen sample is collected that passed out of anterior opening 34 of oral cavity 36 (e.g., was spit out of anterior opening 34) and contains at least a portion of biological material washed off a pharyngeal wall (typically, the posterior pharyngeal wall) into oropharynx 30 of the subject by the oropharyngeal fluid wash. Specimen sample 24 may additionally or alternatively contain at least a portion of biological material washed off one or more of the following locations: posterior pharynx 42, a tonsillar area, soft palate 71, and posterior tongue 73. This method may be performed without intranasally dispensing nasal wash fluid into nasal cavity 22, or it may be performed in combination with such dispensing, such as described hereinabove. This method may be performed in combination with any of the techniques described herein, such as the particulate testing techniques described herein. For some applications, a spray bottle 47 is provided that contains oropharyngeal fluid wash 46; spray bottle 47 may be an element of a kit comprising other elements described herein.

In some applications, the method further comprises, before collecting specimen sample 24, gargling the oropharyngeal fluid wash by the subject. This method may produce a better specimen sample than would be produced by gargling alone, because gargling alone generally results in the gargle fluid having limited contact with the posterior pharyngeal wall. Alternatively, the method does not comprise gargling the oropharyngeal fluid wash by the subject. Further alternatively or additionally, in some applications, the method further comprises, before collecting specimen sample 24, swishing, by the subject, the oropharyngeal fluid wash in oral cavity 36.

In some applications, the method further comprises, before collecting specimen sample 24, orally dispensing oral wash fluid 68 into oral cavity 36 via anterior opening 34 of oral cavity 36 such that oral wash fluid 68 forms a mixture with the oropharyngeal fluid wash in oropharynx 30; and gargling and/or swishing mixture 70 by the subject. In these applications, specimen sample 24 contains at least a portion of mixture 70 and the at least a portion of the biological material washed off the pharyngeal wall into oropharynx 30 by mixture 70.

In some applications, the spraying is performed while the subject says “ah” or “eh.” This may increase the amount of the oropharyngeal fluid wash that is deposited on the posterior pharyngeal wall, such as to build up enough volume to gargle and/or swish.

In some applications, the biological material includes one or more particulates selected from the group consisting of: a virus (e.g., a coronavirus, such as SARS-CoV-2; or an Influenza virus), a bacterium (e.g., Streptococcus bacterium), a protozoan, and a fungus.

In some applications, a total volume of at least 2 mL (e.g. 2-15 mL, e.g. 2-10 mL) of the oropharyngeal fluid wash is sprayed.

In some applications, oropharynx 30 is sprayed with the oropharyngeal fluid wash in a plurality of pulses. For example, each of the pulses may have a volume of 0.05-5 mL.

In some applications, collecting specimen sample 24 comprises expressing (e.g., spitting), by the subject, specimen sample 24 out of oropharynx 30 via the anterior opening of oral cavity 36.

In some applications of the present application, a method is provided for testing for the presence of a particulate. The particulate may be a virus, a bacterium, any of the other particulates described hereinabove, or any of the other biological materials described hereinabove. This method may optionally be combined with any of the techniques described herein, including the techniques for intranasally dispensing nasal wash fluid. Alternatively, this method may be practiced separately from the other techniques described herein, including without intranasally dispensing nasal wash fluid.

The method comprises collecting, from a subject, a liquid specimen sample that that includes orally-dispensed oral wash fluid 68 and potentially contains the particulate. For some applications, the particulate is selected from the group consisting of: a virus, a bacterium, a protozoan, and a fungus. For example, the particulate may be an Influenza virus or a coronavirus, such as Severe acute respiratory syndrome coronavirus 2 (SARS-CoV-2). Alternatively or additionally, the particulate may be an antibody, an antigen, or a leukocyte, or any of the other particulates described herein, and/or the liquid specimen sample potentially contains any of the biological materials described herein.

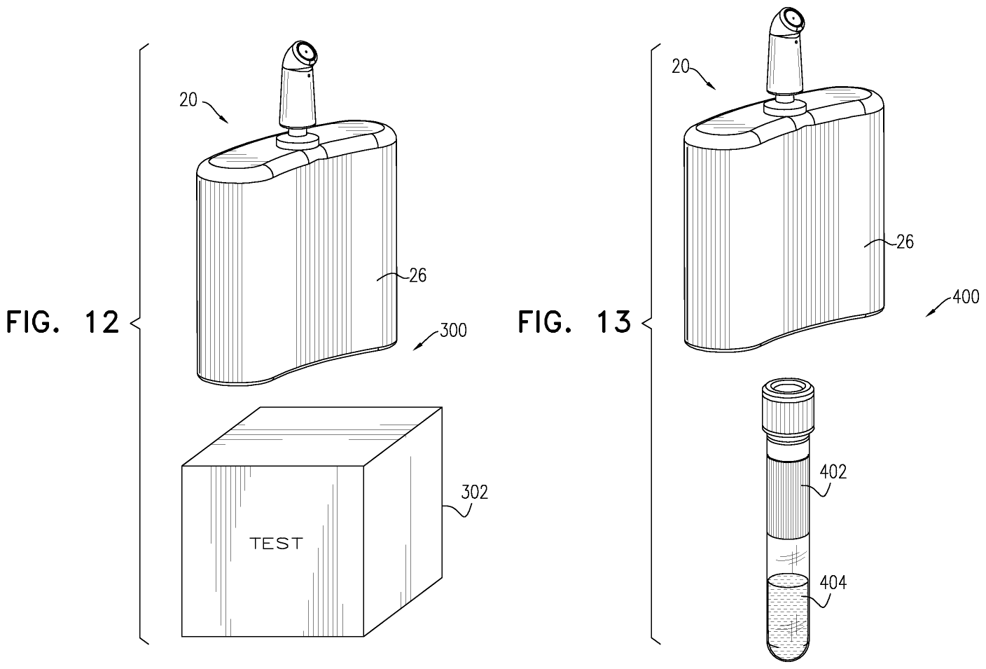

The liquid specimen sample is passed through one or more porous filters to produce a filtrate. For some applications, the one or more porous filters comprise at least one filter having an average absolute or nominal pore size of between 0.1 and 3 microns, such as between 0.2 and 3 microns, such as between 0.2 and 2 microns, e.g., between 0.45 and 2 microns, such as between 0.8 and 2 microns, e.g., between 1 and 2 microns, such as between 1.2 and 2 microns. Alternatively, the average absolute or nominal pore size may be 3-6 microns, 2-10 microns, 6-13 microns, 10-20 microns, 13-20 microns, or 20-40 microns. Alternatively, the average absolute or nominal pore size may be between 0.03 and 0.3 microns. Further alternatively or additionally, the at least one filter may have the pore sizes described hereinbelow with reference to FIGS. 14A-B for large-pored filter(s) 520 of filter units 500A and 500B. Optionally, the above-mentioned at least one filter is at least one first filter, and the one or more porous filters comprise a second filter, downstream from the at least one first filter, the second filter having an average absolute or nominal pore size less than that of the at least one first filter. For example, the average absolute or nominal pore size of the second filter may be between 0.03 and 0.3 microns.

The filtrate is tested for the presence of the particulate (or the other biological materials described herein).

For some applications, collecting the liquid specimen sample comprises:
gargling, by the subject, oral wash fluid 68 to produce the liquid specimen sample that includes oral wash fluid 68 and potentially contains the particulate; and
thereafter, collecting the liquid specimen sample from the subject.

For some applications, collecting the liquid specimen sample comprises:
swishing, by the subject, oral wash fluid 68 in oral cavity 36 of the subject to produce the liquid specimen sample that includes the oral wash fluid and potentially contains the particulate; and
thereafter, collecting the liquid specimen sample from the subject.

For some applications, collecting the liquid specimen sample comprises:
spraying, via anterior opening 34 of oral cavity 36, oropharynx 30 with oral wash fluid 68; and
thereafter, collecting the liquid specimen sample from the subject.

For some applications, testing for the presence of the particulate comprises using spectroscopy to test for the presence of the particulate, using microscopy (e.g., automated digital microscopy) to test for the presence of the particulate, using image processing to test for the presence of the particulate, and/or using three-dimensional (3D) image sensing to test for the presence of the particulate. For some applications, testing for the presence of the particulate comprises using a molecular-based assay (such as nucleic acid amplification, e.g. polymerase chain reaction (PCR) or isothermal amplification, e.g., loop-mediated isothermal amplification (LAMP)) to test for the presence of the particulate. For some applications, testing for the presence of the particulate comprises using another testing technique to test for the presence of the particulate, such as any of the testing techniques described herein. For some applications, testing for the presence of the particulate comprises using artificial intelligence to test for the presence of the particulate. Any of the above-mentioned testing techniques may optionally utilize artificial intelligence.

Typically, testing the filtrate for the presence of the particulate comprises diagnosing a disease.

Typically, the method does not comprise centrifuging the liquid specimen sample before passing the liquid specimen sample through the one or more porous filters.

Typically, the method does not comprise centrifuging the filtrate before testing the filtrate for the presence of the particulate.

Alternatively, the method comprises centrifuging the liquid specimen sample before passing the liquid specimen sample through the one or more porous filters, and/or centrifuging the filtrate before testing the filtrate for the presence of the particulate.

Typically, the method does not comprise culturing the liquid specimen sample before passing the liquid specimen sample through the one or more porous filters.

Typically, the method does not comprise culturing the filtrate before testing the filtrate for the presence of the particulate.

Alternatively, the method comprises culturing the liquid specimen sample before passing the liquid specimen sample through the one or more porous filters, and/or culturing the filtrate before testing the filtrate for the presence of the particulate.

In an application of the present invention, a method is provided for testing for the presence of a particulate. The particulate may be a virus, a bacterium, any of the other particulates described hereinabove, or any of the other biological materials described hereinabove. This method may optionally be combined with any of the techniques described herein, including the techniques for intranasally dispensing nasal wash fluid. Alternatively, this method may be practiced separately from the other techniques described herein, including without intranasally dispensing nasal wash fluid.

The method comprises collecting, from a subject, a liquid specimen sample that that includes orally-dispensed oral wash fluid 68 and potentially contains a particulate selected from the group consisting of: a virus, a bacterium, a protozoan, and a fungus. For example, the particulate may be an Influenza virus or a coronavirus. such as Severe acute respiratory syndrome coronavirus 2 (SARS-CoV-2). Alternatively or additionally, the particulate may be an antibody, an antigen, or a leukocyte, or any of the other particulates described herein, and/or the liquid specimen sample potentially contains any of the biological materials described herein.

The liquid specimen sample is tested for the presence of the particulate using spectroscopy, microscopy (e.g., automated digital microscopy), image processing, three-dimensional (3D) image sensing, and/or artificial intelligence.

For some applications, collecting the liquid specimen sample comprises:
gargling, by the subject, oral wash fluid 68 to produce the liquid specimen sample that includes oral wash fluid 68 and potentially contains the particulate; and
thereafter, collecting the liquid specimen sample from the subject.

For some applications, collecting the liquid specimen sample comprises:
swishing, by the subject, oral wash fluid 68 in oral cavity 36 of the subject to produce the liquid specimen sample that includes the oral wash fluid and potentially contains the particulate; and
thereafter, collecting the liquid specimen sample from the subject.

For some applications, collecting the liquid specimen sample comprises:
spraying, via anterior opening 34 of oral cavity 36, oropharynx 30 with oral wash fluid 68; and
thereafter, collecting the liquid specimen sample from the subject.

For some applications, the liquid specimen sample is passed through one or more porous filters to produce a filtrate, and the filtrate is tested for the presence of the particulate. For example, the one or more filters may have any of the pore sized described hereinabove for the previous method described immediately above.

For some applications, the liquid specimen sample is concentrated by filtration prior to testing for the presence of the particulate, by passing the liquid specimen sample through one or more filters. For example, the one or more filters may have any of the pore sized described hereinabove for the previous method described immediately above. For example, at least one of the one or more filters may have a pore size of 0.03 to 0.3 microns, and/or the pore sizes described hereinbelow with reference to FIGS. 14A-B for large-pored filter(s) 520 of filter units 500A and 500B. Optionally, the liquid specimen sample is tested for the presence of the particulate by analyzing the one or more filters using spectroscopy, microscopy (e.g., automated digital microscopy), image processing, three-dimensional (3D) image sensing, and/or artificial intelligence.

Typically, testing for the presence of the particulate comprises diagnosing a disease.

Typically, the method does not comprise centrifuging the liquid specimen sample before testing for the presence of the particulate.

Typically, when the particulate is a virus, the method does not comprise culturing the liquid specimen sample before testing for the presence of the particulate. Alternatively, for some applications, such as when the particulate is a bacterium, the method further comprises culturing the liquid specimen sample before testing for the presence of the particulate.

Alternatively, the method comprises centrifuging the liquid specimen sample before testing for the presence of the particulate, and/or culturing the liquid specimen sample before testing for the presence of the particulate.

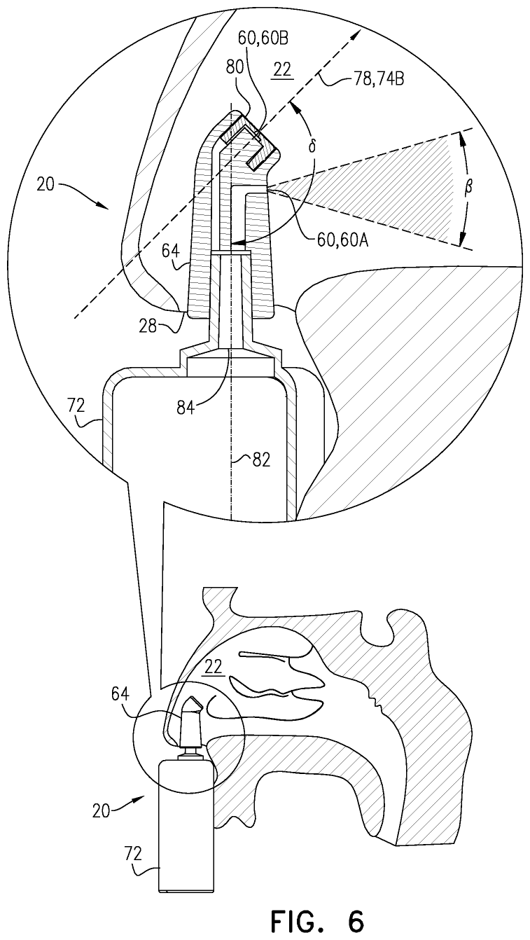

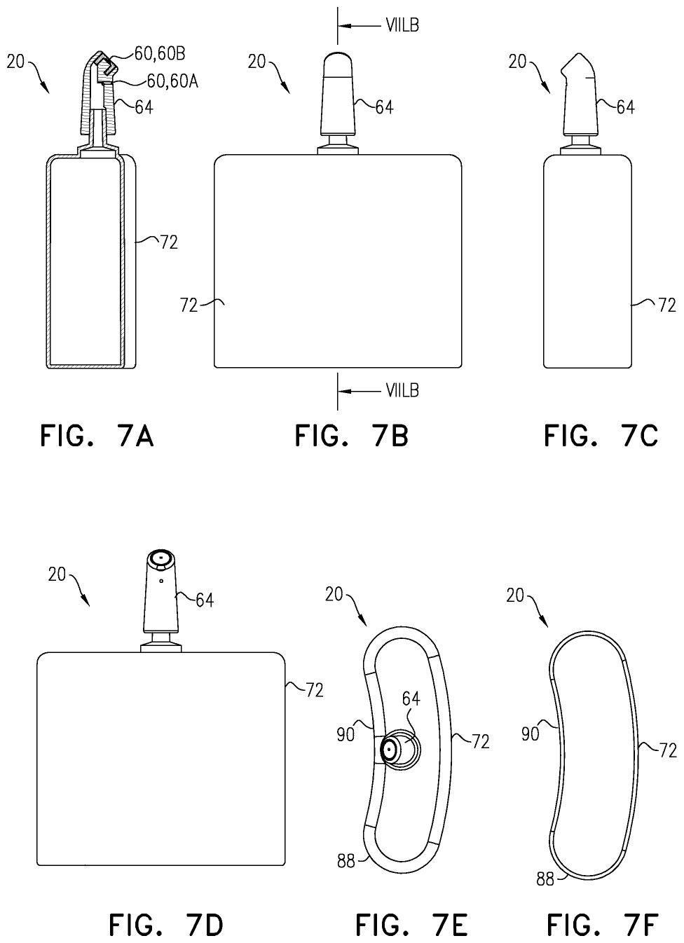

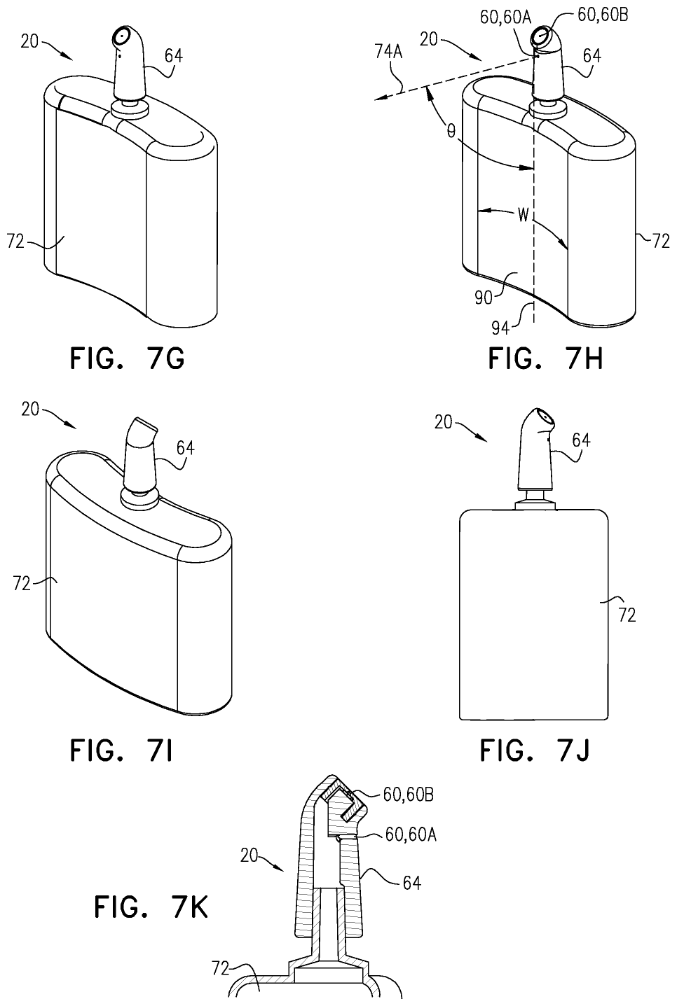

Reference is again made to FIGS. 1, 2A-D, and 3A-B, and is additionally made to FIG. 6, which is a schematic illustration of nasal wash fluid dispenser 20 with tubular applicator 64 inserted into nostril 28, in accordance with an application of the present invention. Reference is also made to FIGS. 7A-K, which are several schematic illustrations of nasal wash fluid dispenser 20 viewed from several different directions, in accordance with an application of the present invention.

In some applications of the present invention, nasal wash fluid dispenser 20 comprises a dispensing container 72, which contains nasal wash fluid 26; and tubular applicator 64, which extends distally from and is in fluid communication with dispensing container 72. Tubular applicator 64 is sized and shaped to be insertable into nostril 28.

The tubular applicator 64 comprises first and second nozzles 60A, 60B, which are configured to dispense nasal wash fluid 26 in first and second fluid discharges, respectively, having first and second centerlines 74A and 74B (labeled in FIG. 3A), respectively, first and second centerlines 74A and 74B defining an offset angle α (alpha) (labeled in FIG. 3A) therebetween of between 10 and 90 degrees (such as between 20 and 80 degrees, e.g., between 60 and 70 degrees, such as between 30 and 60 degrees, e.g., between 40 and 50 degrees). As a result, when tubular applicator 64 is inserted into nostril 28 with first nozzle 60A oriented in posterior direction 48 and second nozzle 60B oriented in superoposterior direction 50, as shown in FIG. 3A, first nozzle 60A directs the first fluid discharge toward nasopharynx 32 and second nozzle 60B directs the second fluid discharge toward olfactory area 52 of nasal cavity 22, such that nasal wash fluid 26 washes biological material into oropharynx 30 from (a) nasal cavity 22, (b) nasopharynx 32, or (c) nasal cavity 22 and nasopharynx 32. First nozzle 60A directs at least a portion of the first fluid discharge under an inferior turbinate 76, so as to reach toward nasopharynx 32.

In some applications, first and second nozzles 60A, 60B are configured to dispense the first and the second fluid discharges of nasal wash fluid 26 with the same volume, sprayed particle size, velocity, and flow rate.

In some applications, first and second nozzles 60A, 60B are configured to dispense the first and the second fluid discharges of nasal wash fluid 26 with respective different fluid flow characteristics, the fluid flow characteristics including one or more characteristics selected from the group consisting of: volume, sprayed particle size, velocity, and flow rate.

In some applications, one of first and second nozzles 60A, 60B is located at a distal end of tubular applicator 64, and the other of first and second nozzles 60A, 60B is located is located away from the distal end of tubular applicator 64. For some of these applications, tubular applicator 64 is shaped so as to define a distal-most central longitudinal axis 78 through a distal end 80 of tubular applicator 64 (labeled in FIG. 6), and the one of first and second nozzles 60A, 60B located at the distal end of tubular applicator 64 is oriented such that the centerline thereof is coaxial with distal-most central longitudinal axis 78.

In some applications, first nozzle 60A is configured to dispense the first discharge with a first spray angle β (beta) of between 5 and 30 degrees (e.g., between 5 and 15 degrees), measured at 3 mm from first nozzle 60A (labeled in FIG. 6). In some applications, second nozzle 60B is configured to dispense the first discharge with a second spray angle of between 20 and 60 degrees (not labeled).

In some applications, tubular applicator 64, including first and second nozzles 60A, 60B, is configured to dispense, via first and second nozzles 60A, 60B, respectively, first and second fractions of a total volume of nasal wash fluid 26 dispensed via tubular applicator 64. The second fraction equals at least two times the first fraction, such as at least five times the first fraction, e.g., at least eight times the first fraction.

In some applications, nasal wash fluid dispenser 20 is configured to dispense nasal wash fluid by manual compression of the fluid dispenser.

In some applications, nasal wash fluid dispenser 20 comprises an atomizer that is configured to dispense nasal wash fluid 26.

In some applications, nasal wash fluid dispenser 20 comprises a pump that is configured to dispense nasal wash fluid 26.

In some applications, nasal wash fluid dispenser 20 comprises a pressurized aerosol generator that is configured to dispense nasal wash fluid 26.

In some applications, dispensing container 72 and tubular applicator 64 are shaped so as to be attachable to a pressure machine. For example, the pressure machine may be configured to apply pressure to dispensing container 72 and tubular applicator 64 so as to dispense a metered amount of nasal wash fluid 26. Optionally, the pressure machine may be configured to dispense the metered amount of nasal wash fluid 26 at adjustable pressures, doses and/or configurations.

In some applications, nostril 28 is a first nostril 28, and tubular applicator 64 is a first tubular applicator 64 that is sized and shaped to be insertable into first nostril 28. Nasal wash fluid dispenser 20 further comprises a second tubular applicator (not shown), which extends distally from and is in fluid communication with dispensing container 72, which is sized and shaped to be insertable into a second nostril 28, and which comprises third and fourth nozzles, which are configured to dispense nasal wash fluid 26 in third and fourth fluid discharges, respectively, having third and fourth centerlines, respectively, the third and the fourth centerlines defining the offset angle therebetween of between 10 and 90 degrees, or the other angles mentioned above. The second tubular applicator may implement any of the features of tubular applicator 64 described hereinabove or hereinbelow.

In some applications, the first tubular applicator does not comprise any nozzles other than first and second nozzles 60A, 60B, the second tubular applicator does not comprise any nozzles other than the third and the fourth nozzles, and nasal wash fluid dispenser 20 does not comprise any nozzles other than the first, the second, the third, and the fourth nozzles. Alternatively, the first tubular applicator, the second tubular applicator, and/or nasal wash fluid dispenser 20 comprises one or more additional nozzles (configuration not shown).

In some applications, tubular applicator 64 is malleable and compressible to fit into and occlude nostril 28. Alternatively, tubular applicator 64 is rigid.

In some applications, tubular applicator 64 comprises a curved portion.

In some applications, tubular applicator 64 is shaped so as to define an applicator centerline that defines a plane, and first and second nozzles 60A, 60B are configured to dispense nasal wash fluid 26 in the first and the second fluid discharges, respectively, such that first and second centerlines 74A, 74B, respectively, fall in the plane.

In some applications, tubular applicator 64 is shaped so as to define distal-most longitudinal axis 78 and a proximal-most central longitudinal axis 82 (labeled in FIG. 6) through distal end 80 and a proximal end 84 of tubular applicator 64, respectively, and distal-most and proximal-most central longitudinal axes 78, 82 form a bend angle δ (delta) of between 100 and 245 degrees therebetween (labeled in FIG. 6).

In some applications, nasal wash fluid dispenser 20 is shaped so as to guide introduction of tubular applicator 64 into nostril 28 at a specific orientation with respect to a maxilla 86 of the subject. In some applications, dispensing container 72 of nasal wash fluid dispenser 20 is shaped so as to guide introduction of tubular applicator 64 into nostril 28 at a specific orientation with respect to maxilla 86 of the subject. In other applications, nasal wash fluid dispenser 20 comprises a housing 88 that is shaped so as to guide the introduction of tubular applicator 64 into nostril 28 at the specific orientation with respect to maxilla 86 of the subject (configuration not shown).

In some applications, such as shown, for example, in FIGS. 3B, 7F, 7G, 7H, and 7I, nasal wash fluid dispenser 20 or dispensing container 72 or housing 88 of nasal wash fluid dispenser 20 is shaped so as to define a concave surface 90 that faces in posterior direction 48 and generally conforms to an outer surface of an upper lip 92 of the subject, and tubular applicator 64 may press the concave surface 90 against upper lip 92 so as to stabilize nasal wash fluid dispenser 20 with respect to maxilla 86. For example, concave surface 90 may have a lateral width W of between 2.5 and 7 cm e.g., 2.5-6 cm, such as 3-5 cm.

Typically, as shown in FIGS. 2A-B, in a method for using this configuration of nasal wash fluid dispenser 20, inserting tubular applicator 64 into nostril 28 comprises pressing concave surface 90 against upper lip 92 so as to stabilize nasal wash fluid dispenser 20 with respect to maxilla 86.

In some applications, concave surface 90 is curved. For example, concave surface 90 may be concave about an axis 94 and flat in a direction along axis 94 (labeled in FIG. 7H). Optionally, axis 94 forms an angle θ (theta) of between 75 and 90 degrees with first centerline 74A of the first fluid discharge, such as between 85 and 90 degrees. In some applications, dispensing container 72 is shaped so as to define concave surface 90. In other applications, nasal wash fluid dispenser 20 comprises housing 88 that is shaped so as to define concave surface 90.

In some applications, first and second nozzles 60A, 60B are configured to dispense nasal wash fluid such that the first and the second fluid discharges, respectively, are respective first and second types of fluid discharges selected from the group consisting of: a fluid stream, a spray, a mist, a pressurized aerosol, steam, and an atomized fluid. The first and the second types of fluid discharges may be the same type or different types.

In some applications, at least one of first and second nozzles 60A, 60B comprises a fan mechanism configured to dispense nasal wash fluid 26 in a circular rotary motion to generate dispersion within nasal cavity 22.

In some applications, first and second nozzles 60A, 60B are configured to dispense a total volume of at least 0.2 mL, no more than 20 mL, and/or 0.2-20 mL of nasal wash fluid 26 into nostril 28, such as 0.6-10 mL, e.g., 0.2-10 mL, such as 0.2-5 mL, e.g., 0.6-5 mL of nasal wash fluid 26 into nostril 28.

In some applications, nasal wash fluid dispenser 20 is configured to dispense nasal wash fluid 26 in a plurality of pulses. For example, nasal wash fluid dispenser 20 may comprise a mechanical pulsator, e.g., comprising one or more rotatable parts, e.g., which are driven to rotate by flow of nasal wash fluid 26 within the dispenser, and/or an electronic pulsator. Alternatively, the user of dispenser 20 may manually dispense nasal wash fluid 26 in pulses by repeatedly activating the dispenser.

In some applications, the pulses are delivered at intervals of between 0.5 and 2 seconds apart, e.g., 1 second apart.

In some applications, nasal wash fluid dispenser 20 is configured to dispense nasal wash fluid 26 in a plurality of pulses having a volume of at least 0.02 mL, no more than 10 mL, and/or 0.02-10 mL per pulse, e.g., 0.02-1 mL per pulse.

In some applications, at least one of first and second nozzles 60A, 60B is configured to dispense nasal wash fluid 26 with a sprayed particle size of 5-50 microns, such as 5-15 microns or 15-20 microns.

In some applications, such as labeled in FIG. 2A, a nasal wash fluid system 100 is provided that comprises nasal wash fluid dispenser 20, and further comprises nasal wash fluid 26, contained in dispensing container 72.

In some applications, nasal wash fluid 26 does not comprise a drug molecule. Alternatively, nasal wash fluid 26 does comprise a drug molecule.

In some applications, nasal wash fluid 26 comprises a non-irritant solution, which may, for example, comprise or consist of water. Alternatively, in some applications, nasal wash fluid 26 of a non-irritant solution consists of a saline solution, such as a phosphate-buffered saline solution.

In some applications, nasal wash fluid 26 comprises a tastant.

Alternatively or additionally, in some applications, nasal wash fluid 26 comprises a color additive. For example, the color additive may have a visually-perceptible color that is dominated by a non-white color other than yellow, such as red, green, or blue, which may be easier for a human subject to perceive in a liquid than yellow. Alternatively, the color additive may be yellow.

Further alternatively or additionally, in some applications, nasal wash fluid 26 comprises a chemical additive, such as described hereinabove with reference to FIGS. 2B and 2C.

In some applications, nasal wash fluid 26 comprises a first color additive, and nasal wash fluid system 100 further comprises an oral wash fluid 68 comprising a second colored additive, the first and the second color additives having different colors.

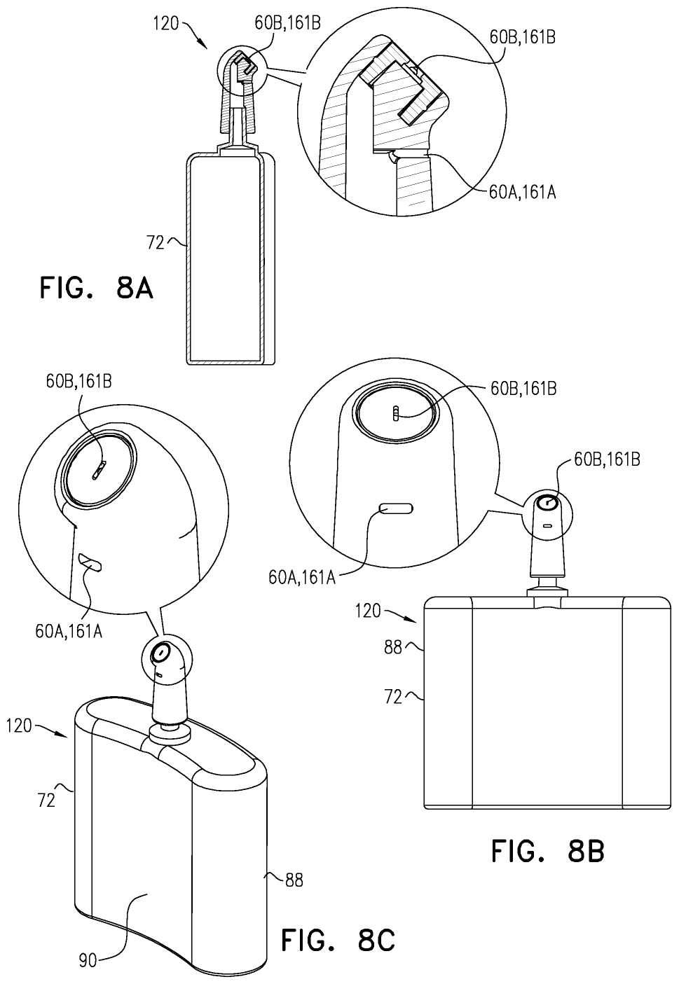

Reference is now made to FIGS. 8A-C, which are schematic illustrations of a nasal wash fluid dispenser 120, in accordance with an application of the present invention. Except as described below, nasal wash fluid dispenser 120 is identical to nasal wash fluid dispenser 20, described hereinabove, and like reference numerals refer to like parts. In nasal wash fluid dispenser 120, at least one of first and second nozzles 60A, 60B comprises a flat nozzle 161 (also known the nozzle art as an “even nozzle”). Optionally, first nozzle 60A comprises a first flat nozzle 161A, and second nozzle 60B comprises a second flat nozzle 161B. For example, the flat nozzles may comprise flat tapered nozzles.

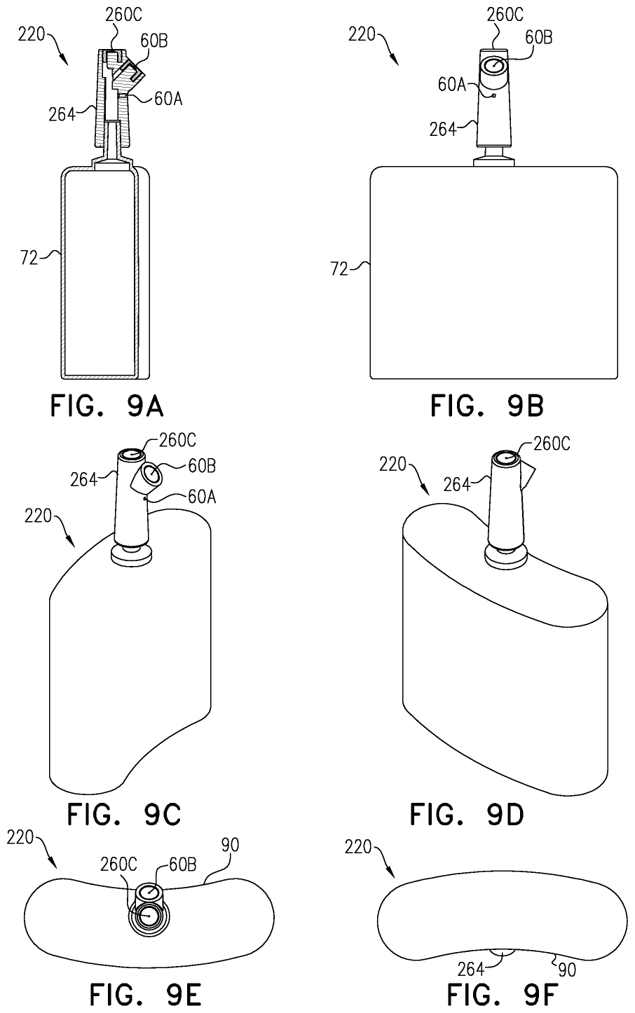

Reference is now made to FIGS. 9A-F, which are schematic illustrations of a nasal wash fluid dispenser 220, in accordance with an application of the present invention. Reference is further made to FIG. 10, which is a schematic cross-sectional illustration of nasal wash fluid dispenser 220 inserted into nasal cavity 22, in accordance with an application of the present invention. Reference is still further made to FIG. 11, which is a schematic cross-sectional illustration of a tubular applicator 264 of nasal wash fluid dispenser 220, in accordance with an application of the present invention. Except as described below, nasal wash fluid dispenser 220 is identical to nasal wash fluid dispenser 20, described hereinabove, and like reference numerals refer to like parts.

Tubular applicator 264 comprises, in addition to first and second nozzles 60A, 60B, a third nozzle 260C, which is configured to dispense nasal wash fluid 26 in a third fluid discharge having a third centerline 274C (labeled in FIG. 10). Typically, first and third centerlines 74A and 274C define an offset angle γ (gamma) (labeled in FIG. 10) therebetween of between 75 and 105 degrees (such as between 85 and 95 degrees, e.g., 90 degrees). As a result, when tubular applicator 264 is inserted into nostril 28 with first nozzle 60A oriented in posterior direction 48 and second nozzle 60B oriented in superoposterior direction 50, such as shown in FIG. 10, third nozzle 260C is oriented in superior direction 62, and thus directs the third fluid discharge at least in superior direction 62, toward the inferior border of the upper lateral cartilage of a nasal vestibule 296 of nasal cavity 22 (labeled in FIG. 10). (Nasal vestibule 296 is the portion of nasal cavity 22 anterior to the anterior head of the inferior turbinate.) Typically, third nozzle 260C has a relatively wide third spray angle ε (epsilon) (labeled in FIG. 10), such that third nozzle 260C additionally directs the third fluid discharge bilaterally (i.e., in both lateral directions within the single nasal cavity 22) toward the septum and a lateral wall of nasal cavity 22 (the lateral wall opposite the septum). For example, the third spray angle ε (epsilon) may between 45 and 135 degrees, such as between 60 and 120 degrees, e.g., about 90 degrees, measured at 3 mm from third nozzle 260C. As a result, nasal wash fluid 26 typically additionally washes biological material into oropharynx 30 from the superior wall, septum, and/or lateral wall of nasal cavity 22. More generally, nasal wash fluid 26 typically additionally washes biological material into oropharynx 30 from nasal vestibule 296, which is at least a portion of the area generally sampled by a conventional nasal swab.

Reference is now made to FIG. 12, which is a schematic illustration of a testing kit 300, in accordance with an application of the present invention. Testing kit 300 comprises nasal wash fluid system 100 (labeled in FIG. 2A), and further comprises a test 302 (e.g., a diagnostic test), which is configured to test for the presence of a particulate in specimen sample 24 collected using the techniques described herein. (As described hereinabove, nasal wash fluid system 100 comprises nasal wash fluid dispenser 20 and nasal wash fluid 26.) Test 302 may optionally be configured to implement testing techniques described herein, in the patent applications incorporated hereinbelow by reference, and/or known in the art. For example, test 302 may further comprise one or more filters configured to concentrate specimen sample 24 by filtration prior to testing for the presence of the particulate. The particulate may be a virus, a bacterium, any of the other particulates described hereinabove, or any of the other biological materials described hereinabove. For some applications, test 302 comprises a lateral flow immunoassay test strip (which may be an element of a chromatographic digital immunoassay, such as the BD Veritor™ system, mentioned immediately below), which is configured to detect the presence of the particulate (such as by detecting protein antigen, e.g., from a virus), and, optionally, one or more reagents. (Lateral flow immunoassay test strips (optionally as an element of a chromatographic digital immunoassay) are well known in the art. For example, they typically contain an antibody specific to an antigen, and the specimen sample fluid migrates up the test strip and reacts with the antibody, thus generating a line on the test strip; the presence of this line indicates a positive test result. For example, the BD Veritor™ system for Rapid Detection of SARS-CoV-2 (Becton, Dickinson and Company, Maryland, USA, Ref: 256082) is a chromatographic digital immunoassay intended for the direct and qualitative detection of SARS-CoV-2 nucleocapsid antigens in nasal swabs.) Alternatively, for some applications, test 302 comprises a rapid molecular test kit, for example, an isothermal nucleic acid amplification (iNAAT) test, such as a rapid molecular test kit that uses a real-time loop mediated amplification reaction, such as the Lucira COVID-19 All-In-One Test Kit (Lucira Health, Inc., Emeryville, Calif., USA), or a nicking enzyme amplification reaction (NEAR) technology, such as the ID NOW™ (Abbott Laboratories, Abbott Park, Ill., USA), or a molecular test kit manufactured by Visby. Further alternatively, test 302 may comprise a CRISPR-based diagnostic test, an ELISA diagnostic test, or a spectroscopy-based diagnostic test.

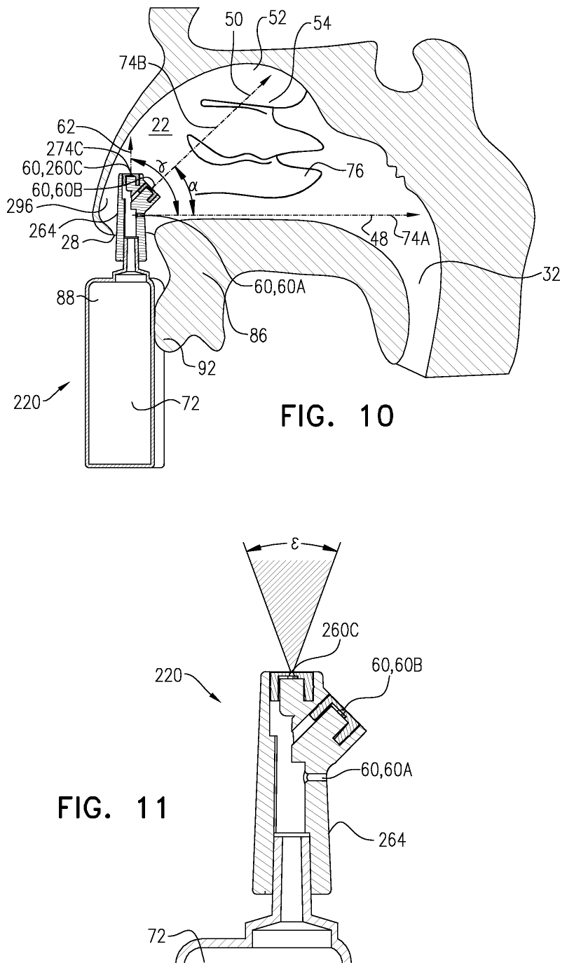

Reference is now made to FIG. 13, which is a schematic illustration of a testing kit 400, in accordance with an application of the present invention. Testing kit 400 comprises nasal wash fluid system 100 (labeled in FIG. 2A), and further comprises a vial 402 and a liquid 404 for bathing at least a portion of specimen sample 24 in the vial, the liquid selected from the group consisting of: a lysis buffer, saline solution (e.g., phosphate buffered saline (PBS)), and transport medium (e.g., universal transport medium or a viral transport medium). Optionally, vial 402 contains liquid 404. Testing kit 400 may be used, for example, for collecting specimen sample 24 from the subject and sending at least a portion of the specimen sample to a remote laboratory in the liquid in the vial, such as for performing Polymerase Chain Reaction (PCR) testing for particulate in the specimen sample. (As described hereinabove, nasal wash fluid system 100 comprises nasal wash fluid dispenser 20 and nasal wash fluid 26.) The particulate may be a virus, a bacterium, any of the other particulates described hereinabove, or any of the other biological materials described hereinabove.

Reference is now made to FIGS. 14A-B, which are schematic illustrations of filter units 500A and 500B, respectively, for testing a liquid specimen sample that includes saliva for the presence of a particulate, in accordance with respective applications of the present invention. The particulate may be a virus, a bacterium, any of the other particulates described hereinabove, or any of the other biological materials described hereinabove.

Filter unit 500A comprises at least one filter chamber 510A (such as exactly one, as shown), which is shaped so as to define an inlet 512, an outlet 514, and a fluid flow path between inlet 512 and outlet 514. Filter unit 500B comprises at least one filter chamber 510B (such as exactly one, as shown), which is shaped so as to define inlet 512, outlet 514, and a fluid flow path between inlet 512 and outlet 514.

Each of the at least one filter chambers 510A and 510B comprises a large-pored filter 520, which is disposed within at the at least one filter chamber in the fluid flow path, and has a nominal pore size.

The at least one filter chamber 510B of filter unit 500A comprises large-pored filter 520, and one or more additional filters 522, which are disposed within the at the at least one filter chamber 510B in series in the fluid flow path, and which have respective nominal pore sizes, which may or may not equal one another, and may or may not equal the nominal pore size of large-pored filter 520. For example, the one or more additional filters 522 may include exactly one additional filter 522 (as shown) or more than one additional filter 522 (configuration not shown). Typically, but not necessarily, each of the respective nominal pore sizes of the one or more additional filters 522 is less than or equal to the nominal pore size of large-pored filter 520.

For some applications, the nominal pore size of large-pored filter 520 is between 30 microns and 1.5 mm.

For some applications, the nominal pore size is representative of a minimum size of spherical particles (e.g., inert spherical particles) necessary for the respective filter to retain 85% of the spherical particles when distilled H2O containing the spherical particles is passed through the respective filter at 20 degrees C. under pressure supplied by a 10 cm water column (of course, without downstream resistance) (herein, the “challenge-test nominal pore size”). It will be appreciated that the spherical particles, distilled H2O, and 10 cm water column are not elements of the device, but are instead non-device elements that are used to characterize certain physical properties of the filter(s), as is conventional for filter characterization in the filter art.

Alternatively, the nominal pore size may be characterized by a bubble point test, as is well known in the filter art, using a capillary flow porometer, such as CFP-1500A Automatic Capillary Flow Porometer (optionally, including options E, X, L, and/or M, if necessary for characterizing certain larger pore sizes) made by Porous Materials Inc. (PMI) (Ithaca, N.Y., USA), and a wetting liquid having a fluid surface tension of 15.9 dynes/cm, such as Galwick (PMI) (herein, the “bubble-point nominal pore size”).

As used in the present application, including in the claims and Inventive Concepts, a “pore” means any opening through a filter through which at least distilled H2O can pass.

For some applications, large-pored filter 520 comprises a depth filter.

For some applications, large-pored filter 520 comprises a mesh filter, such as a woven mesh filter or a non-woven mesh filter.

For some applications, large-pored filter 520 comprises fibers, such as crisscrossing fibers.

For some applications, large-pored filter 520 comprises a screen, such as a woven screen or a non-woven screen.

For some applications, large-pored filter 520 comprises a polymer, such as a polyester.

For some applications, large-pored filter 520 comprises a metal.

For some applications, the challenge-test nominal pore size is at least 40 microns. For some applications, the challenge-test nominal pore size is at least 50 microns. For some applications, the challenge-test nominal pore size is at least 60 microns. For some applications, the challenge-test nominal pore size is at least 100 microns. For some applications, the challenge-test nominal pore size is at least 120 microns. For some applications, the challenge-test nominal pore size is at least 150 microns. For some applications, the challenge-test nominal pore size is at least 200 microns. For some applications, the challenge-test nominal pore size is at least 500 microns.

For some applications, the challenge-test nominal pore size is less than 1 mm. For some applications, the challenge-test nominal pore size is less than 750 microns. For some applications, the challenge-test nominal pore size less than 500 microns. For some applications, the challenge-test nominal pore size less than 250 microns. For some applications, the challenge-test nominal pore size less than 200 microns.

For some applications, the challenge-test nominal pore size is between 30 and 40 microns. For some applications, the challenge-test nominal pore size is between 40 and 60 microns. For some applications, the challenge-test nominal pore size is between 50 and 200 microns, such as between 50 and 150 microns, e.g., between 100 and 150 microns. For some applications, the challenge-test nominal pore size is between 60 and 100 microns. For some applications, the challenge-test nominal pore size is between 100 and 120 microns. For some applications, the challenge-test nominal pore size is between 120 and 150 microns. For some applications, the challenge-test nominal pore size is between 150 and 200 microns. For some applications, the challenge-test nominal pore size is between 200 and 500 microns. For some applications, the challenge-test nominal pore size is between 500 microns and 1 mm, such as between 500 and 750 microns, or between 750 microns and 1 mm.

For some applications, the bubble-point nominal pore size is at least 40 microns. For some applications, the bubble-point nominal pore size is at least 50 microns. For some applications, the bubble-point nominal pore size is at least 60 microns. For some applications, the bubble-point nominal pore size is at least 100 microns. For some applications, the bubble-point nominal pore size is at least 120 microns. For some applications, the bubble-point nominal pore size is at least 150 microns. For some applications, the bubble-point nominal pore size is at least 200 microns.

For some applications, the bubble-point nominal pore size less than 500 microns. For some applications, the bubble-point nominal pore size less than 300 microns. For some applications, the bubble-point nominal pore size less than 250 microns. For some applications, the bubble-point nominal pore size less than 200 microns.

For some applications, the bubble-point nominal pore size is between 30 and 40 microns. For some applications, the bubble-point nominal pore size is between 40 and 60 microns. For some applications, the bubble-point nominal pore size is between 50 and 200 microns, such as between 50 and 150 microns, e.g., between 100 and 150 microns. For some applications, the bubble-point nominal pore size is between 60 and 100 microns. For some applications, the bubble-point nominal pore size is between 100 and 120 microns. For some applications, the bubble-point nominal pore size is between 120 and 150 microns. For some applications, the bubble-point nominal pore size is between 150 and 200 microns. For some applications, the bubble-point nominal pore size is between 200 and 300 microns.

For some applications, filter chambers 510A and 510B are generally cylindrical, as shown, while for other applications they may have different shapes.

For some applications, filter chambers 510A and 510B further comprise one or more filter supports 530, which are disposed within the filter chambers, and are shaped so as to define respective (a) proximal support surfaces that mechanically support respective filters, and (b) respective pluralities of filter-support openings through the filter supports to allow passage of filtrate from the respective filters.

For some applications, the filter unit further comprises a liquid-pressure source 534, which is arranged to apply pressure to drive the liquid specimen sample through the one or more filters. For example, liquid-pressure source 534 may comprise a pump 536, such as shown, by way of example, for filter unit 500A, or a plunger 538, such as shown, by way of example, for filter unit 500B.

For some applications, the filter unit further comprises a waste receptacle 540, which is disposed in fluid communication with outlet 514 of the filter chamber so as to receive filtrate from the one or more filters. For some of these applications, waste receptacle 540 cannot be removed from the filter chamber without damaging the filter unit, such as shown. For other applications, waste receptacle 540 is configured to be decoupled from the filter chamber without damaging the filter unit (configuration not shown).

For some applications, waste receptacle 540 contains a disinfectant 544, such as shown, by way of example, for filter unit 500A, or a liquid-absorbing material 546, such as shown, by way of example, for filter unit 500B.

In some applications of the present invention, a method is provided that comprises:
collecting, from a subject, a liquid specimen sample that includes saliva and orally-dispensed oral wash fluid 68;
passing the liquid specimen sample through a filter (such as large-pored filter 520, described hereinabove with reference to FIGS. 14A-B) to trap at least some of the saliva, the filter having a nominal pore size of between 30 microns and 1.5 mm; and
testing for the presence of a virus in the saliva trapped (i.e., retained) by the filter.

Alternatively, the method comprises testing for the presence of a bacterium trapped by the filter. Further alternatively, the method comprises testing for the presence of another particulate trapped by the filter, such as any of the particulates mentioned hereinabove.

This method may be practiced in combination with any of the techniques described herein for collecting specimen sample 24 from the subject that include gargling by the subject, swishing by the subject, and/or spraying oropharynx 30 via anterior opening 34 of oral cavity 36.

As described below in the section entitled, “Experiment 7,” the inventors found that filters having pore sizes substantially larger than the viral diameter were nevertheless able to trap virus in the saliva of gargled fluid in quantities sufficient to clinically identify the presence of the virus, even using immunoassays, which generally require substantially greater quantities of virus for detection than does PCR. The inventors hypothesize that the large-pore filters were able to trap virus at least in part because of the viscosity of the saliva in typical gargled fluid samples, perhaps in particular because of the viscosity of the mucus and/or other large or aggregated salivary components of the saliva, which themselves contain viral particles, and/or because the viral particles adhered (e.g., directly adhered) to the material of the large-pore filters. Use of large-pore filters may enable a high flow rate and inhibit filter clogging despite high sample viscosity.

For some applications, the challenge-test nominal pore size is at least 40 microns. For some applications, the challenge-test nominal pore size is at least 50 microns. For some applications, the challenge-test nominal pore size is at least 60 microns. For some applications, the challenge-test nominal pore size is at least 100 microns. For some applications, the challenge-test nominal pore size is at least 120 microns. For some applications, the challenge-test nominal pore size is at least 150 microns. For some applications, the challenge-test nominal pore size is at least 200 microns. For some applications, the challenge-test nominal pore size is at least 500 microns.

For some applications, the challenge-test nominal pore size is less than 1 mm. For some applications, the challenge-test nominal pore size is less than 750 microns. For some applications, the challenge-test nominal pore size less than 500 microns. For some applications, the challenge-test nominal pore size less than 250 microns. For some applications, the challenge-test nominal pore size less than 200 microns.

For some applications, the challenge-test nominal pore size is between 30 and 40 microns. For some applications, the challenge-test nominal pore size is between 40 and 60 microns. For some applications, the challenge-test nominal pore size is between 50 and 200 microns, such as between 50 and 150 microns, e.g., between 100 and 150 microns. For some applications, the challenge-test nominal pore size is between 60 and 100 microns. For some applications, the challenge-test nominal pore size is between 100 and 120 microns. For some applications, the challenge-test nominal pore size is between 120 and 150 microns. For some applications, the challenge-test nominal pore size is between 150 and 200 microns. For some applications, the challenge-test nominal pore size is between 200 and 500 microns. For some applications, the challenge-test nominal pore size is between 500 microns and 1 mm, such as between 500 and 750 microns, or between 750 microns and 1 mm.

For some applications, the bubble-point nominal pore size is at least 40 microns. For some applications, the bubble-point nominal pore size is at least 50 microns. For some applications, the bubble-point nominal pore size is at least 60 microns. For some applications, the bubble-point nominal pore size is at least 100 microns. For some applications, the bubble-point nominal pore size is at least 120 microns. For some applications, the bubble-point nominal pore size is at least 150 microns. For some applications, the bubble-point nominal pore size is at least 200 microns.

For some applications, the bubble-point nominal pore size less than 500 microns. For some applications, the bubble-point nominal pore size less than 300 microns. For some applications, the bubble-point nominal pore size less than 250 microns. For some applications, the bubble-point nominal pore size less than 200 microns.

For some applications, the bubble-point nominal pore size is between 30 and 40 microns. For some applications, the bubble-point nominal pore size is between 40 and 60 microns. For some applications, the bubble-point nominal pore size is between 50 and 200 microns, such as between 50 and 150 microns, e.g., between 100 and 150 microns. For some applications, the bubble-point nominal pore size is between 60 and 100 microns. For some applications, the bubble-point nominal pore size is between 100 and 120 microns. For some applications, the bubble-point nominal pore size is between 120 and 150 microns. For some applications, the bubble-point nominal pore size is between 150 and 200 microns. For some applications, the bubble-point nominal pore size is between 200 and 300 microns.

Typically, the method does not comprise centrifuging the liquid specimen sample.

For some applications, the method does not comprise adding to the liquid specimen sample a concentration agent that binds to the virus.

For some applications, the filter is one of a plurality of filters arranged in a series of filters (such as described hereinabove with reference to FIG. 14B). For some applications, the filters have respective challenge-test nominal pore sizes, all of which are between 30 microns and 1.5 mm, or any of the challenge-test nominal pore sizes provided above. For some applications, the filters have respective bubble-point nominal pore sizes, all of which are between 30 and 500 microns, such as between 30 and 300 microns, or any of the bubble-point nominal pore sizes provided above. The method comprises passing the liquid specimen sample through the series of filters, without passing the liquid specimen sample through any other filters. Testing for the presence of the virus in the saliva comprises testing for the presence of the virus in the saliva trapped by at least one of the plurality of filters, such as testing for the presence of the virus in the saliva trapped by at least two of the plurality of filters.

For some applications, the filter is a first filter (such as large-pored filter 520 of filter chamber 510B, described hereinabove with reference to FIG. 14B). Passing the liquid specimen sample through the filter comprises passing the liquid specimen sample through the first filter to produce a filtrate. Testing for the presence of the virus in the saliva trapped by the filter comprises testing for the presence of the virus in the saliva trapped by the first filter. The method further comprises passing the filtrate through a second filter (such as additional filter 522 of filter chamber 510B, described hereinabove with reference to FIG. 14B) to trap at least some of the saliva, and testing for the presence of the virus in the saliva trapped by the second filter. For some of these applications, a nominal pore size of the second filter equals the nominal pore size of the first filter. For some of these applications, a challenge-test nominal pore size of the second filter is between 0.03 and 21 microns, such as between 0.8 and 1.5 microns. For some of these applications, a bubble-point nominal pore size of the second filter is between 0.03 and 21 microns, such as between 0.8 and 1.5 microns. For some of these applications, the second filter may be configured (such as by its nominal pore size) to capture epithelial cells.

For some applications, passing the liquid specimen sample through the filter comprises passing the liquid specimen sample through the filter to trap at least some of the saliva by size-based filtration.

For some applications, passing the liquid specimen sample through the filter comprises passing the liquid specimen sample through the filter to trap at least some of the saliva by adhesion of the saliva to the filter. For some applications, passing the liquid specimen sample through the filter to trap at least some of the saliva by adhesion of the saliva to the filter by interactions that are not specific to the virus (rather than specific interactions, such as ligand or antibody binding). For some applications, the filter is coated with organic molecules, such as lipids, proteins, and/or carbohydrates, to increase adhesion of the saliva to the filter.

For some applications, the filter comprises a depth filter.

For some applications, the filter comprises a mesh filter, such as a woven mesh filter or a non-woven mesh filter.

For some applications, the filter comprises fibers, such as crisscrossing fibers.

For some applications, the filter comprises a screen, such as a woven screen or a non-woven screen.

For some applications, the filter comprises a polymer, such as a polyester.

For some applications, the filter comprises a metal.

For some applications, passing the liquid specimen sample through the filter comprises passing the liquid specimen sample through exactly one filter.

For some applications, testing for the presence of the virus in the saliva trapped by the filter comprises testing the filter for the presence of the virus in the saliva.

For some applications, testing for the presence of the virus in the saliva trapped by the filter comprises inserting the filter into a testing machine. For example, the testing machine may be selected from the group consisting of: a thermal cycler and an isothermal amplification instrument.

For some applications, testing for the presence of the virus comprises performing a lateral flow immunoassay. As described below in the section entitled, “Experiment 7,” the inventors were able to detect the presence of SARS-CoV-2 in the biological material trapped by the filter using lateral flow immunoassays.

For some applications, testing for the presence of the virus in the saliva trapped by the filter comprises, after finishing passing the liquid specimen sample through the filter, bringing the filter, the saliva, and a liquid medium into contact, and testing the liquid medium for the presence of the virus. For some applications, the testing machine is selected from the group consisting of: a thermal cycler and an isothermal amplification instrument.

For some applications, testing the liquid medium for the presence of the virus comprises separating the liquid medium from the filter before testing the liquid medium for the presence of the virus. For some applications, testing for the presence of the virus trapped by the filter comprises agitating the filter in the liquid medium, for example mixing the filter in the liquid medium.

For some applications, testing for the presence of the virus trapped by the filter comprises transferring at least a portion of the liquid medium into a testing machine.

For some applications, the liquid medium is selected from the group consisting of: a transport medium, a culture medium, a purification agent, a stabilizing agent, a lysing buffer, and an extraction agent.

For some applications, testing for the presence of the virus comprises using spectroscopy, using microscopy, using image processing, and/or using three-dimensional (3D) image sensing. For some applications, testing for the presence of the virus comprises using a molecular-based assay. For some of these applications, testing for the presence of the virus comprises using nucleic acid amplification, such as isothermal amplification or polymerase chain reaction (PCR). As described below in the section entitled, “Experiment 7,” the inventors were able to detect the presence of SARS-CoV-2 in the biological material trapped by the filter using PCR.

For some applications, testing for the presence of the virus comprises using artificial intelligence. Any of the above-mentioned testing techniques may optionally utilize artificial intelligence.

For some applications, the method further comprises, before testing for the presence of the virus in the saliva trapped by the filter, transporting the filter while the filter is at least partially immersed in a liquid medium. For some of these applications, testing for the presence of the virus in the saliva trapped by the filter comprises inserting at least a portion of the liquid medium into a thermal cycler. For some of these applications, the filter is placed in the liquid medium, the liquid medium is agitated, and the filter is removed from the liquid medium before transporting the liquid medium to a remote testing site for testing for the presence of the virus in the saliva trapped by the filter.

For some applications, collecting the liquid specimen sample comprises receiving the liquid specimen sample by a healthcare worker from the subject.

For some applications, the method does not comprise swabbing nasal cavity 22. Alternatively or additionally, for some applications, the method does not comprise swabbing a pharynx of the subject.

For some applications, the method does not comprise swabbing any portion of the subject. Alternatively, the method comprises swabbing a portion of the subject, such as using swabbing techniques described herein.

For some applications, collecting the liquid specimen sample comprises collecting the liquid specimen sample that was expressed out of oropharynx 30 via anterior opening 34 of oral cavity 36. For some of these applications, collecting the liquid specimen sample comprises collecting the liquid specimen sample that was spit out of oropharynx 30 via anterior opening 34 of oral cavity 36.

For some applications, collecting the liquid specimen sample comprises aspirating the liquid specimen sample out of oropharynx 30 via anterior opening 34 of oral cavity 36.

For some applications, collecting the liquid specimen sample comprises:
gargling, by the subject, oral wash fluid 68 to produce the liquid specimen sample that includes the saliva; and
thereafter, collecting the liquid specimen sample that passed out of anterior opening 34 of oral cavity 36.

For some applications, collecting the liquid specimen sample comprises:
gargling, by the subject, oral wash fluid 68 to produce the liquid specimen sample that includes the saliva; and
thereafter, expressing, by the subject, out of anterior opening 34 of oral cavity 36, the liquid specimen sample.

For some applications, collecting the liquid specimen sample comprises:
swishing, by the subject, oral wash fluid 68 in oral cavity 36 to produce the liquid specimen sample that includes the oral wash fluid and the saliva; and
thereafter, collecting the liquid specimen sample from the subject.

For some applications, collecting the liquid specimen sample comprises:
swishing, by the subject, oral wash fluid 68 in oral cavity 36 to produce the liquid specimen sample that includes the oral wash fluid and the saliva; and
thereafter, expressing, by the subject, out of anterior opening 34 of oral cavity 36, the liquid specimen sample.

For some applications, collecting the liquid specimen sample comprises:
spraying, via anterior opening 34 of oral cavity 36, oropharynx 30 with oral wash fluid 68; and
thereafter, collecting the liquid specimen sample from the subject.

For some applications, the above-mentioned gargling, swishing, and/or spraying techniques are combined.

For some applications, collecting the gargled fluid from the subject comprises:
intranasally dispensing nasal wash fluid 26 into nasal cavity 22 such that nasal wash fluid 26 washes biological material into oropharynx 30 from (a) nasal cavity 22, (b) nasopharynx 32, or (c) nasal cavity 22 and nasopharynx 32; and
thereafter, collecting the liquid specimen sample that passed out of anterior opening 34 of oral cavity 36 and includes oral wash fluid 68, nasal wash fluid 26, and at least a portion of the biological material washed into oropharynx 30 by nasal wash fluid 26.

For some applications, collecting the liquid specimen sample comprises expressing, by the subject, the liquid specimen sample out of oropharynx 30 via anterior opening 34 of oral cavity 36. For some applications, expressing the liquid specimen sample comprises spitting, by the subject, the liquid specimen sample out of oropharynx 30 via anterior opening 34 of oral cavity 36.

For some applications, collecting the liquid specimen sample comprises:
after intranasally dispensing nasal wash fluid 26, orally dispensing oral wash fluid into oral cavity 36 via anterior opening 34 of oral cavity 36 such that oral wash fluid 68 forms mixture 70 with nasal wash fluid 26 in oropharynx 30;
gargling, by the subject, mixture 70 to produce the liquid specimen samples that includes oral wash fluid 68, nasal wash fluid 26, and the saliva; and
collecting the liquid specimen sample that passed out of anterior opening 34 of oral cavity 36.

For some applications, collecting the liquid specimen sample from the subject comprises:
intranasally dispensing nasal wash fluid into nasal cavity 22 such that nasal wash fluid 26 washes biological material into an oropharynx of the subject from (a) nasal cavity 22, (b) a nasopharynx of the subject, or (c) nasal cavity 22 and the nasopharynx; and
thereafter, expressing, by the subject, out of anterior opening 34 of oral cavity 36, the liquid specimen sample that includes at least a portion of the biological material washed into oropharynx 30 by nasal wash fluid 26.

For some applications, expressing the liquid specimen sample comprises spitting, by the subject, the liquid specimen sample out of oropharynx 30 via anterior opening 34 of oral cavity 36.

For some applications, collecting the liquid specimen sample comprises:
after intranasally dispensing nasal wash fluid 26, orally dispensing oral wash fluid 68 into oral cavity 36 via anterior opening 34 of oral cavity 36 such that oral wash fluid 68 forms mixture 70 with nasal wash fluid 26 in oropharynx 30;
gargling, by the subject, mixture 70 to produce the liquid specimen sample that includes oral wash fluid 68, nasal wash fluid 26, and the saliva; and
expressing, by the subject, the liquid specimen sample out of anterior opening 34 of oral cavity 36.

For some applications, the subject intranasally dispenses nasal wash fluid 26 into nasal cavity 22, while for some other applications, a healthcare worker intranasally dispenses nasal wash fluid 26 into nasal cavity 22.

For some applications, the method further comprises, after intranasally dispensing nasal wash fluid 26, gargling, by the subject, nasal wash fluid 26 washed into oropharynx 30.

For some applications, the virus is a coronavirus, such as severe acute respiratory syndrome coronavirus 2 (SARS-CoV-2); or an Influenza virus.

For some applications, the subject is one of a plurality of subjects, and wherein the liquid specimen sample is one of a plurality of liquid specimen samples. The plurality of liquid specimen samples, which include respective saliva, are collected from the plurality of subjects, respectively. The plurality of liquid specimen samples are combined, and the combined plurality of liquid specimen samples are passed through the filter. For example, the plurality of liquid specimen samples may include at least five liquid specimen samples from at least five subjects, respectively, such as at least ten liquid specimen samples from at least ten subjects, respectively, or at least 25 liquid specimen samples from at least 25 subjects, respectively.

This method of combining liquid specimen samples from a plurality of subjects allows screening a plurality of subjects using a single filter unit and a single test of the material captured by the one or more filters of the single filter unit. If the particulate, e.g., the virus, is detected in the combined sample, the subjects may be individually tested.

For some applications, the plurality of liquid specimen samples have a combined volume of at least 25 cc, such as at least 50 cc, e.g., at least 100 cc, or at least 150 cc.

For some applications, combining the plurality of liquid specimen samples comprises mixing together the plurality of liquid specimen samples.

Any of the method described herein that comprise collecting, filtering, and testing a liquid specimen sample that includes saliva and orally-dispensed oral wash fluid 68 may alternatively comprise collecting, filtering, and testing a liquid specimen sample that includes (a) a biological material other than saliva and (b) orally-dispensed oral wash fluid 68.

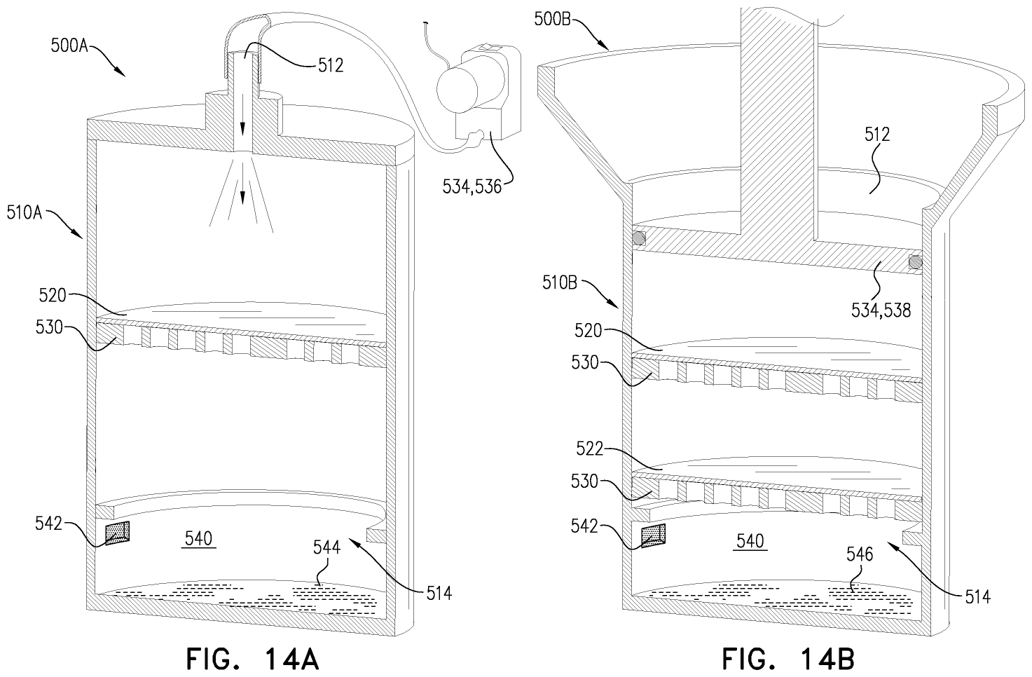

Reference is now made to FIG. 15, which is a schematic illustration of a testing kit 600, in accordance with an application of the present invention. Testing kit 600 comprises a filter unit 610, such as one of filter units 500A or 500B, described hereinabove with reference to FIGS. 14A-B, respectively; and a test 602 (e.g., a diagnostic test), which is configured to test for the presence of a particulate in the virus or other particulate (such as those described herein) trapped by the one or more filters of the filter unit. Test 602 may optionally be configured to implement testing techniques described herein, in the patent applications incorporated hereinbelow by reference, and/or known in the art. For some applications, test 602 comprises a lateral flow immunoassay test strip, which is configured to detect the presence of the particulate (such as by detecting protein antigen, e.g., from a virus), and, optionally, one or more reagents. Alternatively, for some applications, test 602 comprises a rapid molecular test kit, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, test 302 may comprise a CRISPR-based diagnostic test, an ELISA diagnostic test, or a spectroscopy-based diagnostic test.

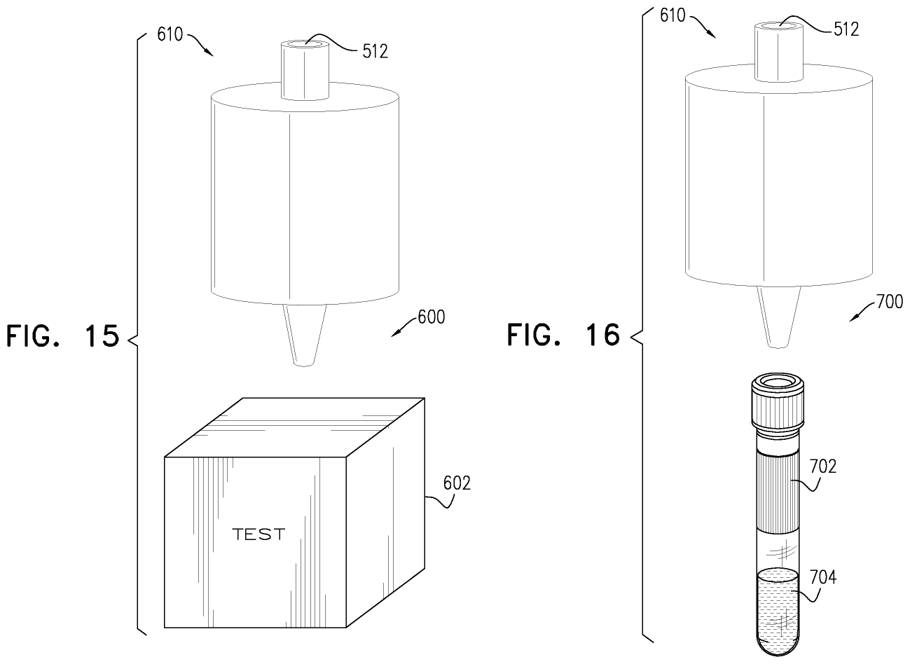

Reference is now made to FIG. 16, which is a schematic illustration of a testing kit 700, in accordance with an application of the present invention. Testing kit 700 comprises:
filter unit 610, such as one of filter units 500A or 500B, described hereinabove with reference to FIGS. 14A-B, respectively;
a vial 702, for containing at least a portion of the saliva captured by at least one of the one or more filters of the filter unit; and
liquid 704 for bathing the at least a portion of the saliva in the vial, the liquid selected from the group consisting of: a lysis buffer, saline solution (e.g., phosphate buffered saline (PBS)), and transport medium (e.g., universal transport medium or a viral transport medium).

Optionally, vial 702 contains liquid 704. Testing kit 700 may be used, for example, for collecting a specimen sample from the subject and sending at least a portion of the specimen sample to a remote laboratory in the liquid in the vial, such as for performing Polymerase Chain Reaction (PCR) testing for particulate in the specimen sample. The particulate may be a virus (e.g., an Influenza virus or SARS-CoV-2), a bacterium (e.g., Streptococcus bacterium), any of the other particulates described hereinabove, or any of the other biological materials described hereinabove. For example, the particulate may be tested for using nucleic acid amplification, such as PCR, e.g., qPCR, and/or by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, test 302 may comprise a CRISPR-based diagnostic test, an ELISA diagnostic test, or a spectroscopy-based diagnostic test.

Reference is made to both FIGS. 15 and 16. For some applications, testing kit 600 or 700 does not comprise any specimen-collection swabs.

For some applications, testing kit 600 or 700 comprises a nasal applicator containing nasal wash fluid 26, such as a conventional nasal dispenser or a nasal wash fluid dispenser described hereinabove. For some of these applications, the testing kit comprises a plurality of nasal applicators containing nasal wash fluid 26.

For some applications, testing kit 600 or 700 comprises a first number of nasal applicators, and a second number of one or more filter units 610, and a ratio of the first number to the second number is at least 2, such as at least 5.

For some applications, testing kit 600 or 700 further comprises a container containing oral wash fluid 68. For some applications, the testing kit comprises a plurality of nasal applicators containing nasal wash fluid 26 and a plurality of containers containing oral wash fluid 68, and the testing kit comprises a first number of nasal applicators containing nasal wash fluid 26, a second number of one or more filter units 610, and a third number of containers containing oral wash fluid 68. The first number equals the third number, and a ratio of the first number to the second number is at least 2, such as at least 5.

For some applications, testing kit 600 or 700 further comprises a container containing oral wash fluid 68. For some of these applications, the test kit comprises a plurality of containers containing oral wash fluid 68. For some of these applications, the testing kit comprises a third number of containers containing oral wash fluid 68, and a second number of one or more filter units 610, and a ratio of the third number to the second number is at least 2, such as at least 5.

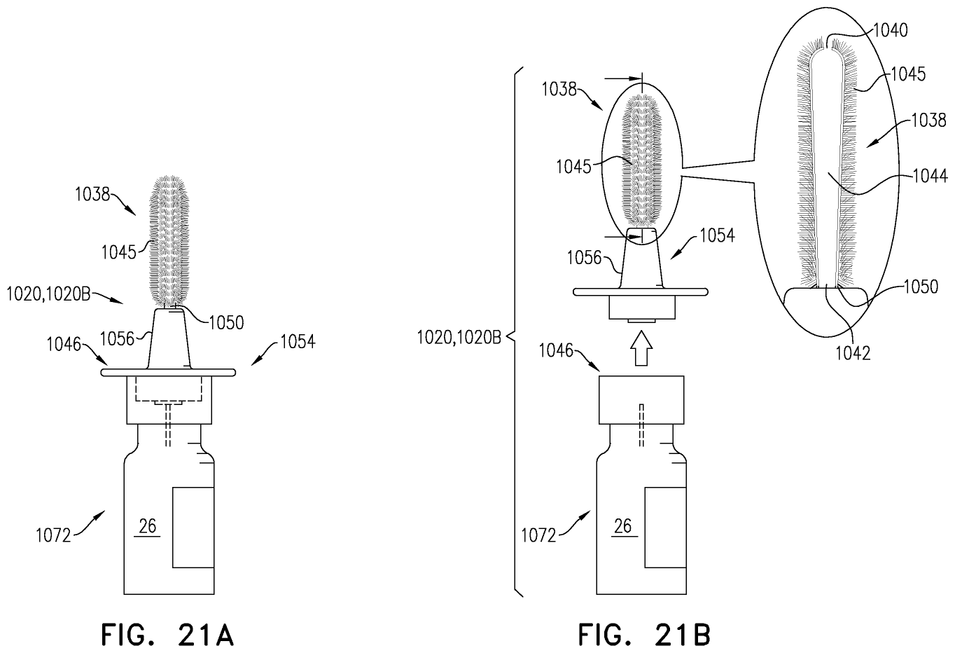

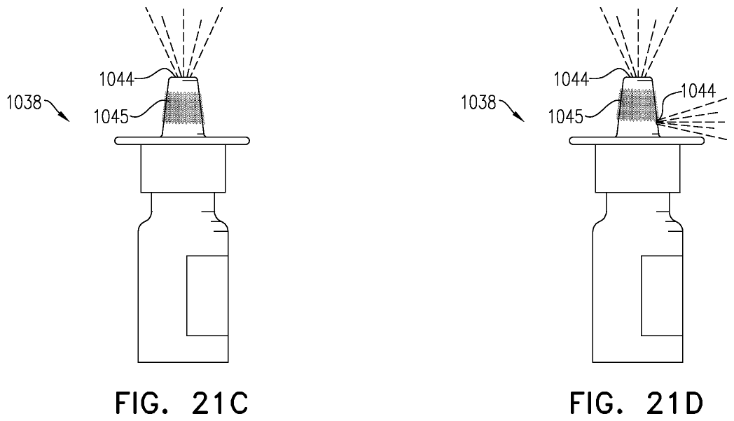

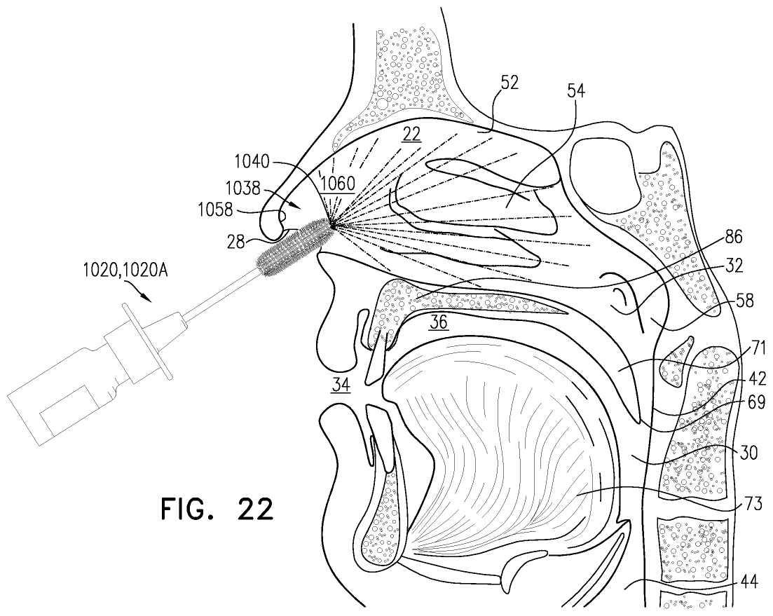

Reference is now made to FIGS. 17A-B, which are schematic illustrations of a method for collecting a specimen sample from a subject, in accordance with an application of the present invention. The method comprises intranasally dispensing nasal wash fluid 26 into nasal cavity 22, by the subject or by somebody else. Nasal wash fluid 26 may be intranasally dispensed using a conventional nasal dispenser 820, such as shown in FIG. 17A; using nasal wash fluid dispenser 20, such as shown in FIG. 2B; using nasal sampling device 1020, such as shown in FIG. 22; or using any nasal wash fluid dispenser known in the art. Nasal wash fluid 26 generally loosens biological material in nasal cavity 22 and/or nasopharynx 32. Thereafter, a specimen sample is collected by performing, by the subject or by somebody else, one or more nasal swabs selected from the group consisting of: an anterior nares swab (such as shown in FIG. 17B) and a mid-turbinate swab (not shown). The nasal swab may be performed using a conventional nasal swab 822, such as shown in FIG. 17B, or using nasal sampling device 1020, such as shown in FIG. 22. The specimen sample is typically collected without performing a nasopharyngeal swab. When the specimen sample is collected by performing the anterior nares swab, the specimen sample is optionally collected without performing a mid-turbinate swab or a nasopharyngeal swab. The nasal swab may be performed in one or both of the nostrils; in the latter case, nasal wash fluid 26 is intranasally dispensed into both nostrils of nasal cavity 22, and the same single swab is typically used in both nostrils. Alternatively or additionally, the specimen sample is collected by performing a nasopharyngeal swab.

In some applications of the present invention, a method is provided that comprises intranasally dispensing nasal wash fluid 26 into nasal cavity 22 of a subject, by the subject or somebody else, such as shown in FIG. 17A. Nasal wash fluid 26 generally loosens biological material in nasal cavity 22 and/or nasopharynx 32. Thereafter, a specimen sampler is inserted into nasal cavity 22 by the subject or by somebody else, such that a farthest-inserted portion of the specimen sampler is positioned in an anterior naris of nasal cavity 22. A specimen sample is collected on the specimen sampler by contacting (e.g., rubbing) a wall of the anterior naris (i.e., the wall's epithelial surface) with the specimen sampler, optionally including rotating the specimen sampler. For example, the specimen sampler may comprise a swab (e.g., a flocked swab) (such as shown in FIG. 17B), a brush, a sponge, or a nasal plug. Typically, but not necessarily, the specimen sampler comprises a tip comprising an absorbent material. For applications in which the specimen sampler comprises a swab, a tip of the swab may, for example, be a flocked swab tip (e.g., comprising nylon), a rayon swab tip, a cotton swab tip, a polyurethane foam swab tip, a polyethylene terephthalate (PET) swab tip, a polyester swab tip, a foam swab tip (e.g., comprising polyurethane) (e.g., an open-cell foam swab), a sponge swab tip (e.g., comprising cellulose), a brush swab tip (e.g., similar to a cytology brush), and a nanofiber swab tip. The specimen sampler may be inserted into a single nostril of nasal cavity 22, or into both of the nostrils, one at a time; in the latter case, nasal wash fluid 26 is intranasally dispensed into both nostrils of nasal cavity 22.

In some applications of the present invention, a method is provided that comprises intranasally dispensing nasal wash fluid 26 into nasal cavity 22 of a subject, by the subject or somebody else, such as shown in FIG. 17A. Nasal wash fluid 26 generally loosens biological material in nasal cavity 22 and/or nasopharynx 32. Thereafter, a specimen sampler is inserted into nasal cavity 22 by the subject or by somebody else. While the specimen sample is inserted into the nasal cavity, a specimen sample is collected on the specimen sampler without using suction, such as shown in FIG. 17B.

In some applications of the present invention, a method is provided that comprises intranasally dispensing nasal wash fluid 26 into nasal cavity 22 of a subject, by the subject or somebody else, such as shown in FIG. 17A. Nasal wash fluid 26 generally loosens biological material in nasal cavity 22 and/or nasopharynx 32. Thereafter, the subject or somebody else inserts, into nasal cavity 22, a specimen sampler that is shaped so as not to define any dispensing openings. While the specimen sampler is inserted into the nasal cavity, a specimen sample is collected on the specimen sampler, such as shown in FIG. 17B.

For some applications, before the specimen sample is collected using one of the techniques described in the previous paragraphs (by performing the nasal swab or using a specimen sampler inserted into the nasal cavity):
before nasal wash fluid 26 is intranasally dispensed, the subject (typically while sitting or standing) tilts back his or her head (typically until the neck is fully extended) and holds the head tilted back while nasal wash fluid 26 is intranasally dispensed, or
after nasal wash fluid 26 is intranasally dispensed, the subject tilts his or her head farther back than while intranasally dispensing the nasal wash fluid and farther back than a neutral position.

In all of these cases, the tilting back may cause nasal wash fluid 26 to loosen biological material in a more posterior (deeper) portion of nasal cavity 22 and/or nasopharynx 32.

Optionally, the subject sniffs after nasal wash fluid 26 is intranasally dispensed and before the specimen sample is collected. In applications in which the subject tilts the head far back, the sniffing may be performed before or after such far back tilting.

For some applications, nasal wash fluid 26 is intranasally dispensed without blowing, by the subject, a nose of the subject immediately prior to intranasally dispensing the nasal wash fluid. Blowing the nose may undesirably remove mucus and other biological material containing high concentrations of particulate of interest. Alternatively, the subject may blow his or her nose before the nasal wash fluid is intranasally dispensed.

Optionally, after the subject tilts his or her head back and after nasal wash fluid 26 is intranasally dispensed, the subject tilts forward his or her head to a neutral or a tilted-forward position; this backward-followed-by-forward tilting may help move biological material anteriorly (i.e., toward the nostril) within nasal cavity 22.

Optionally, the method further comprises, while holding his or her head tilted back, waiting for a period of time between (a) concluding intranasally dispensing nasal wash fluid 26 into the nasal cavity or tilting the head farther back and (b) tilting his or her head forward to the neutral or the tilted-forward position, the period of time at least 2 seconds (e.g., at least 5 seconds, such as at least 10 seconds, e.g., at least 15 seconds), no more than 60 seconds (e.g., no more than 30 seconds), and/or between 2 seconds (e.g., 5 seconds, such as 10 seconds, e.g., at least 15 seconds) and 60 seconds (e.g., 30 seconds).

Optionally, while holding his or her head tilted back, the subject makes a sound, such as a “K” sound, that inhibits flow of nasal wash fluid 26 from nasopharynx 32 to oropharynx 30, helping retain nasal wash fluid 26 in nasopharynx 32.

For some applications, the subject tilts his or her head forward to the neutral or the tilted-forward position after the subject senses (e.g., by feeling and/or tasting) the nasal wash fluid in oropharynx 30. Optionally, nasal wash fluid 26 comprises a tastant, for example, menthol flavoring.

For some applications, after tilting the head forward to the neutral or the tilted-forward position, the subject waits for a period of time before collecting the specimen sample, the period of time at least 5 seconds, e.g., at least 10 seconds, such as at least 30 seconds (and typically less than 120 seconds). This waiting period may allow sufficient time for nasal wash fluid 26 to drip anteriorly in nasal cavity 22, bringing the biological material with it.

Alternatively, for some applications, nasal wash fluid 26 is intranasally dispensed into nasal cavity 22 (either continuously or in a plurality of pulses) until the subject senses (e.g., by feeling and/or tasting) nasal wash fluid 26 in oropharynx 30. Optionally, nasal wash fluid 26 comprises a tastant, for example, menthol flavoring.

Optionally, the method further comprises waiting for a period of time between (a) concluding intranasally dispensing nasal wash fluid 26 into the nasal cavity and (b) collecting the specimen sample, the period of time at least 2 seconds (e.g., at least 5 seconds, such as at least 10 seconds), no more than 30 minutes (e.g., no more than 15 minutes), and/or between 2 seconds (e.g., 5 seconds, such as at least 10 seconds) and 30 minutes (e.g., 15 minutes).

For some applications, nasal wash fluid 26 is intranasally dispensed as one or more types of fluid discharge selected from the group consisting of: a spray, a mist, a pressurized aerosol, steam, and an atomized fluid. For some applications, nasal wash fluid 26 is intranasally dispensed as a fluid stream.

For some applications, nasal wash fluid 26 is dispensed into each of one or both nostrils 28 in a total volume of between 0.15 and 1 mL, such as between 0.15 and 0.5 mL, or between 0.25 and 1 mL, such as between 0.25 and 0.5 mL. Optionally, nasal wash fluid 26 is intranasally dispensed in a plurality of pulses per nostril (e.g., between 3 and 5 pulses per nostril), each having a volume of between 0.05 and 0.2 mL, for example, between 0.05 and 0.15 mL, e.g., 0.1 mL. (If nasal wash fluid 26 is dispensed into both nostrils 28, the volumes above would be doubled.) Dispensing such relatively small volumes reduces the likelihood of nasal wash fluid 26 washing out the biological material of interest from the nostrils.

In some applications, the specimen samples collected using the collection techniques described herein are tested for a particulate using one or more of the techniques described herein, one or more of the techniques described in the patent applications incorporated hereinbelow by reference, or a technique known in the art. The particulate may be a virus (e.g., SARS-CoV-2 or an Influenza virus), a bacterium, any of the other particulates described hereinabove, or any of the other biological materials described hereinabove. For example, the particulate may be tested for the presence of a particulate using nucleic acid amplification, such as PCR, e.g., qPCR, and/or by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, the particulate may be tested the presence of a particulate using a CRISPR-based diagnostic test, an ELISA diagnostic test, or a spectroscopy-based diagnostic test.

These sampling methods may be combined with any other applicable techniques described herein, mutatis mutandis. By way of example and not limitation, these sampling methods may further comprise, after nasal wash fluid 26 is intranasally dispensed, orally dispensing oral wash fluid 68 into oral cavity 36 via anterior opening 34 of oral cavity 36; and gargling, by the subject, oral wash fluid 68. Optionally:
oral wash fluid 68 is orally dispensed into oral cavity 36 such that oral wash fluid 68 forms mixture 70 with nasal wash fluid 26 in oropharynx 30, and, in addition to collecting the specimen sample from the nasal cavity (by swabbing or using the specimen sampler), a specimen sample 24 is collected that passed out of anterior opening 34 of oral cavity 36 and contains at least a portion of mixture 70 and, typically, at least a portion of biological material washed into oropharynx 30 with nasal wash fluid 26; and/or
oral wash fluid 68 is orally dispensed into oral cavity 36, the subject gargles oral wash fluid 68, such that oral wash fluid 68 forms mixture 70 with nasal wash fluid 26, and a specimen sample 24 is collected that passed out of anterior opening 34 of oral cavity 36 and contains at least a portion of mixture 70 and the at least a portion of biological material washed into oropharynx 30 with nasal wash fluid 26.

Alternatively, a specimen sample is not collected that passed out of anterior opening 34 of oral cavity 36, and the gargled fluid is instead spit out and discarded. In this case, the time spent gargling (e.g., 5-15 seconds) may serve to ensure that the subject holds his or her head tilted back for a sufficient period of time for nasal wash fluid 26 to loosen biological material in nasal cavity 22 (such as in a more posterior (deeper) portion of nasal cavity 22 and/or nasopharynx 32). The gargling may alternatively or additionally distract the subject from the unpleasant sensation of post-nasal drip of nasal wash fluid 26 into the throat, so that the subject does not prematurely tilt his or her head forward to the neutral or the tilted-forward position before nasal wash fluid 26 sufficiently loosens the biological material in nasal cavity 22.

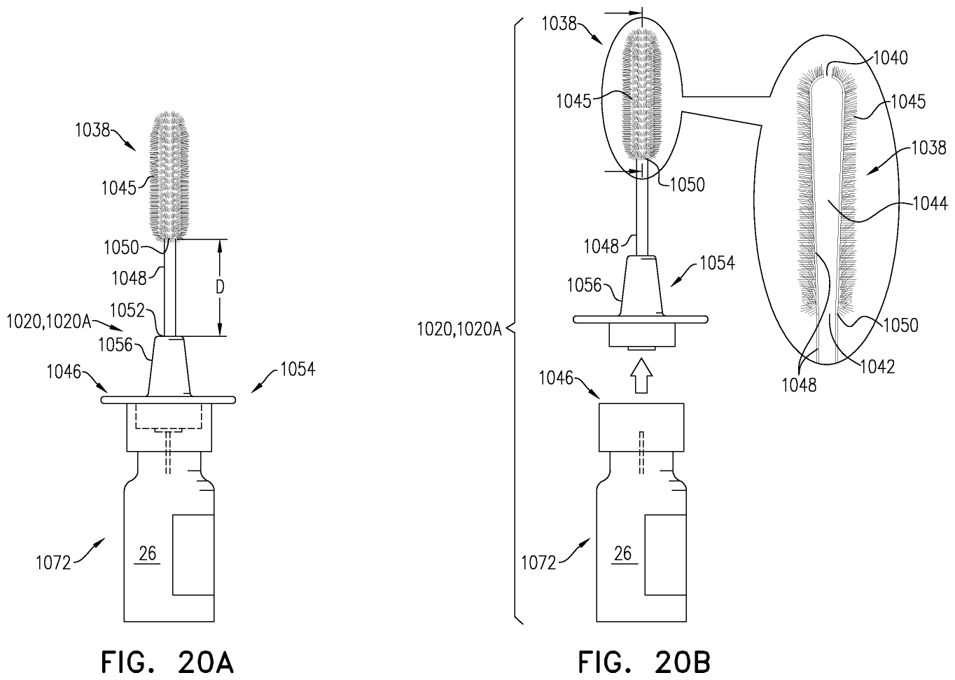

Also by way of example and not limitation, these nasal sampling methods may be performed using any of nasal wash fluid 26 dispensers described herein with reference to FIGS. 1, 2A-C, 3A-B, 6, 7A-K, 8A-C, 9A-F, 10, and/or 11. Alternatively, these nasal sampling methods may be performed using a conventional nasal dispenser, such as shown in FIG. 17A. Further alternatively, these nasal sampling methods may be performed using nasal sampling device 1020, described hereinbelow with reference to FIGS. 20A-B and 21A-B.

Experiments 9 and 10, described hereinbelow, provide evidence of the efficacy of some of these nasal-spray and nasal-sampling techniques for collecting specimen samples for detection of SARS-CoV-2 using quantitative PCR (qPCR).

Experiments 11 and 13, described hereinbelow, provide evidence of the efficacy of some of these nasal-spray and nasal-sampling techniques for collecting specimen samples for detection of SARS-CoV-2 using lateral flow immunoassay strips. The inventors believe that one of ordinary skill in the art would not expect that intranasal dispensation of nasal wash fluid 26 would enhance the sensitivity of detection of SARS-CoV-2 (or any other virus or biological particulate) using a lateral flow immunoassay strip, because of the much lower sensitivity of lateral flow immunoassay strips than nucleic acid amplification tests, such as PCR. Lateral flow immunoassay strips are well known to provide accurate results only when a fairly dry and concentrated sample is immersed in a small volume of reagent liquid, such as 0.2 to 0.4 mL, before application to the strip. In addition, anterior nares samples are known to be weaker than mid-turbinate and nasopharyngeal samples. Therefore, one of ordinary skill in the art would expect that the intranasal dispensation of nasal wash fluid 26 might dilute the biological material captured in the anterior nares specimen sample. In addition, one of ordinary skill in the art would expect that the intranasal dispensation of nasal wash fluid 26 might wash biological material potentially containing the virus off of the wall of the anterior nares before collection of the specimen sample, thereby reducing the amount of virus collected from the anterior nares.

For all of these reasons, one of ordinary skill in the art would expect the intranasal dispensation of nasal wash fluid before collecting the specimen sample would reduce the sensitivity of tests performed using lateral flow immunoassay strips. By contrast, the inventors have found that intranasal dispensation of nasal wash fluid 26 improves the sensitivity of lateral flow immunoassay tests performed on nasal samples collected from the anterior nares.

The dimensions and absorptive properties of swabs that may be useful in the nasal swabbing sample collection techniques described herein, such as for testing using a lateral flow immunoassay strip (e.g., for SARS-CoV-2 or an Influenza virus) were examined on behalf of the inventors. Specifically, flocked nasal swabs (3BY Ltd., Industrial Park Tefen, Israel, Ref: 3BY-001) were examined, because they were used in a number of the experiments described herein, and foam tipped nasal swabs (Puritan, Me., USA, Ref: 25-1506-1PF 100), which were provided with the BD Veritor™ system for Rapid Detection of SARS-CoV-2 (Becton Dickinson and Company, Maryland, USA, Ref: 256082), were examined because they are appropriate for sample collection for use in immunoassays.

Swab tip dimensions were determined using a mechanical caliper, or were provided by swab manufacturers (Table 1). Following saline nasal spraying and swabbing, swabs were found to contain between approximately 76-131 microliters of fluid from a single nostril (Table 2), after administering about 500 microliters of nasal wash into the nostril. Utilizing relatively smaller swab tips for respiratory specimen collection may be beneficial because of the limited volumes of lysing buffer included in commercial immunoassay kits (Table 3), which must be sufficient for the extraction of viral antigens from virions, intact infected cells, or infected cell debris which have been collected by the swab. If swabs were to contain a larger sample volume, the lysing buffer would be diluted beyond the optimal threshold and effective extraction would be unlikely to occur. Moreover, oversized swabs which absorb more fluid than what is recoverable during sampling would further absorb lysing buffer, making it difficult to elute sufficient lysate from the swab for assaying, because standard immunoassay cassettes require 3 drops of lysate (approximately 150 microliters), with dipstick style immunoassays typically absorbing at least 20 microliters of lysate.

TABLE 1
Swab Dimensions

| Swab Type | Flocked | Foam |
|---|---|---|
| Greatest Tip Diameter (mm)^{v} | 4 | 4.775ⁱ |
| Tip Length (mm) | 17 | 16.002ⁱ |
| Tip Surface Area (mm²)ⁱⁱ | 226 | 258 |
| Tip Volume (mm³)ⁱⁱⁱ | 214 | 286 |
| Applicator Thickness at Tip (mm) | 2 | 2.54ⁱ |
| Applicator Length at Tip (mm) | 16.5 | 13 |
| Applicator Volume at Tip (mm2) ⁱⁱⁱ | 52 | 64 |
| Effective Absorptive Volume (mm³)^{iii,iv} | 162 | 222 |

ⁱDimensions provided by manufacturer (Puritan Medical Products Company LLC, Guilford, Maine, USA)
ⁱⁱSurface area was calculated assuming a perfectly cylindrical shape but excluding the bottom of the tip because of the presence of the applicator (2πrh + πr\hat{ }2).
ⁱⁱⁱVolume was calculated assuming a perfectly cylindrical shape.
^{iv}Effective absorptive volume was determined by subtracting the applicator volume at tip from the tip volume.
^{iv}The greatest tip diameter is the diameter of the swab at the widest location along the swab.

TABLE 2
Swab Absorption

| | | Weight | Weight | | |
|---|---|---|---|---|---|
| | | Before | After | | Fluid |
| Swabbing | | Swabbing | Swabbing | Difference | Volume |
| Type | Swab | (g) | (g) | (g) | (microliters)ⁱ |
| Saturation with | Flocked | 0.668 | 0.768 | 0.1 | 100 |
| DPBS | Foam | 0.468 | 0.502 | 0.034 | 34 |
| (−Rotation)ⁱⁱ | | | | | |
| Saturation with | Flocked | 0.691 | 0.795 | 0.104 | 104 |
| DPBS | Foam | 0.476 | 0.651 | 0.175 | 175 |
| (+Rotation)ⁱⁱ | | | | | |
| Bilateral | Flocked | 0.669 | 0.672 | 0.003 | 3 |
| Swabbing | Foam | 0.497 | 0.493 | −0.004 | −4^{iv} |
| Spraying and | Flocked | 0.672 | 0.768 | 0.096 | 96 |
| Unilateral | Foam | 0.472 | 0.548 | 0.076 | 76 |
| Swabbingⁱⁱⁱ | | | | | |
| Spraying and | Flocked | 0.678 | 0.783 | 0.105 | 105 |
| Bilateral | Foam | 0.469 | 0.6 | 0.131 | 131 |
| Swabbingⁱⁱⁱ | | | | | |

ⁱAssumed density of 1 g/cm3 for absorbed liquid.
ⁱⁱSwabs were dipped into a 50 mL conical tube containing Deulbecco's Phosphate Buffered Saline (DBPS), and them either removed (−rotation) or rotated while brushing the walls of the tube five times to simulate anterior nares swabbing (+rotation)
ⁱⁱⁱUnilateral and Bilateral swabbing of the anterior nares were performed by inserting the entire tip of the swab into the nares and rotating the swab around the inner walls of the nares. Saline nasal spraying consisted of 5 sprays of 0.1 mL of saline into one or both nostriles.
^{iv}Negative values are assumed to be a result of either a small amount of the tip separating from the swab during swabbing, or are an artifcat due to inaccuracies in the analytical balance.

TABLE 3
Lysing Buffer Volumes

| | | Weight of | Approximate |
|---|---|---|---|
| | | Lysing | Volume |
| Assay Name | Manufacturer | Buffer (g) | (microliters)ⁱ |
| QuickVue Influenza | Quidel Corporation | 0.245 | 245 |
| A + B Test | (San Diego, CA, | | |
| | Ref: 20183) | | |
| BD Veritor ™ | Becton Dickinson | 0.413 | 413 |
| system for Rapid | and Company | | |
| Detection of SARS- | (Maryland, USA, | | |
| CoV-2 | Ref: 256082) | | |
| COVID-19 Antigen | Beijing Kewei | 0.283 | 283 |
| Rapid Test Cassette | Clinical Diagnostic | | |
| | Reagent Inc | | |
| | (Beijing, China, Ref: | | |
| | 601450) | | |

ⁱAssumed density of 1 g/cm3 for liquid reagents

For some applications, any of the swabs used in the methods described herein, or as elements of apparatus described herein, comprise a tip having one or more of the following characteristics:
a greatest diameter of at least 2 mm, less than 5 mm (e.g., less than 4.5 mm), and/or between 2 and 5 mm (e.g., between 2 and 4.5 mm),
a collection surface area of at least 200 mm2, less than 300 mm2, and/or between 200 and 300 mm2 (the collection surface area includes the absorptive portion of the swab, but excludes the bottom of the tip because of the presence of the applicator),
a volume of at least 200 mm3, less than 300 mm3, and/or between 200 and 300 mm3, and/or
a length of at least 12 mm, less than 20 mm, and/or between 12 and 20 mm.

Optionally, any of the swabs and/or specimen samplers described herein, including, but not limited to, with reference to FIGS. 18, 19, 20A-B, 21A-B, 21C-D, 22, and 23A-B, may have the characteristics described immediately above.

For some applications, in any of the nasal collection techniques described herein, collecting the specimen sample comprises collecting at least 25 microliters, less than 150 microliters (e.g., less than 125 microliters), and/or between 25 and 150 microliters (e.g., between 25 and 125 microliters) of material per specimen sampler, e.g., per swab (such as, for example, flocked swab). The material typically includes a combination of the biological material of interest and a portion of nasal wash fluid 26 dispensed into nasal cavity 22. Alternatively, for some applications, in any of the nasal collection techniques described herein, collecting the specimen sample comprises collecting at least 25 microliters, less than 250 microliters (e.g., less than 200 microliters), and/or between 25 and 250 microliters (e.g., between 25 and 200 microliters) of material per specimen sampler, e.g., per swab (such as, for example, foam swab).

In some applications of the present invention, a method is provided that comprises intranasally dispensing nasal wash fluid 26 into nasal cavity 22 of a subject, by the subject or by somebody else. Nasal wash fluid 26 generally loosens biological material in nasal cavity 22 and/or nasopharynx 32. Thereafter, a specimen sample is collected by blowing a nose, by the subject, into a specimen receptacle. These techniques may be practiced in combination with any of the techniques described herein for intranasally dispensing nasal wash fluid 26 into nasal cavity 22.

Reference is now made to FIG. 18, which is a schematic illustration of a testing kit 800, in accordance with an application of the present invention. Testing kit 800 comprises:
a nasal dispenser (containing nasal wash fluid 26), such as conventional nasal dispenser 820, as shown, or nasal wash fluid dispenser 20, not shown in FIG. 18, but shown in FIG. 2B;
conventional nasal swab 822; and
test 302 (e.g., a diagnostic test), which is configured to test for the presence of a particulate in a specimen sample collected using the techniques described herein; test 302 is described hereinabove with reference to FIG. 12.

Nasal swab 822 comprises a swab tip 838 that comprises an absorbent material 844 for collecting the specimen sample. As used in the present application, including in the claims and Inventive Concepts, an “absorbent material” is a material that collects and retains a specimen sample, such as because of the physical or chemical structure and/or properties of the material (such as, for example, adhesive properties), and/or the structure formed by the material. For example, the fibers of synthetic flocking are arranged to be absorbent, even if the fibers in isolation from one another would not be absorbent. For example, swab tip 838 may be a flocked swab tip (e.g., comprising nylon), a rayon swab tip, a cotton swab tip, a polyurethane foam swab tip, a polyethylene terephthalate (PET) swab tip, a polyester swab tip, a foam swab tip (e.g., comprising polyurethane) (e.g., an open-cell foam swab), a sponge swab tip (e.g., comprising cellulose), a brush swab tip (e.g., similar to a cytology brush), and a nanofiber swab tip.

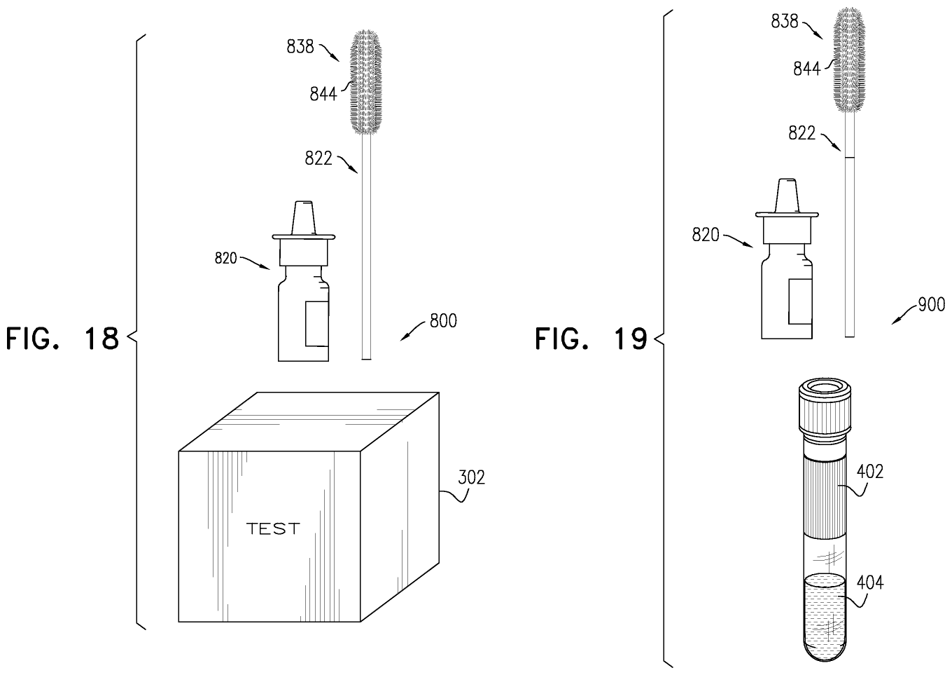

Reference is now made to FIG. 19, which is a schematic illustration of a testing kit 900, in accordance with an application of the present invention. Testing kit 900 comprises:
a nasal dispenser (containing nasal wash fluid 26), such as conventional nasal dispenser 820, as shown, or nasal wash fluid dispenser 20, not shown in FIG. 19, but shown in FIG. 2B;
nasal swab 822; and
vial 402 and liquid 404 for bathing at least a portion of a specimen sample in the vial, the liquid selected from the group consisting of: a lysis buffer, saline solution (e.g., phosphate buffered saline (PBS)), and transport medium (e.g., universal transport medium or a viral transport medium).

Vial 402 and liquid 404 are described hereinabove with reference to FIG. 13.

Nasal swab 822 comprises swab tip 838 that comprises absorbent material 844 for collecting the specimen sample. For example, swab tip 838 may be a flocked swab tip (e.g., comprising nylon), a rayon swab tip, a cotton swab tip, a polyester swab tip, a polyurethane foam swab tip, a polyethylene terephthalate (PET) swab tip, a foam swab tip (e.g., comprising polyurethane) (e.g., an open-cell foam swab), a sponge swab tip (e.g., comprising cellulose), a brush swab tip (e.g., similar to a cytology brush), and a nanofiber swab tip.

Testing kit 900 may be used, for example, for collecting a specimen sample from the subject and sending at least a portion of the specimen sample to a remote laboratory in the liquid in the vial, such as for performing Polymerase Chain Reaction (PCR) testing for particulate in the specimen sample. The particulate may be a virus, a bacterium, any of the other particulates described hereinabove, or any of the other biological materials described hereinabove.

Reference is now made to FIGS. 20A-B and 21A-D, which are schematic illustrations of respective configurations of a nasal sampling device 1020 for collecting a specimen sample, in accordance with respective applications of the present invention. FIGS. 20A-B show a nasal sampling device 1020A and FIGS. 21A-B show a nasal sampling device 1020B. Nasal sampling device 1020A and nasal sampling device 1020B are configurations of nasal sampling device 1020 and are identical to each other except as described hereinbelow.

Nasal sampling device 1020 comprises a dispensing container 1072 and a nasal applicator 1038 (which is sized and shaped to be insertable into an anterior nares of a nostril). Dispensing container 1072 contains nasal wash fluid 26; optionally, nasal wash fluid 26 has any of the properties of nasal wash fluid 26 described hereinabove with reference to FIG. 2B.

Nasal applicator 1038 is shaped so as to define (i) one or more nozzles 1040, (ii) a proximal opening 1042, and (iii) one or more channels 1044 within nasal applicator 1038 that connect the one or more nozzles 1040 in fluid communication with proximal opening 1042. For example, nasal applicator 1038 may be shaped so as define exactly one dispensing opening 1040, which may, for example, be located at a distal end of nasal applicator 1038, such as shown. Alternatively, nasal applicator 1038 may be shaped so as define a plurality of nozzles 1040, which may include, for example, openings facing laterally (such as shown in FIG. 21D) and, optionally, an opening at the distal end of nasal applicator 1038.

At least a portion of an external surface of nasal applicator 1038 comprises an absorbent material 1044 for collecting the specimen sample. For example, absorbent material 1044 may comprise flocking (e.g., comprising nylon), rayon, cotton, polyester, polyurethane foam, polyethylene terephthalate (PET), foam (e.g., comprising polyurethane) (e.g., an open-cell foam), sponge (e.g., comprising cellulose), a brush (e.g., similar to a cytology brush), and nanofibers.

For some applications, nasal applicator 1038 is shaped similarly to a conventional nasal swab.

In the configurations shown in FIGS. 20A-B and 21A-B, an entirety of the external surface of nasal applicator 1038, other than the portion that defines the one or more nozzles 1040, comprises absorbent material 1044. In the configurations shown in FIGS. 21C-D, only a portion of the external surface of nasal applicator 1038 comprises absorbent material 1044.

For some applications, nasal applicator 1038 may implement any of the nozzles described hereinabove with reference to FIGS. 1, 2A-C, 3A-B, 6, 7A-K, 8A-C, 9A-F, 10, and/or 11, including the angles, offset angles, spray angles, number of nozzles, and orientations of the nozzles.

Nasal applicator 1038 is disposed distal to dispensing container 1072, with proximal opening 1042 of nasal applicator 1038 in fluid communication with dispensing container 1072. Nasal sampling device 1020 is configured to intranasally dispense nasal wash fluid 26 from dispensing container 1072 into nasal cavity 22 of the one or more nozzles 1040 of nasal applicator 1038.

For some applications, nasal sampling device 1020 comprises a pump 1046 that is configured to dispense nasal wash fluid 26.

For some applications, nasal sampling device 1020 is configured to dispense nasal wash fluid 26 by manual compression of dispensing container 1072.

For some applications, nasal sampling device 1020 comprises an atomizer that is configured to dispense nasal wash fluid 26.

For some other applications, nasal sampling device 1020 comprises a pressurized aerosol generator that is configured to dispense nasal wash fluid 26.

Reference is made to FIGS. 20A-B. For some applications, nasal sampling device 1020A further comprises a tube 1048, which physically connects a proximal end 1050 of nasal applicator 1038 to dispensing container 1072. Proximal opening 1042 of nasal applicator 1038 is in fluid communication with dispensing container 1072 via tube 1048.

Optionally, tube 1048 extends distally through at least a portion of nasal applicator 1038, so as to define at least a portion of the one or more channels 1044 within nasal applicator 1038, such as shown in FIGS. 20A-B.

For some applications, tube 1048 physically connects proximal end 1050 of nasal applicator 1038 to dispensing container 1072 such that a distance D between proximal end 1050 of nasal applicator 1038 and a distal-most point 1052 of dispensing container 1072 is at least 1 cm, no more than 13 cm, and/or between 1 and 13 cm.

Reference is made to FIGS. 21A-B. For some applications, proximal end 1050 of nasal applicator 1038 of nasal sampling device 1020B is in direct physical contact with dispensing container 1072.

For some applications, proximal end 1050 of nasal applicator 1038 is fixed in the direct physical contact with dispensing container 1072. For other applications, proximal end 1050 of nasal applicator 1038 is removably coupled in the direct physical contact with dispensing container 1072.

Reference is made to FIGS. 20A-B and 21A-B. For some applications, nasal applicator 1038 is removable from dispensing container 1072.

Reference is still made to FIGS. 20A-B and 21A-B. For some applications, nasal sampling device 1020 comprises a user control 1054 that is coupled to dispensing container 1072 and configured to cause dispensing container 1072 to dispense nasal wash fluid 26. For some of these applications, proximal end 1050 of nasal applicator 1038 is fixed to a distal portion 1056 of user control 1054, and at least distal portion 1056 of user control 1054 is removable from dispensing container 1072, so as to remove nasal applicator 1038 from dispensing container 1072. Either the entirety of user control 1054 is removable from dispensing container 1072, or only a portion of user control 1054 is removable from dispensing container 1072, while another portion of user control 1054 remains coupled to dispensing container 1072.

Reference is now made to FIG. 22, which is a schematic illustration of the use of nasal sampling device 1020 for collecting a specimen sample from nasal cavity 22, in accordance with an application of the present invention. Although by way of illustration FIG. 22 shows nasal sampling device 1020A, described hereinabove with reference to FIGS. 20A-B, nasal sampling device 1020B, described hereinabove with reference to FIGS. 21A-B, may be used in the same manner. As illustrated in FIG. 22, nasal sampling device 1020 includes exactly one nozzle 1040.

As shown in FIG. 22, nasal applicator 1038 of nasal sampling device 1020 is introduced into nostril 28, and thereafter nasal wash fluid 26 is intranasally dispensed, by the subject or by somebody else, into nasal cavity 22 from the one or more nozzles 1040, by introducing nasal wash fluid 26 into proximal opening 1042 (from dispensing container 1072). Typically, nasal applicator 1038 is introduced into nostril 28 no more deeply than an anterior naris 1060 of nostril 28.

Thereafter, the specimen sample is collected from nasal cavity 22 by contacting (typically, rubbing) a wall 1058 of anterior naris 1060 with absorbent material 1044 of nasal applicator 1038, by the subject or by somebody else. Typically, no portion of nostril 28 deeper than anterior naris 1060 is contacted by absorbent material 1044 of nasal applicator 1038. Typically contacting wall 1058 of anterior naris 1060 with absorbent material 1044 of nasal applicator 1038 comprises rubbing wall 1058 of anterior naris 1060 with absorbent material 1044 of nasal applicator 1038. Typically, nasal applicator 1038 is not removed from and reinserted into nostril 28 between intranasally dispensing nasal wash fluid 26 and collecting the specimen sample. Alternatively, nasal applicator 1038 is removed from and reinserted into nostril 28 between intranasally dispensing nasal wash fluid 26 and collecting the specimen sample.

The method may be performed in one or both of the nostrils. Optionally, the method is performed in combination with other sampling methods described herein, including, but not limited to gargling after dispensing nasal wash fluid 26.

For some applications, collecting the specimen sample from nasal cavity 22 comprises collecting the specimen sample without using suction. Alternatively, suction is used to collect the specimen sample.

For some applications in which nasal applicator 1038 is removable from dispensing container 1072, after the specimen sample is collected, nasal applicator 1038 is removed from dispensing container 1072. Optionally, nasal applicator 1038 is inserted into a liquid, such as a lysis buffer, saline solution (e.g., phosphate buffered saline (PBS)), transport medium (e.g., universal transport medium or a viral transport medium), or a composition comprising a detergent and a buffering agent for stabilizing RNA (e.g., distributed by DNA Genotek Inc., a subsidiary of OraSure Technologies, Inc. (Bethlehem, Pa., USA), for example, ORAcollect®⋅RNA Saliva Collection Device for SARS CoV-2). The liquid is optionally contained in collection receptacle 40, described hereinabove with reference to FIG. 2D.

For some applications, before the specimen sample is collected using the techniques described with reference to FIG. 22:
before nasal wash fluid 26 is intranasally dispensed, the subject (typically while sitting or standing) tilts back his or her head (typically until the neck is fully extended) and holds the head tilted back while nasal wash fluid 26 is intranasally dispensed, or
after nasal wash fluid 26 is intranasally dispensed, the subject tilts back his or her head farther back than while intranasally dispensing the nasal wash fluid and farther back than a neutral position.

In all of these cases, the tilting back may cause nasal wash fluid 26 to loosen biological material in a more posterior (deeper) portion of nasal cavity 22 and/or nasopharynx 32.

Optionally, after the subject tilts his or her head back and after nasal wash fluid 26 is intranasally dispensed, the subject tilts forward his or her head to a neutral or a tilted-forward position; this backward-followed-by-forward tilting may help move biological material anteriorly (i.e., toward the nostril) within nasal cavity 22.

Optionally, the method further comprises, while holding his or her head tilted back, waiting for a period of time between (a) concluding intranasally dispensing nasal wash fluid 26 into the nasal cavity and (b) tilting his or her head forward to the neutral or the tilted-forward position, the period of time at least 2 seconds (e.g., at least 5 seconds, such as at least 10 seconds, e.g., at least 15 seconds), no more than 60 seconds (e.g., no more than 30 seconds), and/or between 2 seconds (e.g., 5 seconds, such as at least 10 seconds, e.g., at least 15 seconds) and 60 seconds (e.g., 30 seconds).

For some applications, the subject tilts his or her head forward to the neutral or the tilted-forward position after the subject senses (e.g., by feeling and/or tasting) the nasal wash fluid in oropharynx 30. Optionally, nasal wash fluid 26 comprises a tastant, for example, menthol flavoring.

Alternatively, for some applications, nasal wash fluid 26 is intranasally dispensed into nasal cavity 22 (either continuously or in a plurality of pulses) until the subject senses (e.g., by feeling and/or tasting) nasal wash fluid 26 in oropharynx 30. Optionally, nasal wash fluid 26 comprises a tastant, for example, menthol flavoring.

Optionally, the method further comprises waiting for a period of time between (a) concluding intranasally dispensing nasal wash fluid 26 into the nasal cavity and (b) collecting the specimen sample, the period of time at least 2 seconds (e.g., at least 5 seconds, such as at least 10 seconds), no more than 30 minutes (e.g., no more than 15 minutes), and/or between 2 seconds (e.g., 5 seconds, such as at least 10 seconds) and 30 minutes (e.g., 15 minutes).

For some applications, nasal wash fluid 26 is intranasally dispensed as one or more types of fluid discharge selected from the group consisting of: a spray, a mist, a pressurized aerosol, steam, and an atomized fluid. For some applications, nasal wash fluid 26 is intranasally dispensed as a fluid stream.

For some applications, nasal wash fluid 26 is dispensed into each of one or both nostrils 28 in a total volume of between 0.15 and 1 mL, such as between 0.15 and 0.5 mL, or between 0.25 and 1 mL, such as between 0.25 and 0.5 mL. Optionally, nasal wash fluid 26 is intranasally dispensed in a plurality of pulses per nostril (e.g., between 3 and 5 pulses per nostril), each having a volume of between 0.05 and 0.2 mL, for example, between 0.05 and 0.15 mL, e.g., 0.1 mL. (If nasal wash fluid 26 is dispensed into both nostrils 28, the volumes above would be doubled.) Dispensing such relatively small volumes reduces the likelihood of nasal wash fluid 26 washing out the biological material of interest from the nostrils.

In some applications, the specimen samples collected using the collection techniques described herein are tested for the presence of a particulate using one or more of the techniques described in the patent applications incorporated hereinbelow by reference, or a technique known in the art. The particulate may be a virus (e.g., an Influenza virus or SARS-CoV-2), a bacterium (e.g., Streptococcus bacterium), any of the other particulates described hereinabove, or any of the other biological materials described hereinabove. For example, the particulate may be tested for the presence of a particulate using nucleic acid amplification, such as PCR, e.g., qPCR, and/or by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, the particulate may be tested the presence of a particulate using a CRISPR-based diagnostic test, an ELISA diagnostic test, or a spectroscopy-based diagnostic test.

In an application of the present invention, a testing kit is provided that comprises nasal sampling device 1020 and test 302 (e.g., a diagnostic test), which is configured to test for the presence of a particulate in a specimen sample collected using the techniques described herein. Test 302 is described hereinabove with reference to FIG. 12.

In an application of the present invention, a testing kit is provided that comprises nasal sampling device 1020, vial 402, and liquid 404 for bathing at least a portion of a specimen sample in the vial, the liquid selected from the group consisting of: a lysis buffer, saline solution (e.g., phosphate buffered saline (PBS)), and transport medium (e.g., universal transport medium or a viral transport medium). Vial 402 and liquid 404 are described hereinabove with reference to FIG. 13. The testing kit may be used, for example, for collecting a specimen sample from the subject and sending at least a portion of the specimen sample to a remote laboratory in the liquid in the vial, such as for performing Polymerase Chain Reaction (PCR) testing for particulate in the specimen sample. The particulate may be a virus, a bacterium, any of the other particulates described hereinabove, or any of the other biological materials described hereinabove.

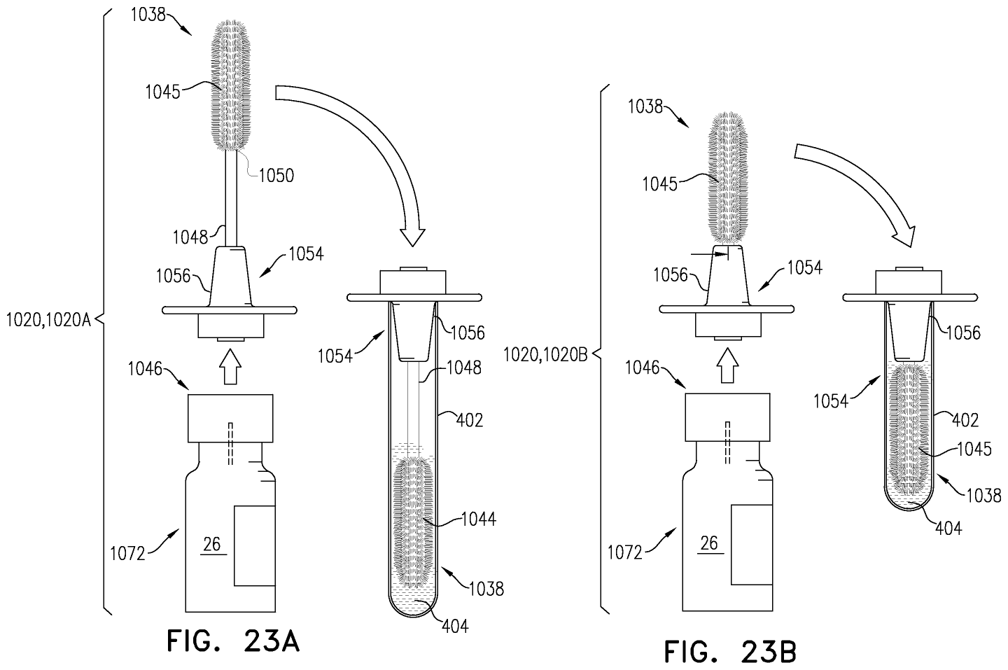

Reference is now made to FIGS. 23A and 23B, which are schematic illustrations of kits that comprise portions of nasal sampling devices 1020A and 1020B, respectively, and vial 402, in accordance with respective applications of the present invention. In these configurations, distal portion 1056 of user control 1054 is configured, after removal from dispensing container, such as described hereinabove with reference to FIGS. 20B and 21B, to be coupled to the opening of vial 402, such that nasal applicator 1038 is disposed within vial 402 and distal portion 1056 serves as a cap for vial 402. Vial 402 contains liquid 404, which bathes at least a portion of nasal applicator 1038 within vial 402. It is noted that distal portion 1056, serving as the cap, generally does not leak through the one or more channels 1044 and proximal opening 1042 of nasal applicator 1038, because proximal opening 1042 is too small to allow leakage.

Typically, after nasal applicator 1038 soaks in liquid 404, a portion of liquid 404 is removed for testing for a particulate, such as by removing the cap comprising distal portion 1056, and optionally coupling a conventional dropper cap to the vial and dispensing several drops of liquid 404, such as is known in the art.

In an application of the present invention, a testing kit is provided that comprises nasal sampling device 1020, vial 402, and liquid 404 for bathing at least a portion of a specimen sample in the vial, the liquid selected from the group consisting of: a lysis buffer, saline solution (e.g., phosphate buffered saline (PBS)), and transport medium (e.g., universal transport medium or a viral transport medium). test 302 (e.g., a diagnostic test), which is configured to test for the presence of a particulate in a specimen sample collected using the techniques described herein. Test 302 is described hereinabove with reference to FIG. 12, and vial 402 and liquid 404 are described hereinabove with reference to FIG. 13. The testing kit may be used, for example, for collecting a specimen sample from the subject and sending at least a portion of the specimen sample to a remote laboratory in the liquid in the vial, such as for performing a molecular test, such as Polymerase Chain Reaction (PCR) testing or isothermal testing for particulate in the specimen sample. The particulate may be a virus, a bacterium, any of the other particulates described hereinabove, or any of the other biological materials described hereinabove.

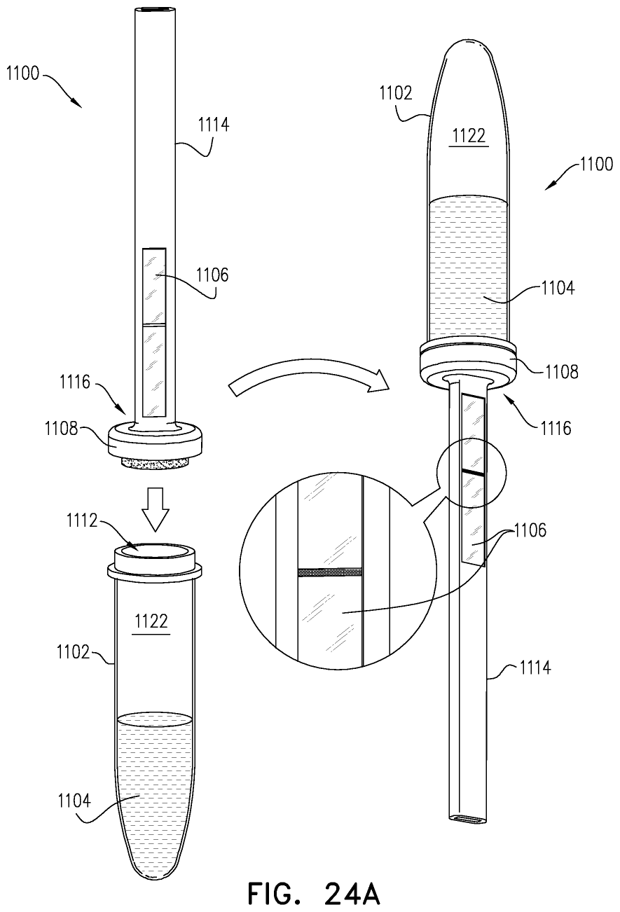

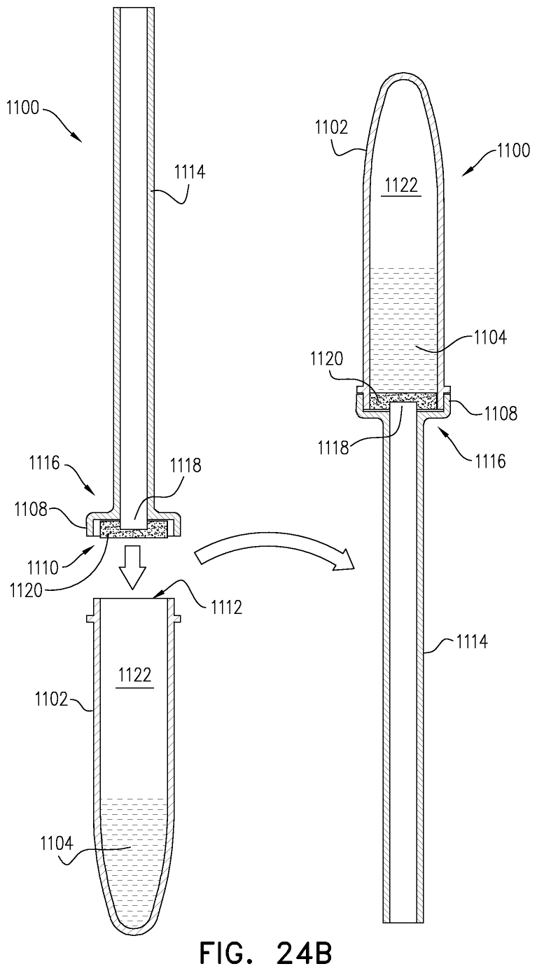

Reference is now made to FIGS. 24A and 24B, which are schematic illustrations of a testing kit 1100 for testing a specimen sample, in accordance with an application of the present invention. Testing kit 1100 comprises:
a vial 1102;
a liquid reagent 1104 (such as a lysing buffer, e.g., a lysing buffer configured to be used with an immunoassay); liquid reagent 1104 may be provided in vial 1102 or in a separate container;
a cap 1108, which is configured to be sealingly coupled, at a distal side 1110 thereof, to a proximal opening 1112 defined by vial 1102;
an elongate test-strip holder 1114, which is coupled to a proximal side 1116 of cap 1108; and
a lateral flow test strip 1106, which is at least partially disposed within elongate test-strip holder 1114, at least partially visible from outside test-strip holder 1114 (for example, test-strip holder 1114 may comprise a transparent material and/or a window, which may optionally include an optical element, such as a magnifying element).

Cap 1108 is shaped so as to define an aperture 1118 that is in fluid communication with (a) distal side 1110 of cap 1108 and (b) lateral flow test strip 1106 within elongate test-strip holder 1114.

Cap 1108 comprises an absorbent material 1120 that is disposed on distal side 1110 of cap 1108 and is in fluid communication (a) with lateral flow test strip 1106 via aperture 1118, and (b) when cap 1108 is sealing coupled to proximal opening 1112 of vial 1102, with an interior 1122 of vial 1102. For example, absorbent material 1120 may comprise a sponge. Absorbent material 1120 is configured to regulate and control the volume of liquid reagent 1104 that flows from vial 1102 to lateral flow test strip 1106.

For some applications, lateral flow test strip 1106 comprises a lateral flow immunoassay test strip.

For some applications, vial 1102 has a volume of at least 3 cc, no more than 5 cc, and/or between 3 and 5 cc. For some applications, liquid reagent 1104 has a volume of at least 0.2 cc, no more than 1 cc, and/or between 0.2 and 1 cc.

During use, the specimen sample is placed in liquid reagent 1104, such as by inserting a swab containing the specimen sample into liquid reagent 1104 in vial 1102. Cap 1108 is sealingly coupled to proximal opening 1112 of vial 1102. Vial 1102 is typically turned over such that liquid reagent 1104 comes in contact with absorbent material 1120 of cap 1108. Absorbent material 1120 brings a small portion of liquid reagent 1104 into contact with lateral flow test strip 1106. The results may be directly observed, as is conventional in the art, and/or analyzed by a chromatographic digital immunoassay, such as the BD Veritor™ system.

### Experimental Results

The inventors performed a number of experiments using some of the techniques described herein.

Although many of the experiments described herein include testing for the presence of the SARS-CoV-2 virus, the inventors believe that the testing techniques used in these experiments would provide similar results for other viruses sharing one or more characteristics with SARS-CoV-2, such as infection of the nasal cavity and viral tropism. Such other viruses include, for example, Influenza viruses, and rhinoviruses.

For example, there is shared tropism in Influenza and SARS-CoV-2 infection. The expression of receptors used in cellular entry is one of the principal limitations on viral tropism, with SARS-CoV-2 and Influenza requiring Angiotensin-converting enzyme-2 (ACE2) and Sialic acid (SA) expression, respectively, to be permissive for infection. The replication of SARS-CoV-2 in upper respiratory tract (URT) tissues is attributed to the expression of ACE2 in olfactory and respiratory epithelial cells. Similarly, the permissiveness of human nasal epithelial cells to Influenza infection in vitro is believed to be due to the cell-surface expression of SA in ciliated epithelial cells. Moreover, SARS-CoV-2 and Influenza have been reported to have similar tissue tropism in the lungs and bronchus. The inventors therefore believe that sampling methods described herein, such as saline nasal spraying and swabbing or saline nasal spraying and gargling, which produce samples with higher SARS-COV-2 viral loads and/or amounts of infected cell debris than traditional sampling methods, would similarly be a more effective sampling procedure for the diagnosis and/or detection of Influenza or any other URT infection which reproduce in nasal epithelial cells, such as human rhinoviruses.

### Experiments 1, 2, and 3

Experiments 1, 2, and 3 demonstrate that at least a portion of intranasally-dispensed nasal wash fluid can be recovered from oral cavity 36 using methods described herein. It is noted that these experiments were performed with conventional, commercially-available nasal dispensers, as described in detail below, rather than with the novel two-nozzle nasal wash fluid dispenser 20 described hereinabove, or the novel three-nozzle nasal wash fluid dispenser 220 described hereinabove, although such use may improve the results of the methods.

### Materials fir Experiments 1, 2, and 3

Standard nasal wash saline solution (hereinbelow, the “standard nasal wash saline solution”): FLO Sinus Care Starter Kit, ENT Technologies Pty Ltd., Hawthorn East Victoria 3123, Australia. (The FLO Sinus Care Starter Kit is marketed and described as a nasal wash saline solution for sinus care.)
Colored nasal wash fluid (hereinbelow, the “colored nasal wash fluid”): A proprietary blue colored nasal wash fluid was made by combining the standard nasal wash saline solution with a standard blue food coloring (made in the USA).
Oral wash fluid (hereinbelow, the “oral wash fluid”): FLO Sinus Care Starter Kit, ENT Technologies Pty Ltd., Hawthorn East Victoria 3123, Australia. (The FLO Sinus Care Starter Kit is marketed and described as a nasal wash saline solution tier sinus care; the inventors of the present application have used it as the novel oral wash fluid described herein.)
Standard nasal spray dispenser (hereinbelow, the “standard nasal spray dispenser”): Afrin Nasal Spray, distributed by MSD Consumer Care Inc., POB 377 Memphis, Tenn., USA, a subsidiary of Merck and co. Inc., White House Station, N.J., USA. The dispenser was emptied of all contents and washed out prior to use.

### Methods for Experiments 1, 2, and 3

Setting up the standard nasal wash fluid syringe dispenser: Nasal wash fluid was transferred into a 5 mL syringe with a distally attached rubber tubing. The rubber tubing was introduced about 1 cm into nasal cavity 22 via one nostril 28 and was positioned along the floor of nasal cavity 22 so as to direct the nasal wash fluid a posterior direction 48 towards nasopharynx 32.

### Experiment 1: Colored Nasal Wash Fluid

In Experiment 1, the nasal wash fluid syringe dispenser was set up with 3.0 mL of the prepared colored nasal wash fluid.
The colored nasal wash fluid was dispensed as a fluid stream into nasal cavity 22 in a posterior direction 48 towards nasopharynx 32 by manually pressing the plunger of the syringe.
Within a few seconds, the colored nasal wash fluid was felt in the throat, which is hypothesized to be the point in time at which the colored nasal wash fluid began draining from nasopharynx 32 into oropharynx 30.
A specimen sample was then expressed out of oral cavity 36 by spitting out the contents of oral cavity 36 through anterior opening 34 of oral cavity 36.

### Results of Experiment 1

The orally expressed specimen sample had a volume of about 0.5 to 0.6 mL and was confirmed to contain at least a portion of the intranasally dispensed colored nasal wash fluid by visually ascertaining a distinct blue color in the specimen sample.

### Conclusions Regarding Experiment 1

At least a portion of an intranasally dispensed colored nasal wash fluid was recovered in orally expressed specimen sample.

### Experiment 2: Testing Different Volumes

In Experiment 2, different volumes of nasal wash fluid were tested.
For each experiment, the nasal wash fluid syringe dispenser was set up with different volumes ranging from 0.5 to 5.0 mL of nasal wash fluid consisting of the standard nasal wash saline solution. Each volume was tested once or multiple times.
For each experiment, a volume of nasal wash fluid was dispensed as a fluid stream into nasal cavity 22 in a posterior direction 48 towards nasopharynx 32 by manually pressing the plunger of the syringe, while head was tilted back about 15-30 degrees.
Within a few seconds, the nasal wash fluid was felt and/or tasted in the throat, which is hypothesized to be the point in time at which the nasal wash fluid began draining from nasopharynx 32 into oropharynx 30.
When possible (as explained below), after draining into oropharynx 30, the nasal wash fluid was gargled.
When possible (as explained below), a specimen sample was then expressed out of oral cavity 36 by spitting out the contents of oral cavity 36 through anterior opening 34 of oral cavity 36.

### Results of Experiment 2

When 0.5 mL was intranasally dispensed (tested 4 times): Fluid was felt in the throat but not enough to be expressed by spitting out
When 1.0 mL was intranasally dispensed (tested 7 times): in some cases, fluid was felt in the throat, but there was not enough to be expressed by spitting out. In other cases, between 0.13 and 0.22 mL of specimen sample was expressed by spitting out. For all cases of 1.0 mL dispensed, it was not possible to gargle.
When 2.0 mL was intranasally dispensed (tested 2 times): It was possible to perform a weak gargle, and between 0.3 and 0.4 mL of specimen sample was expressed by spitting out.
When 3.0 mL was intranasally dispensed (tested 4 times): It was possible to perform a weak gargle, and between 0.55 and 1.2 mL of specimen sample was expressed by spitting out.
When 4.0 mL was intranasally dispensed (tested 2 times): it was possible to perform a gargle, and about 1.0 mL of specimen sample was expressed by spitting out.
When 5.0 mL was intranasally dispensed (tested 2 times): It was possible to perform a gargle, and between 1.2 and 2.1 ml of specimen sample was expressed by spitting out.

### Conclusions Regarding Experiment 2

Specimen samples can be orally expressed when more than about 1 mL of nasal wash fluid is intranasally dispensed. Nasal wash fluid can be gargled prior to orally expressing specimen sample when more than about 2 ml of nasal wash fluid is intranasally dispensed. However, the inventors hypothesize that dispensing a smaller volume of as low as 0.2 mL into nasal cavity 22 may be adequate for draining to oropharynx 30 if the fluid is dispensed at a higher pressure and/or better aimed toward nasopharynx 32 than in the experiments performed.

### Experiment 3: Nasal Wash Fluid+Oral Wash Fluid Gargled

The standard nasal spray dispenser was used to dispense a single pulse of nasal wash fluid consisting of the standard nasal wash saline solution into one nostril 28 in the posterior direction 48 within nasal cavity 22.
Then, after the nasal wash fluid was felt and/or tasted in the throat, 5 mL of the oral wash fluid was added to oral cavity 36 vial anterior opening 34 of oral cavity 36.
The nasal wash fluid and oral wash fluid were gargled together and then expressed out of anterior opening 34 of oral cavity 36 as a first specimen sample by spitting out.
After spitting out the first specimen sample, an additional 5 ml, of oral wash fluid was added to oral cavity 36 vial anterior opening 34 of oral cavity 36 without dispensing any additional nasal wash fluid.
The oral wash fluid was gargled and then expressed out of anterior opening 34 of oral cavity 36 as a second specimen sample by spitting out.
Volumes of both specimen samples were measured.

### Results of Experiment 3

Volume of first specimen sample (gargled nasal wash fluid+oral wash fluid): 4.56 mL
Volume of second specimen sample (gargled oral wash fluid): 4.3 ml.
Volume of nasal wash fluid dispensed in a single pulse: 1.98 mL
   Conclusions regarding Experiment 3

There was a slight increase in specimen sample volume when the nasal wash fluid is dispensed compared to gargling with oral fluid alone, indicating that at least a portion of the nasal wash fluid was washed into oropharynx 30 and was expressed out of oral cavity 36.

### Experiments 4 and 5

Experiments 4 and 5 demonstrate that at least a portion of biological material derived from nasal cavity 22 and/or nasopharynx 32 can be washed into oropharynx 30 by the intranasally-dispensed nasal wash fluid and can then be recovered from oral cavity 36 using methods described herein. It is noted that these experiments were performed with conventional, commercially-available nasal dispensers, as described in detail below, rather than with the novel two-nozzle nasal wash fluid dispenser 20 described hereinabove, or the novel three-nozzle nasal wash fluid dispenser 220 described hereinabove, although such use may improve the results of the methods.

### Materials fir Experiments 4 and 5

Standard nasal wash saline solution (hereinbelow, the “standard nasal wash saline solution”): PLO Sinus Care Starter Kit, ENT Technologies Pty Ltd., Hawthorn East Victoria 3123, Australia.
Standard metered nasal mist dispenser (hereinbelow, the “standard metered nasal mist dispenser”): NasaCort, distributed by Chattem Sanofi Company, Chattanooga, Tenn., USA. The dispenser was emptied of all contents and washed out prior to use.

### Experiment 4: Asymptomatic Subject—Microscopic Analysis of Specimen Sample

The expressed specimen sample collected from Experiment 1, described hereinabove, was centrifuged at 3500 RPM for 10 minutes.
Supernatant was discarded and pellet was isolated. A portion of the pellet was mounted on a glass slide and stained using Gram staining to enable viewing of nuclei and cellular shape.
The slides were analyzed by light microscopy.

### Results of Experiment 4

The slides showed large quantity of squamous epithelial cells, which are morphologically square in shape and are known in the art to be present in the oral cavity. The slides also showed minimal quantity of columnar epithelial cells, which are morphologically elongated and rectangular in shape and are known in the art to be present in the nasal cavity and not present in the oral cavity.

### Conclusions Regarding Experiment 4

Microscopic analysis of orally expressed specimen sample shows cells of nasal origin, confirming that biological material was washed from nasal cavity 22 into oropharynx 30 to be expressed out of oral cavity 36 by spitting out.

### Experiment 5: Symptomatic Subject—Microscopic Analysis of Specimen Sample

Experiment 5 was performed on a subject suffering from seasonal allergic rhinitis.
The standard metered nasal mist dispenser was used to dispense nasal wash fluid consisting of the standard nasal wash saline solution intranasally according to the following procedure:
   (1) Spray twice in the first nostril: once superoposteriorly and once posteriorly,
   (2) Spray twice in the second nostril, once superoposteriorly and once posteriorly.
   (3) Repeat steps (1) and (2) for a total of 8 sprays.
Within a few seconds, the nasal wash fluid was felt and/or tasted in the throat, which is hypothesized to be the point in time at which the nasal wash fluid began draining from nasopharynx 32 into oropharynx 30.
A specimen sample was then expressed out of oral cavity 36 by spitting out the contents of oral cavity 36 through anterior opening 34 of oral cavity 36. The volume of the specimen sample that was collected was about 2 mL.
The expressed specimen sample was centrifuged at 3000 RPM for 10 minutes.
Supernatant was discarded and pellet was isolated, A portion of the pellet was mounted on a glass slide and stained using Gram staining to enable viewing of nuclei and cellular shape.
The slides were by light microscopy.

### Results of Experiment 5

The slides showed large quantity of columnar epithelial cells, which are morphologically elongated and rectangular in shape and are known in the art to be present in the nasal cavity and not present in the oral cavity. The slides also showed minimal quantity of squamous epithelial cells, which are morphologically square in shape and are known in the art to be present in the oral cavity. The slides also showed eosinophils, which are morphologically distinct due to the presence of distinct large granules and are known in the art to be present in the nasal cavity and not present in the oral cavity. Eosinophils were present both as intact cells and as degranulated cells with free granules.

### Conclusions Regarding Experiment 5

Microscopic analysis of orally expressed specimen sample shows high quantity of cells of nasal origin, confirming that biological material was washed from nasal cavity 22 into oropharynx 30 to be expressed out of oral cavity 36 by spitting out.

Asymptomatic subjects with minimal nasal secretions appear to have fewer epithelial cells recovered from the nasal cavity than symptomatic subjects with nasal secretions. The inventors hypothesize that this may be due to symptomatic subjects having comparatively higher quantity of desquamated cells and/or damaged cells that are more easily sloughed off and/or dislodged by the nasal wash fluid.

### Experiment 6

Experiment 6 demonstrates that at least a portion of intranasally-dispensed nasal wash fluid can be recovered from oral cavity 36 using the novel methods and the two-nozzle nasal wash fluid dispenser 20 described herein. It is noted that this experiment was performed with a novel two-nozzle nasal wash dispenser similar to the two-nozzle nasal wash fluid dispenser 20 described herein, rather than with the novel three-nozzle nasal wash fluid dispenser 220 described hereinabove, although such use may improve the results of the methods.

### Materials for Experiment 6

Standard nasal wash saline solution (hereinbelow, the “standard nasal wash saline solution”): FLO Sinus Care Starter Kit, ENT Technologies Pty Ltd., Hawthorn East Victoria 3123, Australia.
Colored nasal wash fluid (hereinbelow, the “colored nasal wash fluid”): A proprietary blue colored nasal wash fluid was made by combining the standard nasal wash saline solution with a standard blue food coloring (made in the USA).
Oral wash fluid (hereinbelow, the “oral wash fluid”): FLO Sinus Care Starter Kit, ENT Technologies Pty Ltd., Hawthorn East Victoria 3123, Australia. (The FLO Sinus Care Starter Kit is marketed and described as a nasal wash saline solution for sinus care; the inventors of the present application have used it as the novel oral wash saline solution described herein.)
Two-nozzle nasal spray dispenser (hereinbelow, the “two-nozzle nasal spray, dispenser”): a custom-manufactured nasal spray dispenser comprising a tubular applicator similar to tubular applicator 64, described hereinabove, that was attached to the distal end of a standard syringe for measured fluid dispensing. The tubular applicator was shaped so as to define two nozzles that enable fluid to be dispensed in two directions simultaneously. The first nozzle directed fluid in the posterior direction within the nasal cavity, and the second nozzle directed fluid in the superoposterior direction within the nasal cavity.

### Methods for Experiment 6

Setting up the two-nozzle nasal spray dispenser: 0.4 mL of the colored nasal wash fluid was transferred into a 2.5 ml, syringe with the two-nozzle nasal spray tip attached to the standard tapered exit port of the syringe.
   Experiment 6: colored Nasal Wash Fluid
In Experiment 6, each nostril was sprayed with 0.4 mL of the colored nasal wash fluid using the two-nozzle nasal spray dispenser, and the subject sniffed the nasal wash fluid further into the nasal cavity after the nasal wash fluid was dispensed into the nostril.
After a few seconds, once at least a portion of the nasal wash fluid was felt draining into oropharynx 30, 4 mL of the colorless oral wash fluid was dispensed into oral cavity 36 through anterior opening 34 of oral cavity 36, and gargled for approximately 10 seconds.
A specimen sample was then expressed out of oral cavity 36 into CUP A by spitting out the contents of oral cavity 36 through anterior opening 34 of oral cavity 36.
Each nostril was then sprayed with an additional 0.4 mL of colorless standard nasal wash saline solution using the two-nozzle nasal spray dispenser.
After a few seconds, once at least a portion of the second nasal wash fluid was felt draining into oropharynx 30, and additional 4 mL of the colorless oral wash fluid was dispensed into oral cavity 36 through anterior opening 34 of oral cavity 36, and gargled for approximately 10 seconds.
A second specimen sample was then expressed out of oral cavity 36 into CUP B by spitting out the contents of oral cavity 36 through anterior opening 34 of oral cavity 36,

### Results of Experiment 6

CUP A: Observed blue color in sample. Sample volume was 3.68 mL.
CUP B: Observed blue color in sample. Sample volume was 4.88 mL.

### Conclusions Regarding Experiment 6

At least a portion of an intranasally dispensed colored nasal wash fluid was recovered in orally expressed specimen sample when using a small volume of nasal wash fluid. A two-nozzle nasal spray is functional and may increase the sample yield. Residual color from the first nasal spray was recoverable when procedure was repeated, which indicates the possibility that repeating the procedure may increase the sample yield.

### Experiment 7

Experiment 7 provides evidence that viral particles can be captured and detected on filters having pore sizes substantially larger than the viral diameter when filtering samples such as gargled fluid, nasal washes, and saliva. The inventors hypothesize that filters retain mucus and other large or aggregated salivary components, which in turn contain viral particles, and/or that the viral particles adhere e.g., directly adhere) to the material of the large-pore filters. It is noted that this experiment was performed with conventional, commercially-available nasal dispensers, as described in detail below, rather than with the novel two-nozzle nasal wash fluid dispenser 20 described hereinabove, or the novel three-nozzle nasal wash fluid dispenser 220 described hereinabove, although such use may improve the results of the methods.

### Materials for Experiment 7

Standard saline solution for nasal and oral wash (hereinbelow, the “standard nasal and oral wash saline solution): 0.9% w/v Sodium Chloride. Catalog Number 364-2828. B. Braun Melsungen AG. Melsungen, Germany.
Nasal spray bottle (hereinbelow, the “standard nasal spray bottle”): Nasal Sprayer 0.1 mL dosage. Catalog number 0591-0410-18. Gil Pack Pharmaceutical Packaging. Ashdod, Israel.
PCR Testing: Sent to external laboratory. Roche Cobus 4800. Two unspecified targets used.
Immunoassay Kits (hereinbelow, the “rapid immunoassay”): One Step Test Device. SARS-CoV-2 Antigen Rapid Test. Nantong Egens Biotechnology Co., Ltd. China.
Transport Medium (hereinbelow, “UTM”): Sample Collection Vial. Universal Transport Medium (UTM) 3 mL. Reference Number BM-801. NovaMed Ltd. Jerusalem, Israel.
Polyester Filters: Polyester Non-Woven Colorful Felt. Ningbo Home-Dollar Imp. & Exp. Company. China. Cut into 25 mm diameter circles. For pore size, see below immediately following Table 6.
Grade C Filters: Glass Fiber Filters, Grade C, 25 mm. 1.2 micron nominal pore size. C2500. Sterlitech. Kent, Wash. USA.
Grade F Filters: Glass Fiber Filters, Grade F, 25 mm. 0.7 micron nominal pore size. F2500. Sterlitech. Kent, Wash. USA.
PES Filters: Polyethersulfone (PES) Membrane Filters, 0.03 micron, 25 mm. PES00325100. Sterlitech. Kent, Wash. USA.

### Methods for Experiment 7

One subject confirmed positive for SARS-CoV-2 infection was tested over the course of a 4-day period.
Combined Nasal Spray & Gargle Procedure (hereinbelow, the “N&G Procedure”): The standard nasal and oral wash saline solution was sprayed 3-5 times (0.1 mL per spray, for a total of 0.3-0.5 mL) into each nostril with the standard nasal spray bottle until fluid was felt by the subject in the back of the throat. The subject sniffed deeply after spraying was complete. The subject then introduced about 5 mL of the standard nasal and oral wash saline solution via anterior opening 34 of oral cavity 36 and gargled the saline solution for about 15 seconds. The subject then spit out the liquid (hereinbelow, the “N&G liquid specimen sample”).
Gargle Alone Procedure (hereinbelow, the “G Alone Procedure”): The subject introduced about 5 mL of the standard nasal and oral wash saline solution via anterior opening 34 of oral cavity 36 and gargled the saline solution for about 15 seconds. The subject then spit out the liquid (hereinbelow, the “G Alone liquid specimen sample”). The nasal and oral wash saline solution was not introduced into the nasal cavity in this procedure.
Filtration procedure: Prior to filtration, the N&G and G Alone liquid specimen samples were swabbed and either tested by rapid immunoassay or placed into UTM for PCR testing. The N&G and G Alone liquid specimen samples were then filtered through a syringe containing one or two polyester filters, one or two grade C filters, and one grade F filter, separated by spacers. The intermediary filtrates were then filtered through a second syringe containing one PES filter. The final filtrates were swabbed and either tested by rapid immunoassay or placed into UTM for PCR testing. All filters were removed and either tested by rapid immunoassay or placed into UTM for PCR testing. All filtrations were conducted using an air pressure pump at 1.4 ATM.

### Results and Discussion of Experiment 7

Table 4 shows qualitative immunoassay results. The samples were taken on two consecutive days (“Day 1” and “Day 2”). All fluid samples were positive for SARS-CoV-2 prior to filtration and all final filtrates were negative. All filters tested positive for SARS-CoV-2. Taken together, the data indicate that filters cohesively retained the virus during filtration, indicating overall successful filtration.

As also can be seen in the comparison between the N&G and G Alone liquid specimen samples in Table 4, the N&G liquid specimens produced substantially more intense test lines on the immunoassay test strips than the G Alone liquid specimens for the fluid before filtration and all of the tested filters except the Grade C filter (which had the same intensity).

TABLE 4
Qualitative Immunoassay Results
(Intensity of Test Lines on Test Strips
on an Arbitrary Scale from 0 to 5)

| Day | Day 1 | Day 2 | |
|---|---|---|---|
| Nasopharyngeal and Throat Swab (Control) | 5 | 5 | |
| Fluid Sample Type | N&G | N&G | G Alone |
| Swab of Fluid Before Filtration | 0.5 | 4 | 2 |
| Polyester Filter | 3 | 1.5 | 1 |
| Grade C Filter | 2.5 | 1.5 | 1.5 |
| Grade F Filter | 1.5 | 3 | 1 |
| PES Filter | 1.5 | 1 | 0.5 |
| Swab of Filtrate | 0 | 0 | 0 |

Table 5 shows results of PCR tests of a single N&G liquid specimen sample based on two targets. The results are expressed in cycle threshold (Ct) values, as is standard in quantitate PCR. As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels. The single N&G liquid specimen sample was taken on “Day 4,” i.e., two days after “Day 2” mentioned above.

As can be seen in Table 5, the fluid sample before filtration had significantly lower Ct values compared to the filtrate, indicating successful filtration, consistent with the immunoassay data presented above in Table 4.

TABLE 5
Qualitative PCR Results (Ct values)

| | Target | |
|---|---|---|
| | Target 1 | Target 2 |
| | Day | |
| | Day 4 | Day 4 |
| Nasopharyngeal Swab (Control) | 27.71 | 28.12 |
| Mid-turbinate Swab (Control) | 29.81 | 30.24 |
| Outer Nares Swab (Control) | 31.25 | 32.02 |
| Swab of Fluid Before Filtration | 30.14 | 30.53 |
| Polyester Filter | 29.09 | 30.12 |
| Grade C Filter | 31.21 | 31.86 |
| Grade F Filter | 31.59 | 32.08 |
| PES Filter | 30.41 | 30.52 |
| Swab of Filtrate | 36.86 | 39.53 |

Table 6 shows the pore size of each filter. Only the PES filter has a pore size that is less than the SARS-CoV-2 diameter (which is generally believed to range between 0.05 and 0.2 microns). However, the PES filter did not show improved viral retention compared to other filters (Table 5). In fact, filter Ct values were not significantly different from each other, indicating that all of the filters retained the virus with similar efficiency (p=0.55, Mann-Whitney test).

TABLE 6
Pore Sizes of Filters

| | Pore Size | Greater or Less than |
|---|---|---|
| Filter Type | (microns) | SARS-CoV-2 diameter |
| Polyester | 122.5 | > |
| Grade C | 1.2 | > |
| Grade F | 0.7 | > |
| PES | 0.03 | < |

The 122.5 micron nominal pore size of the polyester filter was characterized using the bubble point test (i.e., largest pore diameter) using a capillary flow porometer, Model Number CFP-1500A with options E, X, M, and L, made by Porous Materials Inc. (PMI) and Galwick (PMI) as the wetting liquid, which has a fluid surface tension of 15.9 dynes/cm. (The bubble point pressure was 0.054 PSI.)

The polyester filter was also characterized as having a smallest pore diameter of 64.4 microns as determined by capillary flow capillary flow porometry, and a mean pore diameter of 75.5 microns as determined by capillary flow porometry, with a standard deviation of 66.6. The polyester filter was further characterized as having a distribution of pore size values skewed towards the smallest size, with over 40% of pores between 65 and 70 microns.

### Conclusions Regarding Experiment 7

Filters having pores larger than the viral diameter nonetheless retain virus from gargled fluid, saliva, and other types of oral and/or nasal wash fluid. The large-pore filter likely concentrates the virus indirectly via retained or trapped biological materials such as saliva and/or mucus. This concentrated sample may improve test sensitivity compared to unfiltered samples and the large pore size enables high flow rate and inhibits filter clogging despite high sample viscosity.

### Experiment 8

Some of the techniques described hereinabove comprise intranasally dispensing nasal wash fluid into the nasal cavity; thereafter, gargling, by the subject, the nasal wash fluid washed into the oropharynx; and thereafter, collecting a specimen sample that passed out of the anterior opening of the oral cavity. Experiment 8 provides evidence of the efficacy of these techniques for collecting specimen samples for detection of SARS-CoV-2 using quantitative PCR (qPCR).

In summary, Experiment 8 found that:
the combination of nasal wash dispensing and gargling was 40% more likely to be positively identified using qPCR as containing SARS-CoV-2 compared to gargle-only samples, and 22% more likely to be positively identified using qPCR as containing SARS-CoV-2 compared to anterior nares swabs;
the combination of nasal wash dispensing and gargling improved SARS-CoV-2 detection using qPCR by between 543% and 837% depending on the target gene compared to paired gargle samples; and
concentration of nasal gargle samples by filtration further improved SARS-CoV-2 detection, using qPCR, by an addition 373% to 450% compared to unconcentrated nasal gargle samples.

### Detailed Report of Experiment 8: A Novel and Non-Invasive Nasopharyngeal Self-Sampling Methodology for the Detection of SARS-CoV-2

### Section 1: Aim of Study

1. To define the effects of administering a mild nasal spray prior to sample procurement via gargling (hereinbelow, “Nasal Gargle”) on SARS-CoV-2 detection sensitivity.
2. To examine the efficacy of utilizing a hand-held concentration device in concentrating SARS-CoV-2 RNA in gargle samples.

### Section 2: Pivotal Results

1. Nasal Gargle was 40% more likely to be positively identified using qPCR as containing SARS-CoV-2 compared to gargle samples, and 22% more likely to be positively identified using qPCR as containing SARS-CoV-2 compared to anterior nares swabs.
2. Nasal Gargle improved SARS-CoV-2 detection using qPCR by between 543% and 837% depending on the target gene compared to paired gargle samples.
3. Concentration of Nasal Gargle samples by filtration further improved SARS-CoV-2 detection, using qPCR, by an additional 373% to 450% compared to unconcentrated nasal gargle samples.

### Section 3: Background

SARS-CoV-2 diagnostics can be greatly expanded by a sampling technique that is both highly sensitive and allows self-collection. Multiple independent studies performed in Germany, India, British Columbia, and most recently, at the University of Arizona, have demonstrated that oropharyngeal gargle samples obtained by gargling 5 mL to 10 mL of saline are equivalent^{1,2,3}, if not significantly more sensitive (>20%)⁴ than nasopharyngeal (hereinbelow, “NP”) swabbing for SARS-CoV-2 detection. Gargle samples were also found to be more sensitive than other self-sampling techniques such as saliva collection when tested in parallel¹. Furthermore, the FDA has recently approved the OraRisk COVID-19 RT-PCR test for the collection and processing of gargle samples under EUA for SARS-CoV-2 diagnosis⁵. However, despite NP swabbing and equivalent sampling techniques being the current gold-standard for SARS-CoV-2 detection, evidence suggests that these methods are highly variable and cause a concerning number of false-negative results in clinical settings^{6,7,8,9}. Accordingly, we sought to improve the gargle sampling methodology, and to confirm a significant incremental benefit by coupling gargling with a swab-free non-invasive method for sampling the nasopharyngeal region, along with an additional step to further increase sensitivity by means of a novel concentration technique. We have successfully validated the underlying premise of these novel techniques.

### Section 4: Rationale

Observed discrepancies between paired sampling procedures targeting variable regions in the upper respiratory tract suggest that the physiological localization and distribution of SARS-CoV-2 is heterogeneous in nature, and likely a contributing factor for noted variations in testing sensitivity between sampling methodologies⁹. Similarly, self-sampling techniques such as anterior nares swabbing have routinely underperformed when testing asymptomatic individuals¹⁰, indicating differential regional availability of SARS-CoV-2 in the upper respiratory tract between cohorts. Furthermore, saliva and anterior nares swabs have been found to be poor sample candidates for immunoassays, with heavily invasive NP swabbing performed by a trained medical professional preferred¹¹, limiting the potentiality of an effective at-home diagnostic device. We have invented a robust and non-invasive sampling technique which simultaneously targets both the nasopharyngeal and oropharyngeal regions, in which nasal spray is administered for the sampling of virus-rich nasopharyngeal regions, which by post-nasal drip is accessible through an oropharyngeal gargle. We posited that by utilizing the combined Nasal Gargle as a sample type, patients that have viral replication primarily localized to specific sites which would be otherwise inaccessible to current self-sampling techniques such as saliva or gargle collection, or anterior nares swabbing alone, would be more readily detected, increasing the sensitivity of any downstream molecular or immunological tests performed. Moreover, we examined the additive effects of subsequent concentration of Nasal Gargle samples by filtration to further optimize SARS-CoV-2 detection.

### Section 5: Methods

### 5.1 Sample Collection

In preparing gargle specimens, inpatients (n=21) in the coronavirus ward at Laniado Hospital (Netanya, Israel) were asked to gargle 5-10 mL of normal saline for approximately 9-15 seconds, and then spit the gargle fluid into a sterile collection receptacle. For the generation of Nasal Gargle specimens, the patients first sprayed 0.1 mL of normal saline 3-5 times into each nostril. Once the saline was felt in the back of the throat, patients were requested to sniff, followed by performing a gargle by introducing 5-10 mL of normal saline into the oral cavity via the anterior opening of the oral cavity and gargling for approximately 9-15 seconds, and then spitting the gargled fluid into a sterile collection receptacle. A flocked swab (3BY Ltd., Industrial Park Tefen, Israel, Ref: 3by-001) was waved through the Nasal Gargle or Gargle specimens for approximately 10 seconds, in a manner such that all internal surfaces of the collection receptacle were contacted by the swab. The swab was then placed into tubes containing 3 mL of Universal Transport Medium (UTM) (Biological Industries, Israel). Bilateral anterior nares swabbing (3BY Ltd., Industrial Park Tefen, Israel, Ref: 3by-001) was performed by hospital staff in parallel to gargle and Nasal Gargle sample collection (n=12). Anterior nares swabs were similarly placed into tubes containing 3 mL of UTM (Biological Industries, Israel). Tubes were left at room-temperature for up to 6 hours or stored at 4° C. for up to 72 hours prior to qPCR testing. For 3 of the 12 patients, Nasal Gargles were produced twice in succession. Pairwise comparisons between Nasal Gargle and swabbing or Nasal Gargle and gargle were performed with the first Nasal Gargle sample produced. Prior to concentration (described in Section 5.2) the paired Nasal Gargles were pooled together and then re-aliquoted. In addition to the anterior nares swab, for 5 of the 12 patients, mid-turbinate swabbing was performed by hospital staff prior to gargle sample production. The clinical study was approved by the Laniado Hospital investigation review board (IRB) Helsinki committee.

### 5.2 Concentration

5-10 mL of collected Nasal Gargle was transferred into a hand-held concentration device using a serological pipette and passed through two of the Polyester Filters described and characterized hereinabove in the description of Experiment 7, while the two filters were disposed within the device. The filters were removed from the device, placed into a tube containing 3 mL of UTM (Biological Industries, Israel), and tubes were vortexed for 30-60 seconds. Tubes were left at room-temperature for up to 6 hours, or stored at 4° C. for up to 72 hours, prior to qPCR testing.

### 5.3 Molecular Testing

Prior to RNA extraction, tubes were vortexed for 30-60 seconds. RNA extraction was performed using a MagCore Viral nucleic acid extraction kit (High Sensitivity) (Ref: MVN400-06) on an Automated Nucleic Acid Extractor-MagCore Super (RBC Bioscience, Taiwan). The Allplex SARS-CoV-2 Assay (Seegene, Seoul, South Korea) was used for RNA detection on a CFX96 Real-Time System (BIO RAD, USA). Positive, presumptive positive, and negative SARS-CoV-2 scoring was preformed automatically by the assay's data analyzer, and both positive and presumptive positives were assigned as true positives. RNA extraction and qPCR were performed by the site study laboratory. The Wilcoxon Signed-Rank Test was used to determine statistical significance (p<0.05).

### Section 6: Results

### 6.1: A Comparative Evaluation Between Nasal Gargle and Gargle Sampling Techniques

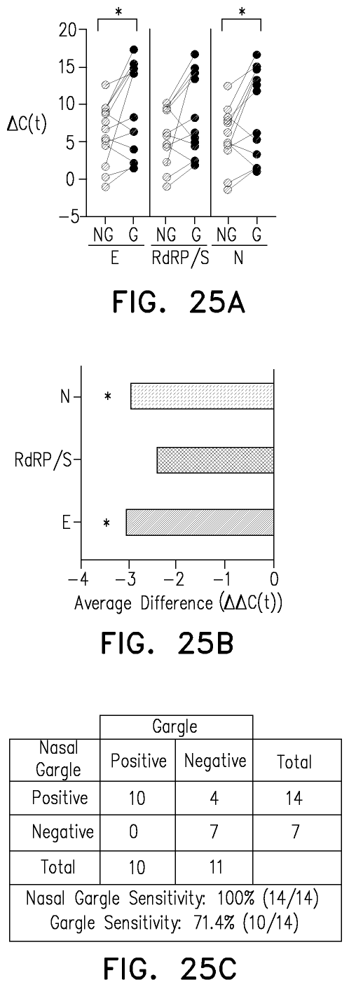

Paired Nasal Gargle and gargle samples were evaluated for the presence of SARS-CoV-2 by qPCR. Out of the 21 patients tested, 14 were found to be positive, with detection from Nasal Gargle outperforming that of gargle in 9 (RdRP/S) or 10 (E and N genes) of the 14 patients (FIG. 25A) based on relative ΔCt values. (As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels.) Despite the small sample size, there was a statistically significant difference between the Nasal Gargle ΔCt values and the gargle ΔCt values for the E gene (p=0.02661) and the N gene (p=0.02148), with the RdRP/S gene approaching statistical significance (p=0.05225). Averaged differences between the two sample types showed a clear preference for Nasal Gargle, with ΔΔCt values of −2.442 (RdRP/S), −3.064 (E), or −3.006 (N) (FIG. 25B). This equates to a 5.434 to 8.365-fold (2^{-ΔΔCt}) increase of viral material sampled by adding nasal spray prior to gargling. Moreover, the sensitivity of testing Nasal Gargle samples was 100%, compared to 71.4% when testing gargle samples (FIG. 25C).

Description of FIG. 25 (A Direct Comparison Between Nasal Gargle and Gargle): FIG. 25A) Paired Nasal Gargle (NG=left endpoint) and gargle (G=right endpoint) ΔCt values of target genes displayed after being normalized to a housekeeping gene tested in parallel. ΔCt values of NG tend to be less than relative G samples. Statistically significant differences in ΔCt values (p<0.05) between NG and G are indicated with an asterisk (*). FIG. 25B) The average difference between NG and G ΔCt values range from −2.48 to −3.15, indicating a larger amount of virus in NG samples across all gene targets tested. In gene targets where both sample types were negative, an assumed ΔCt difference was given as 0. Statistically significant differences in ΔΔCt values (p<0.05) are indicated with an asterisk (*). FIGS. 25A-B) Graphical representation includes data wherein at least 1 sample type was found to be positive for containing SARS-CoV-2, and negative Ct values were assigned a value of 40. FIG. 25C) NG sensitivity was 100%, whereas G sensitivity was 28.6% lower, i.e., NG sensitivity was 40% higher.

### 6.2: A Comparison Between Nasal Gargle and Anterior Nares Swabbing Sensitivity

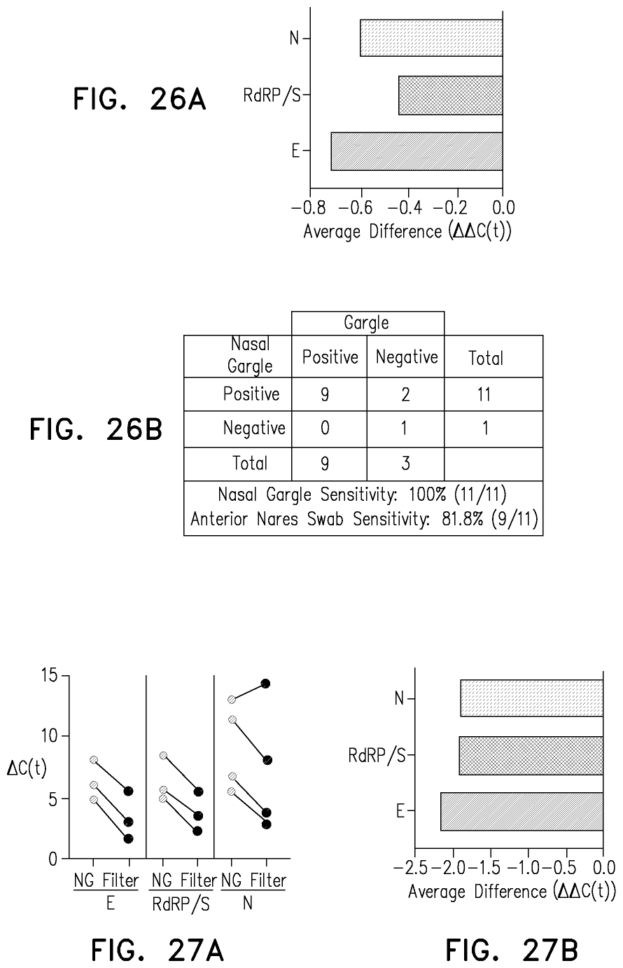

Paired Nasal Gargle and anterior nares swabs (n=12) were compared by qPCR, with lower ΔCt values associated with Nasal Gargle over anterior nares swabbing (FIG. 26A). This result is of particular interest as anterior nares swabbing occurred prior to Nasal Gargle procurement, and without a wait period to permit SARS-CoV-2 containing material to re-establish, indicating that the Nasal Gargle was effective at sampling nasal regions that are generally inaccessible to anterior nares swabbing. Moreover, positive identification of SARS-CoV-2 occurred 22% more often in Nasal Gargle samples than in anterior nares swabs (FIG. 26B), demonstrating preferential sensitivity for the Nasal Gargle sampling technique.

Description of FIG. 26 (A Preliminary examination of Nasal Gargle Efficacy Over Anterior Nares Swabs): FIG. 26A) The average difference between Nasal Gargle and anterior nares swabs ΔCt values range from −0.45 to −0.727, indicating a larger amount of virus in Nasal Gargle samples across all gene targets tested. Graphical representation includes data wherein at least 1 sample type was found to be positive for containing SARS-CoV-2, and negative Ct values were assigned a value of 40. In gene targets where both sample types were negative, an assumed ΔCt difference was given as 0. FIG. 26B) Nasal Gargle sensitivity was 22% higher than anterior nares swab sensitivity.

### 6.3: Concentration of Nasal Gargle

To further increase the detection of SARS-CoV-2 in Nasal Gargle samples, Nasal Gargles were processed by a manual concentration device including two of the Polyester Filters described and characterized hereinabove in the description of Experiment 7. The subsequent and pairwise testing of Nasal Gargles before concentration, and material eluted from the concentration device by qPCR (n=4) demonstrated higher levels of viral material present after concentrating Nasal Gargles compared to unconcentrated Nasal Gargles. The average ΔΔCt values were −2.17 (RdRP/S), −2.17 (E), or −1.8975 (N) (FIG. 27B), corresponding to an estimated viral material increase of 3.857, 4.5, or 3.726-fold, respectively.

Paradoxically, in one patient, SARS-CoV-2 was detected weakly (38.03 Ct value) in a single gene target in unconcentrated Nasal Gargle, but in no gene targets after concentration. The high Ct value in one gene and negative readings for the other gene targets indicates that the viral load of the sample was at or near the limit of detection of the assay. It is likely that the swabbing of the Nasal Gargle prior to filtration, as described hereinabove in Section 5.1, selectively removed a significant portion of the virus, resulting in a sample that contained no or very little virus despite subsequent concentration.

Description of FIG. 27 (Additive Effects of the Concentration of Nasal Gargle Samples): FIG. 27A) Target gene ΔCt values of Nasal Gargle samples before concentration (NG=left endpoint) were paired with respective filters after NG concentration (Filter=right endpoint), after being normalized to a housekeeping gene tested in parallel. Filter ΔCt values tend to be less than unconcentrated NGΔCt values. FIG. 27B) The average difference between NG and filter ΔCt values range from −1.89 to −2.17, indicating a greater presence of virus due to concentration across all gene targets tested compared to unconcentrated NG. In gene targets where both sample types were negative, an assumed ΔCt difference was given as 0. FIGS. 27A-B) Graphical representation includes data wherein at least 1 sample type was found to be positive for containing SARS-CoV-2, and negative Ct values were assigned a value of 40.

### Section 7: Discussion

The striking difference in the observed percentage of SARS-CoV-2 positive samples from Nasal Gargle compared to gargle or anterior nares swabs supports previous observations that viral replication does not always lead to the aggregation of viral material in the same physiological sites⁹, and suggests that Nasal Gargle is effective at sampling a greater number of virus containing regions, such as the nasopharynx, which are otherwise inaccessible to other self-sampling techniques. It therefore follows that utilizing the Nasal Gargle sampling method for current molecular and immunological devices would likely decrease the number of false negative tests. Specifically, our data supports the preferential usage of Nasal Gargle in PCR-based assays in order to prevent false negative test results, which can be damaging to public health and safety.

The preliminary clinical results demonstrate clear trends that have reached, or approach, statistical significance, of more desirable Ct values and sensitivity associated with Nasal Gargle sampling over that of gargle despite the small sample size. Furthermore, with similar results observed both by immunoassay and qPCR testing, we hypothesize that Nasal Gargle should provide a significant increase in sensitivity across a wide range of testing platforms. Impressively, Nasal Gargle outperformed gargle despite the patient sampling protocol requiring the procurement of anterior nares swabs, followed by gargle samples, prior to Nasal Gargle sampling, which might be expected to artificially decrease the amount of viral material in the oropharynx available for sampling by the subsequent Nasal Gargle. It is reasonable to assume that when paired samples are not being procured, such as during standard clinical testing, Nasal Gargle might demonstrate further increased sensitivity. Similarly, SARS-CoV-2 viral load peaks are associated with or before the presentation of symptoms, whereas severe symptoms generally develop one to two-weeks later, accounting for the observation that patients with severe symptoms present with a decreased viral load¹². As our study population exclusively consisted of inpatients who were at an advanced stage of illness, it is therefore reasonable to assume that Nasal Gargle sampling might perform with further increased sensitivity during testing of the general population due to relatively higher levels of viral shedding. Moreover, we did not impose any restrictions on eating, drinking, brushing teeth, or smoking prior to sample collection, contrary to other studies evaluating the efficacy of gargle sampling¹, which accentuates the increase in detection sensitivity observed with Nasal Gargle, as well as likely increasing sampling accessibility¹³.

Lastly, the observed correlation between concentration of Nasal Gargle and decreased Ct values is quite promising, especially when examined with the accumulative decrease of Ct values associated with the use of Nasal Gargle as the sample type. Furthermore, the increased sensitivity observed in Nasal Gargles, combined with an effective concentration protocol could overcome current difficulties in testing pooled gargle samples due to the dilution of SARS-CoV-2 containing material^{14,15}, providing an effective medium for public screening.

### Section 8: References

[1] Goldfarb, D. M., Tilley, P., Al-Rawahi, G. N., Srigley, J., Ford, G., Pedersen, H., Pabbi, A., Hannam-Clark, S., Charles, M. K., Dittrick, M., Gadkar, V. J., Pernica, J. M., & Hoang, L. (2020). Self-collected Saline Gargle Samples as an Alternative to Healthcare Worker Collected Nasopharyngeal Swabs for COVID-19 Diagnosis in Outpatients. MedRxiv, 2020.09.13.20188334.
[2] Mittal, A., Gupta, A., Kumar, S., Surjit, M., Singh, B., Soneja, M., Soni, K. D., Khan, A. R., Singh, K., Naik, S., Kumar, A., Aggarwal, R., Nischal, N., Sinha, S., Trikha, A., & Wig, N. (2020). Gargle lavage as a viable alternative to swab for detection of SARS-CoV-2. Indian Journal of Medical Research, 152(1), 77.
[3] Maricic, T., Nickel, O., Aximu-Petri, A., Essel, E., Gansauge, M., Kanis, P., Macak, D., Richter, J., Riesenberg, S., Bokelmann, L, Zeberg, H., Meyer, M., Borte, S., & Pääbo, S. (2020). A direct RT-qPCR approach to test large numbers of individuals for SARS-CoV-2. PloS One, 15(12), e0244824-e0244824. PubMed.
[4] Stolte, D. (2020, October 26). Swish, Gargle, Repeat. UArizona Researcher Explores Mouth Rinse Test as Alternative to COVID-19 Nasal Swab. University of Arizona News.
[5] Federal Drug Administration (FDA). (2020). Access Genetics, LLC-OraRisk COVID-19 RT-PCR EUA Summary—Updated Sep. 25, 2020.
[6] Berenger, B. M., Fonseca, K., Schneider, A. R., Hu, J., & Zelyas, N. (2020). Sensitivity of Nasopharyngeal, Nasal and Throat Swab for the Detection of SARS-CoV-2. MedRxiv, 2020.05.05.20084889.
[7] Little, B. P. (2021). False-Negative Nasopharyngeal Swabs and Positive Bronchoalveolar Lavage: Implications for Chest CT in Diagnosis of COVID-19 Pneumonia. Radiology, 204471.
[8] Piras, A., Rizzo, D., Uzzau, S., De Riu, G., Rubino, S., & Bussu, F. (2020). Inappropriate Nasopharyngeal Sampling for SARS-CoV-2 Detection Is a Relevant Cause of False-Negative Reports. Otolaryngology—Head and Neck Surgery, 163(3), 459-461.
[9] Kojima, N., Turner, F., Slepnev, V., Bacelar, A., Deming, L., Kodeboyina, S., & Klausner, J. (2020). Self-Collected Oral Fluid and Nasal Swabs Demonstrate Comparable Sensitivity to Clinician Collected Nasopharyngeal Swabs for Covid-19 Detection. MedRxiv, 2020.04.11.20062372.
[10] Prince-Guerra, J. L., Almendares, O., Nolen, L. D., Gunn, J. K. L., Dale, A. P., Buono, S. A., Deutsch-Feldman, M., Suppiah, S., Hao, L., Zeng, Y., Stevens, V. A., Knipe, K., Pompey, J., Atherstone, C., Bui, D. P., Powell, T., Tamin, A., Harcourt, J. L., Shewmaker, P. L., Bower, W. A. (2021). Evaluation of Abbott BinaxNOW Rapid Antigen Test for SARS-CoV-2 Infection at Two Community-Based Testing Sites—Pima County, Ariz., Nov. 3-17, 2020. MMWR. Morbidity and Mortality Weekly Report, 70(3), 100-105. PubMed.
[11] Agulló, V., Fernández-González, M., Ortiz de la Tabla, V., Gonzalo-Jiménez, N., García, J. A., Masiá, M., & Gutiérrez, F. (2020). Evaluation of the rapid antigen test Panbio COVID-19 in saliva and nasal swabs in a population-based point-of-care study. The Journal of Infection, S0163-4453(20)30768-4. PubMed.
[12] Argyropoulos, K. V., Serrano, A., Hu, J., Black, M., Feng, X., Shen, G., Call, M., Kim, M. J., Lytle, A., Belovarac, B., Vougiouklakis, T., Lin, L. H., Moran, U., Heguy, A., Troxel, A., Snuderl, M., Osman, I., Cotzia, P., & Jour, G. (2020). Association of Initial Viral Load in Severe Acute Respiratory Syndrome Coronavirus 2 (SARS-CoV-2) Patients with Outcome and Symptoms. The American journal of pathology, 190(9), 1881-1887.
[13] Kelly, A., & Steacy, L. (2021 Feb. 7). Gargle and spit COVID-19 test important tool to prevent “testing fatigue”: B.C. doctor. CityNews 1130.
[14] Babady, N. E., McMillen, T., Jani, K., Viale, A., Robilotti, E. V., Aslam, A., Diver, M., Sokoli, D., Mason, G., Shah, M. K., Korenstein, D., & Kamboj, M. (2021). Performance of Severe Acute Respiratory Syndrome Coronavirus 2 Real-Time RT-PCR Tests on Oral Rinses and Saliva Samples. The Journal of Molecular Diagnostics: JMD, 23(1), 3-9.
[15] Lüsebrink, J., Schildgen, V., & Schildgen, O. (2020). Pooling is an insufficient strategy to avoid healthcare staff to patient transmission of severe acute respiratory coronavirus virus 2 (SARS-CoV-2). Infection Control & Hospital Epidemiology, 1-3. Cambridge Core.

In some applications, any of the techniques described in the report of Experiment 8 above are used in combination with the other techniques described herein.

### Experiment 9

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Experiment 9 provides evidence of the efficacy of these techniques for collecting specimen samples for detection of SARS-CoV-2 using quantitative PCR (qPCR).

In summary, Experiment 9 found that spraying saline solution into the nasal cavity and gargling resulted in between a 3.4-fold (340%), and 5.6-fold (560%) increase in viral material after saline nasal spraying and gargling compared to before nasal spraying and gargling, depending on the gene target, after a nucleic acid extraction protocol consisting of including heating samples. Experiment 9 also found that spraying saline solution into the nasal cavity and gargling resulted in between a 2.7-fold (270%) and 8.7-fold (870%) increase in viral material after saline nasal spraying and gargling compared to before nasal spraying and gargling, depending on the gene target, after a robust magnetic bead based nucleic acid extraction protocol.

### Detailed Report of Experiment 9: Benefits of Saline Nasal Spraying Before Anterior Nares Swabbing in SARS-CoV-2 Detection

### Section 1: Materials and Methods

### Section 1.1: Subject Sampling

Bilateral anterior nares swabs were performed by a healthcare professional on 18 subjects belonging to the health care service provider Leumit (Tel-Aviv, Israel), who had previously tested positive for SARS-CoV-2. The subjects then sprayed 0.1 mL of normal saline 3-5 times into each nostril (some while their heads were tilted back and others while their heads were in a neutral position), and then gargled 10 mL of normal saline for 10-15 seconds to produce a Nasal Gargle (hereinbelow, “NG”) specimen. Following spitting out of the NG sample, a second bilateral anterior nares swab was performed by the healthcare professional, while the subjects' heads were in a neutral position or tilted slightly back. Swabs were then placed into tubes containing 3 mL of Universal Transport Medium (UTM) (Biological Industries, Israel). Two subjects were excluded from the study due to an inability to gargle. For 3 subjects (Subjects 5-7), a bilateral mid-turbinate swab was performed by the healthcare professional prior to the production of the NG sample. Bilateral anterior nares swabbing was performed by inserting swabs into the nares and then removing the swabs without rotating the swabs against the inner wall of the nares. Samples were transported on ice to either the Leumit Health Services Central Laboratory (Or Yehuda, Israel), or to Hero Scientific Ltd. (Jerusalem, Israel) for processing. The clinical study was approved by the investigational review board (hereinbelow, “IRB”) at Assaf Harofeh (Shamir) Medical Center (Tel Aviv, Israel).

It is important to note that the anterior nares swab sample collection was performed by inserting the swab about 1.5 cm into the nostril and spinning the swab vertically as is typically performed for a mid-turbinate swab sample collection. All anterior nares swab sample collection procedures performed for all other experiments described herein (Experiments 10, 11, 12, 13, 14, and 15) were performed by inserting the swab about 1.5 cm into the nostril and rubbing the swab in a circular motion so as to abrasively rub against the wall of the anterior naris. The inventors hypothesize that this experiment may have been negatively impacted by this non-ideal anterior nares swab sample collection procedure. In particular, the anterior nares swab sample performed after saline nasal administration may have been unable to properly collect the saline dripping down the wall of the anterior naris and thus not yield the benefits of saline nasal administration in increasing the viral load collected. Conversely, the inventor hypothesize that other experiments described herein (Experiments 10, 11, 12, 13, 14, and 15) are more adequate representations of the benefit of anterior nares swab sampling after saline nasal administration.

### Section 1.2: Molecular Testing

Section 1.2.1: qPCR Following Heat Extraction

Samples from 14 of the 16 subjects were processed by quantitative reverse transcription polymerase chain reaction (hereinbelow, “qPCR”) at Hero Scientific Ltd. (Jerusalem, Israel). Samples were first inactivated by boiling at 70° C. for 45 minutes in a heat block with aluminum beads. All samples were processed using the primers and probe sequences from the Center for Disease Control's (hereinbelow, “CDC”) diagnostic panel provided in the 2019-nCoV RUO Kit (CAT #10006713, IDT, Belgium) on an MX3000P (Stratagene, USA) qPCR thermocycler. The reaction mixture for samples from 3 of the 14 subjects (Subjects 5-7) were prepared using the GE50 Xpert One-Step Fast Probe (GRiSP Research Solutions, Portugal), and contained 10 microliters of Fast qPCR Mastermix (GRiSP Research Solutions, Portugal), 0.5 microliters of RTase Mix (GRiSP Research Solutions, Portugal), 2 microliters of primer mix (CAT #10006713, IDT, Belgium), 4 microliters of inactivated sample, and 3.5 microliters of Molecular Grade Water (Biological Industries, Israel). Negative template controls (hereinbelow, “NTCs”) were performed by testing 4 microliters of Molecular Grade Water (Biological Industries, Israel) instead of the samples for all primer/probe mixtures tested. Similarly, positive controls were performed by testing 1 microliter of positive control plasmid, 2019-nCoV_N_Positive Control (CAT #10006625, IDT, Belgium), and 3 microliters of Molecular Grade Water (Biological Industries, Israel) instead of the samples for all primer/probe mixtures tested. The thermal profile conditions were 45° C. for 15 minutes, 95° C. for 3 minutes, and then 40 cycles of 95° C. for 5 seconds and 58° C. for 30 seconds. Prior to use of the GE50 Xpert One-Step Fast Probe, 2 microliters of 100×ROX was added to the Fast qPCR Mastermix (Probe) as per the manufacturer's instructions, and cycle threshold (hereinbelow, “Ct”) values were normalized to ROX. (As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels.)

Samples from 12 of the 14 subjects (including Subjects 1-4 and 8-11) were processed using the PrimeDirect Probe RT-qPCR Mix (Takara Bio inc., Japan). Reaction mixtures contained 12.5 microliters of the PrimeDirect Probe RT-qPCR Mix (Takara Bio inc., Japan), 4 microliters of sample, 2 microliters of the primer/probe mix (IDT, Belgium), and 6.5 microliters of Molecular Grade Water (Biological Industries, Israel). NTCs were performed for each qPCR run by testing 4 microliters of Molecular Grade Water instead of the samples for all primer/probe mixtures tested. Similarly, positive controls were performed by testing 1 microliter of positive control plasmid, 2019-nCoV_N_Positive Control (CAT #10006625, IDT, Belgium), and 3 microliters of Molecular Grade Water (Biological Industries, Israel) instead of the samples for all primer/probe mixtures tested. The thermal profile conditions were 90° C. for 3 minutes, 60° C. for 5 minutes, and then 40 cycles of 95° C. for 5 seconds and 60° C. for 30 seconds.

Section 1.2.2: qPCR Following Magnetic Bead Extraction

Samples from 2 of the 16 subjects (Subjects 12 and 13) were processed by qPCR at the Leumit Health Services Central Laboratory (Or Yehuda, Israel) using the Allplex 2019 n-CoV Assay (Seegene, Seoul, South Korea), following manufacturer's instructions, on a CFX96 Real-Time System (BIO RAD, USA). Prior to qPCR, samples were extracted using the STARMag 96×4 Viral DNA/RNA 200 C Kit (Seegene, Seoul, South Korea) on a MICROLAB STAR^{let} (Hamilton, Switzerland) automated liquid handling platform.

### Section 2: Results and Discussion

Out of the 16 subjects, 13 (n=13) were found to be positive for SARS-CoV-2 on at least one gene target for either of the paired anterior nares swabs. Average Ct value differences between (a) anterior nares swabs sampled before saline nasal spraying and (b) anterior nares swabs samples after saline nasal spraying and gargling for SARS-CoV-2 gene targets, after a nucleic acid extraction protocol including heating samples, ranged from −1.75 to −2.5, corresponding to between a 3.4-fold (340%), and 5.6-fold (560%) increase in viral material after saline nasal spraying and gargling compared to before nasal spraying and gargling, depending on the gene target (Table 7.1). Similarly, average ΔCt value differences between (a) anterior nares swabs sampled before saline nasal spraying and (b) anterior nares swabs samples after saline nasal spraying and gargling for SARS-CoV-2 gene targets, after a robust magnetic bead based nucleic acid extraction protocol, ranged from −1.45 to −3.12, corresponding to between a 2.7-fold (270%) and 8.7-fold (870%) increase in viral material after saline nasal spraying and gargling compared to before nasal spraying and gargling, depending on the gene target (Table 7.2). This phenomenon is likely due to the saline introduced during NG sampling dripping from within the nasal cavity back into the anterior nares, increasing the amount of viral material available for subsequent swabbing. It is therefore expected that performing nasal spray prior to anterior nares swabbing would lead to an increase in sensitivity in any downstream molecular or immunological tests performed using the swabbed specimen samples. Interestingly, similar results were observed across different RNA extraction and qPCR protocols, indicating that a combined nasal spray and anterior nares swab sampling methodology would be effective at increasing diagnostic sensitivity even when used in conjunction with devices where less-robust nucleic acid extraction methods are employed.

TABLE 7.1
A Comparison Between Anterior Nares Swabbing Before And After
Nasal Gargling Following Heat Extraction

| | | 1^{st} | 2^{nd} | | |
|---|---|---|---|---|---|
| | | Anterior | Anterior | | |
| | | Nares | Nares | | Average Ct |
| Gene | Subject | Swab Ct | Swab Ct | Ct Value | Value |
| Target | # | Valueⁱ | Valueⁱ | Differenceⁱⁱ | Differenceⁱⁱ |
| N1 | 1 | 32.73 | 34.84 | 2.11 | −1.7536364 |
| | 2 | 34.91 | 40 | 5.09 | |
| | 3 | 39.05 | 31.84 | −7.21 | |
| | 4 | 40 | 40 | 0 | |
| | 5ⁱⁱⁱ | 31.7 | 24.15 | −7.55 | |
| | 6ⁱⁱⁱ | 17.95 | 26.54 | 8.59 | |
| | 7ⁱⁱⁱ | 31.27 | 24.14 | −7.13 | |
| | 8 | 39.52 | 38.2 | −1.32 | |
| | 9 | 27.62 | 27.92 | 0.3 | |
| | 10 | 40 | 34.23 | −5.77 | |
| | 11 | 31.98 | 25.58 | −6.4 | |
| N2 | 1 | 40 | 40 | 0 | −2.4954545 |
| | 2 | 40 | 40 | 0 | |
| | 3 | 40 | 31.72 | −7.01 | |
| | 4 | 40 | 39.23 | −0.77 | |
| | 5ⁱⁱⁱ | 36.58 | 25.06 | −11.52 | |
| | 6ⁱⁱⁱ | 19.69 | 28.81 | 9.12 | |
| | 7ⁱⁱⁱ | 34.53 | 26.84 | −7.69 | |
| | 8 | 40 | 38.68 | −1.32 | |
| | 9 | 32.81 | 29.32 | −3.49 | |
| | 10 | 40 | 38.62 | −1.38 | |
| | 11 | 33.69 | 30.3 | −3.39 | |

ⁱNegative Ct values, corresponding to a value of ≥40, were assigned an actual value of 40.
ⁱⁱIn instances where both swabs were negative, the average Ct value difference was assumed to bo 0.
ⁱⁱⁱThe NTC accompanying the tests for Subjects 5-7 had evidence of human DNA contamination. However, if human DNA contamination were to occur in subject samples, it would be unlikely to affect results for SARS-CoV-2 specific gene targets, and so the reults were included, but not normalized to the reference gene, RNase P.

TABLE 7.2
A Comparison Between Anterior Nares Swabbing before and
after Nasal Gargling Following Magnetic Bead Extraction

| | | 1^{st} | 2^{nd} | Normalized | Normalized | | |
|---|---|---|---|---|---|---|---|
| | | Anterior | Anterior | 1^{st} Anterior | 2^{nd} Anterior | | |
| | | Nares | Nares | Nares | Nares | Normalized | Average |
| Gene | Subject | Swab Ct | Swab Ct | Swab ΔCt | Swab ΔCt | ΔCt Value | ΔCt Value |
| Target | # | Value | Value | Valueⁱ | Valueⁱ | Differenceⁱⁱ | Difference |
| E | 12 | 24.83 | 26.72 | −1.4 | 0.23 | 1.63 | −1.73 |
| | 13 | 35.04 | 29.57 | 8.5 | 3.41 | −5.09 | |
| RdRP/S | 12 | 26.93 | 28.55 | 0.7 | 2.06 | 1.36 | −3.12 |
| | 13 | 39.19 | 31.21 | 12.65 | 5.05 | −7.6 | |
| N | 12 | 27.77 | 29.42 | 1.54 | 2.93 | 1.39 | −1.45 |
| | 13 | 37.16 | 32.49 | 10.62 | 6.33 | −4.29 | |

ⁱNormalized ΔCt value is calculated by subtracting the Ct value of a reference gene from the Ct value of the target gene.
ⁱⁱNormalized ΔCt value difference is calculated by subtracting the normalized ΔCt value of the anterior nares swab performed prior to the NG (1^{st}) from the normalized ΔCt value of the anterior nares swab performed after the NG (2^{nd}).

In some applications, any of the techniques described in the report of Experiment 9 above are used in combination with the other techniques described herein.

### Experiment 10

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using PCR. Experiment 10 provides evidence of the efficacy of these techniques for collecting specimen samples for detection of SAILS-CoV-2 using quantitative PCR (qPCR).

In summary, Experiment 10 found that eleven out of 15 subjects tested (73.3%) had lower normalized cycle threshold (Ct) values in samples from anterior nares swabs performed after saline nasal spraying and gargling than from anterior nares swabs performed before saline nasal spraying and gargling. Furthermore, 7 out of the 15 subjects (46.7%) were found to have lower normalized Ct values in samples from anterior nares swabs performed after saline nasal spraying and gargling than in mid-turbinate swabs. In cases where the mid-turbinate swab outperformed the anterior nares swabs sampled after saline nasal spraying and gargling, anterior nares swabs sampled after saline nasal spraying and gargling were still within 1 ΔCt value of the mid-turbinate swab in 5 out 8 subjects (62.5%).

### Detailed Report of Experiment 10: Benefits of Saline Nasal Spraying Prior to Anterior Nares Swabbing in SARS-CoV-2 Detection on the Cobas 6800 System

### Section 1: Materials and Methods

### Section 1.1: Subject Sampling

Bilateral anterior nares swabbing was performed by a healthcare professional on 15 subjects belonging to the health care service provider Leumit (Tel-Aviv, Israel), who had previously tested positive for SARS-CoV-2. A bilateral mid-turbinate swab was then performed by the healthcare professional. The subjects then sprayed 0.1 mL of normal saline 3-5 times into each nostril, and then gargled 10 mL of normal saline for 10-15 seconds to produce a Nasal Gargle (hereinbelow, “NG”) specimen. Following spitting out of the NG sample, a second bilateral anterior nares swab was performed by the healthcare professional. Swabs were then placed into tubes containing 3 mL of Universal Transport Medium (UTM) (Biological Industries, Israel). Samples were transported on ice to the Leumit Health Services Central Laboratory (Or Yehuda, Israel), for processing. The clinical study was approved by the investigational review board (IRB) at Assaf Harofeh (Shamir) Medical Center (Tel Aviv, Israel). All swabbing was performed with flocked swabs (3BY Ltd., Industrial Park Tefen, Ref #: 3by-001).

### Section 1.2: Molecular Testing

RNA extraction and qPCR were performed using the Cobas SARS-CoV-2 Test (Roche, Switzerland) on a Cobas 6800 system (Roche, Switzerland), in accordance with manufacturer instructions.

### Section 2: Results and Discussion

All 15 subjects were positive for both ORF1 a/b and E gene targets (Table 8). (As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels.)

Fourteen out of the 15 subjects (93.3%) were found to have lower Ct values, indicative of higher viral levels, for both gene targets in samples from anterior nares swabs performed after saline nasal spraying and gargling than from anterior nares swabs performed before saline nasal spraying and gargling. Furthermore, 7 out of the 15 subjects (46.7%) were found to have lower Ct values, indicative of higher viral levels, across both gene targets in samples from anterior nares swabs performed after saline nasal spraying and gargling than in mid-turbinate swabs. In cases where the mid-turbinate swab outperformed the anterior nares swabs sampled after saline nasal spraying and gargling on at least one gene target, anterior nares swabs sampled after saline nasal spraying and gargling were still within 1 Ct value of the mid-turbinate swab in 4 out 8 subjects (62.5%), with 2 of the subjects outperforming mid-turbinate swabs on the second target.

On average, Ct values for anterior nares swabs performed after saline nasal spraying decreased by 2.35 for the ORF1 a/b gene and by 2.70 for the E gene compared to Ct values for anterior nares swabs performed before saline nasal spraying (Table 9). This decrease was statistically significant (ORF1 a/b gene, p=0.00014; E gene, p=0.00125) (Student's t-test).

This data suggest that dispensing nasal spray and gargling before anterior nares swabbing increases collected viral material when compared to anterior nares swabs without a prior nasal spray and gargle, and in some cases, when compared to mid-turbinate swabs. It is therefore expected that administering a nasal spray prior to anterior nares swabbing as a sampling method would result in an increase in molecular and immunological testing sensitivity.

TABLE 8
Ct value results

| Subject | Sample¹ | ORF1 a/b gene | E gene | ICⁱⁱ |
|---|---|---|---|---|
| 1 | AN1 | 20.38 | 20.45 | 32.91 |
| | MT | 19.53 | 19.66 | 33.12 |
| | AN2 | 19.42 | 19.66 | 32.9 |
| 2 | AN1 | 34.05 | 39.74 | 32.83 |
| | MT | 30.8 | 31.6 | 33.04 |
| | AN2 | 29.78 | 30.51 | 32.81 |
| 3 | AN1 | 24.36 | 24.14 | 32.33 |
| | MT | 23.27 | 23.27 | 32.68 |
| | AN2 | 23.32 | 23.14 | 32.59 |
| 4 | AN1 | 32.73 | 33.79 | 33.13 |
| | MT | 28.71 | 29.19 | 32.75 |
| | AN2 | 30.54 | 31.35 | 32.77 |
| 5 | AN1 | 27.04 | 27.1 | 32.27 |
| | MT | 27.51 | 27.7 | 32.68 |
| | AN2 | 28.11 | 28.51 | 32.59 |
| 6 | AN1 | 26.62 | 26.87 | 32.68 |
| | MT | 25.24 | 25.46 | 32.53 |
| | AN2 | 23.87 | 24.07 | 32.42 |
| 7 | AN1 | 29.89 | 30.44 | 32.89 |
| | MT | 24.82 | 24.76 | 32.35 |
| | AN2 | 26.89 | 27.24 | 32.55 |
| | AN1 | 28.19 | 28.54 | 32.66 |
| 8 | MT | 29.52 | 30.1 | 32.76 |
| | AN2 | 26.43 | 26.64 | 32.23 |
| 9 | AN1 | 21.24 | 21.48 | 32.59 |
| | MT | 20.98 | 21.29 | 32.91 |
| | AN2 | 19.09 | 19.1 | 33.5 |
| 10 | AN1 | 30.05 | 30.69 | 33.11 |
| | MT | 30.17 | 30.99 | 33.11 |
| | AN2 | 29.12 | 29.96 | 32.72 |
| 11 | AN1 | 34.26 | 35.8 | 32.82 |
| | MT | 29.5 | 30.48 | 33.38 |
| | AN2 | 29.07 | 30.01 | 32.73 |
| 12 | AN1 | 30.62 | 31.18 | 32.89 |
| | MT | 23.37 | 23.59 | 32.7 |
| | AN2 | 25.17 | 25.7 | 34.19 |
| 13 | AN1 | 30.04 | 30.43 | 32.94 |
| | MT | 27.27 | 27.57 | 32.61 |
| | AN2 | 27.67 | 27.97 | 32.37 |
| 14 | AN1 | 27.84 | 28.34 | 32.7 |
| | MT | 27.03 | 27.64 | 32.81 |
| | AN2 | 24.4 | 24.88 | 32.76 |
| 15 | AN1 | 35.18 | 36.79 | 32.89 |
| | MT | 33.02 | 34.4 | 32.82 |
| | AN2 | 34.33 | 36.49 | 32.73 |

ⁱAN1 = anterior nares swab performed prior to saline nasal spraying and gargling, MT = mid-turbinate swab, AN2 = anterior nares swab performed after saline nasal spraying and gargling
ⁱⁱIC = internal control

TABLE 9
Average Ct values and Average Difference between AN1 and AN2

| | AN1 | | AN2 | | Delta: AN2 − AN1 | |
|---|---|---|---|---|---|---|
| | ORF1 a/b | | ORF1 a/b | | ORF1 a/b | |
| Subject | gene | E gene | gene | E gene | gene | E gene |
| 1 | 20.38 | 20.45 | 19.42 | 19.66 | −0.96 | −0.79 |
| 2 | 34.05 | 39.74 | 29.78 | 30.51 | −4.27 | −9.23 |
| 3 | 24.36 | 24.14 | 23.32 | 23.14 | −1.04 | −1 |
| 4 | 32.73 | 33.79 | 30.54 | 31.35 | −2.19 | −2.44 |
| 5 | 27.04 | 27.1 | 28.11 | 28.51 | 1.07 | 1.41 |
| 6 | 26.62 | 26.87 | 23.87 | 24.07 | −2.75 | −2.8 |
| 7 | 29.89 | 30.44 | 26.89 | 27.24 | −3 | −3.2 |
| 8 | 28.19 | 28.54 | 26.43 | 26.64 | −1.76 | −1.9 |
| 9 | 21.24 | 21.48 | 19.09 | 19.1 | −2.15 | −2.38 |
| 10 | 30.05 | 30.69 | 29.12 | 29.96 | −0.93 | −0.73 |
| 11 | 34.26 | 35.8 | 29.07 | 30.01 | −5.19 | −5.79 |
| 12 | 30.62 | 31.18 | 25.17 | 25.7 | −5.45 | −5.48 |
| 13 | 30.04 | 30.43 | 27.67 | 27.97 | −2.37 | −2.46 |
| 14 | 27.84 | 28.34 | 24.4 | 24.88 | −3.44 | −3.46 |
| 15 | 35.18 | 36.79 | 34.33 | 36.49 | −0.85 | −0.3 |
| Average | 28.83 | 29.72 | 26.48 | 27.01 | −2.35 | −2.70 |

In some applications, any of the techniques described in the report of Experiment 10 above are used in combination with the other techniques described herein. It is noted that although Experiment 10, as reported above, included mid-turbinate swabbing of the subjects, this mid-turbinate swabbing served for comparison with anterior nares swabbing. Some techniques of the present invention include performing only an anterior nares swab, without performing a mid-turbinate swab.

### Experiment 11

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay. Experiment 11 provides evidence of the efficacy of these techniques for collecting specimen samples for detection of SARS-CoV-2 using lateral flow immunoassay strips.

In summary, Experiment 11 found that two of the four subjects tested positive for SARS-CoV-2 antigens both on the mid-turbinate swab and on the anterior flares swab performed after saline nasal spraying, but negative on the anterior nares swab performed before saline nasal spraying, indicating that saline nasal spraying can increase anterior nares swab sensitivity with immunoassays. The remaining two of the four subjects tested negative for SARS-CoV-2 antigens on all sample types (anterior nares swabbing before saline nasal spraying, mid-turbinate swabbing, and anterior nares swabbing after saline nasal spraying).

### Detailed Report of Experiment 11: Benefits of Saline Nasal Spraying Prior to Anterior Nares Swabbing in SARS-CoV-2 Detection by Immunoassay

### Section 1: Materials and Methods

### Section 1.1: Subject Sampling

Bilateral anterior nares swabbing was performed by a healthcare professional on 20 subjects belonging to the health care service provider Leumit (Tel-Aviv, Israel), who had previously tested positive for SARS-CoV-2. The subjects then sprayed 0.1 mL of normal saline 3-5 times into each nostril, and then gargled 10 mL of normal saline for 10-15 seconds to produce a Nasal Gargle specimen. Following spitting out of the Nasal Gargle sample, a second bilateral anterior nares swab was performed by the healthcare professional. Lastly, a bilateral mid-turbinate swab was performed by the healthcare professional. Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001). The clinical study was approved by the investigational review board (IRB) at Assaf Harofeh (Shamir) Medical Center (Tel Aviv, Israel).

### Section 1.2: Immunological Testing

Immunoassays were performed using either the BD Veritor™ system for Rapid Detection of SARS-CoV-2 (Becton Dickinson and Company, Maryland, USA, Ref: 256082), or the COVID-19 Antigen Rapid Test Cassette (Beijing Kewei Clinical Diagnostic Reagent Inc., Beijing, China, Ref: 601450) in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs, and the COVID-19 Antigen Rapid Test Cassette is a solid phase immunochromatographic assay for the in vitro qualitative detection of SARS-CoV-2 antigen in nasopharyngeal, nasal, and throat swab specimens. Due to the limited availability of the BD Veritor™ system for Rapid Detection of SARS-CoV-2, in 4 subjects the anterior nares performed prior to nasal spraying and gargling was tested on the COVID-19 Antigen Rapid Test Cassette, with all other sample types tested on the BD Veritor™ system for Rapid Detection of SARS-CoV-2. In order to determine semi-quantitative differences in viral load between sample types, band intensity was scored on a scale of 0-3, with 0 corresponding to a negative test.

### Section 2: Results and Discussion

7 subjects tested negative and 4 subjects tested positive for SARS-CoV-2 antigens on all sample types (anterior nares swabbing before saline nasal spraying and gargling, mid-turbinate swabbing, and anterior nares swabbing after saline nasal spraying and gargling).

6 out of the 9 subjects with discordant results (67%) tested positive for SARS-CoV-2 antigens both on the mid-turbinate swab and on the anterior nares swab performed after saline nasal spraying and gargling, but negative on the anterior nares swab performed before saline nasal spraying and gargling, indicating that saline nasal spraying increases anterior nares swab efficacy in viral material sampling and sensitivity with immunoassays. In a like manner, 1 out of the 9 subjects with discordant results tested clearly positive for SARS-CoV-2 antigens both on the mid-turbinate swab and on the anterior nares swab performed after saline nasal spraying and gargling; however, on the anterior nares swab prior to saline nasal spraying and gargling, it was unclear whether or not there was a faint band corresponding to the presence of SARS-CoV-2 antigens. Regarding the remaining 2 subjects with discordant results, 1 subject tested positive for SARS-CoV-2 antigens on the mid-turbinate swab, but negative on both anterior nares swabs, and 1 subject tested positive for SARS-CoV-2 antigens on the anterior nares swab performed after saline nasal spraying and gargling, but negative on both the mid-turbinate and anterior nares swab performed prior to nasal spraying and gargling. Similarly, positive band intensity for SARS-CoV-2 antigens in anterior nares swab samples performed after saline nasal spraying and gargling was equal to that of the mid-turbinate swab samples for 2 out of the 15 subjects (13.3%) that presented a positive on both the anterior nares swab performed after saline nasal spraying and gargling and the mid-turbinate swab. Moreover, positive band intensity for SARS-CoV-2 antigens was higher in anterior nares swab samples performed after saline nasal spraying and gargling than that of the mid-turbinate swab samples for 3 out of the 15 subjects (20%) that presented a positive on both the anterior nares swab performed after saline nasal spraying and gargling and the mid-turbinate swab, indicating that by coupling nasal spray with anterior nares swabbing, anterior nares swabs in some cases collect more viral material than mid-turbinate swabs.

In some applications, any of the techniques described in the report of Experiment 11 above are used in combination with the other techniques described herein. It is noted that although Experiment 11, as reported above, included mid-turbinate swabbing of the subjects, this mid-turbinate swabbing served for comparison with anterior nares swabbing. Some techniques of the present invention include performing only an anterior nares swab, without performing a mid-turbinate swab.

### Experiment 12

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using PCR. Experiment 12 provides evidence of the efficacy of these techniques for collecting specimen samples for detection of SARS-CoV-2 using quantitative PCR (qPCR).

### Detailed Report of Experiment 12: Benefits of Saline Nasal Spraying Before Anterior Nares Swabbing in SARS-CoV-2 Detection

### Section 1: Materials and Methods

### Section 1.1: Subject Sampling

Three bilateral anterior nares swab samplings were performed approximately one minute apart by a healthcare professional on 12 subjects belonging to the health care service provider Leumit (Tel-Aviv, Israel), who had previously tested positive for SARS-CoV-2. The subjects then sprayed 0.1 mL of normal saline 3-5 times into each nostril and gargled 10 mL of normal saline. Then a fourth bilateral anterior nares swab was performed by the healthcare professional. Bilateral anterior nares swabbing was performed by inserting swabs into the nares and then removing the swabs without rotating the swabs against the inner wall of the nares. Swabs were then placed into tubes containing 3 mL of Universal Transport Medium (UTM) (Biological Industries, Israel). Samples were transported on ice to the Leumit Health Services Central Laboratory (Or Yehuda, Israel), for processing. The clinical study was approved by the investigational review board (IRB) at Assaf Harofeh (Shamir) Medical Center (Tel Aviv, Israel). All swabbing was performed with flocked swabs (3BY Ltd., Industrial Park Tefen, Israel, Ref: 3by-001).

### Section 1.2: Molecular Testing

Samples were processed by qPCR at the Leumit Health Services Central Laboratory (Or Yehuda, Israel) using the Allplex 2019 n-CoV Assay (Seegene, Seoul, South Korea), following manufacturer's instructions, on a CFX96 Real-Time System (BIO RAD, USA). Prior to qPCR, samples were extracted using the STARMag 96×4 Viral DNA/RNA 200 C Kit (Seegene, Seoul, South Korea) on a MICROLAB STARlet (Hamilton, Switzerland) automated liquid handling platform. Statistical analysis was performed using the Wilcoxon signed rank test to determine p-values.

### Section 2: Results and Discussion

Lower Ct values were observed in anterior nares swabs performed after nasal spraying and gargling than before nasal spraying and gargling on average across all SARS-CoV-2 gene targets (Tables 10.1, 10.2), indicative of higher viral levels after nasal spraying and gargling than before nasal spraying and gargling independent of possible contributions from a mid-turbinate swab.

Average Ct values of the fourth anterior nares swab, which were preceded by nasal spraying and gargling, were lower across all gene targets than that of the other paired anterior nares swabs performed prior to nasal spraying and gargling (Table 10.2). The reversal of the observed aforementioned trend highlights the benefit of performing nasal spraying and gargling prior to anterior nares swabbing in SARS-CoV-2 detection, specifically in light of comparisons between the third anterior nares swab performed prior to nasal spraying and gargling and the fourth anterior nares swab performed following nasal spraying and gargling. The difference between the averages of the third nares swab performed prior to nasal spraying and gargling and the fourth anterior nares swab performed following nasal spraying and gargling reached statistical significance across all gene targets tested (Table 10.3) despite the relatively small sample size.

TABLE 10.1
Ct calue results

| | | C(t) values for each Gene Target^{ψ} | | | |
|---|---|---|---|---|---|
| Subject # | Sampleⁱ | E | RdRP/S | N | ICⁱⁱ |
| 1 | AN1 | 40.0 | 40.0 | 40.0 | 25.6 |
| | AN2 | 33.7 | 36.0 | 36.0 | 27.1 |
| | AN3 | 40.0 | 40.0 | 40.0 | 27.4 |
| | AN4 (after NG) | 34.7 | 35.1 | 37.8 | 25.8 |
| 2 | AN1 | 25.6 | 27.5 | 37.8 | 26.7 |
| | AN2 | 28.8 | 31.5 | 27.8 | 26.9 |
| | AN3 | 26.2 | 28.7 | 31.1 | 25.7 |
| | AN4 (after NG) | 24.8 | 26.8 | 26.9 | 25.8 |
| 3 | AN1 | 25.0 | 26.9 | 27.2 | 25.7 |
| | AN2 | 24.3 | 26.1 | 26.5 | 25.4 |
| | AN3 | 23.0 | 25.0 | 24.4 | 24.8 |
| | AN4 (after NG) | 20.1 | 21.7 | 22.3 | 27.1 |
| 4 | AN1 | 40.0 | 40.0 | 40.0 | 40.0 |
| | AN2 | 40.0 | 40.0 | 40.0 | 40.0 |
| | AN3 | 40.0 | 40.0 | 40.0 | 40.0 |
| | AN4 (after NG) | 40.0 | 40.0 | 40.0 | 40.0 |
| 5 | AN1 | 36.4 | 40.0 | 39.5 | 26.7 |
| | AN2 | 40.0 | 40.0 | 36.9 | 25.5 |
| | AN3 | 40.0 | 40.0 | 40.0 | 26.7 |
| | AN4 (after NG) | 40.0 | 40.0 | 40.0 | 25.2 |
| 6 | AN1 | 28.4 | 30.5 | 31.3 | 26.7 |
| | AN2 | 29.2 | 30.9 | 31.7 | 25.5 |
| | AN3 | 30.4 | 32.6 | 33.0 | 26.7 |
| | AN4 (after NG) | 28.0 | 29.4 | 30.4 | 25.2 |
| 7 | AN1 | 40.0 | 40.0 | 37.9 | 25.7 |
| | AN2 | 40.0 | 36.7 | 40.0 | 25.6 |
| | AN3 | 40.0 | 40.0 | 40.0 | 26.9 |
| | AN4 (after NG) | 40.0 | 40.0 | 40.0 | 27.1 |
| 8 | AN1 | 40.0 | 40.0 | 40.0 | 25.6 |
| | AN2 | 40.0 | 40.0 | 40.0 | 27.1 |
| | AN3 | 40.0 | 40.0 | 40.0 | 26.9 |
| | AN4 (after NG) | 40.0 | 37.1 | 40.0 | 25.4 |
| 9 | AN1 | 40.0 | 40.0 | 40.0 | 27.2 |
| | AN2 | 40.0 | 40.0 | 40.0 | 27.3 |
| | AN3 | 40.0 | 40.0 | 40.0 | 27.1 |
| | AN4 (after NG) | 40.0 | 39.2 | 40.0 | 27.3 |
| 10 | AN1 | 40.0 | 40.0 | 40.0 | 40.0 |
| | AN2 | 40.0 | 40.0 | 40.0 | 40.0 |
| | AN3 | 40.0 | 40.0 | 40.0 | 40.0 |
| | AN4 (after NG) | 40.0 | 40.0 | 40.0 | 40.0 |
| 11 | AN1 | 32.3 | 34.2 | 33.7 | 25.8 |
| | AN2 | 32.8 | 34.5 | 33.7 | 25.5 |
| | AN3 | 31.9 | 33.3 | 34.4 | 26.9 |
| | AN4 (after NG) | 28.3 | 29.6 | 30.3 | 26.0 |
| 12 | AN1 | 24.8 | 26.6 | 26.7 | 25.4 |
| | AN2 | 29.6 | 31.1 | 31.6 | 25.5 |
| | AN3 | 26.4 | 28.0 | 28.8 | 25.7 |
| | AN4 (after NG) | 25.9 | 27.8 | 28.3 | 25.8 |

ⁱAN1, AN2, AN3 = Three anterior nares swabs performed in succession prior to nasal spraying and gargling (NG); AN4 = Anterior nares swab performed after NG
ⁱⁱIC = Internal control used in molecular assay
^{ψ}Negative Ct values are expressed as 40

TABLE 10.2
Ct value aver usesⁱⁱⁱ

| | C(t) value averages | | |
|---|---|---|---|
| | for each Gene Target | | |
| Sample | E | RdRP/S | N |
| AN1 | 31.56 | 33.21 | 34.27 |
| AN2 | 32.30 | 33.36 | 33.04 |
| AN3 | 32.24 | 33.45 | 33.95 |
| AN4 (after NG) | 30.21 | 31.29 | 31.99 |

ⁱⁱⁱFor the purpose of making a meaningful comparison between swab samples, average Ct values exclude subjects with less than two positive gene targets across all samples (excluded: subject 4, subject 8, subject 9, and subject 10)

TABLE 10.3
Statistical Significance: p-values^{iv}

| | p-values for each Gene Target | | |
|---|---|---|---|
| Paired Samples | E | RdRP/S | N |
| AN1-AN2 | 0.447 | 0.866 | 0.554 |
| AN1-AN3 | 0.344 | 0.590 | 0.933 |
| AN1-AN4 | 0.272 | 0/142 | 0.195 |
| AN2-AN3 | 0.675 | 0/800 | 0.272 |
| AN2-AN4 | 0.059 | 0.076 | 0.272 |
| AN3-AN4 | 0.036* | 0.036* | 0.036* |

^{iv}The Wilcoxon signed rank test was used to determine p-values. For the purpose of making a meaningful comparison between swab samples, statistical tests exclued subjects with less than two positive gene targets across all samples (excluded: subject 4, subject 8, subject 9, and subject 10)
*Indicates statistically significant values

In some applications, any of the techniques described in the report of Experiment 12 above are used in combination with the other techniques described herein.

### Experiment 13

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay. Experiment 13 provides evidence of the efficacy of these techniques for collecting specimen samples for detection of SARS-CoV-2 using lateral flow immunoassay strips.

In summary, Experiment 13 found that, in a single subject, the SARS-CoV-2 antigen band intensity for an anterior nares swab performed following nasal saline spraying was greater than that of both an anterior flares swab performed prior to nasal saline spraying and a mid-turbinate swab, indicating a greater quantity of virus recovered on an anterior nares swabs performed following nasal saline spraying than on anterior flares swab performed prior to nasal saline spraying and a mid-turbinate swab.

### Detailed Report of Experiment 13: Further Evaluation of the Benefits of Saline Nasal Spraying without Gargling Prior to Anterior Nares Swabbing in SARS-CoV-2 Detection by Immunoassay

### Section 1: Materials and Methods

### Section 1.1: Subject Sampling

Bilateral anterior nares swabbing was performed by a healthcare professional on 7 subjects belonging to the health care service provider Leumit (Tel-Aviv, Israel), who had previously tested positive for SARS-CoV-2. The subjects then sprayed 0.1 mL of normal saline 3-5 times into each nostril with their heads in a neutral position or slightly tilted back. Subjects then significantly tilted their heads backwards by extending their necks as far as comfortably possible and maintained this head positioning for 5-10 seconds, similar to the conditions that normally occur during gargling. The subjects then returned their heads to a neutral position and a second bilateral anterior nares swabbing was performed by the healthcare professional. Lastly, bilateral mid-turbinate swabbing was performed by the healthcare professional. Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001). The clinical study was approved by the investigational review board (IRB) at Assaf Harofeh (Shamir) Medical Center (Tel Aviv, Israel).

### Section 1.2: Immunological Testing

Immunoassays were performed on all sample types using the BD Veritor™ system for Rapid Detection of SARS-CoV-2 (Becton Dickinson and Company, Maryland, USA, Ref: 256082), in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs. In order to determine semi-quantitative differences in viral load between sample types, band intensity was scored on a scale of 0-3, with 0 corresponding to a negative test.

### Section 2: Results and Discussion

Six of the seven subject samples were close to their 10-day quarantine release date, or had already been released, and unsurprisingly tested negative on all immunoassays performed. The single remaining subject who had tested positive for SARS-CoV-2 by qPCR 4 days prior to sample collection tested positive for the presence of SARS-CoV-2 antigen on all sample types (anterior nares swab prior to nasal saline spraying, anterior nares swab following nasal saline spraying, and the mid-turbinate swab) by immunoassay. The SARS-CoV-2 antigen band intensity for the anterior nares swab performed following nasal saline spraying (score: 3) was greater than that of both the anterior nares swab performed prior to nasal saline spraying (score: 2) and the mid-turbinate swab (score: 2.5), indicating a greater quantity of virus recovered on an anterior nares swab performed following nasal saline spraying than on an anterior nares swab performed prior to nasal saline spraying and a mid-turbinate swab.

This observation supports the fact that coupling nasal saline spraying to anterior nares swabbing increases immunoassay testing sensitivity. As subjects did not gargle saline as in the previous experiments described hereinabove in Experiments 9, 10, 11, and 12, it appears that saline spraying alone is sufficient for increasing anterior nares testing sensitivity.

In some applications, any of the techniques described in the report of Experiment 13 above are used in combination with the other techniques described herein.

### Experiment 14

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay or using isothermal amplification. Experiment 14 provides evidence of the efficacy of these techniques for collecting specimen samples for detection of SARS-CoV-2 using lateral flow immunoassay strips and using isothermal amplification.

In summary, Experiment 14 found that the sensitivity of lateral flow immunoassay strips improve for anterior nares swabs performed following nasal saline spraying compared to anterior nares swabs performed prior to nasal saline spraying and that, on average, the SARS-CoV-2 antigen band intensity for anterior nares swabs performed following nasal saline spraying were greater than that of anterior flares swabs performed prior to nasal saline spraying, indicating a greater quantity of virus recovered on anterior nares swabs performed following nasal saline spraying than on anterior nares swab performed prior to nasal saline spraying, in support of Experiment 13 hereinabove. Experiment 14 additionally found that the sensitivity of isothermal amplification SARS-CoV-2 detection tests also improve for anterior nares swabs performed following nasal saline spraying compared to anterior nares swabs performed prior to nasal saline spraying.

### Detailed Description of Experiment 14: Further Evaluation of Saline Nasal Spraying without Gargling Prior to Anterior Nares Swabbing in SARS-CoV-2 Detection by Immunoassay and Isothermal Amplification and Effects of Head Positioning

### Section 1: Materials and Methods

### Section 1.1: Patient Population

This experiment was performed by collecting samples from 14 subjects belonging to the health care service provider Leumit (Tel-Aviv, Israel), who had previously tested positive for SARS-CoV-2. Of the 14 subjects, 6 subjects were subsequently retested the following day (n=20). The clinical study was approved by the investigational review board (IRB) at Assaf Harofeh (Shamir) Medical Center (Tel Aviv, Israel).

### Section 1.2: Subject Sampling

Two alternative sample collection procedures were implemented during subject sampling. First, a unilateral anterior naris swabbing of the right nostril was performed by a healthcare professional. Subjects then sprayed 0.1 mL of normal saline 3-5 times into the right nostril with their heads in a neutral position or slightly tilted back, sniffed in deeply, and then tilted their heads backwards by extending their necks as far as comfortably possible and maintained this head positioning for approximately 15 seconds. The subjects then returned their heads to a neutral position and a second unilateral anterior naris swabbing of the right nostril and then a mid-turbinate swabbing of the right nostril were performed by a healthcare professional. A unilateral anterior naris swabbing of the left nostril was then performed by a healthcare professional. As with the right nostril, subjects then sprayed 0.1 mL of normal saline 3-5 times into the left nostril with their heads in a neutral position or slightly tilted back. Subjects then maintained the neutral or slightly tilted back head positioning for approximately 10 seconds. Lastly, a second unilateral anterior naris swabbing of the left nostril was performed by a healthcare professional.

Alternatively, following the unilateral anterior naris swabbing of the right nostril and then saline nasal spraying into the right nostril as described above, subjects maintained the neutral or slightly tilted back head positioning for approximately 10 seconds followed by a second unilateral anterior naris swabbing and then a mid-turbinate swabbing of the right nostril by the healthcare professional. Following the unilateral anterior naris swabbing of the left nostril and then saline nasal spraying into the left nostril as described above, subjects then tilted their heads backwards by extending their necks as far as comfortably possible and maintained this head positioning for approximately 15 seconds. The subjects then returned their heads to a neutral position and a second unilateral anterior naris swabbing of the left nostril was performed by a healthcare professional.

Six subjects were resampled the following day such that the head positioning maintained following saline nasal spraying was reversed for each nostril during resampling compared to during original sampling (Table 11.1).

Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001).

### Section 1.3: Testing

Most swab samples were tested by lateral flow immunoassay strips, except for four swab samples: two pairs of unilateral outer naris swab samples that were collected from the left nostril of two subjects with different head positionings were tested by isothermal amplification instead of by lateral flow immunoassay strips (Table 11.1).

Immunoassays were performed using the BD Veritor™ system for Rapid Detection of SARS-CoV-2 (Becton Dickinson and Company, Maryland, USA, Ref: 256082), in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs. In order to determine semi-quantitative differences in viral load between sample types, band intensity was scored on a scale of 0-3, with 0 corresponding to a negative test.

Isothermal amplification tests were performed using the Lucira™ COVID-19 All-In-One Test Kit system for Rapid Detection of SARS-CoV-2 (Lucira Health, Emeryville, Calif.), in accordance with manufacturer instructions. The Lucira™ COVID-19. All-In-One Test Kit system for Rapid Detection of SARS-CoV-2 is isothermal amplification detection test that utilizes RT-LAMP technology to detect RNA of the N gene for SARS-CoV-2. A positive qualitative result is reported if an RNA signal is detected within 30 minutes and a negative qualitative result is reported if an RNA signal is not detected within 30 minutes. In order to determine semi-qualitative differences in viral load between sample types, the inventors considered the amount of time until results displayed as a relative indicator, with relatively less time until results displayed as an indicator of greater viral load and relatively more time until results displayed as an indicator of lower viral load.

TABLE 11.1
Overview of Head Positionins Durins Subject Sanwlins,
Detection Test Used, and Procedural Deviations

| | | Head | Detection Test |
|---|---|---|---|
| Subject | Nostril | Position¹ | Used |
| 1 | Right | Neutral | Immunoassay |
| | Left | Back | Immunoassay |
| 2 | Right | Back | Immunoassay |
| | Left | Neutral | Immunoassay |
| 3 | Right | Neutral | Immunoassay |
| | Left | Back | Immunoassay |
| 4 | Right | Back | Immunoassay |
| | Left | Neutral | Immunoassay |
| 5 | Right | Neutral | Immunoassay |
| | Left | Back | Immunoassay |
| 6 | Right | Back | Immunoassay |
| | Left | Neutral | Immunoassay |
| 7^{§} | Right | Neutral | Immunoassay |
| | Left | Back | Immunoassay |
| 8^{§} | Right | Back | Immunoassay |
| | Left | Neutral | Immunoassay |
| 9 | Right | Neutral | Immunoassay |
| | Left | Back | Immunoassay |
| 10 | Right | Back | Immunoassay |
| | Left | Neutral | Immunoassay |
| 11 | Right | Neutral | Immunoassay |
| | Left | Back | Immunoassay |
| 12 | Right | Back | Immunoassay |
| | Left | Neutral | Immunoassay |
| 13^{Ω} | Right | Back | Immunoassay |
| | Left | Neutral | Immunoassay |
| 14 | Right | Neutral | Immunoassay |
| | Left | Back | Immunoassay |
| 4* | Right | Neutral | Immunoassay |
| | Left | Back | Immunoassay |
| 5* | Right | Back | Immunoassay |
| | Left | Neutral | Isothermal |
| | | | Amplification |
| 6* | Right | Neutral | Immunoassay |
| | Left | Back | Isothermal |
| | | | Amplification |
| 7* | Right | Back | Immunoassay |
| | Left | Neutral | Immunoassay |
| 9* | Right | Back | Immunoassay |
| | Left | Neutral | Immunoassay |
| 11* | Right | Back | Immunoassay |
| | Left | Neutral | Immunoassay |

iHead positioning immediately following saline nasal spraying. “Back” indicates that the subject maintained a completely tilted back head position for approximately 15 seconds, “Neutral” indicated that the subject maintained a neutral or slightly tilted of back head position for approximately 10 seconds.
^{Ω}Subject did not sniff in after saline nasal spraying.
*Resampling subjects with switched head positioning.
^{§}Immunoassay band intensity corresponding to SARS-CoV-2 antigen presence was not scored

### Section 2: Results and Discussion

Immunoassay results were obtained from a total of 38 paired unilateral anterior naris swabs collected before and after saline nasal spraying: 19 paired unilateral anterior naris swabs for each head position (neutral or slightly tilted, and completely tilted back). Isothermal amplification results were obtained from a total of 2 paired unilateral anterior naris swabs collected before and after saline nasal spraying: 1 paired unilateral anterior naris swab for each head position (neutral or slightly tilted, and completely tilted back). Because of expected differences in viral loads between nostrils caused by the nasal cycle, internal comparisons between sampling methodology by pairing nostrils on a patient-by-patient basis were not made, but rather samples from each nostril were treated as samples from independent subjects. Similarly, conclusions were not drawn from comparisons between original samples produced from subjects and samples collected after resampling with alternate methodologies due to temporal changes in nostril dominance, but rather were treated as samples from independent subjects.

### Section 2.1: Immunoassay Sensitivity Data

11 out of the 19 paired unilateral anterior naris swabs collected from subjects following the sampling protocol in which the head was completely tilted back after saline nasal spraying (hereinbelow, “Head Tilt Protocol”), and 9 out of the 19 paired unilateral anterior naris swabs collected from subjects following the sampling protocol in which the head was neutral or slightly tilted after saline nasal spraying (hereinbelow, “Head Neutral Protocol”), were negative for SARS-CoV-2 antigens on both unilateral anterior naris swabs collected before and after saline nasal spraying. 5 out of the remaining 8 paired unilateral anterior naris swabs collected from subjects following the Head Tilt Protocol, and 4 out of the remaining 10 paired unilateral anterior naris swabs collected from subjects following the Head Neutral Protocol, tested positive for both unilateral anterior naris swabs collected before and after saline nasal spraying.

Discordant results were reported for 3 paired unilateral anterior naris swabs collected before and after saline nasal spraying from subjects following the Head Tilt Protocol. For all 3 discordant swabs, the unilateral anterior naris swab samples taken after saline nasal spraying were positive for SARS-CoV-2 antigen, and the unilateral anterior naris swab samples taken before saline nasal spraying were negative for SARS-CoV-2 antigen. As seen in Table 11.2, following the Head Tilt Protocol, unilateral anterior naris swabs taken before saline nasal spraying were observed to be 62.5% sensitivity (5/8), while unilateral anterior naris swabs taken after saline nasal spraying had 100% sensitivity (8/8), indicating a 60% increase in detection sensitivity associated with unilateral anterior naris swabbing after saline nasal spraying following the Head Tilt Protocol relative to unilateral anterior naris swabbing performed prior to saline nasal spraying.

Discordant results were reported for 6 paired unilateral anterior naris swabs collected before and after saline nasal spraying from subjects following the Head Neutral Protocol. For 5 of the 6 discordant results, the unilateral anterior naris swab samples taken after saline nasal spraying was positive for SARS-CoV-2 antigen, and the unilateral anterior naris swab samples taken before saline nasal spraying were negative for SARS-CoV-2 antigen. Conversely, for the single discordant result following the Head Neutral Protocol, the unilateral anterior naris swab sample taken after saline nasal spraying was negative for SARS-CoV-2 antigen, and the unilateral anterior naris swab sample taken prior to saline nasal spraying was positive for SARS-CoV-2 antigen. As seen in Table 11.2, following the Head Neutral Protocol, unilateral anterior naris swabs taken before saline nasal spraying were observed to be 50% sensitivity (5/10), while unilateral anterior naris swabs taken after saline nasal spraying had 90% sensitivity (9/10), indicating an 80% increase in detection sensitivity associated with unilateral anterior naris swabbing after saline nasal spraying following the Head Neutral Protocol relative to unilateral anterior naris swabbing performed prior to saline nasal spraying.

Because saline nasal spraying increased the sensitivity of the unilateral anterior naris swabs sampled after saline nasal spraying compared to the paired unilateral anterior naris swabs sampled before saline nasal spraying in both Head Neutral and Head Tilt Protocols, it appears that the saline nasal spraying can increase the presence of SARS-CoV-2 virus in the anterior nares independent of head positioning. Furthermore, these observations support previous findings reported in Experiment 13, described hereinabove, that saline nasal spraying benefits SARS-CoV-2 detection on anterior nares swabs in a gargle-independent manner Results reported here appear to support the validity of the Head Neutral Protocol over that of the Head Tilt Protocol. However, given that unilateral anterior naris swabs sampled prior to saline nasal spraying were 25% more sensitive for those sampled during testing of the Head Tilt Protocol relative to those sampled during testing of the Head Neutral Protocol (62.5% and 50%, respectively), despite differences between protocols only effecting samples taken after nasal spraying (unilateral anterior naris swabbing after saline nasal spraying), it is likely that relative differences between protocols are at least partially due to the small sample size. Moreover, and more importantly, as unilateral anterior naris swabbing was performed immediately after returning the head to a normal or slightly tilted back position in the Head Tilt Protocol, it is possible that saline which sampled the virus-rich nasopharyngeal regions had not had sufficient time to drip back into the anterior nares for sampling, as opposed to the Head Neutral Protocol, where the head positioning was maintained for 10 seconds and was thus conducive to saline drippage.

TABLE 11.2
Immunoassay Sensitivity

| | Head Neutral Protocol | | | Head Tilt Protocol | | |
|---|---|---|---|---|---|---|
| Subject | Nostril | AN1 | AN2 | Nostril | AN1 | AN2 |
| 1 | Right | 0 | 0 | Left | 0 | 0 |
| 2 | Left | 0 | 0 | Right | 0 | 0 |
| 3 | Right | 0 | 0 | Left | 0 | 0 |
| 4 | Left | 0 | 1 | Right | 0 | 0 |
| 5 | Right | 0 | 0 | Left | 0 | 0 |
| 6 | Left | 0 | 0 | Right | 0 | 0 |
| 7 | Right | 0 | 1 | Left | 0 | 1 |
| 8 | Left | 1 | 0 | Right | 0 | 0 |
| 9 | Right | 0 | 1 | Left | 1 | 1 |
| 10 | Left | 0 | 0 | Right | 0 | 0 |
| 11 | Right | 0 | 0 | Left | 1 | 1 |
| 12 | Left | 1 | 1 | Right | 0 | 0 |
| 13 | Left | 1 | 1 | Right | 1 | 1 |
| 14 | Right | 0 | 1 | Left | 0 | 0 |
| 4* | Right | 0 | 0 | Left | 1 | 1 |
| 5* | Left | NA | NA | Right | 0 | 0 |
| 6* | Right | 0 | 0 | Left | NA | NA |
| 7* | Left | 0 | 1 | Right | 0 | 1 |
| 9* | Left | 1 | 1 | Right | 1 | 1 |
| 11* | Left | 1 | 1 | Right | 0 | 1 |
| Sensitivity | | 50% | 90% | | 62.5% | 100% |
| | | (5/10)ⁱ | (9/10) | | (5/8) | (8/8) |

ⁱSamples were assumed true positive if at least one of the paired unilateral anterior naris swabs before and/or after saline nasal spraying was positive for SARS-CoV-2 antigen. Samples taken from different nostrils from the same subject were assessed as separate subjects.
*Resampling subjects with switched head positioning.

### Section 2.2: Immunoassay Band Intensity Data

With regards to the Head Tilt Protocol, for the 5 subjects for whom both of the paired unilateral anterior naris swabs were positive for SARS-CoV-2 antigens, 60% of the time (3/5) band intensity was greater for unilateral anterior naris swabs sampled after saline nasal spraying than unilateral anterior naris swabs sampled before nasal spraying. Similarly, for the 4 subjects for whom both of the paired unilateral anterior naris swabs were positive for SARS-CoV-2 antigens following the Head Normal Protocol, 75% of the time (3/4) band intensity was greater for unilateral anterior naris swabs sampled after saline nasal spraying than unilateral anterior naris swabs sampled before nasal spraying, with equal band intensities for both paired unilateral anterior naris swabs for the remaining subject. The overall trend of stronger band intensities associated with unilateral anterior naris swabs sampled after saline nasal spraying, indicating sampling of more viral material further demonstrates the efficacy of incorporating saline nasal spraying into anterior nares swab sampling methods independent of head positioning.

### Section 2.3: Isothermal Amplification Sensitivity Data

As seen in Table 11.3, one of the two paired unilateral anterior naris swabs collected from subjects following either the Head Tilt Protocol or Head Neutral Protocol tested positive for both unilateral anterior naris swabs collected before and after saline nasal spraying. Discordant results were reported for the one remaining paired unilateral anterior naris swabs collected before and after saline nasal spraying. For this one pair of discordant swabs, the unilateral anterior naris swab sample taken after saline nasal spraying was positive for SARS-CoV-2 antigen, and the unilateral anterior naris swab sample taken before saline nasal spraying was negative for SARS-CoV-2 antigen.

Furthermore, the one pair of unilateral anterior naris swabs with non-discordant results showed a difference in the amount of time until the isothermal amplification detection test displayed results. The tested unilateral anterior naris swab sample taken before saline nasal spraying displayed results after 19 minutes, while the tested unilateral anterior naris swab sample taken after saline nasal spraying displayed results after only 15 minutes, indicating that the unilateral anterior naris swab sample taken after saline nasal spraying had a relatively greater viral load compared to the unilateral anterior naris swab sample taken before saline nasal spraying.

TABLE 11.3
Isothermal Amplification Results

| | | | | | Time Until Result |
|---|---|---|---|---|---|
| Subject | Nostril | Head Position | Swab | Result | Displayed (min) |
| 5* | Left | Neutral | AN1 | 0 | 30 (maximum) |
| | | | AN2 | 1 | 22 |
| 6* | Left | Back (Tilt) | AN1 | 1 | 19 |
| | | | AN2 | 1 | 15 |

### Section 3: Concluding Remarks

The evidence presented herein shows that the sampling methods described herein that comprise saline nasal administration (e.g., by spraying) prior to anterior nares swabbing not only increase immunoassay sensitivity, but also antigen band intensities compared to anterior nares swabbing alone, which, for example, may help in examination of immunoassay results even without the aid of an automated immunoassay reader.

Additionally, the evidence presented herein shows that the sampling methods described herein that comprise saline nasal administration (e.g., by spraying) prior to anterior nares swabbing increase isothermal amplification testing sensitivity and decrease the amount of time until positive results are displayed. As in Experiment 13, subjects did not gargle saline as in Experiments 9, 10, 11, and 12 described hereinabove, indicating that saline spraying alone is sufficient for increasing anterior nares testing sensitivity. This data also suggest the benefit of saline nasal administration prior to anterior nares swabbing independent of head positioning, since sensitivity increased when using a saline nasal spraying protocol involving a neutral head positioning and when using a saline nasal spraying protocol involving a tilted head positioning. However, the evidence presented herein is insufficient for determining which head positioning protocol is superior, since results were likely confounded by procedural variations between examined protocols (i.e., unilateral anterior naris swabbing was performed immediately after returning the head to a normal or slightly tilted back position in the Head Tilt Protocol, so it is possible that saline which sampled the virus-rich nasopharyngeal regions had not had sufficient time to drip back into the anterior nares for sampling, as opposed to the Head Neutral Protocol, where the head positioning was maintained for 10 seconds and was thus conducive to saline drippage).

In some applications, any of the techniques described in the report of Experiment 14 above are used in combination with the other techniques described herein.

### Experiment 15

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using quantitative PCR (qPCR). Experiment 15 provides evidence of the efficacy of these techniques for collecting specimen samples for detection of SARS-CoV-2 using qPCR.

In summary, Experiment 15 found that detection test sensitivity increases when the subject tilts his or her head as far back as comfortably possible after saline nasal administration prior to anterior nares swabbing compared to when saline nasal administration is performed with a head neutral positioning prior to anterior nares swabbing. Experiment 15, unlike Experiment 14, included a wait time between returning the head to a neutral or slightly tilted back position and performing an anterior nares swab sampling, which improved viral load in the sample and improved test sensitivity.

### Detailed Report of Experiment 15: Effects of Head Positioning on Saline Nasal Spraying without Gargling Prior to Anterior Nares Swabbing in SARS-CoV-2 Detection by qPCR

### Section 1: Materials and Methods

### Section 1.1: Patient Population

This experiment was performed by collecting samples from 4 subjects belonging to the health care service provider Leumit (Tel-Aviv, Israel), who had previously tested positive for SARS-CoV-2. The clinical study was approved by the investigational review board (IRB) at Assaf Harofeh (Shamir) Medical Center (Tel Aviv, Israel).

### Section 1.2: Subject Sampling

A unilateral anterior naris swabbing of the right nostril was performed by a healthcare professional. Subjects then sprayed 0.1 mL of normal saline 3-5 times into the right nostril with their heads in a neutral position or slightly tilted back, sniffed in deeply, and then tilted their heads backwards by extending their necks as far as comfortably possible and maintained this head positioning for approximately 15 seconds. The subjects then returned their heads to a neutral or slightly tilted back position and waited approximately 10 seconds before a second unilateral anterior naris swabbing of the right nostril was performed by the healthcare professional. A mid-turbinate swabbing of the right nostril was then performed by a healthcare professional. A unilateral anterior naris swabbing of the left nostril was then performed by a healthcare professional. As with the right nostril, subjects then sprayed 0.1 mL of normal saline 3-5 times into the left nostril with their heads in a neutral position or slightly tilted back. Subjects then maintained the neutral or slightly tilted back head positioning for approximately 10 seconds. Lastly, a second unilateral anterior naris swabbing of the left nostril was performed by a healthcare professional. A single subject felt unwell and refused the final unilateral anterior nares swabbing (left nostril, after saline nasal spraying).

All swabs were placed into tubes containing 3 mL of Universal Transport Medium (UTM) (Biological Industries, Israel) and transported on ice to the Leumit Health Services Central Laboratory (Or Yehuda, Israel), for processing via qPCR.

### Section 1.3: Molecular Testing

RNA extraction and qPCR were performed using the Cobas SARS-CoV-2 Test (Roche, Switzerland) on a Cobas 6800 system (Roche, Switzerland), in accordance with manufacturer instructions.

### Section 2: Results and Discussion

Average Ct score difference between unilateral anterior nares swabs collected before saline nasal spraying and unilateral anterior nares swabs collected after saline nasal spraying were greater when subjects tilted their heads back (“Head Tilting Protocol”) after saline nasal spraying compared to when subjects maintained a neutral or slightly tilted back head position (“Head Neutral Protocol”) after saline nasal spraying (Table 12.3).

TABLE 12.1
aPCR resultsii

| | | Ct Scores | |
|---|---|---|---|
| Subject | Sampleⁱ | ORF1a/b | E |
| 1 | Tilt-AN1 | 36.45 | 38.08 |
| | Tilt-AN2 | 36.67 | 40 |
| | Neutral-AN1 | 31.98 | 33.32 |
| | Neutral-AN2 | 31.46 | 32.67 |
| 2 | Tilt-AN1 | 35.16 | 36.83 |
| | Tilt-AN2 | 29.66 | 30.36 |
| | Neutral-AN1 | 29.31 | 29.75 |
| | Neutral-AN2ⁱⁱⁱ | N/A | N/A |
| 3 | Tilt-AN1 | 33.0 | 34.42 |
| | Tilt-AN2 | 31.53 | 32.38 |
| | Neutral-AN1 | 33.27 | 34.77 |
| | Neutral-AN2 | 33.6 | 35.19 |
| 4 | Tilt-AN1 | 27.86 | 28.36 |
| | Tilt-AN2 | 22.85 | 23.24 |
| | Neutral-AN1 | 27.31 | 27.8 |
| | Neutral-AN2 | 24.59 | 24.89 |

i″Tilt″-swabbing was performed on right nostril, wherein the head was tilted after saline nasal spraying. ″Neutral″-swabbing was performed on the left nostril, wherein the head remained in a neutral or slightly tilted back position following saline nasal spraying. ″AN1″-Unilateral anterior nares swabbing performed prior to saline nasal spraying. ″AN2″-Unilateral anterior nares swabbing performed after saline nasal spraying.
ⁱⁱNegative PCR results are represented by a Ct score of 40.
ⁱⁱⁱSubject refused sampling.

TABLE 13.2
Ct Score Differences

| | Head | Ct Score Differenceⁱ | |
|---|---|---|---|
| Subject | Positionⁱⁱ | ORF1a/b gene | E gene |
| 1 | Tilt | 0.22 | 1.92 |
| | Neutral | −0.52 | −0.65 |
| 2 | Tilt | −5.5 | −6.47 |
| | Neutral | N/A | N/A |
| 3 | Tilt | −1.47 | −2.04 |
| | Neutral | 0.33 | 0.42 |
| 4 | Tilt | −5.01 | −5.12 |
| | Neutral | −2.72 | −2.91 |

ⁱCt score differences were calculated by subtracting Ct scores of unilateral anterior nares swabs performed prior to saline nasal spraying from Ct scores of paired unilateral anterior nares swabbing performed after saline nasal spraying.
ⁱⁱ“Tilt”- swabbing was performed on right nostril, wherein the head was tilted after saline nasal spraying. “Neutral”- swabbing was performed on the left nostril, wherein the head remained in a neutral or slightly tilted back position following saline nasal spraying.

TABLE 12.3
Average Ct Score Difference

| Head | Average Ct Score Difference | |
|---|---|---|
| Positionⁱ | ORF1/a/b gene | E gene |
| Tilt | −2.94 | −2.93 |
| Neutral | −0.97 | −1.04 |

ⁱ″Tilt″-swabbing was performed on right nostril, wherein the head was tilted after saline nasal spraying. ″Neutral″-swabbing was performed on the left nostril, wherein the head remained in a neutral or slightly tilted back position following saline nasal spraying.

### Section 3: Concluding Remarks

Although Experiment 14, described hereinabove, may indicate that the Head Tilting Protocol is inferior to the Head Neutral Protocol for virus sampling, it is likely that the addition of a wait period before subjects return their heads to a neutral or slightly tilted position from the tilted back position, and/or the addition of a wait time after the subjects returned their heads to the neutral or slightly tilted back position, before swabbing (which was not included in the sampling protocol for Experiment 14 but was included in the sampling protocol for this Experiment 15) facilitates the dripping of saline from within the nasal cavity into the anterior nares. It therefore appears that a combination of the Head Tilting Protocol and a wait time before subjects return their heads to a neutral or slightly tilted back position, and/or a wait time after the subjects return their heads to the neutral or slightly tilted back position, and performing an anterior nares swab sampling improves viral load in the sample and improves test sensitivity.

In some applications, any of the techniques described in the report of Experiment 15 above are used in combination with the other techniques described herein.

In an embodiment, the techniques and apparatus described herein are combined with techniques and apparatus described in one or more of the following patent applications, which are assigned to the assignee of the present application and are incorporated herein by reference: PCT Publication WO 2018/158768 to Fruchter et al.; US Patent Application Publication 2019/0381498 to Fruchter et al.; U.S. Provisional Application 62/727,208, filed Sep. 5, 2018; U.S. Provisional Application 62/727,268, filed Sep. 5, 2018; PCT Publication WO 2020/049566 to Fruchter et al.; PCT Publication WO 2020/049569 to Fruchter et al., and U.S. application Ser. No. 17/270,544 in the national stage thereof; US Patent Application Publication 2021/0102876 to Fruchter et al. U.S. Provisional Application 62/896,295, filed Sep. 5, 2019 U.S. Provisional Application 62/988,145, filed Mar. 11, 2020; U.S. Provisional Application 62/988,259, filed Mar. 11, 2020; U.S. Provisional Application 63/020,723, filed May 6, 2020; U.S. Provisional Application 63/037,707, filed Jun. 11, 2020; U.S. Provisional Application 63/067,535, filed Aug. 19, 2020; U.S. Provisional Application 63/117,294, filed Nov. 23, 2020; U.S. Provisional Application 63/156,843, filed Mar. 4, 2021; U.S. Provisional Application 63/158,005, filed Mar. 8, 2021; U.S. Provisional Application 63/166,378, filed Mar. 26, 2021; U.S. Provisional Application 63/176,565, filed Apr. 19, 2021; PCT Publication WO 2021/044417 to Holtz et al; International Application PCT/IB2021/052055, filed Mar. 11, 2021; and International Application PCT/IB2021/052056, filed Mar. 11, 2012.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and
thereafter, collecting a specimen sample that passed out of an anterior opening of an oral cavity of the subject and contains at least a portion of the biological material washed into the oropharynx by the nasal wash fluid.

2. The method according to claim 1, wherein the biological material includes one or more particulates selected from the group consisting of: a virus, a bacterium, a protozoan, and a fungus.

3. The method according to claim 1, wherein intranasally dispensing comprises intranasally dispensing, by the subject, the nasal wash fluid into the nasal cavity.

4. The method according to claim 1, further comprising, after intranasally dispensing the nasal wash fluid, tilting a head of the subject farther back than while intranasally dispensing the nasal wash fluid and farther back than a neutral position.

5. The method according to claim 1, wherein intranasally dispensing the nasal wash fluid comprises intranasally dispensing the nasal wash fluid as one or more types of fluid discharge selected from the group consisting of: a fluid stream, a spray, a mist, a pressurized aerosol, and an atomized fluid.

6. The method according to claim 1, wherein intranasally dispensing the nasal wash fluid comprises intranasally dispensing the nasal wash fluid as steam.

7. The method according to claim 1, wherein intranasally dispensing the nasal wash fluid into the nasal cavity includes intranasally dispensing a total volume of 0.2-20 mL of the nasal wash fluid into the nasal cavity.

8. The method according to claim 7, wherein intranasally dispensing the nasal wash fluid into the nasal cavity comprises intranasally dispensing the total volume of the nasal wash fluid in a plurality of pulses.

9. The method according to any one of claims 1-8, wherein collecting the specimen sample comprises expressing, by the subject, the specimen sample out of the oropharynx via the anterior opening of the oral cavity.

10. The method according to claim 9, wherein expressing the specimen sample comprises spitting, by the subject, the specimen sample out of the oropharynx via the anterior opening of the oral cavity.

11. The method according to claim 9, wherein expressing the specimen sample comprises expressing the specimen sample out of the oropharynx via the anterior opening of the oral cavity after sensing, by the subject, the nasal wash fluid in the oropharynx.

12. The method according to claim 11, wherein expressing the specimen sample comprises expressing the specimen sample out of the oropharynx via the anterior opening of the oral cavity after tasting, by the subject, the nasal wash fluid in the oropharynx.

13. The method according to claim 12, wherein the nasal wash fluid includes a tastant, and wherein expressing the specimen sample comprises expressing the specimen sample out of the oropharynx via the anterior opening of the oral cavity after tasting, by the subject, the tastant in the oropharynx.

14. The method according to any one of claims 1-8, wherein collecting the specimen sample comprises drawing the specimen sample out of the oropharynx via the anterior opening of the oral cavity using an absorbent material, without swabbing the oral cavity or the oropharynx.

15. The method according to any one of claims 1-8, wherein intranasally dispensing comprises intranasally dispensing, by a healthcare worker, the nasal wash fluid into the nasal cavity.

16. The method according to any one of claims 1-8, further comprising, after intranasally dispensing the nasal wash fluid and before collecting the specimen sample, sniffing, by the subject, the biological material with the nasal wash fluid from the nasal cavity posteriorly toward the nasopharynx.

17. The method according to any one of claims 1-8, further comprising, after intranasally dispensing the nasal wash fluid and before collecting the specimen sample, snorting, by the subject, the biological material with the nasal wash fluid from the nasal cavity toward the nasopharynx.

18. The method according to any one of claims 1-8, further comprising, after collecting the specimen sample, testing the specimen sample for the presence of a particulate present in at least the biological material washed into the oropharynx by the nasal wash fluid.

19. The method according to claim 18, wherein testing the specimen sample for the presence of the particulate comprises diagnosing a disease.

20. The method according to claim 18, wherein testing the specimen sample for the presence of the particulate comprises concentrating the specimen sample by filtration prior to testing for the presence of the particulate, by passing the specimen sample through one or more filters.

21. The method according to claim 18, wherein testing the specimen sample for the presence of the particulate comprises using a molecular-based assay to test the specimen sample for the presence of the particulate.

22. The method according to claim 21, wherein testing the specimen sample for the presence of the particulate comprises using nucleic acid amplification to test the specimen sample for the presence of the particulate.

23. The method according to claim 22, wherein testing the specimen sample for the presence of the particulate comprises using polymerase chain reaction (PCR) to test the specimen sample for the presence of the particulate.

24. The method according to claim 22, wherein testing the specimen sample for the presence of the particulate comprises using isothermal amplification to test the specimen sample for the presence of the particulate.

25. The method according to claim 18, wherein testing the specimen sample for the presence of the particulate comprises performing an immunoassay to test the specimen sample for the presence of the particulate.

26. The method according to claim 18, wherein the particulate includes one or more particulates selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.

27. The method according to claim 18, wherein the particulate includes one or more particulates selected from the group consisting of: a virus, a bacterium, a protozoan, and a fungus.

28. The method according to claim 18, wherein the particulate includes a virus.

29. The method according to claim 28, wherein the virus is an Influenza virus.

30. The method according to claim 28, wherein the virus is a coronavirus.

31. The method according to claim 30, wherein the coronavirus is Severe acute respiratory syndrome coronavirus 2 (SARS-CoV-2).

32. The method according to claim 18, wherein the particulate includes a bacterium.

33. The method according to claim 32, wherein the bacterium is a Streptococcus bacterium.

34. The method according to claim 18, wherein the particulate includes one or more antigens selected from the group consisting of: a viral antigen, a bacterial antigen, and a fungal antigen.

35. The method according to any one of claims 1-8, wherein intranasally dispensing the nasal wash fluid into the nasal cavity comprises intranasally dispensing the nasal wash fluid unidirectionally within the nasal cavity.

36. The method according to any one of claims 1-8, wherein intranasally dispensing the nasal wash fluid into the nasal cavity comprises intranasally dispensing the nasal wash fluid multidirectionally within the nasal cavity.

37. The method according to claim 36, wherein intranasally dispensing the nasal wash fluid multidirectionally within the nasal cavity comprises dispensing the nasal wash fluid into the nasal cavity:
in a posterior direction toward the nasopharynx, and
in a superoposterior direction toward an olfactory area of the nasal cavity.

38. The method according to claim 36, wherein intranasally dispensing the nasal wash fluid multidirectionally within the nasal cavity comprises intranasally dispensing the nasal wash fluid via a plurality of nozzles.

39. The method according to claim 38, wherein intranasally dispensing the nasal wash fluid multidirectionally within the nasal cavity via the plurality of nozzles comprises intranasally dispensing the nasal wash fluid into the nasal cavity:
in a posterior direction toward the nasopharynx via a first subset of one or more of the plurality of nozzles, and
in a superoposterior direction toward an olfactory area of the nasal cavity via a second subset of one or more of the plurality of nozzles, the first and the second subsets not including any nozzles common to both subsets.

40. The method according to claim 39,
wherein the plurality of nozzles includes only first and second nozzles, and wherein the first and the second subsets consist of the first and the second nozzles, respectively, and
wherein intranasally dispensing the nasal wash fluid into the nasal cavity comprises intranasally dispensing the nasal wash fluid into the nasal cavity:
in the posterior direction toward the nasopharynx via the first nozzle, and
in the superoposterior direction toward the olfactory area of the nasal cavity via the second nozzle.

41. The method according to any one of claims 1-8, wherein intranasally dispensing the nasal wash fluid into the nasal cavity comprises intranasally dispensing nasal wash fluid via both nostrils of the subject simultaneously.

42. The method according to any one of claims 1-8, wherein the nasal wash fluid does not include a drug molecule.

43. The method according to any one of claims 1-8, wherein the nasal wash fluid includes a tastant.

44. The method according to any one of claims 1-8, wherein the nasal wash fluid includes a color additive, and collecting the specimen sample comprises confirming that the specimen sample contains at least a portion of the nasal wash fluid by visually ascertaining that the specimen sample includes the color additive.

45. The method according to any one of claims 1-8, further comprising, after intranasally dispensing and before collecting the specimen sample, gargling, by the subject, the nasal wash fluid washed into the oropharynx.

46. The method according to any one of claims 1-8,
wherein the method further comprises, before collecting the specimen sample:
orally dispensing oral wash fluid into the oral cavity via the anterior opening of the oral cavity; and
swishing, by the subject, the oral wash fluid in the oral cavity, and
wherein collecting comprises collecting the specimen sample that passed out of the anterior opening of the oral cavity and contains at least a portion of the oral wash fluid and the at least a portion of the biological material washed into the oropharynx with the nasal wash fluid.

47. The method according to any one of claims 1-8,
wherein the method further comprises, before collecting the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and
wherein collecting comprises collecting the specimen sample that passed out of the anterior opening of the oral cavity and contains at least a portion of the mixture and the at least a portion of the biological material washed into the oropharynx with the nasal wash fluid.

48. The method according to any one of claims 1-8,
wherein the method further comprises, before collecting the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and
wherein collecting comprises collecting the specimen sample that passed out of the anterior opening of the oral cavity and contains at least a portion of the mixture and the at least a portion of the biological material washed into the oropharynx with the nasal wash fluid.

49. The method according to any one of claims 1-8,
wherein the method further comprises, before collecting the specimen sample:
orally dispensing oral wash fluid into the oral cavity via the anterior opening of the oral cavity; and
gargling, by the subject, the oral wash fluid, and
wherein collecting comprises collecting the specimen sample that passed out of the anterior opening of the oral cavity and contains at least a portion of the oral wash fluid and the at least a portion of the biological material washed into the oropharynx with the nasal wash fluid.

50. The method according to any one of claims 47, 48, and 49,
wherein the nasal wash fluid includes a first color additive,
wherein the oral wash fluid includes a second colored additive, the first and the second color additives having different colors, and
wherein collecting the specimen sample comprises confirming that the specimen sample contains at least a portion of the nasal wash fluid and at least a portion of the oral wash fluid by visually ascertaining that the specimen sample has a color produced by a combination of the first and the second color additives.

51. The method according to any one of claims 47, 48, and 49, further comprising, after collecting the specimen sample, testing the specimen sample for the presence of a particulate.

52. The method according to any one of claims 1-8,
wherein the specimen sample is a first specimen sample that contains at least a first portion of the nasal wash fluid and at least a first portion of the biological material washed into the oropharynx, and
wherein the method further comprises:
collecting from the nasal cavity, via one or two nostrils of the subject, a second specimen sample that contains at least a second portion of the nasal wash fluid and at least a second portion of the biological material in the nasal cavity; and
combining the first and the second specimen samples to produce a combined specimen sample.

53. The method according to any one of claims 1-8,
wherein intranasally dispensing the nasal wash fluid into the nasal cavity comprises inserting a tubular applicator of a nasal wash fluid dispenser into a nostril of the subject and intranasally dispensing nasal wash fluid from the tubular applicator, and
wherein the nasal wash fluid dispenser is shaped so as to guide introduction of the tubular applicator into the nostril at a specific orientation with respect to a maxilla of the subject.

54. The method according to any one of claims 1-8, wherein the method does not comprise swabbing the nasal cavity.

55. The method according to any one of claims 1-8, wherein collecting the specimen sample comprises drawing the specimen sample out of the oral cavity via the anterior opening of the oral cavity using an absorbent material.

56. A nasal wash fluid dispenser for intranasally dispensing nasal wash fluid into a nasal cavity of a subject, the nasal wash fluid dispenser comprising:
a dispensing container, which contains the nasal wash fluid; and
a tubular applicator, which (a) extends distally from and is in fluid communication with the dispensing container, (b) which is sized and shaped to be insertable into a nostril of the subject, and (c) which comprises first and second nozzles, which are configured to dispense the nasal wash fluid in first and second fluid discharges, respectively, having first and second centerlines, respectively, the first and the second centerlines defining an offset angle therebetween of between 10 and 90 degrees, such that, when the tubular applicator is inserted into the nostril with the first nozzle oriented in a posterior direction and the second nozzle oriented in a superoposterior direction, the first nozzle directs the first fluid discharge toward a nasopharynx of the subject and the second nozzle directs the second fluid discharge toward an olfactory area of the nasal cavity, such that the nasal wash fluid washes biological material into an oropharynx of the subject from (i) the nasal cavity, (ii) the nasopharynx, or (iii) the nasal cavity and the nasopharynx.

57. A nasal wash fluid dispenser for intranasally dispensing nasal wash fluid into a nasal cavity of a subject, the nasal wash fluid dispenser comprising:
a dispensing container, which contains the nasal wash fluid; and
a tubular applicator, which extends distally from and is in fluid communication with the dispensing container, which is sized and shaped to be insertable into a nostril of the subject, and which comprises a nozzle, which is configured to dispense the nasal wash fluid in a fluid discharge having a centerline, such that, when the tubular applicator is inserted into the nostril with the nozzle oriented in a posterior direction, the nozzle directs the fluid discharge toward a nasopharynx of the subject, so as to wash biological material, with some of the nasal wash fluid, from the nasal cavity into an oropharynx of the subject via the nasopharynx,
wherein the nasal wash fluid dispenser is shaped so as to guide introduction of the tubular applicator into the nostril at a specific orientation with respect to a maxilla of the subject.

58. A nasal wash fluid system, comprising:
a nasal wash fluid, which comprises a color additive; and
a nasal wash fluid dispenser, which comprises:
a dispensing container, which contains the nasal wash fluid; and
a tubular applicator, which extends distally from and is in fluid communication with the dispensing container, and which is sized and shaped to be insertable into a nostril of a subject.

59. A method comprising:
intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and
thereafter, collecting a specimen sample by drawing the specimen sample out of an anterior opening of an oral cavity of the subject using an absorbent material, the specimen sample containing at least a portion of the biological material washed into the oropharynx by the nasal wash fluid.

60. A method comprising:
intranasally dispensing nasal wash fluid into a nasal cavity of a subject such that the nasal wash fluid passes from the nasal cavity into an oropharynx of the subject via a nasopharynx of the subject; and
thereafter, collecting a specimen sample by drawing the specimen sample out of an anterior opening of an oral cavity of the subject using an absorbent material, the specimen sample containing at least a portion of the nasal wash fluid.

61. A method comprising:
intranasally dispensing nasal wash fluid into a nasal cavity of a subject such that the nasal wash fluid passes from the nasal cavity into an oropharynx of the subject via a nasopharynx of the subject; and
thereafter, collecting a specimen sample that passed out of an anterior opening of an oral cavity of the subject and contains at least a portion of the nasal wash fluid.

62. A method comprising:
intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx; and
thereafter, expressing, by the subject, out of an anterior opening of an oral cavity of the subject, a specimen sample that contains at least a portion of the biological material washed into the oropharynx by the nasal wash fluid.

63. A method comprising:
collecting, from a subject, a liquid specimen sample that includes saliva and an orally-dispensed oral wash fluid;
passing the liquid specimen sample through a filter to trap at least some of the saliva, the filter having a nominal pore size of between 30 microns and 1.5 mm, the nominal pore size representative of a minimum size of spherical particles necessary for the filter to retain 85% of the spherical particles when H2O containing the spherical particles is passed through the filter at 20 degrees C. under pressure supplied by a 10 cm water column; and
testing for the presence of a virus in the saliva trapped by the filter.

64. A method comprising:
collecting, from a subject, a liquid specimen sample;
passing the liquid specimen sample through a filter having a nominal pore size of between 30 microns and 1.5 mm, the nominal pore size representative of a minimum size of spherical particles necessary for the filter to retain 85% of the spherical particles when H2O containing the spherical particles is passed through the filter at 20 degrees C. under pressure supplied by a 10 cm water column; and
testing for the presence of a virus that adhered to the filter during the passing of the liquid specimen sample through the filter.

65. A method comprising:
collecting, from a subject, a liquid specimen sample that includes saliva and an orally-dispensed oral wash fluid;
passing the liquid specimen sample through a filter to trap at least some of the saliva, the filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and
testing for the presence of a virus in the saliva trapped by the filter.

66. A method comprising:
collecting, from a subject, a liquid specimen sample;
passing the liquid specimen sample through a filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and
testing for the presence of a virus that adhered to the filter during the passing of the liquid specimen sample through the filter.

67. A method comprising:
intranasally dispensing nasal wash fluid into a nasal cavity of a subject;
thereafter, collecting a specimen sample by performing one or more nasal swabs selected from the group consisting of: an anterior nares swab and a mid-turbinate swab; and
testing the specimen sample for the presence of a virus using a lateral flow immunoassay test strip.

68. A method comprising:
intranasally dispensing nasal wash fluid into a nasal cavity of a subject;
thereafter, tilting a head of the subject farther back than while intranasally dispensing the nasal wash fluid and farther back than a neutral position; and
thereafter, collecting a specimen sample by performing one or more nasal swabs selected from the group consisting of: an anterior nares swab and a mid-turbinate swab.

69. A method comprising:
intranasally dispensing nasal wash fluid into a nasal cavity of a subject;
tilting a head of the subject back until a neck of the subject is fully extended; and
thereafter, collecting a specimen sample by performing one or more nasal swabs selected from the group consisting of: an anterior nares swab and a mid-turbinate swab.

70. The method according to any one of claims 67, 68, and 69, wherein performing the one or more nasal swabs comprises performing the anterior nares swab.

71. The method according to claim 70,
wherein intranasally dispensing the nasal wash fluid into the nasal cavity comprises:
introducing, into a nostril of the subject, a swab tip of a nasal sampling device, the swab tip shaped so as to define (i) one or more nozzles, (ii) a proximal opening, and (iii) one or more channels within the swab tip that connect the one or more nozzles in fluid communication with the proximal opening; and
thereafter, intranasally dispensing the nasal wash fluid into the nasal cavity from the one or more nozzles, by introducing the nasal wash fluid into the proximal opening, and
wherein performing the anterior nares swab comprises performing the anterior nares swab by contacting a wall of an anterior naris of the nasal cavity with an absorbent material of the swab tip.

72. The method according to any one of claims 67, 68, and 69, wherein collecting the specimen sample comprises collecting between 25 and 150 microliters of material on a single swab.

73. The method according to claim 72, wherein collecting the specimen sample comprises collecting between 25 and 125 microliters of material on the single swab.

74. The method according to claim 72, wherein the single swab comprises a flocked swab tip, and wherein collecting the specimen sample comprises collecting between 25 and 150 microliters of the material on the flocked swab tip.

75. A method comprising:
intranasally dispensing nasal wash fluid into a nasal cavity of a subject;
thereafter, inserting a specimen sampler into the nasal cavity such that a farthest-inserted portion of the specimen sampler is positioned in an anterior naris of the nasal cavity; and
collecting a specimen sample on the specimen sampler by rubbing a wall of the anterior naris with the specimen sampler.

76. The method according to claim 75, wherein rubbing the wall of the anterior naris comprises rotating the specimen sampler while rubbing the wall of the anterior naris with the specimen sampler.

77. The method according to claim 75, wherein the specimen sampler includes a head comprising an absorbent material, and wherein rubbing the wall of the anterior naris with the specimen sampler comprises rubbing the wall of the anterior naris with the absorbent material of the head of the specimen sampler.

78. A method comprising:
intranasally dispensing nasal wash fluid into a nasal cavity of a subject;
inserting a specimen sampler distally into the nasal cavity such that a farthest-inserted portion of the specimen sampler is positioned in an anterior naris of the nasal cavity;
collecting a specimen sample on the specimen sampler by contacting a wall of the anterior naris with the specimen sampler; and
testing the specimen sample for the presence of a virus using a lateral flow immunoassay test strip.

79. The method according to claim 78, wherein the virus is Severe acute respiratory syndrome coronavirus 2 (SARS-CoV-2).

80. The method according to claim 78, wherein the virus is an Influenza virus.

81. The method according to any one of claims 67-78, further comprising, after intranasally dispensing the nasal wash fluid and before collecting the specimen sample, tilting a head of the subject farther back than while intranasally dispensing the nasal wash fluid and farther back than a neutral position.

82. The method according to claim 81, further comprising, after tilting the head farther back and before collecting the specimen sample, tilting the head forward to the neutral position or a tilted-forward position.

83. The method according to claim 82, further comprising, while the subject holds the head tilted back, waiting for a period of time between (a) tilting the head farther back and (b) tilting the head forward to the neutral or the tilted-forward position, the period of time at least 2 seconds.

84. The method according to claim 83, wherein the period of time is at least 5 seconds.

85. The method according to claim 84, wherein the period of time is at least 10 seconds.

86. The method according to claim 83, wherein the period of time is a first period of time, and wherein the method further comprises, after tilting the head forward to the neutral or the tilted-forward position, waiting for a second period of time before collecting the specimen sample, the period of time at least 5 seconds.

87. The method according to claim 82, further comprising, after tilting the head forward to the neutral or the tilted-forward position, waiting for a period of time before collecting the specimen sample, the period of time at least 5 seconds.

88. The method according to claim 87, wherein the period of time is at least 10 seconds.

89. The method according to claim 82, wherein tilting the head forward comprises tilting the head forward to the neutral or the tilted-forward position after sensing, by the subject, the nasal wash fluid in an oropharynx of the subject.

90. The method according to any one of claims 67-78, wherein intranasally dispensing the nasal wash fluid comprises:
tilting a head of the subject back until a neck of the subject is fully extended; and
while the subject holds the head tilted back with the neck fully extended, intranasally dispensing nasal wash fluid into a nasal cavity of the subject.

91. The method according to claim 90, further comprising, after intranasally dispensing the nasal wash, tilting the head forward to a neutral position or a tilted-forward position.

92. The method according to claim 91, further comprising, while the subject holds the head tilted back with the neck fully extended, waiting for a period of time between (a) concluding intranasally dispensing the nasal wash fluid into the nasal cavity and (b) tilting the head forward to the neutral or the tilted-forward position, the period of time at least 2 seconds.

93. The method according to claim 92, wherein the period of time is at least 5 seconds.

94. The method according to claim 93, wherein the period of time is at least 10 seconds.

95. The method according to claim 92, wherein the period of time is a first period of time, and wherein the method further comprises, after tilting the head forward to the neutral or the tilted-forward position, waiting for a second period of time before collecting the specimen sample, the period of time at least 5 seconds.

96. The method according to claim 91, further comprising, after tilting the head forward to the neutral or the tilted-forward position, waiting for a period of time before collecting the specimen sample, the period of time at least 5 seconds.

97. The method according to claim 96, wherein the period of time is at least 10 seconds.

98. The method according to any one of claims 67-78, wherein intranasally dispensing the nasal wash fluid comprises intranasally dispensing the nasal wash fluid into the nasal cavity while a head of the subject is held in a neutral position or a tilted-forward position.

99. The method according to any one of claims 67-78, further comprising, after intranasally dispensing the nasal wash fluid and before collecting the specimen sample, sniffing, by the subject, the nasal wash fluid.

100. A method comprising:
intranasally dispensing nasal wash fluid into a nasal cavity of a subject;
thereafter, tilting a head of the subject farther back than while intranasally dispensing the nasal wash fluid and farther back than a neutral position;
inserting a specimen sampler into the nasal cavity such that a farthest-inserted portion of the specimen sampler is positioned in an anterior naris of the nasal cavity; and
collecting a specimen sample on the specimen sampler by contacting a wall of the anterior naris with the specimen sampler.

101. The method according to claim 100, wherein tilting the head farther back comprises tilting the head back until a neck of the subject is fully extended.

102. The method according to claim 100, further comprising, after tilting the head farther back and before collecting the specimen sample, tilting the head forward to the neutral position or a tilted-forward position.

103. The method according to claim 102, further comprising, while the subject holds the head tilted back, waiting for a period of time between (a) tilting the head farther back and (b) tilting the head forward to the neutral or the tilted-forward position, the period of time at least 2 seconds.

104. The method according to claim 103, wherein the period of time is at least 5 seconds.

105. The method according to claim 104, wherein the period of time is at least 10 seconds.

106. The method according to claim 103, wherein the period of time is a first period of time, and wherein the method further comprises, after tilting the head forward to the neutral or the tilted-forward position, waiting for a second period of time before collecting the specimen sample, the period of time at least 5 seconds.

107. The method according to claim 102, further comprising, after tilting the head forward to the neutral or the tilted-forward position, waiting for a period of time before collecting the specimen sample, the period of time at least 5 seconds.

108. The method according to claim 107, wherein the period of time is at least 10 seconds.

109. The method according to claim 102, wherein tilting the head forward comprises tilting the head forward to the neutral or the tilted-forward position after sensing, by the subject, the nasal wash fluid in an oropharynx of the subject.

110. The method according to claim 100, wherein intranasally dispensing the nasal wash fluid comprises intranasally dispensing the nasal wash fluid into the nasal cavity while the head is held in the neutral position or a tilted-forward position.

111. The method according to claim 100, further comprising, after intranasally dispensing the nasal wash fluid and before collecting the specimen sample, sniffing, by the subject, the nasal wash fluid.

112. A method comprising:
tilting a head of a subject back until a neck of the subject is fully extended;
while the subject holds the head tilted back with the neck fully extended, intranasally dispensing nasal wash fluid into a nasal cavity of the subject;
inserting a specimen sampler into the nasal cavity such that a farthest-inserted portion of the specimen sampler is positioned in an anterior naris of the nasal cavity; and
collecting a specimen sample on the specimen sampler by contacting a wall of the anterior naris with the specimen sampler.

113. The method according to claim 112, further comprising, after intranasally dispensing the nasal wash, tilting the head forward to a neutral position or a tilted-forward position.

114. The method according to claim 113, further comprising, while the subject holds the head tilted back with the neck fully extended, waiting for a period of time between (a) concluding intranasally dispensing the nasal wash fluid into the nasal cavity and (b) tilting the head forward to the neutral or the tilted-forward position, the period of time at least 2 seconds.

115. The method according to claim 114, wherein the period of time is at least 5 seconds.

116. The method according to claim 115, wherein the period of time is at least 10 seconds.

117. The method according to claim 114, wherein the period of time is a first period of time, and wherein the method further comprises, after tilting the head forward to the neutral or the tilted-forward position, waiting for a second period of time before collecting the specimen sample, the period of time at least 5 seconds.

118. The method according to claim 113, further comprising, after tilting the head forward to the neutral or the tilted-forward position, waiting for a period of time before collecting the specimen sample, the period of time at least 5 seconds.

119. The method according to claim 118, wherein the period of time is at least 10 seconds.

120. The method according to claim 112, further comprising, after intranasally dispensing the nasal wash fluid and before collecting the specimen sample, sniffing, by the subject, the nasal wash fluid.

121. The method according to any one of claims 78-120, wherein the specimen sampler includes a head comprising an absorbent material, and wherein contacting the wall of the anterior naris with the specimen sampler comprises contacting the wall of the anterior naris with the absorbent material of the head of the specimen sampler.

122. The method according to any one of claims 75-120, wherein the specimen sampler comprises a swab.

123. The method according to claim 122, wherein the swab comprises a flocked swab tip.

124. The method according to claim 122, wherein the swab comprises a foam swab tip.

125. The method according to claim 122, wherein the swab comprises a tip having a greatest diameter of between 2 and 5 mm.

126. The method according to claim 122, wherein the swab comprises a tip having a collection surface area of between 200 and 300 mm2.

127. The method according to claim 122, wherein the swab comprises a tip having a volume of between 200 and 300 mm3.

128. The method according to claim 122, wherein the swab comprises a tip having a length of between 12 and 20 mm.

129. The method according to any one of claims 75-120, wherein collecting the specimen sample comprises collecting the specimen sample on the specimen sampler without using suction.

130. The method according to any one of claims 67-120, wherein intranasally dispensing the nasal wash fluid comprises intranasally dispensing the nasal wash fluid without blowing, by the subject, a nose of the subject immediately prior to intranasally dispensing the nasal wash fluid.

131. The method according to any one of claims 75-120, wherein collecting the specimen sample on the specimen sampler comprises collecting between 25 and 150 microliters of material on the specimen sampler.

132. The method according to claim 131, wherein collecting the specimen sample on the specimen sampler comprises collecting between 25 and 125 microliters of material on the specimen sampler.

133. The method according to claim 131, wherein the specimen sampler comprises a swab comprising a flocked swab tip, and wherein collecting the specimen sample on the specimen sampler comprises collecting between 25 and 150 microliters of the material on the flocked swab tip.

134. The method according to any one of claims 67-120, further comprising, after intranasally dispensing the nasal wash fluid into the nasal cavity:
orally dispensing oral wash fluid into an oral cavity of the subject via an anterior opening of the oral cavity; and
gargling, by the subject, the oral wash fluid.

135. The method according to any one of claims 67-120, wherein intranasally dispensing the nasal wash fluid comprises intranasally dispensing the nasal wash fluid as one or more types of fluid discharge selected from the group consisting of: a spray, a mist, a pressurized aerosol, steam, and an atomized fluid.

136. The method according to any one of claims 67-120, wherein intranasally dispensing the nasal wash fluid comprises intranasally dispensing the nasal wash fluid as a fluid stream.

137. The method according to any one of claims 67-120, wherein collecting the specimen sample comprises collecting the specimen sample after sensing, by the subject, the nasal wash fluid in an oropharynx of the subject.

138. The method according to any one of claims 68-77 and 801-821, further comprising testing the specimen sample for the presence of a particulate.

139. The method according to claim 138, wherein the particulate is selected from the group consisting of: a virus, a bacterium, a protozoan, and a fungus.

140. The method according to claim 139, wherein the particulate is the virus.

141. The method according to claim 140, wherein the virus is Severe acute respiratory syndrome coronavirus 2 (SARS-CoV-2).

142. The method according to claim 140, wherein the virus is an Influenza virus.

143. The method according to claim 140, wherein testing for the presence of the virus comprises using nucleic acid amplification.

144. The method according to claim 143, wherein testing for the presence of the virus comprises using polymerase chain reaction (PCR).

145. The method according to claim 140, wherein testing for the presence of the virus comprises performing an immunoassay.

146. The method according to claim 145, wherein performing the immunoassay comprises using a lateral flow immunoassay test strip.

147. The method according to claim 146, wherein performing the immunoassay comprises performing a chromatographic digital immunoassay.

148. A method comprising:
intranasally dispensing nasal wash fluid into a nasal cavity of a subject; and
collecting a specimen sample by performing one or more nasal swabs selected from the group consisting of: an anterior nares swab, a mid-turbinate swab, and a nasopharyngeal swab.
